(19) United States
(12) Patent Application Publication
(10) Pub. No.: US 2024/0373840 A1
(43) Pub. Date: Nov. 14, 2024

(54) METHODS, SYSTEMS AND COMPOSITIONS FOR RESTORATION AND PRESERVATION OF INTACT ORGANS IN A MAMMAL

(71) Applicant: Yale University, New Haven, CT (US)

(72) Inventors: Nenad Sestan, Madison, CT (US); Zvonnimir Vrselja, New Haven, CT (US); David Andrijevic, New Haven, CT (US)

(73) Assignee: Yale University, New Haven, CT (US)

(21) Appl. No.: 18/690,785

(22) PCT Filed: Sep. 16, 2022
(86) PCT No.: PCT/US2022/043816

§ 371 Date: Mar. 11, 2024

Related U.S. Application Data
(60) Provisional application No. 63/245,632, filed on Sep. 17, 2021.

(51) Int. Cl. A01N 1/02

(52) U.S. Cl. CPC A01N 1/021; A01N 1/0247; A01N 1/0252; A01N 1/0284

(57) ABSTRACT

The invention provides a system for hypothermic, restoration and preservation of organs in a mammal. In certain aspects, the system is capable of preserving organs, maintaining cellular integrity and cellular function for hours postmortem or after global ischemia. The invention also provides synthetic organ perfusate formulations, including a novel perfusate autologous blood mixture, which is able to reduce reperfusion injury, stimulate recovery from hypoxia, metabolically support the energy needs of organs and prevent rigor mortis.

### CROSS-REFERENCE TO RELATED APPLICATIONS

The present application claims priority under 35 U.S.C. § 119(e) to U.S. Provisional Patent Application No. 63/245,632, filed Sep. 17, 2021, which application is incorporated herein by reference in its entirety.

### STATEMENT REGARDING FEDERALLY SPONSORED RESEARCH OR DEVELOPMENT

This invention was made with government support under MH117064 and MH117064-01S1, awarded by the National Institute of Mental Health. The government has certain rights in the invention.

### SUMMARY OF THE INVENTION

In one aspect, the invention provides an isolated perfusate mixture comprising: an inorganic salt solution; an artificial oxygen carrier; and autologous blood.

In another aspect, the invention provides a system for the hypothermic preservation of organs in a mammal, the system comprising:

a perfusion device for the perfusion of an isolated perfusate mixture into the mammal, the perfusion device comprising: a perfusion loop; and a controller programmed to regulate at least a perfusate temperature within the perfusion loop to maintain hypothermic conditions; and the isolated perfusate mixture as described elsewhere herein.

In yet another aspect, the invention provides a mammal perfused with the isolated perfusate composition as described elsewhere herein, wherein mammalian organs are perfused under hypothermic conditions.

In yet another aspect, the invention provides perfused organs in a diseased mammal, wherein the perfused organs maintain one or more properties selected from the group consisting of an in vivo level of cell function and viability, and an in vivo level of morphology

In certain embodiments, the one or more artificial oxygen carriers is selected from the group consisting of hemoglobin glutamer-250, isolated cell-free hemoglobin, cross-linked hemoglobin, polymerized hemoglobin, encapsulated hemoglobin, and perfluorocarbon oxygen carriers. In certain embodiments, the artificial oxygen carrier is hemoglobin glutamer-250.

In certain embodiments, the one or more inorganic salts are selected from the group consisting of sodium chloride, sodium bicarbonate, magnesium chloride, and calcium chloride.

In certain embodiments, the perfusate mixture comprises a priming solution containing one or more sugars. In certain embodiments, the one or more sugars are glucose or dextrane.

In certain embodiments, the isolated perfusate mixture further comprises one or more amino acids. In certain embodiments, the one or more amino acids are selected from the group consisting of glycine, L-alanyl-glutamine, L-arginine, L-cysteine, L-histidine, L-isoleucine, L-leucine, L-lysine, L-methionine, L-phenylalanine, L-serine, L-threonine, L-tryptophan, L-tyrosine, L-valine and salts and solvates thereof.

In certain embodiments, the perfusate mixture further comprises one or more vitamins.

In certain embodiments, the one or more vitamins are selected from the group consisting of choline, D-calcium pantothenate, folic acid, niacinamide, pyridoxine, riboflavin, thiamine, i-inositol and salts and solvates thereof.

In certain embodiments, the perfusate mixture further comprises, ferric nitrate, magnesium sulfate, potassium chloride, sodium phosphate, and derivatives thereof.

In certain embodiments, the perfusate mixture further comprises an anti-clotting agent. In certain embodiments, the anti-clotting agent is heparin.

In certain embodiments, the percentage of autologous blood in the mixture is between 10% and 50%. In certain embodiments, the percentage of autologous blood in the mixture is approximately 28%.

In certain embodiments, the mixture is dialyzed against a solution comprising inorganic salts. In certain embodiments, the mixture is dialyzed against plasma.

In certain embodiments, the mixture comprises electrolytes and oncotic agents at levels comparable to those in autologous blood. In certain embodiments, the perfusate further comprises cytoprotective agents.

In certain embodiments, the cytoprotective agents are selected from the group consisting of 2-Iminobiotin, Necrostatin-1, sodium 3-hydroxybutryate, glutathione, minocycline, lamotrigine, QVE-Oph, methylene blue, and/or any salts, solvates, tautomers, and prodrugs thereof.

In certain embodiments, the mixture further comprises antibiotics. In certain embodiments, the antibiotic is ceftriazone. In certain embodiments, the mixture comprises one or more anti-inflammatory agents.

In certain embodiments, the one or more the anti-inflammatory agents is dexamathazone or cetirizine.

In certain embodiments, the temperature of the mixture is approximately 28° C.

In certain embodiments, the perfusion loop further comprises at least one pulse generator programmed to generate a pressure pulse within the perfusate within the perfusion loop.

In certain embodiments, the perfusion loop comprises a venous loop, a filtration loop and an arterial loop, wherein:

the venous loop comprises at least one perfusion pump;
the filtration loop comprises at least one perfusion pump, and at least one hemodiafiltration unit adapted and configured to equilibrate the perfusate;
the arterial loop comprises at least one gas exchange source and at least one gas mixer adapted and configured to supply oxygen and carbon dioxide to the perfusate;
wherein the mammal, the venous loop, the filtration loop and the arterial loop are in fluidic communication such that the perfusate can be carried from the mammal, through the venous loop, through the filtration loop, through the arterial loop and back to the mammal.

In certain embodiments, the one or more components selected from the group consisting of the venous loop, the filtration loop and the arterial loop further comprise a reservoir containing excess perfusate.

In certain embodiments, the one or more components selected from the group consisting of the mammal, the venous loop, the filtration loop and the arterial loop further comprise one or more elements selected from the group consisting of:

one or more valves adapted and configured to regulate the flow of the perfusate;

one or more filters adapted and configured to filter the perfusate; and

one or more sensors for measuring one or more properties of the perfusate selected from the group consisting of pH, dissolved oxygen concentration, dissolved carbon dioxide concentration, dissolved metabolite concentration, temperature, pressure, and flow rate.

In certain embodiments, the one or more sensors measure the concentration of at least one dissolved metabolite selected from the group consisting of nitric oxide, lactate, bicarbonate, oxygen, carbon dioxide, total hemoglobin, methemoglobin, oxyhemoglobin, carboxyhemoglobin, sodium, potassium, chloride, calcium, glucose, urea, ammonia, and creatinine.

In certain embodiments, the mammal perfusion apparatus comprises one or more sensors for measuring one or more properties of the perfusate selected from the group consisting of a pressure and a flow rate.

In certain embodiments, the one or more components selected from the group consisting of the mammal, the venous loop, the filtration loop and the arterial loop comprise one or more heat exchange units comprising:

one or more heat exchangers;
one or more temperature regulation units;
one or more temperature regulating pumps;
a thermoregulation fluid; and
one or more pipes configured and adapted to transport the thermoregulation fluid, wherein the one or more pipes are in fluidic communication with the one or more heat exchangers, the one or more temperature regulation units and the one or more temperature regulating pumps.

In certain embodiments, the one or more components selected from the group consisting of the brain enclosure unit, the venous loop, the filtration loop and the arterial loop comprise one or more sensors adapted and configured to measure the temperature within the perfusion device.

In certain embodiments, the one or more sensors are adapted and configured to measure the temperature within the perfusion device, the one or more temperature regulation units and the one or more temperature regulating pumps are in electronic communication with a computer programmed to regulate the temperature of the thermoregulation fluid and the specified flow rate of the one or more temperature regulating pumps to maintain a specified temperature within the perfusion device.

In certain embodiments, the hemodiafiltration unit is adapted and configured to supply one or more nutrients to the perfusate, selected from the group consisting of Glycine, L-Alanyl-Glutamine, L-Arginine hydrochloride, L-Cystine, L-Histidine hydrochloride-H2O, L-Isoleucine, L-Leucine, L-Lysine hydrochloride, L-Methionine, L-Phenylalanine, L-Serine, L-Threonine, L-Tryptophan, L-Tyrosine, L-Valine, Choline chloride, D-Calcium pantothenate, Folic Acid, Niacinamide, Pyridoxine hydrochloride, Riboflavin, Thiamine hydrochloride, i-Inositol, Calcium Chloride (CaCl2)-2H2O), Ferric Nitrate (Fe(NO3)3 9H2O), Magnesium Sulfate (MgSO4-7H2O), Potassium Chloride (KCl), Sodium Bicarbonate (NaHCO₃), Sodium Chloride (NaCl), Sodium Phosphate monobasic (NaH2PO4-2H2O), D-Glucose (Dextrose), Phenol Red, Sodium Pyruvate, free fatty acids, cholesterol and nucleic acid constitutes.

In certain embodiments, the system is configured to perfuse the mammal with the perfusate at a cardiac pulsatile pressure of about 20 mmHg to about 140 mmHg.

In certain embodiments, the system is configured to perfuse the organs in the mammal with the perfusate through the pulse generator at a rate of about 40 to about 180 beats per minute.

In certain embodiments, the system further comprises a controller in electronic communication with one or more elements of the system.

In certain embodiments, the mammal is a deceased mammal. In certain embodiments, the mammal is a human. In certain embodiments, the deceased mammal is deceased for longer than 1 hour. In certain embodiments, the deceased mammal has been deceased for longer than 4 hours. In certain embodiments, the mammal died of cardiac arrest.

In certain embodiments, the organs in the deceased mammal are ischemic prior to perfusion with the isolated perfusate mixture.

In certain embodiments, rigor mortis is prevented. In certain embodiments, rigor mortis is reversed.

In certain embodiments, the perfusate mixture flows into the ophthalmic artery.

In certain embodiments, the perfusate mixture flows into the renal intralobular arteries.

### BACKGROUND OF THE INVENTION

Cells lack meaningful oxygen-storage capacity, leading to obligate oxygen-dependence. At the cellular level, in just minutes following ischemia, intracellular acidosis and edema develop, and cause secondary damage to cellular membranes, organelles, and ultimately cell death. At the whole-body scale, there is a systemic release of hormones and cytokines, and activation of autonomic nervous, immune, and coagulation systems, leading to end-organ injury and feedback onto cellular injury cascades that culminate in systemic metabolic acidosis and hyperkalemia.

Reperfusion of the whole-body with autologous blood has several deleterious issues, including coagulation, microvascular plugging, inflammation, and blood-intrinsic cellular dysfunction. These obstacles have limited whole-body reperfusion and recovery of large mammals to 20 minutes of warm ischemia. Appropriate interventions are needed for molecular and cellular recovery across all vital organs in the large mammalian body following prolonged warm ischemia. The present invention addresses this need.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1G show overview of the OrganEx technology and experimental workflow. FIG. 1A shows connection of the porcine body to the OrganEx perfusion system (or ECMO, not shown) via cannulation of the femoral artery and vein. FIG. 1B is a simplified schematic view of the OrganEx perfusion device. The system is equipped with a centrifugal pump, pulse generator, hemodiafiltration gas infusion, drug delivery systems, and sensors to measure metabolic and circulation parameters. FIG. 1C is a schematic of the experimental workflow and conditions. FIG. 1D, the lid of the flow chamber that is connected to the electronic pressure regulator via an air line and the barb connector. FIG. 1E, the bottom view of the lid showing in green space occupied by air. FIG. 1F, the isometric view of the body, in green is the fluid path (fluid space) occupied by the mixture of autologous blood and the perfusate. FIG. 1G, the side view of the lid, body and the membrane that separates two parts.

FIGS. 2A-2E show circulation and blood/perfusate properties during the perfusion protocols. FIGS. 2A-2B are representative images of abdominal fluoroscopy (FIG. 2A, n=9) and ophthalmic and renal ultrasound (FIG. 2B, n=6) at 3h of perfusion. ECMO is depicted on upper and OrganEx on lower panels. FIGS. 2C-2E show changes in total flow rate, brachial arterial pressure (FIG. 2C), percentage of venous O₂ saturation (FIG. 2D), K⁺ concentration, and pH in serum (FIG. 2E) throughout the perfusion protocols; n=6. Data presented are mean S.E.M. Two-tailed unpaired t-test was performed. **P<0.01, ***P<0.001, NS: not significant.

FIGS. 3A-3K show analysis of tissue integrity across experimental conditions and organs. FIG. 3A show representative confocal images of immunofluorescent staining for neurons (RBFOX3/NeuN), astrocytes (GFAP), and microglia (IBA1) counterstained with DAPI nuclear stain in hippocampal CA1 region; n=3. Quantification of NeuN immunoreactivity intensity (FIG. 3B), number of GFAP fragments (FIG. 3C), and microglia number in CA1 (FIG. 3D). FIG. 3E are representative images of H&E staining in heart, liver, and kidney. FIGS. 3F-3H are related to evaluation of histopathological criteria that include nuclear pyknosis (arrow), tissue integrity (*), hemorrhage/congestion (empty arrowhead), cell vacuolization (full arrowhead), and tissue edema (double arrow) in heart (FIG. 3F), liver (FIG. 3G) and kidney (FIG. 3H); n=5. FIGS. 3F-3H are representative confocal images of immunofluorescent staining for ACTB in kidney (FIG. 3I) and its quantification in glomerulus (FIG. 3J) and proximal convoluted tubule (PCT) (FIG. 3K); n=3. Scale bars, 40 m. Data presented are mean S.E.M. One-way ANOVA with post-hoc Dunnett's adjustments was performed. *P<0.05, **P<0.01, ***P<0.001, NS: not significant.

FIGS. 4A-4P show analysis of cell death across experimental conditions and organs. FIGS. 4A, 4F, 4K, and 4N, Representative confocal images of immunofluorescent staining for activated caspase 3 (actCASP3) and TUNEL assay in heart, liver, kidney, and brain. FIGS. 4B-4D, Quantification of actCASP3 immunolabeling signal intensity in heart (FIG. 4B), liver (FIG. 4C), and kidney (FIG. 4D). FIGS. 4G-4J, Normalized total intensity of TUNEL signal in heart (FIG. 4G), liver (FIG. 4H), and kidney (FIG. 4I). FIGS. 4L and 4M, Percentage of actCASP3 positively stained nuclei in the CA1 (FIG. 4L) and PFC (FIG. 4M). FIGS. 4O and 4P, Normalized total intensity of TUNEL signal in CA1 (FIG. 4O) and PFC (FIG. 4P). Scale bars, 50 m. Data presented are mean S.E.M. One-way ANOVA with post-hoc Dunnett's adjustments was performed. For more detailed information on statistics and reproducibility, see methods. *P<0.05, **P<0.01, ***P<0.001, NS: not significant.

FIG. 5A-5K depict functional characterization and metabolic activity of selected organs. FIGS. 5A-5C, complexes in ECMO and OrganEx treated animals 3 hours into the perfusion (left); Chi-squared test was performed. Representative EKG trances in OrganEx and ECMO group 3 hours into the perfusion (right). FIG. 5E, Measurement of cardiomyocyte contraction velocity in acute heart slices in the Oh WIT, OrganEx, and ECMO groups (left), and cardiomyocyte contraction duration (right); n=5. FIG. 5F, Representative confocal images of immunolabeling for albumin in liver. Scale bars, 50 μm. FIG. 5G, Measurement of normalized immunolabeling signal intensity of albumin in liver demonstrating similar expression between OrganEx and Oh WIT groups; n=3. FIG. 5H, Representative images of organotypic hippocampal slices after 14 days in culture. Scale bar, 500 m. FIG. 5I, Quantification of hippocampal slice integrity; n=4-5. FIG. 5J, Representative confocal images of newly synthesized proteins (AHA, Click-iT chemistry) with DAPI counterstaining in the long-term organotypic hippocampal slice culture. Scale bar, 100 m. FIG. 5K, Quantification of AHA relative intensity in the CA1; n=3-5. Data presented are mean±S.E.M. One way ANOVA was performed with post-hoc Dunnett's adjustments was performed. *P<0.05, **P<0.01, ***P<0.001, NS: not significant. AU, arbitrary units.Measurement of 2-NBDG uptake in heart (FIG. 5A), kidney (FIG. 5B), and brain (FIG. 5C); n=3. FIG. 5D, Observed QRS.

FIGS. 6A-6D show organ and cell type-specific transcriptomic changes assessed by snRNA-seq across various warm ischemia intervals and different perfusion interventions. Upper panels: UMAP layout showing major t-types in the hippocampus (FIG. 6A), heart (FIG. 6B), liver (FIG. 6C), and kidney (FIG. 6D). Middle panels: comparison of averaged Augur AUC scores across t-types indicating which cell type underwent the most transcriptomic changes; Lower panels: dot plots depicting P values of gene set enrichment of gene sets important in cellular recovery and specific cellular functions in major respective t-types. **P<0.01, ***P<0.001.

FIGS. 7A-7E show analysis of circulation and blood/perfusate properties after 1 h of warm ischemia and perfusion interventions. FIG. 7A, Representative fluoroscopy images of autologous blood flow (ECMO intervention, up) or a mixture of autologous blood and the perfusate (OrganEx intervention, below) in the head captured after 3 and 6 hours respectively of perfusion, showing robust restoration of the circulation in the OrganEx group. A contrast catheter was placed in the left common carotid artery (CCA), except in the ECMO group at 6 hours timepoint where contrast catheter could not be advanced beyond aortic arch in to the left CCA due to pronounced vasoconstriction, thus resulting in bilateral CCA filling. n=6. FIG. 7B, Representative color Doppler images of the CCA demonstrating robust flow in OrganEx group. Ultrasound waveform analysis demonstrated that OrganEx produced pulsatile, biphasic flow pattern (lower panel). SCM, sternocleidomastoid muscle. n=6. FIG. 7C, Longitudinal change in arterial and venous cannula pressures throughout the perfusion demonstrating robust perfusion in OrganEx group. FIG. 7D, Time-dependent changes in oxygen delivery and consumption demonstrating increased oxygen delivery and stable oxygen consumption over the perfusion period in OrganEx group. FIG. 7E, Presence of classical signs of death (rigor and livor mortis) in ECMO as compared to OrganEx group at the experimental endpoint. Data presented are mean S.E.M. Two-tailed unpaired t-test was performed. For more detailed information on statistics and reproducibility, see methods. *P<0.05, **P<0.01, ***P<0.001, NS: not significant.

FIGS. 8A-8L show Nissl staining and immunohistochemical analysis of the hippocampal CA1 region and the prefrontal cortex (PFC). FIG. 8A, Representative images of Nissl staining of the CA1 (up) and PFC (below). FIGS. 8B and 8C, Quantification of the number of cells per standardized area (FIG. 8B) and percentage of ellipsoid cells per area (FIG. 8C) in the CA1 between the experimental groups. FIGS. 8D and 8E, Quantification of the number of cells per standardized area (FIG. 8D) and percentage of ellipsoid cells per area (FIG. 8E) in the PFC between the experimental groups. FIGS. 8F and 8H, Representative confocal images of immunofluorescent staining for neurons (RBFOX3/NeuN), astrocytes (GFAP), and microglia (IBA1) counterstained with DAPI nuclear stain in CA1 (FIG. 8F) and PFC (FIG. 8H). FIG. 8G, Quantification of GFAP immunoreactivity in hippocampal CA1 region depicting comparable immunoreactivity between OrganEx and Oh WIT group, with a significant increase compared to the other groups. FIGS. 8I-8L Quantification of NeuN immunolabeling intensity (FIG. 8I), number of GFAP+ fragments (FIG. 8J), and number of GFAP+ cells (k) depict similar trends between the groups as seen in the CAL. Microglia number (FIG. 8L) shows comparable results between OrganEx and Oh WIT with different dynamics seen in the ECMO group. Scale bars, 50 m. Data presented are mean S.E.M. One-way ANOVA with post-hoc Dunnett's adjustments was performed. For more detailed information on statistics and reproducibility, see methods. *P<0.05, **P<0.01, ***P<0.001, NS: not significant.

FIGS. 9A-9J show representative images of H&E staining across assessed peripheral organs and kidney periodic acid-Schiff (PAS) staining and immunolabeling for HACVR1 and Ki-67. FIG. 9A, Representative images of the H&E staining in heart, kidney, liver, pancreas, and lungs. Arrows point to nuclear damage, asterisks point to disrupted tissue integrity, empty arrowheads point to hemorrhage, full arrowheads point to cell vacuolization, double arrows point to tissue edema. FIGS. 9B and 9C, H&E histopathological scores in lungs (FIG. 9B) and pancreas (FIG. 9C). FIG. 9D, Representative images of PAS staining of the kidney. Arrows point to disrupted brush border, full arrowheads point to the presence of casts, asterisks point to tubular dilation, double arrows point to the Bowman space dilation. FIG. 9E, Kidney PAS histopathological damage score. FIG. 9F and FIG. 9H, Representative confocal images of immunofluorescent staining for HAVCR1 and Ki-67 in kidney, respectively. FIG. 9G, Quantification of HAVCR1 immunolabeling signal intensity. FIG. 9I and FIG. 9J, Quantification of the kidney Ki-67 positive staining. HACVR1 and Ki-67 immunolabeling quantification results follow a similar pattern seen with other organs with comparable results between Oh WIT and OrganEx group and significant decrease in the 7h WIT and ECMO groups. Scale bars, 100 μm. Data presented are mean±S.E.M. One-way ANOVA with post-hoc Dunnett's adjustments was performed. For more detailed information on statistics and reproducibility, see methods. *P<0.05, **P<0.01, NS: not significant.

FIGS. 10A-10O show evaluation of different cell death pathways by immunohistochemical staining for important molecules in pyroptosis (IL1B), necroptosis (RIPK3) and ferroptosis (GPX4) across the experimental conditions. FIGS. 10A, 10F, 10K, Representative confocal images of immunofluorescent staining for pyroptosis marker IL1B, necroptosis marker RIPK3, and ferroptosis marker GPX4, each co-stained with DAPI nuclear stain in CA1, heart, liver, and kidney. FIGS. 10B-10E, Quantification of IL1B immunolabeling signal intensity in CA1 (FIG. 10B), heart (FIG. 1C), liver (FIG. OD), and kidney (FIG. 10E). FIGS. 10G-10J, Quantification of RIPK3 positive intranuclear co-staining in CA1 (FIG. 10G), and immunolabeling signal intensity heart (FIG. 10H), liver (FIG. 10I), kidney (FIG. 10J). FIGS. 10L-10O, Quantification of GPX4 immunolabeling signal intensity in CA1 (FIG. 10L), heart (FIG. 10M), liver (FIG. 10N), and kidney (FIG. 10O). Scale bars, 50 μm left and right panels. Data presented are mean S.E.M. One-way ANOVA with post-hoc Dunnett's adjustments was performed. For more detailed information on statistics and reproducibility, see methods. *P<0.05, **P<0.01, ***P<0.001, NS: not significant, IN: intranuclear.

FIGS. 11A-11O EEG setup and recordings, click-iT chemistry and immunohistochemical analysis of factor V and troponin I. FIG. 11A, Placement of EEG electrodes on the porcine scalp. FIG. 11B, Representative snapshot of the EEG recordings after administration of anesthesia and before the induction of cardiac arrest by ventricular fibrillation. FIG. 11C, Representative snapshot of the EEG recordings immediately following the ventricular fibrillation. FIG. 11D, Representative snapshot of the EEG during ECMO intervention at around 2h of perfusion protocol. FIG. 11E, Representative snapshot of the EEG during OrganEx intervention at around 2h of perfusion protocol, showing a light pulsatile artefact. FIGS. 11F and 11G, Representative snapshot of the EEG recordings following contrast injection at 3h in ECMO and OrganEx animals, respectively. OrganEx EEG snapshot is consistent with a possible muscle-movement artefact. GND, ground electrode; REF, reference electrode. FIGS. 11H and 11I, Representative confocal images of AHA through Click-iT chemistry in newly synthesized proteins with DAPI nuclear stain in the long-term organotypic hippocampal slice culture in CA3 (FIG. 11H) and DG (FIG. 11AI) subregions. FIGS. 11J and 11K, Relative intensity of nascent protein around nuclei in hippocampal CA3 (FIG. 11J) and DG (FIG. 11K) region showing comparable protein synthesis between OrganEx and Oh WIT up to 14 days in culture. FIG. 11L, Representative confocal images of immunofluorescent staining for troponin I in the heart. FIG. 11M, Quantification of troponin I immunolabeling signal intensity in heart. A decreased trend in immunolabeling intensity was observed with ischemia time and a significant decrease in immunolabeling intensity in ECMO compared to the OrganEx group. FIG. 11N, Representative confocal images of immunofluorescent staining for factor V in liver. FIG. 11O, Quantification of factor V immunolabeling signal intensity in liver follows a similar pattern seen with other organs with comparable results between Oh WIT, 1h WIT, and OrganEx group and a significant decrease in 7h WIT and ECMO groups. Scale bars, 50 m. Data presented are mean S.E.M. For more detailed information on statistics and reproducibility, see methods. *P<0.05, **P<0.01, NS. not significant. AU, arbitrary units.

FIGS. 12A-12F Quality control of snRNA-seq data in healthy and varying ischemic conditions in the hippocampus, heart, liver, and kidney. Through transcriptomic integration and iterative clustering, a taxonomy of t-types in healthy organs and brain, heart, liver, and kidney that experienced ischemia (1h WIT, 7h WIT, ECMO and OrganEx) were generated, representing presumptive major cell types across organs of interest. These major t-types were further subdivided into high-resolution subclusters that were transcriptomically comparable to publicly available human and mouse single-cell datasets and that were marked by distinct expression profiles. FIG. 12A, Bar plot showing the number of cells/nuclei across organs and biological replicates. FIG. 12B, Violin plot showing the distribution of the number of unique molecular identifiers—UMIs (upper panel) and genes (lower panel) detected across all biological replicates.

FIGS. 12C-12F, respective analyses of snRNA-seq in the hippocampus (FIG. 12C), heart (FIG. 12D), liver (FIG. 12E), and kidney (FIG. 12F). The left upper corner depicts detailed UMAP layout showing all t-types in the respective organs. The right side depicts the expression of top t-type markers. The left lower corner depicts transcriptomic correlation between the t-type taxonomy defined in this study and that of previous human and mouse datasets.

FIGS. 13A-13D Single-nucleus transcriptome analysis in healthy and varying ischemic conditions in the hippocampus (FIG. 13A), heart (FIG. 13B), liver (FIG. 13C), and kidney (FIG. 13D). FIGS. 13A-13D, From left to right: UMAP layout showing major t-types; UMAP layout, colored by Augur cell type prioritization (AUC) between Oh WIT compared to 1h (up) and 7h WIT (down); statistical comparison of Augur AUC scores between Oh WIT and 1h (up) and 7h (down) of WIT; Volcano plot showing top DEGs in major annotated t-types between Oh and 1h WIT (up), or Oh and 7h WIT (down); GO terms associated with the genes up and downregulated in detected nuclei between Oh and 1h WIT (up), or Oh and 7h WIT (down) with their nominal P-value in respective major annotated t-types.

FIGS. 14A-14H show hippocampal single-nucleus transcriptome analysis comparing OrganEx to other experimental conditions. FIG. 14A, AUC scores of the Augur cell type prioritization between OrganEx and other groups. FIG. 14B, Volcano plot showing DEGs in hippocampal neurons between OrganEx and Oh WIT, 1h WIT, 7h WIT, and ECMO. FIG. 14C, Trajectories of hippocampal neurons. Color indicates different experimental groups. FIG. 14D, Sankey plot showing perfusate components and violin plots showing their effects on hippocampal neurons between the OrganEx and ECMO groups. FIG. 14E, Hierarchical clustering of the top DEGs across experimental groups and derived functional gene modules (upper left). Eigengene average expression trends exhibit distinct trends between ECMO and OrganEx groups (upper right) of modules whose enriched GO terms are predominantly related to cellular function (below) (FIG. 34). FIG. 14F, Expression of the genes involved in cell-death pathways in neurons. FIG. 14G, Gene expression enrichment of the genes involved in cell-death pathways in neurons. FIG. 14H, Overall signaling patterns across all experimental conditions. Genes important in inflammation are highlighted gray. i, Stacked bar plot showing relative information flow for each signaling pa pathway across experimental group pairs. Significant signaling pathways were ranked based on differences in the overall information flow within the inferred networks between OrganEx and Oh WIT, 1h WIT, 7h WIT, and ECMO. Genes important in inflammation are highlighted gray. Necro-1, necrostatin-1; Mino, minocycline; DEXA, dexamethasone; Met. B, methylene blue; GEE, Glutathione Ethyl Ester. *P<0.05, **P<0.01, ***P<0.001, NS: not significant.

FIGS. 15A-15H show heart single-nucleus transcriptome analysis comparing OrganEx to other experimental conditions. FIG. 15A, AUC scores of the Augur cell type prioritization between OrganEx and other groups. FIG. 15B, Volcano plot showing the DEGs in cardiomyocytes between OrganEx and Oh WIT, 1h WIT, 7h WIT, and ECMO. FIG. 15C, Trajectories of hippocampal neurons. Color indicates different experimental groups. FIG. 15D, Sankey plot showing perfusate components and violin plots showing their effects on cardiomyocytes between the OrganEx and ECMO groups. FIG. 15E, Hierarchical clustering of the top DEGs across experimental groups and derived functional gene modules (upper left). Eigengene average expression trends exhibit distinct trends between ECMO and OrganEx groups (upper right) of modules whose enriched GO terms are predominantly related to cellular function (below) (FIG. 34). FIG. 15F, Expression of the genes involved in cell-death pathways in cardiomyocytes. FIG. 15G, Gene expression enrichment of the genes involved in cell-death pathways in cardiomyocytes. FIG. 15H, Overall signaling patterns across all experimental conditions. Genes important in inflammation are highlighted gray. i, Stacked bar plot showing relative information flow for each signaling pathway across experimental group pairs. Significant signaling pathways were ranked based on differences in the overall information flow within the inferred networks between OrganEx and Oh WIT, 1h WIT, 7h WIT, and ECMO. Genes important in inflammation are highlighted gray. HIP, hippocampus; Necro-1, necrostatin-1; Mino, minocycline; DEXA, dexamethasone; Met. B, methylene blue; GEE, Glutathione Ethyl Ester. *P<0.05, **P<0.01, ***P<0.001, NS: not significant.

FIGS. 16A-16I show liver single-nucleus transcriptome analysis comparing OrganEx to other experimental conditions. FIG. 16A, AUC scores of the Augur cell type prioritization between OrganEx and other groups. FIG. 16B, Volcano plot showing DEGs in hepatocytes between OrganEx and Oh WIT, 1h WIT, 7h WIT, and ECMO. FIG. 16C, Trajectories of hippocampal neurons. Color indicates different experimental groups. FIG. 16D, Sankey plot showing perfusate components and violin plots showing their effects on hepatocytes between the OrganEx and ECMO. FIG. 16E, Hierarchical clustering of the top DEGs across experimental groups and derived functional gene modules (upper left). Eigengene average expression trends exhibit distinct trends between ECMO and OrganEx groups (upper right) of modules whose enriched GO terms are predominantly related to cellular function or cell death (below) (Table 23). FIG. 16F, Expression of the genes involved in cell-death pathways in hepatocytes. FIG. 16G, Gene expression enrichment of the genes involved in cell-death pathways in hepatocytes. FIG. 16H, Overall signaling patterns across all experimental conditions. Genes important in inflammation are highlighted gray. FIG. 16A I, Stacked bar plot showing relative information flow for each signaling pathway across experimental group pairs. Significant signaling pathways were ranked based on differences in the overall information flow within the inferred networks between OrganEx and Oh WIT, 1h WIT, 7h WIT, and ECMO. Genes important in inflammation are highlighted gray. Necro-1, necrostatin-1; Mino, minocycline; DEXA, dexamethasone; Met. B, methylene blue; GEE, Glutathione Ethyl Ester. *P<0.05, **P<0.01, ***P<0.001, NS: not significant.

FIGS. 17A-17I show kidney single-nucleus transcriptome analysis comparing OrganEx to other experimental conditions. FIG. 17A, AUC scores of the Augur cell type prioritization between OrganEx and other groups. FIG. 17B, Volcano plot showing DEGs in PCT between OrganEx and Oh WIT, 1h WIT, 7h WIT, and ECMO. FIG. 17C, Trajectories of hippocampal neurons. Color indicates pseudotime progression and different cell states, respectively. FIG. 17D, Sankey plot showing perfusate components and violin plots showing their effects on PCT between the OrganEx and ECMO groups. FIG. 17E, Hierarchical clustering of the top DEGs across experimental groups and derived functional gene modules (upper left). Eigengene average expression trends exhibit distinct trends between ECMO and OrganEx groups (upper right) of modules whose enriched GO terms are predominantly related to cellular function or cell death (below) (FIG. 34). FIG. 17F, Expression of the genes involved in cell-death pathways in PCT. FIG. 17G, Gene expression enrichment of the genes involved in cell-death pathways in PCT. PCT, proximal convoluted tubule. FIG. 17H, Overall signaling patterns across all experimental conditions. Genes important in inflammation are highlighted gray. FIG. 17I, Stacked bar plot showing relative information flow for each signaling pathway across experimental group pairs. Significant signaling pathways were ranked based on differences in the overall information flow within the inferred networks between OrganEx and Oh WIT, 1h WIT, 7h WIT, and ECMO. Genes important in inflammation are highlighted gray. PCT, proximal convoluted tubules; DCT, distal convoluted tubules; Necro-1, necrostatin-1; Mino, minocycline; DEXA, dexamethasone; Met. B, methylene blue; GEE, Glutathione Ethyl Ester. *P<0.05, **P<0.01, ***P<0.001, NS: not significant.

### DETAILED DESCRIPTION OF THE INVENTION

The invention provides a novel system for restoration and preservation of an intact mammalian organs. In certain aspects, the system is capable of preserving organs in the mammalian body and restoring and maintaining cellular integrity and cellular function for hours post mortem or after global ischemia. The invention also provides novel synthetic organ perfusate formulations, and methods of mixing the perfusate with blood, for example, autologous blood, derived from the mammal.

The invention includes surgical methods and procedures to connect the mammal to the OrganEx system. In combination, the system, perfusate, and surgical method attenuate organ cell death, preserve anatomical and cellular integrity and restore cellular function as indicated by active metabolism. The invention also provides means to reduce reperfusion injury, stimulate recovery from hypoxia, and metabolically support the energy needs of organ function. The invention further provides methods of using the system and blood perfusate mixture, to prevent the collapse of organ vasculature and to allow for better perfusion of the organs. In addition, the invention provides methods to prevent the onset of rigor mortis.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described. As used herein, each of the following terms has the meaning associated with it in this section.

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “hypoxic” refers to a concentration of dissolved oxygen less than about 13%, corresponding to a partial pressure of about 100 mmHg, the physiologic partial pressure of oxygen in the alveoli of the lung.

As used herein, the term “cellular hypoxia” refers to a cellular response to exposure to a hypoxic environment, often resulting in apoptosis, or cellular death.

As used herein, the term “anaerobic metabolism” refers to the cellular consumption of glucose to produce two molecules of lactate, with the lactate remaining in dissolved in solution. The ratio of lactate produced to glucose consumed will be 2:1.

As used herein, the term “aerobic metabolism” refers to the cellular consumption of glucose to produce two molecules of lactate, both of which will be consumed through the Krebs cycle in the presence of sufficient levels of oxygen. The ratio of lactate produced to glucose consumed will be 0:1.

As used herein, the term “hypothermic” refers to a body temperature substantially below normal bounds. Hypothermic temperatures include, but are not limited to, temperatures between 10° and 32° C., between 20° C. and 30° C., and about 28° C.

As used herein, the term “salt” embraces addition salts of free acids or free bases that are compounds useful within the invention. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, b-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, sodium and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

As described herein, rigor mortis refers to process that sets in upon the death of a mammal. Rigor mortis is characterized by stiffening of the muscles. Stiffening occurs as a result of ATP depletion. ATP is depleted in ischemic tissues where there is insufficient oxygen available for mitochondria to drive ATP formation. As a result, the bridges between actin and myosin fibers are no longer broken, causing muscle stiffness.

As used herein, OrganEx refers to a system that consists of a perfusion system and synthetic perfusate. The perfusion system consists of a computer driven custom-made pulse generator connected to a centrifugal pump, which enables reproduction of physiological pressure and flow waveforms, together with automated hemodiafiltration, gas mixer, and drug delivery systems which allow control of blood coagulation and supplementation of the perfusate. To ensure homeostasis and maintain the targeted perfusion parameters, the perfusion system is also equipped with sensors for electrolytes, blood gases, metabolic parameters, hemoglobin, vessels and cannulas pressures, and total circulatory flow rate. In the OrganEx system, prior to the initiation of the perfusion protocol, autologous blood of the mammal is drained into the OrganEx system and mixed with the perfusate, which is then used to perfuse the animal.

As used herein, ECMO refers to a clinical standard of a heart-and-lung substitution perfusion device—extracorporeal membrane oxygenation system (ECMO). In the ECMO system, the animals are perfused with autologous blood.

The following abbreviations are used herein:
ABG arterial blood gasses
ACT activated clotting time
actCASP3 activated caspase 3
AHA azidohomoalanine amino acid
AUC Augur cell type prioritization
CA1 hippocampal subregion
CA3 hippocampal subregion
CCA common carotid artery
CYP cytochrome microsomal
DAPI 4′,6-diamidino-2-phenylindole
DEG Differentially expressed genes
DG hippocampal subregion
DMEM Dulbecco's Modified Eagle Medium
DNA deoxyribonucleic acid
ECMO extracorporeal membrane oxygenation system
EEG electroencephalogram
EKG electrocardiogram
GFAP Glial fibrillary acidic protein
GO Gene ontology
HEPES 4-(2-hydroxyethyl)-1-piperazineethanesulfonic acid
IBA-I marker for microglia
IL Interlobular artery
2-NBDG glucose analog
OA Ophthalmic Artery
PBS Phosphate buffered saline
PCT proximal convoluted tubule
PCA principal component analysis
PFC prefrontal cortex
PMI post-mortem intervals
RA right atrium
RBFOX3/NeuN marker for mature neurons
RC Renal cortex
RM Renal medulla
RO Retro orbital
ROI region of interest
RT room temperature
SCM sternocleidomastoid muscle
SCT subcutaneous tissue
TUNEL Terminal deoxynucleotidyl transferase dUTP Nick End Labeling
UMAP uniform manifold approximation and projection
UMI unique molecular identifier
VFib ventricular fibrillation
WIT warm ischemia time

### OrganEx Perfusate and Uses

The invention provides a novel OrganEx technology and its experimental application that has the potential for recovery of key molecular and cellular processes in multiple porcine organs after prolonged warm ischemia.

The data presented herein demonstrate that mammalian cells are more resilient to ischemic injury than previously understood. Further, the data establish that cellular deterioration is a more protracted process that is not scripted within narrowly-defined sequences or timeframes.

In one embodiment, the application of the OrganEx technology can halt the process of cell demise. In another embodiment, the application of the OrganEx technology can shift cellular states towards recovery at molecular and cellular levels. In another embodiment, the application of the present invention can shift cellular states towards recovery, even following prolonged warm ischemia. In another embodiment, the invention provides a comprehensive single-cell transcriptomic analysis of the brain and vital peripheral organs over varying warm ischemic intervals. In another embodiment, the comprehensive single-cell transcriptomic analysis of the brain and vital peripheral organs over varying warm ischemic intervals is obtained utilizing perfusion with either autologous blood, the OrganEx perfusate, or a mixture of autologous blood and the OrganEx perfusate.

In some embodiments, the invention provides a transcriptome dataset and a unique resource for future basic and translational studies on cell-types, organs and ischemia.

In one embodiment, the OrganEx platform and acellular perfusate, connected to an intact dead mammal, provides a solution to the problem of ischemic stress in tissue culture and isolated organs.

The invention provides a means to reinstate circulation and systemic metabolic parameters across multiple organs in an intact animal. In one embodiment, the invention provides for the removal of deleterious processes and lack of oxygen, crucial for the control of multiple non-specific injury mechanisms affecting end-organ recovery and overall prognosis after global ischemia.

In one embodiment, the invention facilitates or enables repair responses at the molecular and cellular level in several or all organs. In another embodiment, the repair at the molecular and cellular level translate to processes supporting recovery of organs. In another embodiment, recovery of organs last for an extended period of time. In another embodiment, rigor mortis can be prevented in an animal following warm ischemia. In another embodiment, rigor mortis can be reversed. In another embodiment, dead spots in the heart can be prevented. In another embodiment, electrical activity can be measured in the heart. In another embodiment, contractile activity can be measured in the heart. In another embodiment, electrical activity can be measured in the brain. In another embodiment, the movements can be initiated in the animal. In another embodiment, the animal may regain consciousness. In another embodiment, the animal may regain the ability to move.

In one embodiment, enduring effects on cellular recovery post perfusion occurs in slices from the tissue most susceptible to ischemia, the hippocampus. In another embodiment, long term in vivo recovery of the hippocampus, and other organs is observed at the organ level.

In one embodiment, long OrganEx perfusions of the whole-body can prevent organs from undergoing ischemic injury. In another embodiment, long OrganEx perfusion can lead to recovery of vital organs, including the brain, the hearth, the kidney, the pancreas, or the liver.

In another embodiment, the OrganEx technology can be used in combination with mammals that are still alive. In one embodiment, the live mammal is a human. In one embodiment, the live mammal being treated has experienced a stroke or a heart attack prior to perfusion with the blood perfusate mixture. In one embodiment, the human recovers and/or recovers more quickly from the stroke or the heart attack. In one embodiment, the invention preserves and recovers brain function. In one embodiment, the recovery from ischemic injury in the brain is measurable by magnetic resonance imaging (MRI). In one embodiment, long OrganEx perfusion can be applied to the mammal, e.g., a human, following a cardiac event, e.g., a heart attack. In one embodiment, the invention preserves and recovers heart function. In one embodiment, the mammal recovers and/or recovers more quickly from the heart attack. In one embodiment, the recovery from ischemic injury in the heart is measurable with an electrocardiogram (EKG). In another embodiment, death of a mammal may be reversed.

In one embodiment, the technology provides for new avenues for whole-body global ischemia research. In another embodiment, the invention provides a means to conduct clinical resuscitation science or transplantation medicine. In one embodiment, the invention provides for larger donor organ pools by recovering previously marginalized organs.

The invention includes a novel perfusion composition for the preservation of organs in the mammalian body. In certain embodiments, the perfusion composition can be used to preserve organs in a mammal after warm ischemia. In certain embodiments, the blood perfusate mixture can preserve the brain, the liver, lung, heart, pancreas, kidney, and the like.

In certain embodiments, the perfusion composition is a perfusate comprising a solution comprising one or more artificial oxygen carrier compounds and one or more compounds selected from the group consisting of anti-cytotoxic compounds, antioxidants, anti-inflammatory compounds, antiepileptic compounds, anti-apoptotic compounds, antibiotics, cell death inhibitors, neuroprotectants and oxidative/nitrosative stress inhibitors.

In certain embodiments, the perfusate comprises priming solution. In certain embodiments, the priming solution comprises sodium chloride, sodium bicarbonate, magnesium chloride, calcium chloride, glucose and dextrane. In certain embodiments, the concentrations of the components in the priming solutions is as shown in Table 1.

In certain embodiments, the perfusate comprises hemodiafiltration exchange solution. In certain embodiments, the hemodiafiltration exchange solution comprises one or more amino acids selected from the group consisting of glycine, L-alanyl-glutamine, L-arginine, L-cysteine, L-histidine, L-isoleucine, L-leucine, L-lysine, L-methionine, L-phenylalanine, L-serine, L-threonine, L-tryptophan, L-tyrosine, L-valine and salts and solvates thereof; one or more vitamins selected from the group consisting of choline, D-calcium pantothenate, folic acid, niacinamide, pyridoxine, riboflavin, thiamine, i-inositol and salts and solvates thereof; and one or more inorganic salts selected from the group consisting of calcium chloride, ferric nitrate, magnesium sulfate, potassium chloride, sodium bicarbonate, sodium chloride, sodium phosphate and salts and solvates thereof. In certain embodiments, the concentration of the components in the hemodiafiltration solution is as shown in Table 2.

In certain embodiments, the perfusate contains hemoglobin glutamer-250 (Hemopure @(HBOC-250)) or an alternative oxygen carrier, one or more cytoprotective agents selected from Hexahydro-2-imino-1H-thieno[3,4-d]imidazole-4-pentanoic acid (2-Iminobiotin), 5-(1H-Indol-3-ylmethyl)-3-methyl-2-thioxo-4-Imidazolidinone (Necrostatin-1), Sodium 3-Hydroxybutyric Acid, Glutathione Monoethyl Ester, Minocycline, Lamotrigine, 5-(2,6-Difluorophenoxy)-3-[[3-methyl-I-oxo-2-[(2-quinolinylcarbonyl)amino]butyl]amino]-4-oxo-pentanoic acid hydrate (QVD-Oph), Methylene Blue, and one or more antibiotics and anti-inflammatory agents selected from Ceftriaxone, Dexamethasone, and Cetirizine. In certain embodiments, the concentrations of the components recited above is listed in Table 3.

In certain embodiments, the perfusate comprises one or more artificial oxygen carrier compounds. In certain embodiments, the one or more artificial oxygen carrier compounds are hemoglobin derivatives. The hemoglobin derivatives can be one or more compounds selected from the group consisting of isolated, cell-free hemoglobin, cross-linked hemoglobin, polymerized hemoglobin, encapsulated hemoglobin and functionalized hemoglobin. In certain embodiments, the artificial oxygen carrier compound can be hemoglobin glutamer-250 (HEMOPURE®), a cross-linked hemoglobin tetramer comprising two alpha hemoglobin and two beta hemoglobin subunits cross-linked by a carbon linker. In other embodiments, the one or more artificial oxygen carrier compounds can be artificial red blood cell substitutes, such as ERYTHROMER™, PolyHeme, Oxyglobin, PolyHb-SOD-CAT-CA, PolyHb-Fibrinogen, Hemspan or MP4. In other embodiments, the artificial oxygen carrier compound can be any blood substitute compound known in the art.

In certain embodiments, the perfusion composition comprises one or more compounds selected from the group consisting of anti-cytotoxic compounds, antioxidants, anti-inflammatory compounds, antiepileptic compounds, anti-apoptotic compounds, antibiotics, cell death inhibitors, neuroprotectants and nitrite stress inhibitors. In certain embodiments, the perfusion composition comprises at least one imaging contrast agent. In other embodiments, the perfusion composition further comprises one or more ultrasound contrast agents, that also can be used under certain ultrasound settings as clot disintegrator and blood-brain barrier opener. In some embodiments, the one or more ultrasound contrast agents can be micrometer-sized air-filled polymeric particles. In yet other embodiments, the perfusion composition comprises at least one MRI contrast agent or CT contrast agent. In other embodiments, the perfusion composition comprises one or more compounds selected from the group consisting of the compounds of Tables. 1-3, or salts, solvates, tautomers, and prodrugs thereof.

### Perfusion System

The invention provides a novel system for in situ hypothermic preservation of organs in a mammalian body.

In certain embodiments, the invention provides a system for the hypothermic, in situ preservation system, the system comprising: a perfusion device for the perfusion of a mammalian body, comprising a means for regulating the temperature, flow, pressure, dissolved gases, and concentration of metabolites in the system; and the perfusate composition of the invention. In certain embodiments, the means for regulating the temperature of the system comprises a controller programmed to regulate at least a perfusate temperature within the system to maintain hypothermic conditions. In certain embodiments, the means for regulating the flow, pressure, dissolved gases, and metabolite concentrations in the system comprises a controller programmed to regulate these parameters within the system to maintain constant or alterable levels/concentrations.

In certain embodiments, the system is configured to introduce oxygen to a perfusate of the invention and circulate the perfusate through mammal. In certain embodiments, the perfusion unit is adapted and configured to introduce oxygen and carbon dioxide to the perfusate. In other embodiments, the perfusion unit is adapted and configured to dialyze the perfusate.

In certain embodiments, the system comprises an intrarenal arterial cannula, an animal input line, a pressure sensor, a flow sensor, a pulse generator, an arterial oxygenator, an exchange solution, one or more roller pumps, a hemodiafiltration membrane, one or more reservoirs with perfusate drugs, one or more perfusate reservoirs, a pressure senor, a centrifugal pump, a hemoglobin sensor a return line from the mammal.

In certain embodiments, the electronic components of the perfusion system are built in a modular manner. Each sensor, motor or electronic component that requires external control has a separate logical controller built on Arduino Uno platform, local circuits and software. Each logical controller has a serial output and input and is connected via com port to the computer. In certain embodiments, the computer has modulus, scripts and functions written in Python that either control or collect data from these logical controllers.

In certain embodiments, the pulse generator consists of (1) 3D printed flow chamber (fluid space for blood/perfusate passage, and air space that is separated by a membrane from the fluid space and can change volume against fluid space) connected to the main circuit, downstream from the centrifugal pump, (2) high resolution electronic pressure regulator (0-10 PSI) which is connected to the air source on one end and to the flow chamber on the opposite end, (3) logical controller regulating electronic pressure regulator, (4) and a computer with control software. The Logical controller is made out of Arduino Uno microprocessor board and has a DAC converter (MCP 4725) with an operational amplifier (LT1215 CN8); it produces continuous analog output (0-10V) for electronic pressure regulator. Logical controller has a script which runs the electronic pressure regulator, based on sinusoidal function, and it can receive input from a computer via serial port to regulate variables within sinusoidal function (e.g. amplitude, iteration/looping speed, baseline value). In certain embodiments, continuous output is produced and sent to the electronic pressure regulator which then controls flow chamber volume, by moving a membrane which separates fluid space from the air space, thus producing pulsatility and oscillations in venous and arterial pressures throughout the perfusion system. Computer is connected to the logical controller, and custom-made scripts in Python allow for manual or automatic control. In certain embodiments, the software also monitors flow and pressure in the arterial and venous cannula and stores the data.

In certain embodiments, the automated hemodiafiltration system consists of (1) two peristaltic pumps which have separate logical controllers and are connected via serial ports to the computer, (2) liquid level solid-state sensor with a resistive output (0-5V), in a clear, elliptical polycarbonate tube integrated with logical controller, for monitoring fluid level in exchange solution canister, connected to the computer. Logical controllers for peristaltic pumps and level sensor have custom made scripts which allow for higher-order language control. In certain embodiments, the computer governs two peristaltic pumps and collects data from the sensor via Python script. The script performs proportional control over peristaltic pumps and allows for continuous dialysis while keeping the animal euvolemic.

### Perfused Organs in the Mammalian Body

The invention provides methods of preserving organs in the mammalian body. In some embodiments, the organs are perfused with a solution comprising a priming solution, a hemodialysis solution and a solution comprising pharmacological components. The constituents and concentrations of the components in the priming solution, hemodialysis solution and solution comprising the pharmacological components are as shown in Tables 1-3. In certain embodiments, the mammalian body is perfused with a mixture of the perfusate and autologous blood. In some embodiments, the autologous blood is mixed with any of the components of the perfusate solution before perfusion of the mammalian body. In some embodiments, one or more artificial oxygen carriers are present in the mixture.

In some embodiments, the mammalian organs maintain morphofunctional integrity under hypothermic conditions after perfusion with the blood perfusate mixture.

In certain embodiments, the organs in the mammal are ischemic prior to perfusion with the blood perfusate mixture.

In certain embodiments, the organs are perfused while the mammal is still alive. In other embodiments, the organs are perfused immediately upon death of the mammal. In other embodiments, there is a 20 minute delay between death and perfusion of the organs. In other embodiments, the time between death and perfusion is at least one hour. In other embodiments, the time between death and perfusion is 2, 3, 4, 5, 6, 7, 8, 9, 10, 11 or 12 hours.

In certain embodiments, the perfused organs are organs that can be transplanted from one mammal to another, including, but not limited to the kidney, the pancreas, the heart, the lung, the intestine, the corneas, the middle ear, bone, bone marrow, heart valves, connective tissue, skin, uterus, muscles, blood vessels, nerves and connective tissue. In some embodiments, the organs are removed from the mammal following perfusion with the blood perfusate mixture.

In certain embodiments, perfusion with the blood perfusate mixture helps maintain an in vivo rate of cellular metabolism and preserves functional responses of cells. The blood perfusate perfused organs can maintain longer viability than organs perfused with the ECMO system.

In certain embodiments, the perfused organs belong to any mammal. Non-human mammals include, for example, livestock and pets, such as ovine, bovine, porcine, canine, feline and murine mammals. Mammals can also include primates, including humans. In certain embodiments, the perfused mammal is a human.

In some embodiments, rigor mortis is prevented by perfusion of the deceased mammal. In one embodiment, perfusion of the deceased mammal with the technologies described herein prevents stiffening of the muscles. In another embodiment, the tissues in the perfused mammal continue or regain the consumption of. In another embodiment, the tissues in the perfused mammalian continue or regain the ability to produce ATP.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in practicing the following examples are here described:

### Materials and Methods

### Overview of the OrganEx Perfusion System and Perfusate

### Overview of the OrganEx Perfusion System

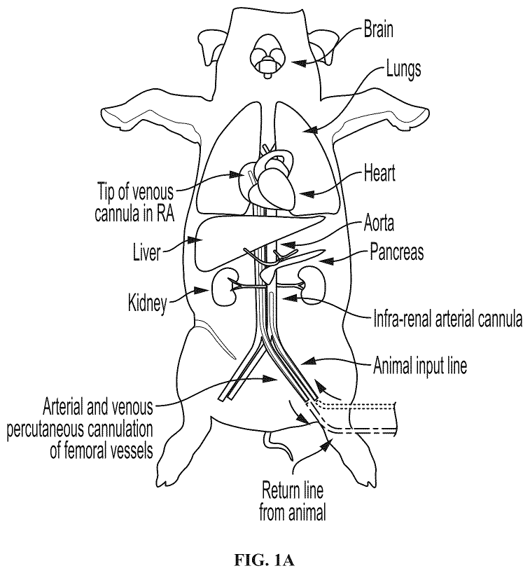

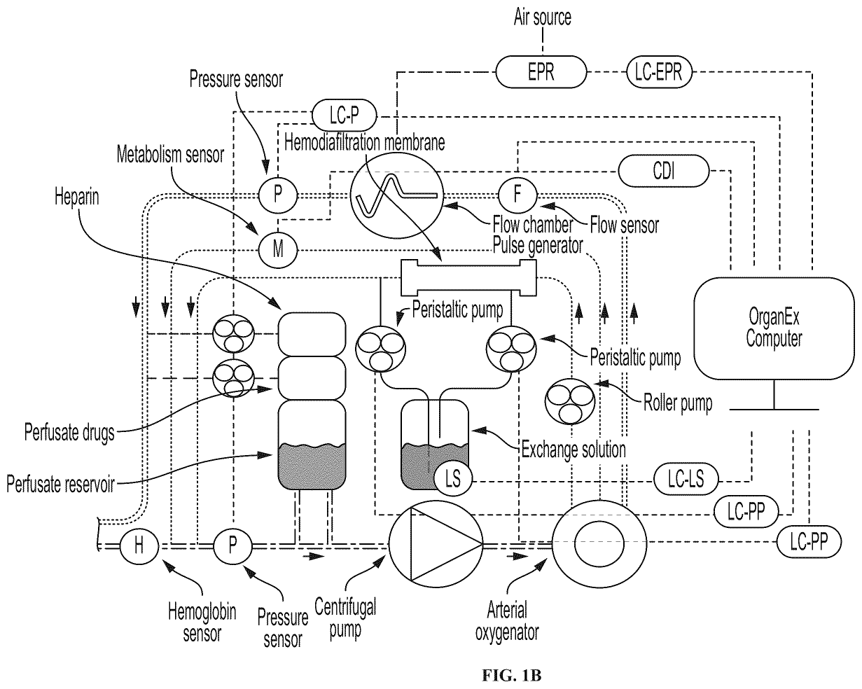

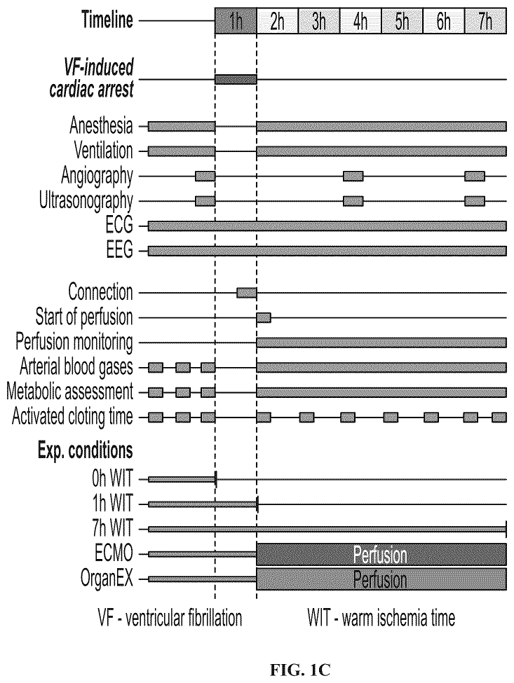

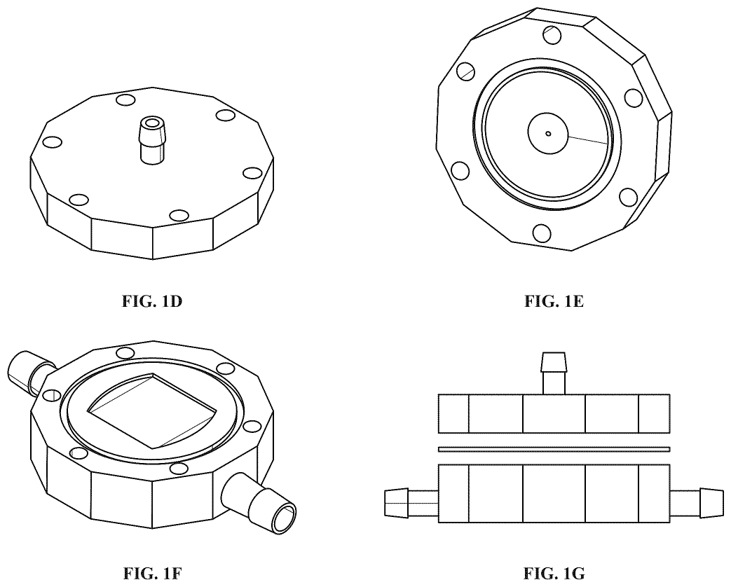

The perfusion system consists of the main closed-loop circuit directly connected to an animal, and it includes a centrifugal pump (Medtronic Bio-Console 560, Medtronic, Minneapolis, MN) that drives the mixture of autologous blood and OrganEx perfusate through the oxygenator (Affinity Fusion, Medtronic), and custom-made pulsatility generator into animal arterial system. The oxygenator is connected to a refrigerated bath (Polystat, Cole-Parmer, Niles, IL) for temperature control and the gas blender (Sechrist Industries, Anaheim, CA), for control of dissolved gases and anesthesia infusion. The perfusion system has a fluid reservoir, which is used to prime the system and hold the supplement fluid. In parallel, an automated hemodiafiltration system and a reservoir are connected to the main circuit (FIG. 1). The automated hemodiafiltration system is used to exchange plasma fraction against custom-made dialysis exchange solution. The hemodiafiltration system consists of a roller-pump (Cobe Shiley, Stockert, Lakewood, CO), dialyzer (Diacap Pro 13H, Braun, Melsungen, Germany) and two peristaltic pumps (Masterflex L/S, Cole-Parmer) integrated with level sensor (eTape, Milone Technologies, Sewell, NJ) and custom-made logical controller. Two infusion pumps (Sigma Spectrum, Baxter Healthcare Corporation, Deerfield, IL) are connected to the arterial side of the main circuit supplementing heparin and pharmacological compounds of the perfusate. The CDI blood parameter module and the hematocrit/oxygen saturation probe (Terumo Cardiovascular Systems Corp., Elkton, MD) are connected on the arterial and venous side, respectively, along with pressure (PendoTECH, Princeton, NJ) and flow sensors (Bio-Probe TX50, Medtronic). OrganEx perfusion system components, logical controllers and sensors are connected to a computer for automated control and data gathering. Detailed schematics available upon request.

### Preparation and Application of the OrganEx Perfusate

The OrganEx perfusate is a final mixture of a custom-made priming solution (Table 1), Hemopure (HbO2 Therapeutics, Waltham, MA), custom-made dialysis exchange solution (Table 2) and the solution of pharmacological compounds (Table 3).

TABLE 1
Components of the priming solution
Exchange Solution

| | | Concen- | Concen- | |
|---|---|---|---|---|
| | M.W. | tration | tration | Volume |
| Components | (g/mole) | (g/L) | (mM) | (mL) |
| Sodium Chloride | 58.44 | 5.844 | 140 | N/A |
| Sodium Bicarbonate | 84.007 | 3.36 | 40 | N/A |
| Magnesium Chloride | 203.3 | 0.305 | 1.5 | N/A |
| Calcium Chloride | 147.01 | 0.147 | 1 | N/A |
| Glucose | 180.156 | 0.9 | 5 | N/A |
| Dextrane | 504.4 | 25 | 100 | N/A |

TABLE 2
Components of the hemodiafiltration exchange solution:
Exchange Solution

| | | | |
|---|---|---|---|
| | | Concen- | Concen- |
| | M.W. | tration | tration |
| Components | (g/mole) | (mg/L) | (mM) |

Amino Acids

| | | | |
|---|---|---|---|
| Glycine | 75 | 18 | 0.24 |
| L-Alanyl-Glutamine | 217 | 350.4 | 1.614746544 |
| L-Arginine hydrochloride | 211 | 50.4 | 0.238862559 |
| L-Cystine | 313 | 37.5 | 0.119808307 |
| L-Histidine hydrochloride-H₂O | 210 | 25.2 | 0.12 |
| L-Isoleucine | 131 | 63 | 0.480916031 |
| L-Leucine | 131 | 63 | 0.480916031 |
| L-Lysine hydrochloride | 183 | 87.6 | 0.478688525 |
| L-Methionine | 149 | 18 | 0.120805369 |
| L-Phenylalanine | 165 | 39.6 | 0.24 |
| L-Serine | 105 | 25.2 | 0.24 |
| L-Threonine | 119 | 57 | 0.478991597 |
| L-Tryptophan | 204 | 9.6 | 0.047058824 |
| L-Tyrosine | 181 | 62.274 | 0.344055249 |
| L-Valine | 117 | 56.4 | 0.482051282 |

Vitamins

| | | | |
|---|---|---|---|
| Choline chloride | 140 | 2.4 | 0.017142857 |
| D-Calcium pantothenate | 477 | 2.4 | 0.005031447 |
| Folic Acid | 441 | 2.4 | 0.005442177 |
| Niacinamide | 122 | 2.4 | 0.019672131 |
| Pyridoxine hydrochloride | 206 | 2.4 | 0.011650485 |
| Riboflavin | 376 | 0.24 | 0.000638298 |
| Thiamine hydrochloride | 337 | 2.4 | 0.007121662 |
| i-Inositol | 180 | 4.32 | 0.024 |

Inorganic Salts

| | | | |
|---|---|---|---|
| Calcium Chloride | 147 | 120 | 0.816326531 |
| Ferric Nitrate | 404 | 0.06 | 0.0001485 |
| Magnesium Sulfate | 246 | 58.602 | 0.238219512 |
| Potassium Chloride | 75 | 240 | 3.2 |
| Sodium Bicarbonate | 84 | 2220 | 26.42857143 |
| Sodium Chloride | 58 | 3840 | 66.20689655 |
| Sodium Phosphate monobasic | 156 | 65.4 | 0.419230769 |

TABLE 3
Pharmacological components of OrganEx Perfusate
Organ Ex Perfusate

| | | | | |
|---|---|---|---|---|
| | | Concen- | Concen- | |
| | M.W. | tration | tration | Volume |
| Components | (g/mole) | (mg/L) | (mM) | (mL) |

Gas-Exchange

| | | | | |
|---|---|---|---|---|
| Hemopure ® (HBOC-250) | 250k | 130k | 0.52 | 750 |
| | (average) | | | |

Cytoprotective Agents

| | | Concen- | Concen- | |
|---|---|---|---|---|
| | M.W. | tration | tration | Volume |
| | (g/mole) | (mg/kg) | (mM) | (mL) |
| Hexahydro-2-imino-1H-thieno[3,4-d]imidazole- | 243 | 0.303 | N/A | N/A |
| 4-pentanoic acid (2-Iminobiotin) | | | | |
| 5-(1H-Indol-3-ylmethyl)-3-methyl-2-thioxo-4- | 259 | 1.515 | N/A | N/A |
| Imidazolidinone (Necrostatin-1) | | | | |
| Sodium 3-Hydroxybutyric Acid | 126 | 43.636 | N/A | N/A |
| Glutathione Monoethyl Ester | 335 | 6.061 | N/A | N/A |
| Minocycline | 494 | 6.061 | N/A | N/A |
| Lamotrigine | 256 | 18.182 | N/A | N/A |
| 5-(2,6-Difluorophenoxy)-3-[3-methyl-1-oxo-2- | 513 | 0.758 | N/A | N/A |
| [(2-quinolinylcarbonyl)amino]butyl]amino]-4- | | | | |
| oxo-pentanoic acid hydrate (QVD-Oph) | | | | |
| Methylene Blue | 320 | 1.515 | N/A | N/A |

Antibiotics and Anti-inflammatory

| | | | | |
|---|---|---|---|---|
| Ceftriaxone | 661 | 121.212 | N/A | N/A |
| Dexamethasone | 392 | 10.606 | N/A | N/A |
| Cetirizine | 388 | 3.03 | N/A | N/A |

In detail, prior to connecting an animal, the OrganEx perfusion system is flooded and primed with 2200 mL of custom-made priming solution (5000 mL), followed by infusion of 1000 mL of Hemopure into the system. These solutions are left to mix and equilibrate throughout the perfusion system, after which 600 mL is extracted from the perfusion system to achieve desired concentrations of electrolytes and oncotic agents in the perfusion system and prepare it for addition of the autologous blood.

Prior to initiation of the perfusion protocol, animal femoral vessels are cannulated and connected to the main circuit. At 30 minutes of WIT, 5 000 USP units of heparin (Sigma-Aldrich, St Louis, MO) is administered into the system, followed by approximately 1000 mL of venous blood from the dead animal, which is drained into the perfusion system. At this point, circulatory volume in the OrganEx perfusion system is approximately 3600 mL, out of which 2600 mL is the priming solution and 1000 mL of autologous blood. Next, the mixture of the perfusate and autologous blood is left to equilibrate, and counter dialyzed against the residual priming solution over 30 minutes to allow for correction of metabolic derangements in the drained venous blood. In parallel, approximately 1600 mL of fluid is filtered out of the perfusion system over 30 minutes while the residual fluid is cooled to 28° C., yielding a final volume of 2000 mL in the OrganEx perfusion system. Next, at 1h of WIT, 1000 mL of the perfusate and autologous blood mixture is infused back into the animal, ensuring circulatory system filling following venous drainage, and the perfusion protocol is initiated. The remaining 1000 mL of the mixture is stored in the reservoir and used for fluid supplementation, if required. Following infusion of the perfusate and autologous blood mixture, pharmacological compounds and dialysis exchange solution, containing amino acids, vitamins and inorganic salts, are continuously infused into the main perfusion circuit by infusion pump and hemodiafiltration system, respectively. The OrganEx perfusion system utilizes automated hemodiafiltration circuit which corrects and maintains certain metabolic and electrolyte parameters by performing 1:1 (vol:vol) exchange of solutes and particles smaller than 40 kDa against a custom dialysis exchange solution (20,000 mL), while maintaining euvolemia. Hemodiafiltration flux was kept at 30-35 mL/kg/hr throughout 6-hour perfusion.

### Overview of the ECMO Perfusion System

The ECMO perfusion system was assembled according to clinical standard. ECMO perfusion system has the main closed-loop circuit directly connected to an animal and consists of a centrifugal pump (Bio-Console 560, Medtronic) that drives autologous blood through the oxygenator (Affinity Fusion, Medtronic) into animal arterial system. The oxygenator is connected to a refrigerated bath (Polystat, Cole-Parmer) for temperature control and the gas blender (Sechrist Industries), for control of dissolved gases and anesthesia infusion. The perfusion system has a fluid reservoir, which is used to prime the system and hold the supplement fluid. Furthermore, ECMO perfusion system contained the CDI blood parameter module, and the hematocrit/oxygen saturation probe (Terumo Cardiovascular Systems Corp.) are connected on the arterial and venous side, respectively, along with pressure (PendoTECH) and flow sensors (Bio-Probe TX50, Medtronic). All probes and sensors from the ECMO perfusion system are connected to a computer to allow data gathering. Detailed schematics available upon request.

The ECMO perfusion system is primed with 1000 mL 0.9% Sodium Chloride (Baxter Healthcare Corporation, Deerfield, IL) and 5000 USP units of heparin (Sigma-Aldrich). Upon initiation of the perfusion protocol, the reservoir is taken out of the main circuit and the residual priming solution is stored for later supplementation.

### Animal Anesthesia and Surgical Protocol

This research project was approved and overseen by Yale's Institutional Animal Care and Use Committee (IACUC) and guided by an external advisory and ethics committee. Experimental animals were procured from the local farm breeder, female domestic pigs (Sus scrofa domesticus; ˜30-35 kg). All animals were housed at Yale School of Medicine Division of Animal Care's facilities at a minimum of 3 days before the experiment.

Prior to experimental protocol, all animals received a Fentanyl patch, 50 ug/hr (Duragesic, Henry Schein, Melville, New York, NY, USA) for sedation. To induce anesthesia, 6 mg/kg of Telazol (Henry Schein) and 2.2 mg/kg of Xylazine (Henry Schein) were administered. Next, animals were intubated and connected to the ventilator utilizing FiO2 of 40% and FiN2 of 60% with standard parameters of tidal volume 10-15 ml/kg and frequency of 14-16 breaths per minute, along with 1-2% isoflurane (Henry Schein). Following ventricular fibrillation and induction of cardiac arrest, ventilation was stopped for 1h. Upon initiation of the perfusion protocol, in ECMO and OrganEx groups, ventilation was continued utilizing a tidal volume of 3-4 ml/kg, frequency 5 breaths per minute, positive end-expiratory pressure (PEEP) of 10 cm H2O, low inflation pressure, FiO2 50%, FiN2 50%. During the 6-hour perfusion protocol, 0.5% isoflurane was administered through the vaporizer connected to the gas blender.

Cardiac arrest and subsequent circulatory collapse were induced by ventricular fibrillation through the substernal window by applying a 9V battery to the myocardial wall. Prior to the ventricular fibrillation, animals received 7 000 USP units of heparin (Sigma-Aldrich). In order to connect the circulatory system of the animal to ECMO or OrganEx perfusion system, an incision was made in the right inguinal region exposing femoral artery and vein (FIGS. 1A-1G). Both, arterial and venous cannulas were inserted into femoral artery and vein, respectively. Artery was cannulated with 14 Fr and the vein with 19 Fr cannula (Edwards Lifesciences LLC, Irvine, CA). The tip of the venous cannula was placed in the inferior vena cava opening of the right atrium, and arterial cannula was positioned inferior to renal arteries.

### Perfusion Protocol and Monitoring of Physiologic and Metabolic Parameters

At the start of the perfusion protocol in the OrganEx group, a mixture of the perfusate and autologous blood was slowly infused over 5 minutes resulting in an average flow rate of 600 mL/min at the end of infusion. Following this step, the flow rate was gradually increased over the next 20 minutes to a targeted flow rate of 80-100 mL/kg/min or highest possible flow rate without introducing overspinning of the centrifugal pump. Throughout the flow rate ramping-up process, pulsatility was set to oscillate around the mean flow rate at approximately ±10% of the given total flow rate. Residual mixture of the synthetic perfusate and autologous blood which was stored in the reservoir was used for fluid supplementation at 1-2 mL/kg/hr. Targeted arterial pressure was set to 50-80 mmHg, and it was controlled with phenylephrine, not more than 2 mg/hr. Similarly, in the ECMO group, flow rate was gradually increased over 25 minutes, targeting flow rates and arterial pressures as in the OrganEx group. Ringer's lactate (Baxter Healthcare Corporation) was used as a supplementation fluid at 3-4 ml/kg/hr. In both, ECMO and OrganEx group hypothermic perfusion protocol at 28° C. was utilized throughout the entire 6-hours of the perfusion protocol.

The animals in both, ECMO and OrganEx groups received 50 mL of 8.4% sodium bicarbonate (Henry Schein) during the first hour of perfusion. Glucose was supplemented according to the blood levels with the goal of maintaining euglycemia. Protamine, 25 mg, was administered immediately following initiation of the perfusion protocol to control activated clotting time, which was maintained between 180 and 220 seconds with titrated heparin administration. In both perfusion groups, partial pressure of arterial CO₂ and O₂ were targeted to 35 and 250 mmHg via gas blender, respectively.

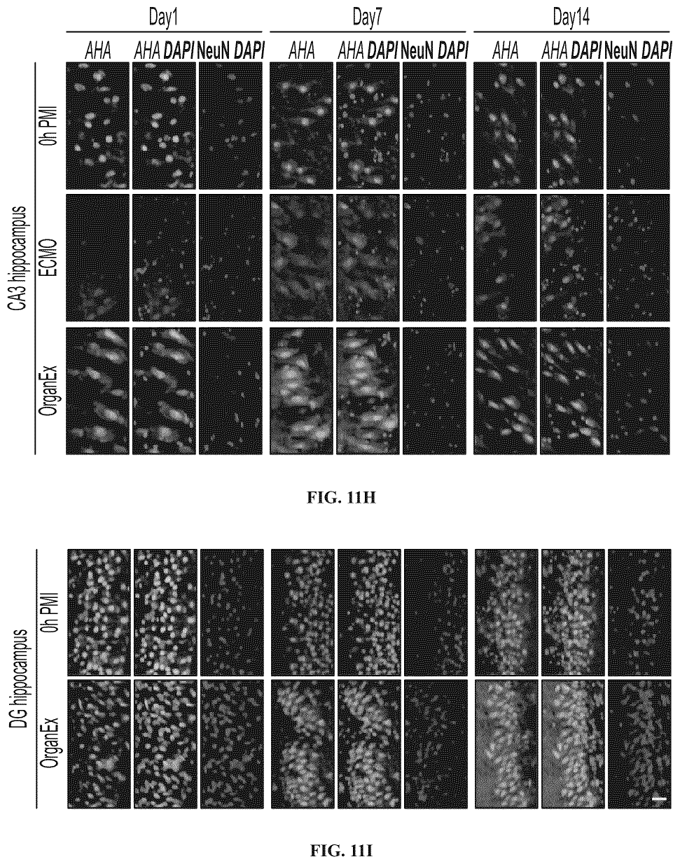

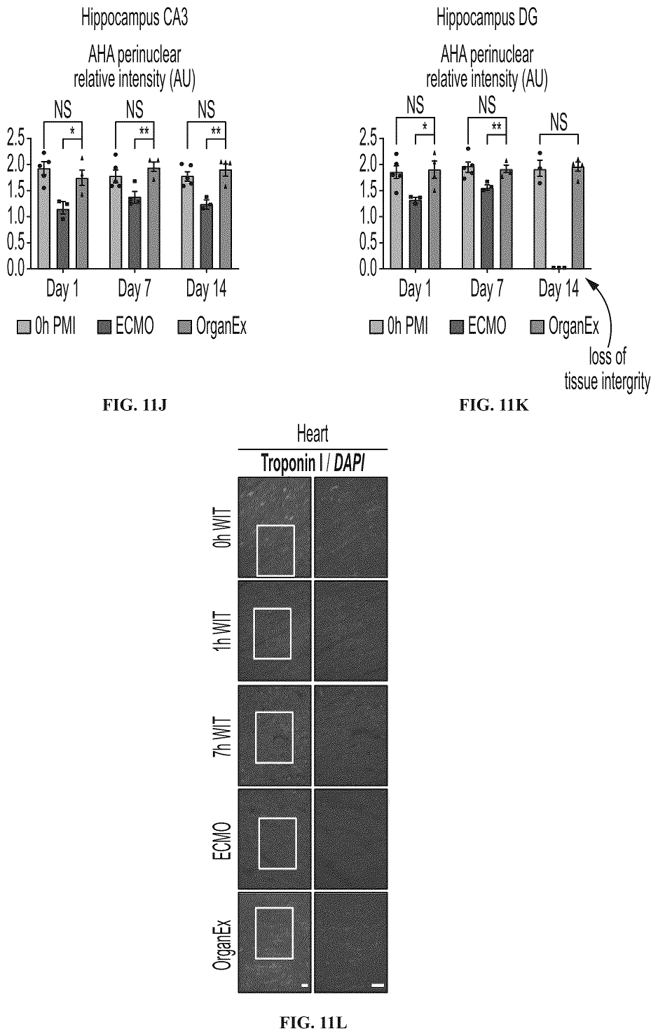

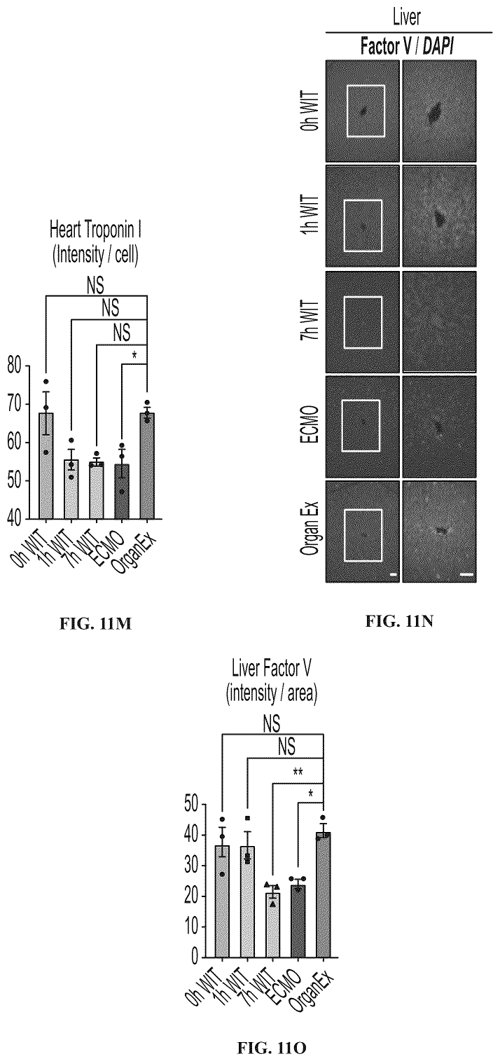

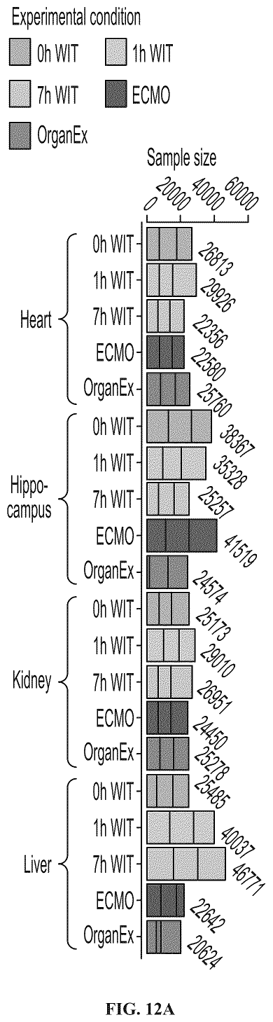

Electrocardiogram (EKG) assessment was done with 4 leads placed at each corner of the trunk. Real time arterial and central venous pressure monitoring was done through cutdown of the brachial artery and jugular vein, respectively. Urine output was measured via Foley catheter. Animal core temperature was continuously monitored with a rectal probe. Monitoring of EKG, pressure and temperature was done utilizing Philips IntelliVue MP50 (Philips, Eindhoven, NL). During the preoperative procedure temperature was kept at 37° C. using a heating pad, which was turned off following ventricular fibrillation. Electroencephalogram (EEG) was monitored with Natus long-term monitoring (LTM) system and EMU40 breakout box (Natus Medical Inc., San Carlos, CA). Six electrodes were placed subcutaneously along the scalp (FIG. 11A) at the start of the sedation. EEG monitoring was conducted throughout the entire 6-hour perfusion protocol. Baseline and hourly arterial and venous samples were collected from the arterial and venous cannulas respectively. Sixty microliters of each sample were immediately analyzed using the GEM4000 clinical blood analyzer system (Instrumentation Laboratory, Bedford, MA). Continuous monitoring of blood electrolytes and hemoglobin concentration and saturation were done with CDI-500 (Terumo Cardiovascular Systems Corp.).

### Radiographic and Ultrasound Imaging of Circulation

### Fluoroscopy

Imaging of the abdominal and head blood vessels was performed using Philips Allura Xper FD20 system. In selected animals that underwent fluoroscopy, baseline physiological imaging was performed prior to the induction of ventricular fibrillation in both ECMO and OrganEx experimental protocol. The contrast-injecting catheter was introduced through the femoral artery cutdown and positioned in suprarenal aorta for renal and in the common carotid artery for brain imaging. Omnipaque Contrast 350 mg/mL (General Electric Inc., Boston, MA), 24 mL and 45 mL were introduced utilizing Medrad power injector (Bayer Vital GmbH, Leverkusen, Germany) for brain and kidney imaging acquisition. Following baseline imaging, all animals underwent additional fluoroscopy at hour 3 of perfusion. In both, ECMO and OrganEx group, imaging of abdominal blood vessels was modified by placing the contrast-injecting catheter in the infrarenal aorta due to the reversal of arterial flow direction, a consequence of femoral artery/vein perfusion approach in both, ECMO and OrganEx groups. The reconstructed images were saved in DICOM format and further post-processed using RadiAnt DICOM Viewer software (Medixant; Poznan, Poland).

### Ultrasonography

Perfusion dynamics were monitored via Triplex Ultrasonography (Spectral Doppler, Colour Doppler, and B-mode) using the LOGIQe portable ultrasound system (General Electric) and an 8L-RS linear array probe (General Electric). In all assessed animals, left ophthalmic artery, common carotid artery and intrarenal arteries were used to profile perfusion dynamics. Power waveform analysis was done using Frq 4.4 MHz, Gn 17, SV 2 and DR 40.

### Cell Nuclei Isolation

Following the appropriate experimental workflow, regions of interest were extracted 30 from each organ and frozen at −80° C. To ensure consistency between the specimens, all dissections were performed by the same person. Cell nuclei isolation from each organ (brain, heart, liver, kidney) were treated the same according to our already established protocol with some modifications in order to acknowledge each organ's specific structural qualities and to have identical buffers to enable inter-organ comparison within the same experimental animal. To avoid experimental bias nuclei isolation was done by the same person blinded for the replicates of experimental conditions. Furthermore, to randomly and fully represent the full tissue section, each tissue was pulverized to fine powder in liquid nitrogen with mortar and pestle (Coorstek, Golden, CO). All reagents were molecular biology grade and sourced from Sigma unless stated otherwise. Small amounts of pulverized tissue (5-10 mg) were then added into 1 ml of ice-cold lysis buffer (“Buffer A” is 250 mM sucrose, 25 mM KCl, 5 mM MgCl2, 10 mM NaCl, 10 mM Tris-HCl (pH 7.4), protease inhibitors w/o EDTA (Roche), RNAse inhibitor (80 U/ml) (Roche), 1 mM DTT, 1% BSA (m/v) (Gemini Bio-Products, Woodland, CA), 0.1% NP-40 (v/v), 0.1% Tween-20 (v/v) (Bio-Rad), 0.01% Digitonin (m/v) (Thermo-Fisher, Cleveland, OH). For lysis of heart, 0.1% TX-100 (v/v) was additionally added. DTT, RNAse Protector, protease inhibitors, and all detergents were added immediately before use. The suspension was transferred to 2 ml Dounce tissue homogenizer and lysed with constant pressure and without introduction of air with pestle A (30×) and pestle B (30×). The homogenate was strained through pre-wetted 40 μm tube top cell strainer (Thermo-Fisher). All subsequent centrifugation was performed in a refrigerated, bench-top centrifuge with swing-out rotor (Eppendorf, Hamburg, Germany). Heart lysate was centrifuged at 100 g for 5 min at 4° C., pellet of myofibrils and non-dissociated connective tissue was discarded, and supernatant saved. All lysates (brain, liver, kidney) and heart supernatant (post 100 g) were centrifuged at 1000 g, 10 min, 4° C., pellets were saved, and resuspended in 0.4 ml resuspension buffer (“Buffer B” is “Buffer A” w/o detergents). Final 0.4 ml of solution was mixed with 0.4 ml (1:1) of Optiprep solution (Buffer “C” is iodixanol 50% (v/v), 25 mM KCl, 5 mM MgCl2, 10 mM NaCl, 10 mM Tris-HCl (pH 7.4), protease inhibitors w/o EDTA, RNAse inhibitor (80U/ml), 1 mM DTT, 1% BSA (m/v)). The suspension (25% iodixanol final) was mixed 10× head over head and overlayed on 0.6 ml of 29% iodixanol cushion (appropriate mix of Buffer “B” and “Buffer “C”). The tubes were then centrifuged at 3000 g, for 30 min at 4° C. Following centrifugation, the supernatant was removed and total of 1 ml of wash buffer (“Buffer D” is 25 mM KCl, 5 mM MgCl2, 10 mM NaCl, 10 mM Tris-HCl (pH 7.4), RNAse inhibitor (80 U/ml), 1 mM DTT, 1% BSA (m/v), 0.1% Tween 20 (v/v), in DPBS (w/o Ca2+ and Mg2+) (Gibco)) was added in tubes and centrifuged at 1000 g, for 10 min at 4° C. Supernatants were then completely removed, pellets were gently dissolved by adding 100 ul of resuspension buffer (“Buffer E” is “Buffer D” w/o detergent) and pipetting 30× with lml pipette tip, pooled and filtered through 20 micrometer cell strainer. Finally, nuclei were counted on hemocytometer and diluted to 1 million/ml.

Single Nuclei Microfluidic Capture and cDNA Synthesis

Extracted nuclei samples were placed on ice and processed in the laboratory within 15 minutes for single nucleus RNA sequencing with targeted nuclei recovery of 10000 nuclei, respectively, on microfluidic Chromium System (10× Genomics, Pleasanton, CA) by following the manufacturer's protocol (CG000315_ChromiumNextGEMSingleCell3′_GeneExpression_v3.1 (DualIndex)_RevA), with Chromium Single Cell 3′ GEM, Library & Gel Bead Kit v3.1, (PN-1000268, 10×Genomics) and Chromium Single Cell G Chip Kit (PN-1000120, 10× Genomics), Dual Index Kit TT Set A (PN-1000215, 10×Genomics) on Chromium Controller (10×Genomics). Since the input material were nuclei, cDNA was amplified for 14 cycles.

### Single-Nucleus RNA-Seq Library Preparation and Sequencing

Post cDNA amplification cleanup and construction of sample-indexed libraries and their amplification followed manufacturer's directions (CG000315_ChromiumNextGEMSingleCell3′_GeneExpression_v3.1 (DualIndex)_RevA), with the amplification step directly dependent on the quantity of input cDNA.

In order to reach sequencing depth of 20000 raw reads per nucleus, single nucleus libraries were run using paired end sequencing with single indexing on the NovaSeq 6000 S4 (Illumina) by following manufacturer's instructions (Illumina, San Diego, CA; 10× Genomics). To avoid lane bias, multiple uniquely indexed samples were mixed and distributed over several lanes.

### Single-Nucleus Transcriptome Analysis

### Quality Control and Analysis of Single Nuclei Transcriptome Data

Sequencing reads were aligned to the reference pig genome (susScr11) with the combined exon-intron gene annotations from NCBI RefSeq using CellRanger 5.0.1. pipeline, which also performed UMI counting, barcode counting and distinguishing true cells from background. The filtered count matrices were then moralized by library size using the “NormalizeData” function in Seurat. To compare cellular and transcriptomic changes across conditions, feature selection was first performed for each batch and the features from the same conditions were summarized using “SelectIntegrationFeatures”. The union of the highly variable genes across conditions were then passed to the data integration pipeline in Seurat to generate a batch-corrected expression matrix. To reveal the cellular diversity among the cells, we scaled the integrated data followed by dimension reduction using principal component analysis (PCA) and selecting principal components via elbow plot. The cell clusters in the k-nearest neighbor graph were then identified and visualized clustering results by Uniform Manifold Approximation and Projection (UMAP). The initial clustering analysis revealed some low-quality clusters with low number of unique molecular identifiers (UMIs), and normally high mitochondria percentage and no meaningful cluster markers, which were all removed for downstream analysis. To annotate the identity of the cell clusters, the pig data was then integrated with published human data from the same organ using the same data integration pipeline described above. The cluster markers were calculated using “FindMarkers” function and manually removed the doublet clusters that showing high expression of markers of two different type of cells. To gain more accurate cell annotations and clearer UMAP visualizations, the same pipelines of data integration, dimension reduction and cell clustering were reperformed on the filtered data.

Global Transcriptomic Comparisons with Public Datasets.

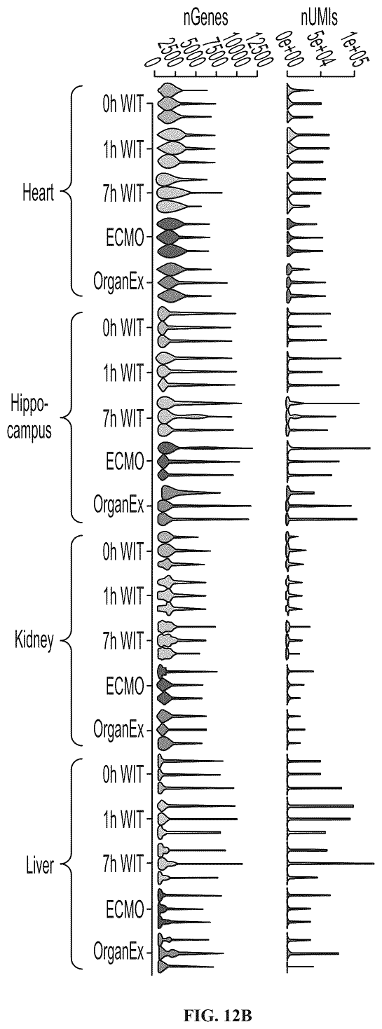

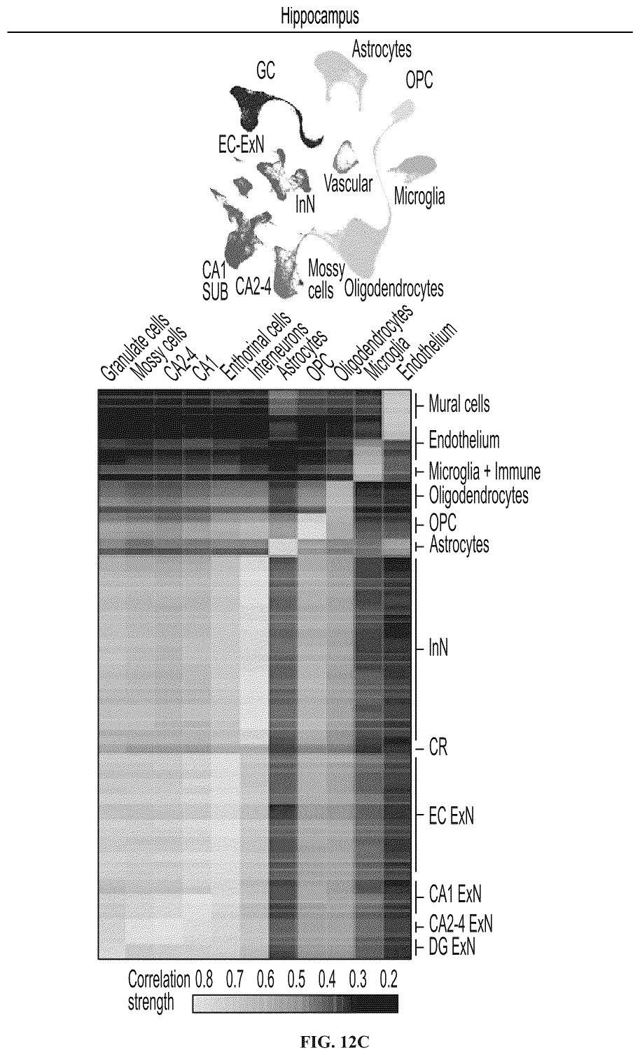

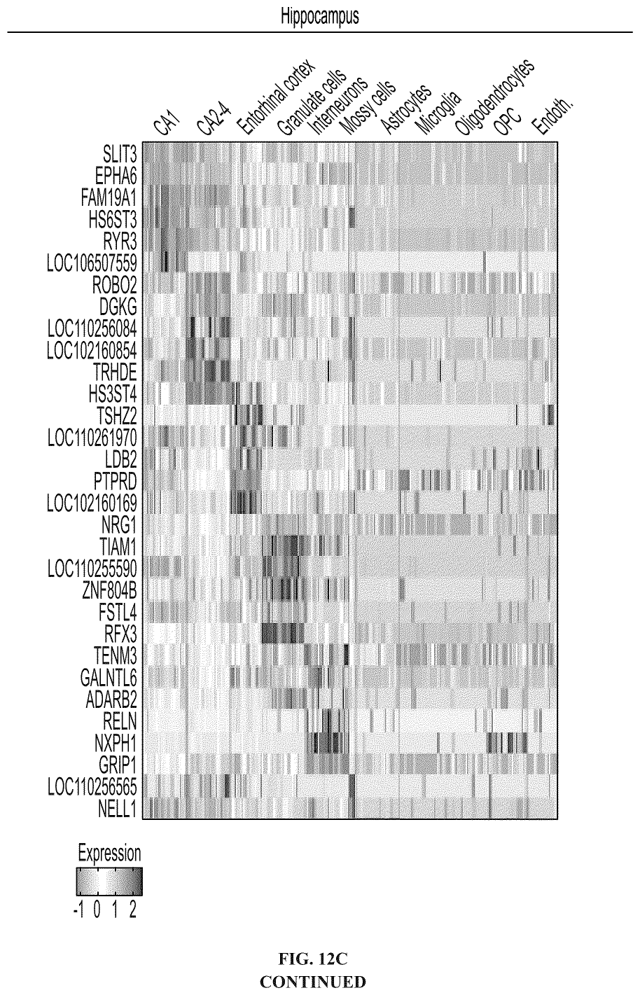

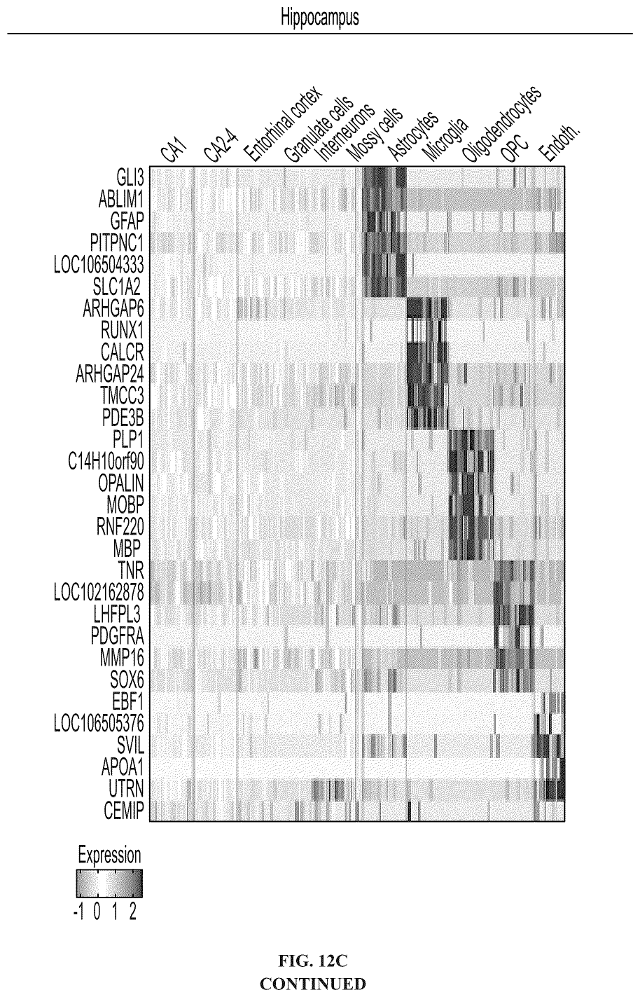

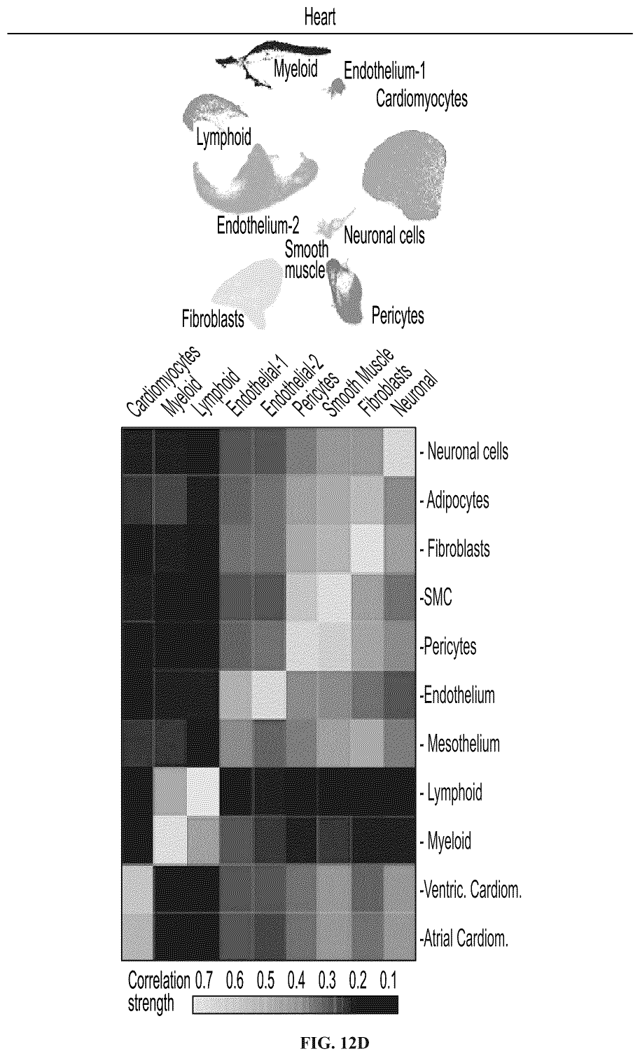

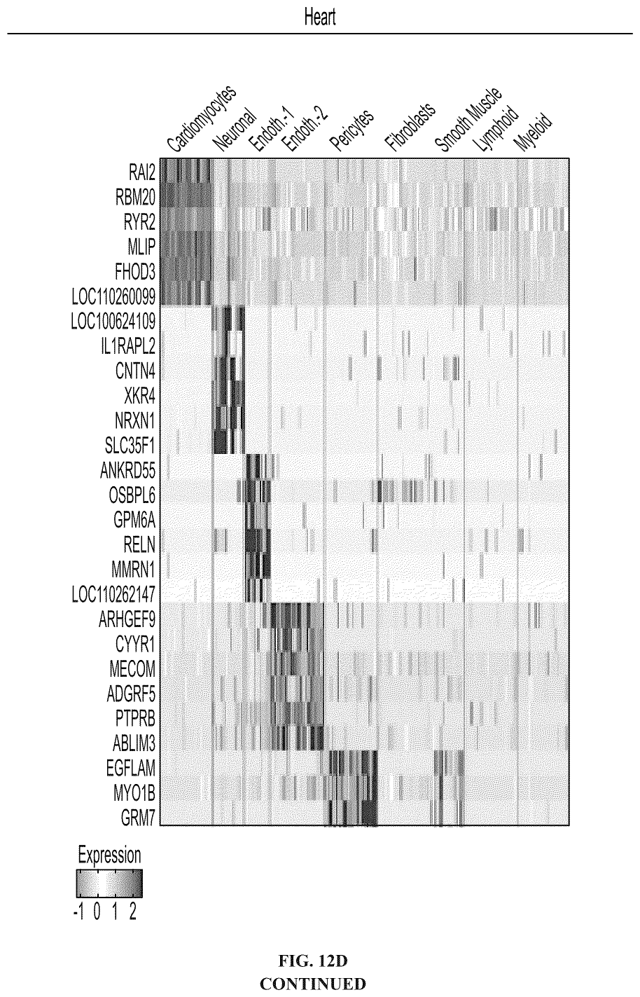

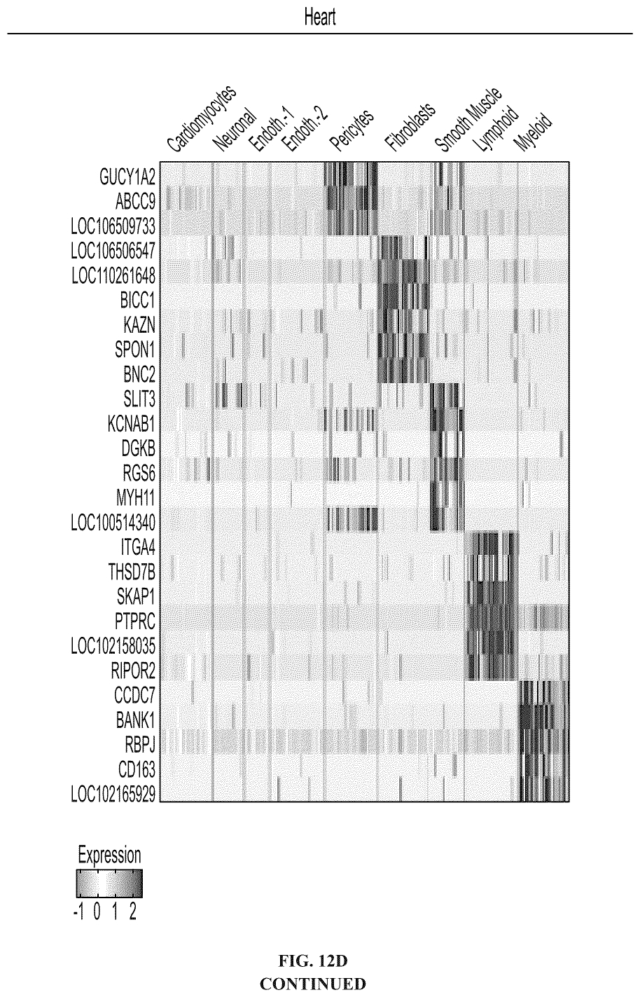

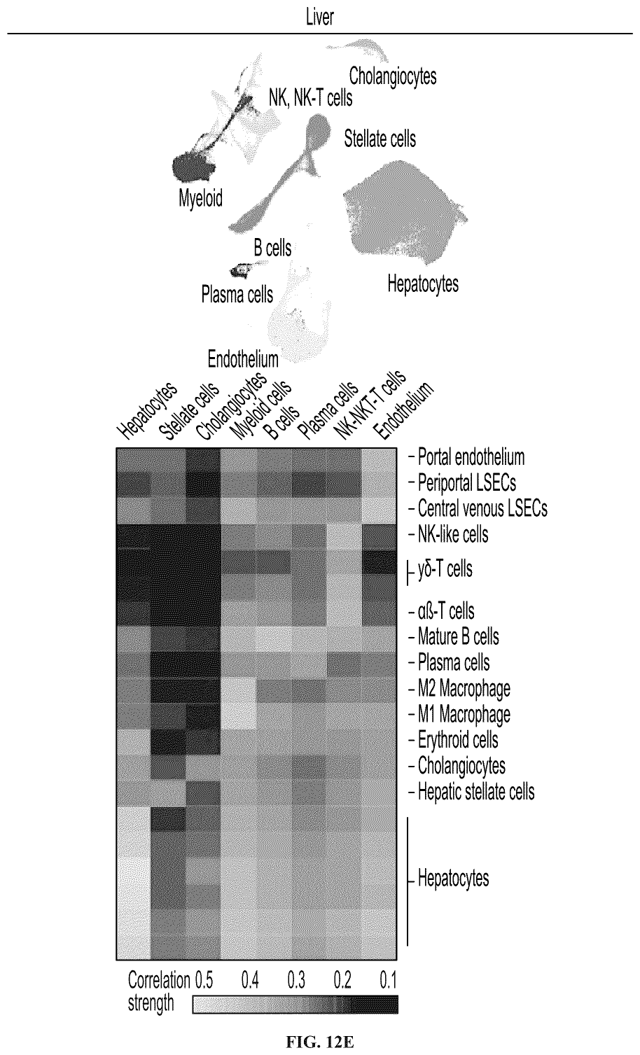

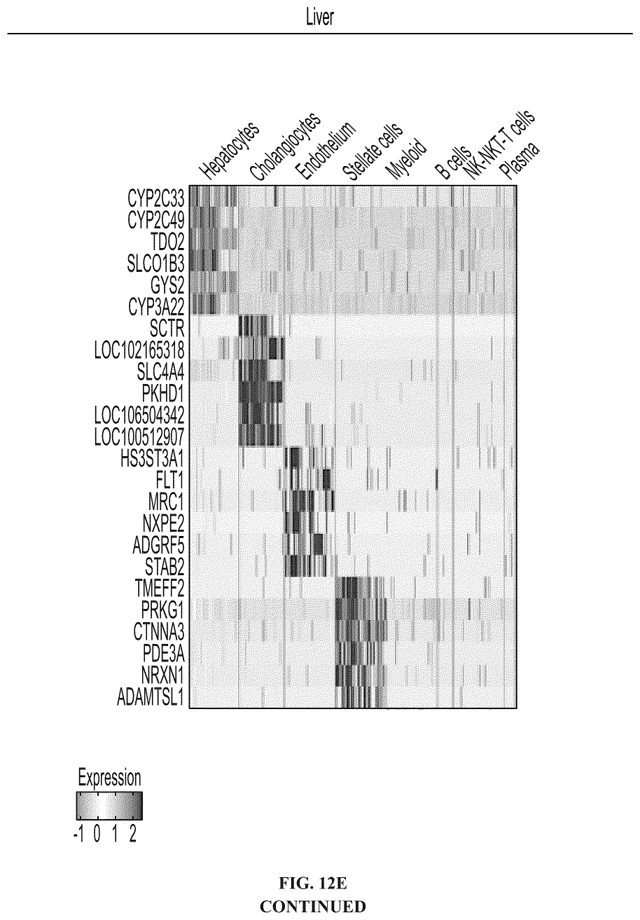

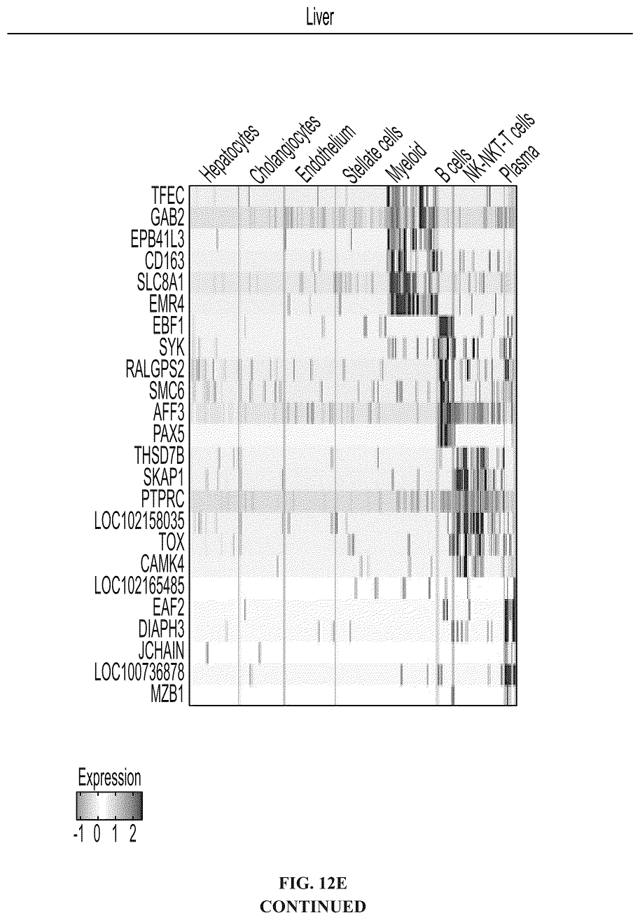

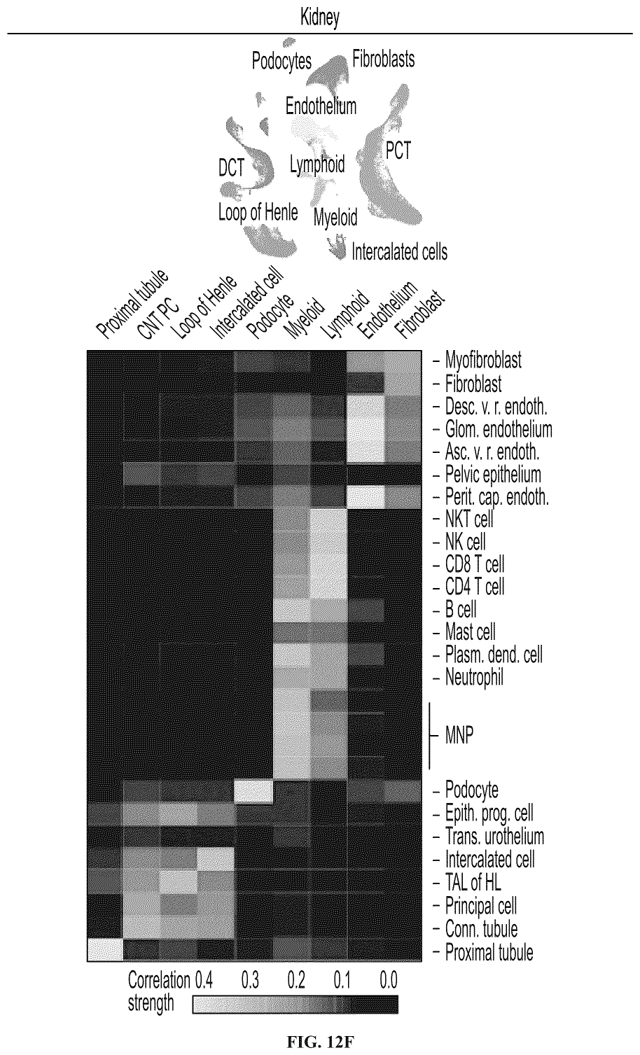

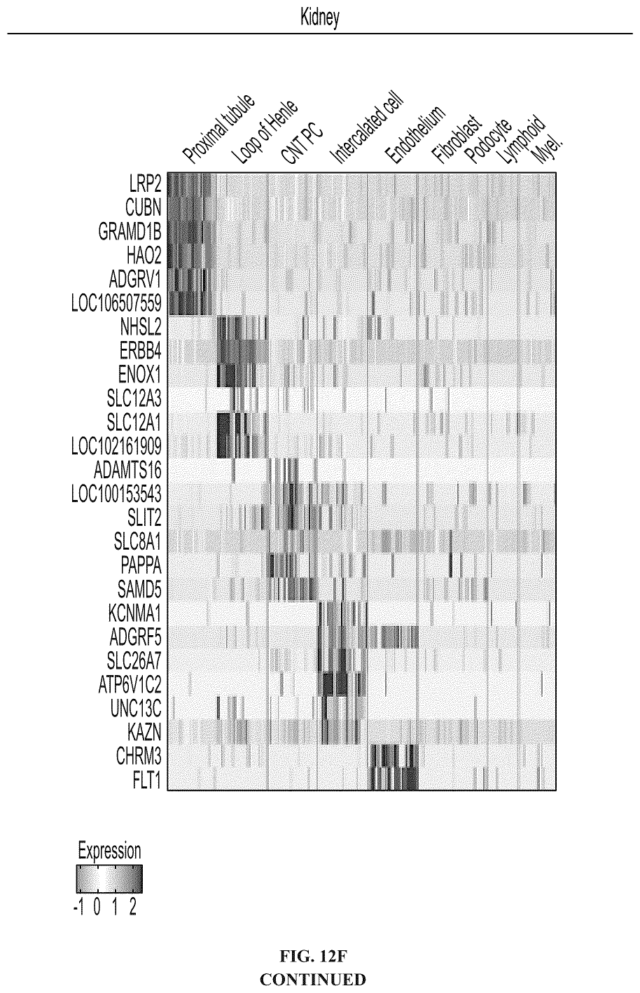

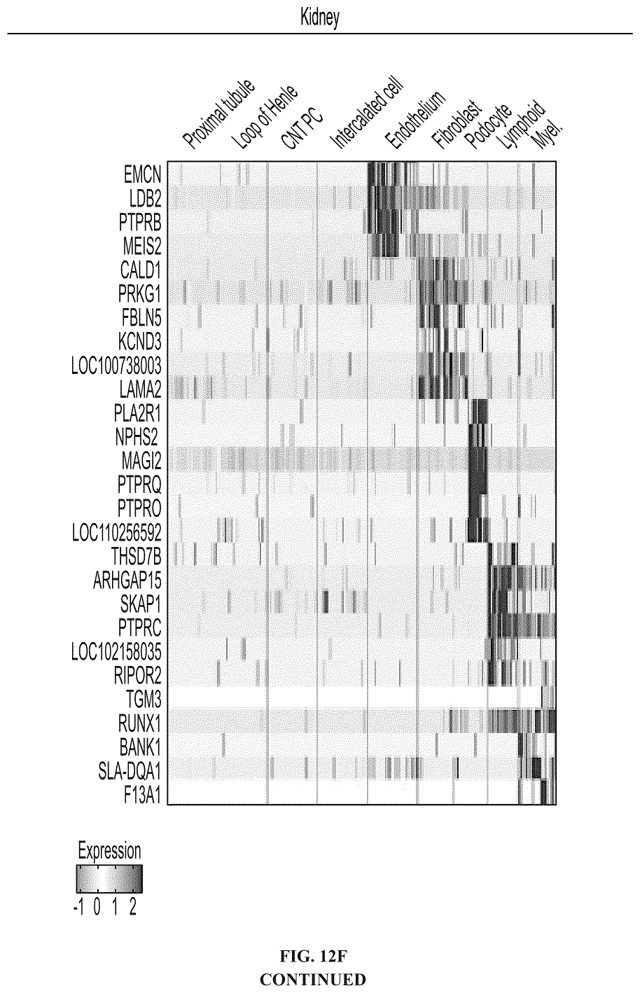

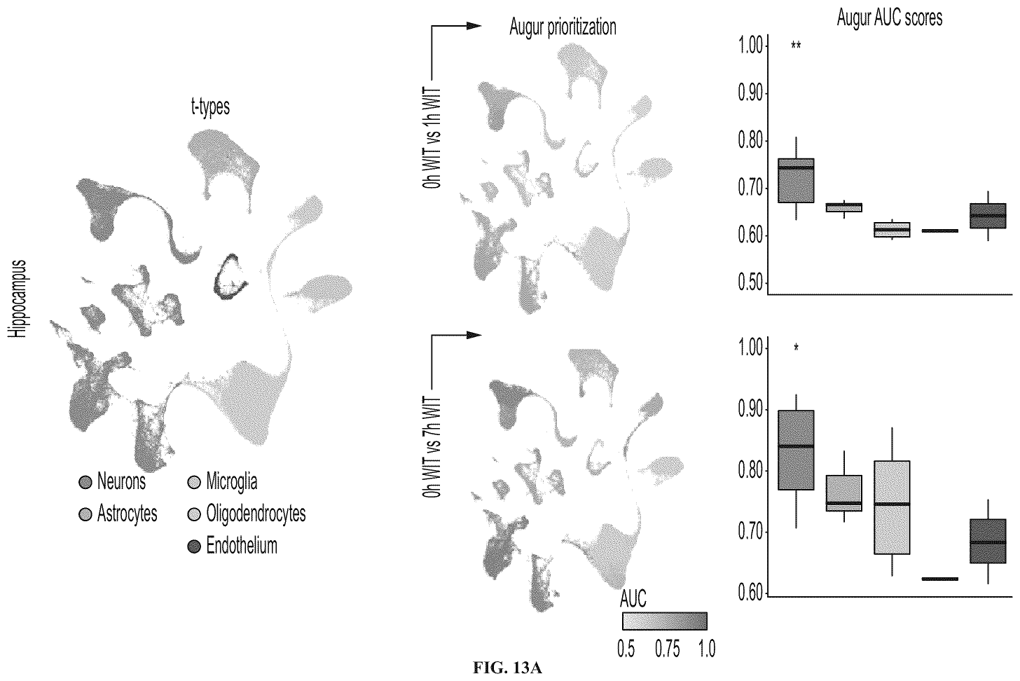

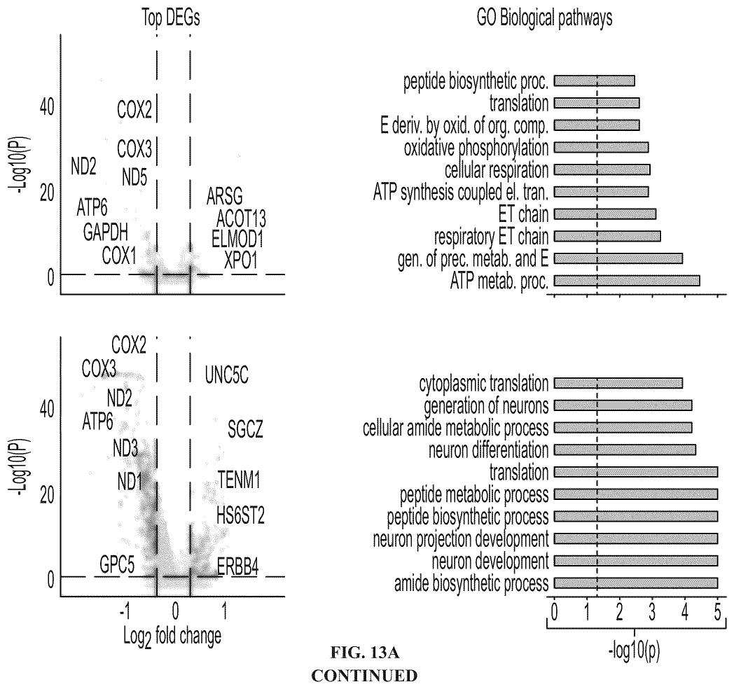

To validate the cluster annotation of the data, global transcriptomic comparison with public reference datasets for the four organs was performed. For each organ, the log-transformed average expression for each cell clusters was calculated followed by performing pairwise Pearson correlation between the clusters in the present dataset and the corresponding dataset using the highly variable genes. The resulted correlation coefficients were visualized on gradient heatmaps (FIGS. 12C-12F).

### Cell Type Annotations.

The cell type classification for hippocampus data (FIG. 12C) was based on the gene markers derived from recent data in adult human hippocampus and entorhinal cortex. The cells were initially classified into several major groups based on marker gene expression: excitatory neurons (SLCI7A7+), inhibitory neurons (GAD1+), oligodendrocyte progenitor cells (PDGFRA+), oligodendrocytes (PLP1+), astrocytes (AQP4+), microglia (PTPRC+), vascular cells (COL1A1+). Because of the high heterogeneity present in excitatory and inhibitory neuron populations, these two populations were further subclustered. For excitatory neurons, they were classified to mature granule cells (PROX1+), mossy cells (ADCYAP1+), CA2-4 excitatory neurons (FREM1+/GALNT3+), CA1 and subiculum excitatory neurons (SATB2+/BCL11B+/TLE−), entorhinal cortex upper layer (CUX2+) and deep layer (TLE4+) excitatory neurons. For inhibitory neurons, they were classified based on their developmental origins, either derived from medial ganglionic eminence (MGE, LHX6+) and caudal ganglionic eminence (CGE, ARADB2+). For a small group of cells connecting granule cells on the UMAP that are devoid of all these markers but marked by DCX and CALB2 expression, they were annotated as neuroblast cells, intermediate cell populations in pig adult neurogenesis.

The heart data (FIG. 12D) was annotated based on the gene markers derived from recent data in adult human heart. We classified the cells based on marker expression: cardiomyocyte (MYH7+/FHL2+), immune cells (PTPRC+), pericytes (RGS5+/ABCC9+), smooth muscle cells (MYH11+/ACTA2+), endothelial cells (CDH5+/PECAM1+), fibroblast-like cells (DCN+/GSN+) and neuronal cells (NRX7N+/XKR4+). The endothelial cells have two subgroups that have differential expression of VWF and TBXJ. Immune cells were further classified to myeloid cells (BANK1+/C1QA+) and lymphoid cells (SKAP1+/CD8A+).

The kidney data (FIG. 12E) was annotated based on the gene markers derived from recent data in adult human kidney. We classified the cells based on marker expression: proximal tubule (CUBN+/LRP2+), connecting tubule and principal cells (SAMD5+/LINGO2+), loop of Henle (NHSL2+/UMOD2+), intercalated cells (HEPACAM2+/SLC26A7+), podocyte (PTPRQ+/PTPRO+), immune cells (PTPRC+), endothelium (CHRM3+/PTPRB+) and fibroblasts (PRKGI+/FBLN5+). Immune cells were further classified to myeloid cells (BANK1+/MARCHJ+) and lymphoid cells (SKAP1+/THSD7B+).

The liver data (FIG. 12F) was annotated based on the gene markers derived from recent data in adult mouse liver. The cells were classified based on marker expression: hepatocytes (APOB+/PCK1+), stellate cells (RELN+/ACTA2+), cholangiocytes (CFTR+/PKHDI+), immune cells (PTPRC+), endothelial cells (FLT1+/PECAM1+). The immune cells were further classified to multiple subgroups: B cells (MS4A1+), plasma cells (JCHAIN+/MZBI+), natural killer cell and T cells (SKAP1+), myeloid cells (CD163+/EMR4+, which are predominantly Kupffer cells).

### Heterogeneity of Cells.

FindMarkers function from Seurat was used to determine marker genes for high resolution clusters. P-value adjustment is performed using Bonferroni correction with the cutoff set at 0.05. Top 10 genes for each cluster were ranked by fold changes and were visualized on a heatmap by using DoHeatmap function (Seurat). The dataset was randomly sampled to have 1000 cells per condition for each t-type prior to differential expression analysis.

### Cell Type Prioritization Using Augur

In order to find the cell populations that exhibit high degree of transcriptomic changes, Augur was applied to prioritize the cell types between each pair of conditions. Since there are three samples per condition, the Augur analysis was performed on all of the nine sample pairs in each condition pair using the high-resolution cell clusters identified via Seurat. The median of the calculated area under curve (AUC) scores of each cluster were then visualized on the UMAP layout. Comparison of the AUC scores for each specific cell type and a given condition pair were done by comparing the specific cell type of interest AUC score and AUC scores of all the other cell types in that given condition pair by using Wilcoxon Rank Sum test (one tailed).

### Differential Expression Analysis and Gene Ontology Analysis

In order to find differentially expressed genes (DEGs) between OrganEx and other conditions (0h PMI, 1h PMI, 7h PMI, ECMO) in major cell-types for each organ Seurat “FindMarkers” function was used. DEGs were defined at cut-off criteria of adjusted P-value (Bonferroni)<0.05, expression ratio greater than 0.1 in one condition and average log 2 fold change (log 2FC) greater than 0.2 in the same condition. Top 15 DEGs ranked by absolute values of log 2FC, were visualized using Bioconductor EnhancedVolcano package. Top 100 DEGs were used for HumanBase Functional Module Detection. Gene symbols starting with ma and LOC were excluded since HumanBase Functional Module Detection does not recognize those gene symbols. Identifying enriched biological pathways between OrganEx and all other groups in major cell-types for each organ, was performed using “enrichGO” function from clusterProfiler. Multiple testing was adjusted by false discovery rate (FDR) with the cutoff set at 0.2. Top 15 biological processes ranked by P-value were visualized by in-house made ggplot2 script.

### Gene Set Enrichment Analysis

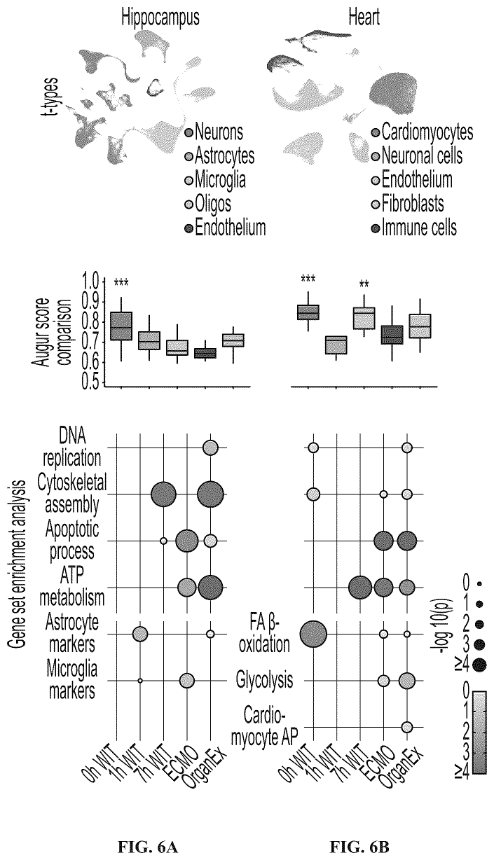

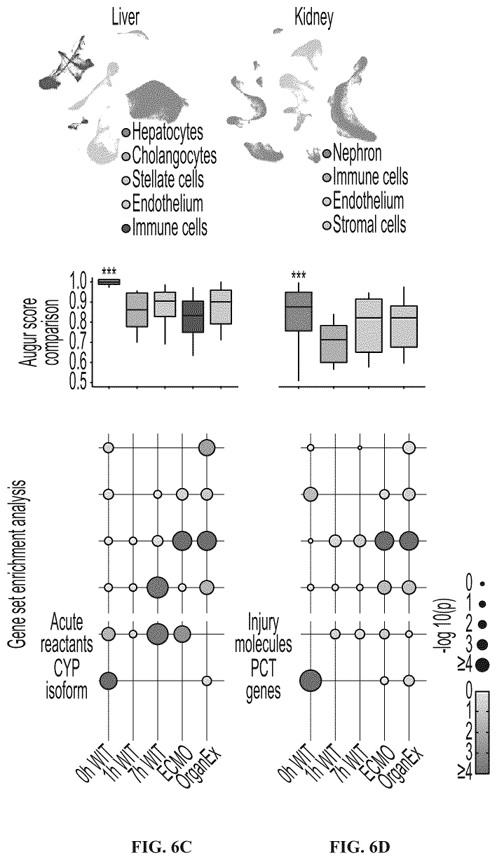

In order to assess whether gene sets of interest are upregulated in a specific condition (e.g., 0h WIT, 1h WIT, 7h WIT, ECMO and OrganEx), gene set enrichment analysis was performed in hippocampal neurons, astrocytes and microglial cells, cardiomyocytes in the heart, hepatocytes in the liver and proximal convoluted tubule cells in the kidney. This method is commonly used in Gene Ontology enrichment analysis and has been widely applied in multiple published studies. Specifically, all the expressed genes (expressed in at least one cell) were set as the gene universe and considered each set of condition-enriched genes as a sampling from the gene universe. The gene set enrichment, performed by Hypergeometric test (also named one-tailed Fisher's Exact test), is an assessment of whether genes from a given gene sets are overrepresented in condition-enriched genes than drawing from the gene universe by chance. To identify condition-enriched genes in the above-mentioned t-types, differential expression (DE) analysis was performed using Seurat FindMarkers function. In brief, one condition group was taken, its expression profiles were compared with the rest of the conditions using Wilcoxon Rank Sum test. For any given comparison, genes with false discovery rate (FDR) smaller than 0.01 were considered statistically significant and were kept. Because Wilcoxon Rank Sum test can be biased by the differences of cell numbers, that is, more cells lead to more differentially expressed genes, the datasets were randomly sampled to have 1000 cells per condition in each t-type prior to differential expression. With the condition-enriched genes and certain selected gene sets downloaded from GeneOntology (http://geneontology.org/), it was possible to assess the significance of gene set enrichment in a given condition. P-value of less than 0.05 was considered significant. Significance of the enrichment was visualized in a dot plot where size and color of the dot shows significance as −log 10(p-value). As shown in the FIG. 6A-6I), enrichment in all conditions in all organs was tested for positive regulation of DNA repair (GO:0045739), negative regulation of apoptotic process (GO:0043066), positive regulation of cytoskeleton organization (GO:0051495) and ATP metabolic process (GO:0046034). The same approach was used in assessment of functional enrichment analysis for each organ. Therefore, in hippocampus microglial cells were tested in enrichment for pro-inflammatory markers, and in astrocytes for pan-reactive markers as shown in FIG. 6A. Cardiomyocytes in heart were tested for Cardiac Muscle Cell Action Potential (GO:0086001), Fatty Acid Beta-Oxidation (GO:0006635) and Glycolysis (GO:0006096) as can be seen in FIG. 6B. In the liver, Hepatocytes enrichment of acute phase reactants and all expressed CYP isoforms (FIG. 6C) were tested in, and at last, proximal tubule cells in the kidney were tested for injury molecule genes and PCT transporter genes (FIG. 6D).

### Gene Set Expression Enrichment Analysis.

The expression dynamics of selected cell death-related gene set across conditions was also evaluated. For each gene of the given gene set, its expression enrichment was tested in each condition by comparing its expression in the given condition to that of other conditions. Similarly, Wilcoxon Rank Sum test was used to measure the significance. The resulted log-transformed P-values (−log 10[P-value]) across conditions were visualized in a bar plot.

### Hierarchical Clustering for Top DEGs.

To acquire the dynamic changes of transcriptome among different conditions and time points for each tissue, the top differential expressed genes for all the paired conditions were identified: 0h vs 1h, 0h vs 7h, 0h vs ECMO, 0h vs OrganEx, 1h vs 7h, 1h vs ECMO, 1h vs OrganEx, 7h vs ECMO, 7h vs OrganEx, and ECMO vs OrganEx using FindMarkers( ) function in Seurat. For hippocampus, significant DEG was selected with average log 2FC greater than 0.5 or less than −0.5. The top 50 upregulated genes were merged with 50 downregulated differentially expressed genes (DEGs, in total 100 genes) from each paired condition for the following analysis. To identify gene expression patterns, the average expression of the merged DEGs across t-types and experimental groups was calculated and the correlation coefficients subtracted from 1 as gene-gene distances was defined, which was passed to hierarchical clustering using the hclust function in R with ward.D2 algorithm. To define gene modules (clusters), the genes were parcellated using the cutreeDynamic function from R WGCNA package with a setting minClusterSize=45, sensitivity=2. The scaled eigengenes were the plotted, the first principal component of the expression matrix of each module to show the expression trend of the genes in each module. Using TopGO, the gene ontology analysis was performed for the genes in each module, and used fisher's exact test to calculate the P values. For heart, kidney and liver, the same methods with hippocampal data to select the genes were used. To keep the numbers of selected gene are comparable with hippocampus samples, significantly DEG with average log 2FC greater than 0.75 or less than −0.75 in heart data, significantly DEG with average log 2FC greater than 1.00 or less than −1.00 in kidney data and selected significantly DEG with average log 2FC greater than 1.75 or less than −1.75 in liver data. Then the same setting was used to build the gene expression network using hierarchical clustering. The scaled eigengenes of each module was plotted and used the same method (TopGO) for gene ontology analysis.

### Evaluation of the Perfusate Components Effects.

To evaluate the effects of the perfusate components on OrganEx, the expression enrichment of the related pathways (cell death, inflammation, and oxidative response) between OrganEx and ECMO conditions (Tables 4, 5, and 6) were compared. Specifically, the AUC (area under curve) scores were calculated using the AUCell package and a one-sided Wilcoxon Rank-Sum test was performed to evaluate the significance of the pathway enrichment.

### Trajectory Analysis.

The monocle2 was used and in-house R scripts to conduct pseudotime analysis for hippocampus, heart, liver, and kidney. The recommended analysis protocol was followed, except using FindMarkers function from Seurat package to perform pairwise comparison across different conditions to find the statistically significant up- and down-regulated genes. To reduce false positive results, some parameters were customized based on computational permutation. For examples, it was required that minimum percentage of expressed cells for each gene in either condition is larger than 0.1, and fold change larger than 1.25. Maximum number of cells in either condition was down sampled to 1000 cells to balance the comparison. Consequently, the identified differentially expressed genes by Seurat were used as the informative genes to order cells using setOrderingFilter function from monocle2, and the advanced nonlinear reconstruction algorithm called DDRTree was chosen to execute data dimensional reduction.

### Cell-Cell Communication Analysis.

Cell-cell interactions based on the expression of known ligand-receptor pairs in different t-types were inferred using CellChat (v.1.1.3). The official CellChat workflow was followed for analyzing multiple datasets (0h WIT, 1h WIT, 7h WIT, ECMO and OrganEx). each dataset was first randomly down sampled to 1000 cells per t-type to to balance the comparison. Next, normalized counts were loaded into CellChat. After that, CellChatDB human database was selected for cell-cell communication analysis. The preprocessing was then applied as functions identifyOverExpressedGenes and identifyOverExpressedInteractions with standard parameters set. Next, communication probability was computed between interacting cell groups with truncated mean set at 0.1. After that filterCommunication, computeCommunProbPathway, and aggregateNet were applied using standard parameters. To identify conserved and context-specific signaling pathways rankNet function was applied on the netP data slot which showed in a stacked bar plot overall information flow of each signaling pathway. To determine strength of the reactions and t-types involved in each signaling pathway, netAnalysis_signalingRole_heatmap was performed and visualized overall signaling by aggregating outgoing and incoming signaling together.

### Pseudotime Analysis

The monocle2 and in-house R scripts were used to conduct pseudotime analysis for all major organs, including hippocampus, heart, liver, and kidney. The recommended analysis protocol was followed, except using FindMarkers function from Seurat package to perform pairwise comparison across different conditions to find the statistically significant up- and down-regulated genes. To reduce false positive results, some parameters were customized based on computational permutation. For examples, it was required that minimum percentage of expressed cells for each gene in either condition is larger than 0.1, and fold change larger than 1.25. Maximum number of cells in either condition was down sampled to 1000 cells to balance the comparison. Consequently, the identified differentially expressed genes by Seurat were used as the informative genes to order cells using setOrderingFilter function from monocle2, and the advanced nonlinear reconstruction algorithm called DDRTree was chosen to execute data dimensional reduction.

### Tissue Processing and Histology

### Tissue Preparation

Following the completion of each experimental protocol brain, heart, lungs, kidney, liver, and pancreas were extracted and immersion-fixed in a solution containing 10% (w/v) neutral buffered formalin with gentle shaking. After fixation each tissue piece was processed and embedded into a paraffin block using the Excelsior tissue processor (Thermo Scientific, Waltham, MA).

Tissue Staining with Hematoxylin and Eosin (H&E) and TUNEL Assay

Heart, lungs, kidney, liver and pancreas paraffin blocks were trimmed on the Shandon Finesse 325 microtome (Thermo Scientific) to 5 μm sections. Sections were mounted on TruBond 380 adhesive slides and allowed to dry overnight at room temperature. All slide were then stained simultaneously for H&E using the automatic Shandon Linistain slide stainer (Thermo Scientific).

For Nissl staining, brain sections (hippocampus and prefrontal cortex) were stained with 0.1% cresyl violet solution (Abcam, ab246816) for 5 min and were rinsed quickly in 1 change of distilled water. Then these sections were dehydrated quickly in absolute alcohol and later cleared in Histo-Clear II and finally cover-slipped with Prolong Gold Antifade Mountant (ThermoFisher, P36934).

All slides for the TUNEL Assay (Millipore, S7101, 3542625) were processed simultaneously. They were fist deparaffinized, rehydrated and incubated with proteinase K (20 g/mL in PBS) for 30 min at 37° C. Slides were rinsed with PBS and incubated with 3% H2O2 in PBS for 10 min at room temperature to block endogenous peroxidase activity, followed by PBS washing and incubation in 0.1% Triton X-100 in 0.1% sodium citrate for 2 min on ice (4° C.). Sections were incubated with a mixture of TdT solution and fluorescein isothiocyanate dUTP solution in a humidified chamber at 37° C. for 60 min. This was followed by washings with PBS and incubation with antifluorescein antibody Fab fragments conjugated with horseradish peroxidase in a humidified chamber at 37° C. for 30 min. After washing with PBS, methyl green counterstain was applied to stain for nuclei.

### Immunohistochemistry

All slides used for a specific immunohistochemistry staining were processed simultaneously. Slides containing formalin-fixed paraffin-embedded histological sections were first deparaffinized in 2 changes of Histo-Clear II (64111-04, Electron Microscopy Sciences, Hatfield, PA) for 10 minutes each. Slides were then transferred to 100% alcohol, for two changes, 10 minutes each, and then transferred once through 95%, 70%, and 50% alcohol respectively for 5 minutes each. Slides were then rinsed in water and washed in wash buffer (0.05% Tween 20 in 1×PBS) for 10 minutes. Slides were then placed into a chamber filled with antigen retrieval buffer (lOX R-Buffer A diluted to 1× by water, pH 6, 62706-10, Electron Microscopy Sciences). Slides then underwent heat-mediated antigen retrieval in the Unique Retriever system (Electron Microscopy Sciences). After antigen retrieval, slides were washed in wash buffer and blocked for 1 hour in 10% normal goat serum diluted in wash buffer at room temperature. Slides were then incubated in primary antibodies at 4 degrees C. overnight at the following dilutions: rabbit anti-NeuN (1/1000, Abcam, ab177487, GR3275112-10), mouse anti-GFAP (1/1000, Sigma-Aldrich, G3893-100UL, 0000082460), rabbit anti-Iba1 (1/500, Wako, 019-19741, PTR2404), mouse anti-albumin (1/500, Abcam, 4A1C11, GR3215248-15), mouse anti-beta actin (1/500, Invitrogen, AC-15, 01003256), rabbit cleaved caspase-3 (1/50, R&D Systems, MAB835, KHK0821021). The next day, slides were washed and then incubated with fluorescently tagged secondary anti-rabbit (Cell Signaling Technology, 8889S, 12) and anti-mouse (Abcam, ab150113, GR3370569-1) antibodies at a dilution of 1/500 for 1 hour at room temperature. After staining with secondary antibodies, slides were washed and incubated in DAPI (1/1000 for 5 minutes) at room temperature. Finally, slides were washed and mounted with coverslips using Prolong Gold Antifade Mountant (P36934, ThermoFisher).

### Microscopy and Image Processing

Tissue sections were imaged using an LSM880 confocal microscope (Zeiss; Jena, Germany) equipped with a motorized stage using 10× (0.3 NA) or 20× (0.8 NA) objective lenses with identical settings across all experimental conditions. Lasers used: argon 458, 488, and 514; diode 405; and DPSS 561-10. The DPSS 561-10 laser intensity was increased during imaging of the control perfusate samples for the intravascular hemoglobin fluorescence study in order to obtain a background signal comparable to other groups. Images were acquired at either 1,024×1,024 or 2,048×2,048-pixel resolution. Images are either representative confocal tile scans, high-magnification maximum intensity Z-stack projections (approximately 7-9-μm stacks; −1 μm per Z-step), or high magnification confocal images. Alternatively, histological images were acquired using an Aperio CS2 Pathology Slide Scanner (Leica; Wetzlar, Germany) as described above. Image adjustments were uniformly applied to all experimental conditions in Zeiss Zen. Digitized images were assembled in Zeiss Zen, ImageScope, and Adobe Illustrator.

### Histological Data Analysis and Quantification

### H&E Staining—Pathology Injury Score

All H&E slides were scanned. Four images were randomly selected from each slide and from the corresponding areas. Blinded observers scored each image accordingly. Criteria for heart were nuclear damage, myocyte vacuolization, widened spaces between myofibers and edema. Criteria for lungs were nuclear damage, pneumocyte vacuolization and hemorrhage. Criteria for kidney were nuclear damage, tubule vacuolization, hemorrhage, and tubule damage. Criteria for liver were nuclear damage, tissue vacuolization, hepatocyte vacuolization, and congestion. Criteria for pancreas were nuclear damage, cell vacuolization, hemorrhage, edema.

### Cresyl Violet (Nissl) Staining Injury Score.

Two images were taken per region of interest (hippocampal CA1 region or PFC) and were evaluated by blinded observer using the cell counter function in ImageJ according to already established criteria.

### Kidney Periodic Acid-Schiff (PAS) Staining—Pathology Injury Score.

All kidney PAS-stained slides were examined by blinded kidney pathologist. Score (0-3) was assigned depending on the severity of the damage which included: Bowman space dilation, tubular dilation, tubular vacuolization, brush border disruption and presence of the casts.

### Brain Immunofluorescence Cell Analysis and Quantification

All images were normalized to the regions of interests (hippocampal CA1, CA3 and DG) using ImageJ and randomized. The number of labeled astrocytes (GFAP+ cells), microglia (IBA1+ cells) cells were quantified manually by a blinded observer using the cell counter plugin and averaged based on the acquired area/cells. The particle analysis of astrocytes was performed using a custom pipeline in the open-source software, CellProfiler56, where a uniform threshold was set on GFAP to identify GFAP+ skeleton objects. The number of GFAP+ fragments in each area were then auto-counted and averaged according to the number of the astrocytes. The mean intensity of NeuN per neuron were quantified by a custom pipeline where neurons were identified based on the DAPI segmented objects expressing NeuN. Data were analyzed and plotted in GraphPad Prism9 software. All data are shown as mean±SEM. “n” refers to the number of biological replicates.

### Kidney, Liver, and Heart Immunofluorescence Analysis.

In the kidney, three images were taken randomly of the kidney cortex. All images were randomized and analyzed by a blinded observer. Glomerulus and the proximal convoluted tubule were manually selected as the regions of interest in the four same-size circles respectively in corresponding regions across images. The mean intensity of ACTB and TIMI was calculated using ImageJ software. The number of Ki-67 positive cells were manually counted.

In the liver, immunolabelling quantification was done the same for albumin and factor V. Three images were randomly taken from the same region of the liver. The mean intensity of albumin and factor V was automatically quantified using ImageJ across the whole slide.

In the heart, three images were taken randomly from the left ventricle just slightly below the epicardium. All images were randomized, after which a blinded observer selected cardiac muscle tissue as a region of interest. The amount of cytoplasmic troponin I was then quantified using mean intensity function in ImageJ.

Cell Death Pathway Quantification and Analysis (actCASP3, IL1B, RIPK3, and GPX4).

For kidney, liver, heart, and pancreas, three images were taken randomly from each slide and from the corresponding areas of each organ. actCASP3+ cell intensity was quantified manually by a blinded observer using ImageJ. Mean immunolabelling intensity of IL1B, RIPK3 and GPX4 in kidney, liver and heart was quantified using ImageJ with background subtraction and measure functions. In the brain, the number of actCASP3+ and RIPK3+ cells were counted manually in the hippocampal CA1 region and prefrontal cortex. Expression of ILIB and GPX4 in hippocampal CA1 was measured in cells that were manually selected in the granule cell layer using ImageJ measure function.

### TUNEL Quantification and Analysis.

All images were taken with a bright field microscope followed by a random same-size snapshots of the slides. A blinded observer analyzed randomized images using Fiji to separate channels of nuclei staining (hematoxylin) and DNA fragments (DAB). Total DNA fragments were quantified based on the total DAB intensity using CellProfiler.

### Functional Organ Assessments

### Heart Contractility Measurements

Small piece of the apical portion of the hearts' left ventricle was excised and placed in the carbonated Tyrode's solution (140 mM NaCl, 6 mM KCl, 10 mM glucose, 10 mM HEPES, 1 mM MgCl2, 1.8 mM CaCl₂), pH=7.4). The tissue was then dissected into 1-2 cm3 cubes and placed on a holder with the epicardium facing down so the cutting align with the cardiac myofibril orientation. Using a vibratome, the heart was sliced at 150 μm thickness at 4° C. After slicing, spontaneous rate and rhythm of heart contraction were characterized and recorded using a slice microscope with temperature control set to 37° C. (Scientifica SliceScope, Uckfield, UK). Beating frequency was quantified within the time course of 30 seconds.

### Glucose Assays

At the end of each perfusion, two identical tissue samples with 5 mm length were acquired from each organ (kidney, heart, and cerebral cortex) using a 3 mm biopsy punch (Miltex, Integra, York, PA) and were placed in cold PBS (fresh) and PFA (fix overnight) solutions respectively. The fresh tissues and the fixed controls were incubated in 2-NBDG working solution (Cayman 11046, 100 μM, Ann Arbor, MI) for 30 min at 37° C. followed by 15 min of wash in PBS. The images were acquired immediately using widefield microscope (Zeiss) under 2.5× objective (Ex: 465-495 nm, Em: 5210-560 nm) with exposure time of 295 ms (kidney and heart) and 50 ms (cerebral cortex). Images were analyzed using ImageJ to quantify the mean intensity of the ROI with normalization based on the fixed controls.

### Organotypic Hippocampus Culture and Nascent Protein Synthesis Assay

Hippocampus was isolated at the end of appropriate experimental protocol and sectioned with a vibratome at 250 μm thickness in carbonated NMDG-HEPES aCSF solution (92 mM NMDG, 2.5 mM KCl, 1.25 mM NaH2PO4, 30 mM NaHCO₃, 20 mM HEPES, 25 mM glucose, 2 mM thiourea, 5 mM Sodium ascorbate, 3 mM Sodium pyruvate, 0.5 mM CaCl2)·2H2O, and 10 mM MgSO4·7H2O, pH=7.3). Hippocampal slices were then collected and transferred onto culture membranes (Falcon culture insert, 0.4 m) and cultured in a six-well culture dish with 1.5 ml medium (48% DMEM/F-12 (Gibco), 48% Neurobasal (Gibco), 1× N−2 (Gibco), 1×B−27 (Gibco), 1× Glutamax (Gibco), 1×NEAA (Gibco), 1× Pen Strep (Gibco). The plates were maintained in an incubator at 37° C. with 5% CO2 for up to 2 weeks. The amino acid methionine analog, azidohomoalanine (AHA, ThermoFisher), was added to the hippocampal slice at a final concentration of 50 μM in HEPES buffered solution (HBS) followed by the incubation of 6 hours at 37° C. Slices were washed with PBS and immediately fixed in 4% paraformaldehyde at 4° C. overnight. Detection of nascent protein synthesis was performed with the modified Click-iT 1-azidohomoalanine (AHA) Alexa Fluor 488 Protein Synthesis HCS Assay kit (ThermoFisher). To visualize neurons simultaneously, the slices were then blocked with 5% NDS for 1 hour at room temperature, and incubated with Rabbit anti-NeuN antibody (abcam, 1:1000) overnight at 4° C. followed by the incubation of anti-rabbit AlexaFluor647 (1:500) for 1 hour at room temperature. The slices were then co-stained with DAPI to visualize nuclei. Images were acquired using Zeiss LSM800 confocal microscope with 20× objective. The combined Z-stack images of DAPI, AlexaFluor 488 and AlexaFluor 647 channels were acquired with optical slice thickness of around 20 μm and images of maximum projection are shown. Proteins detected around the nucleus (perinuclear) are the most abundant newly synthesized proteins in cells28, and were quantified using ImageJ intensity measurement function.

### Image Visualization

Depiction of the pig in FIG. 1A, and pig's head in FIG. 11A, were adapted from BioRender.com with postprocessing in Adobe Illustrator and Adobe Photoshop.

### Statistical Analysis and Reproducibility

All data are reported as mean±standard error of mean with data analysis being conducted using one-way ANOVA with Dunnett's post hoc multiple comparisons in reference to the OrganEx perfusion group, or unpaired t-test for comparisons between two groups. Fisher's exact test was used to compare occurrence of QRS complexes in OrganEx and ECMO groups during the perfusion protocol. For the number of replicates in each experimental group together with appropriate statistical analyses please see below. Significance was set at P<0.05. All statistical analysis and plotting were done in GraphPad 9 (GraphPad Software, San Diego, CA) or in Python. All figures were created using Adobe Illustrator (Adobe Systems, San Jose, CA).

### Further Information on Statistical Analyses

Further information regarding statistical values and reproducibility of the results is given below.

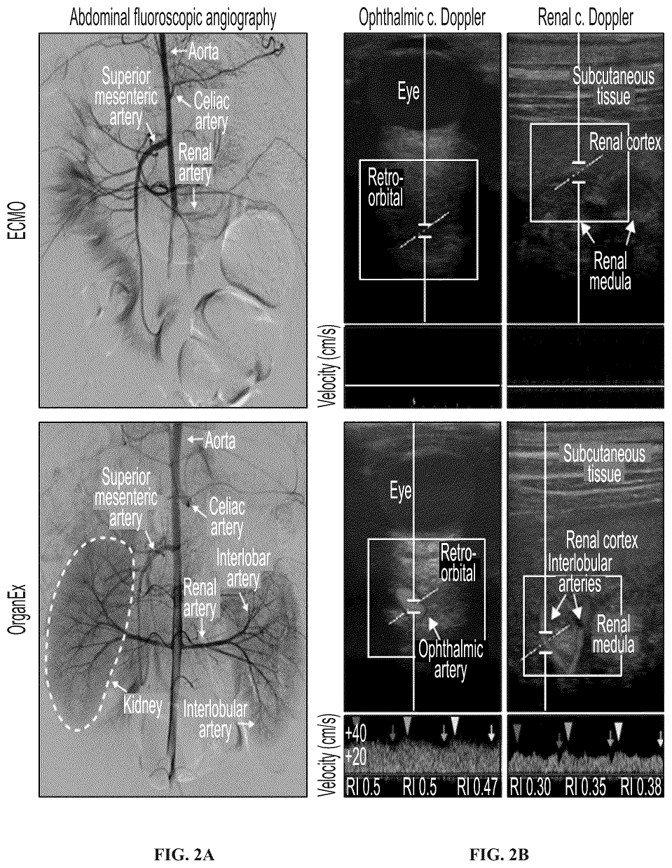

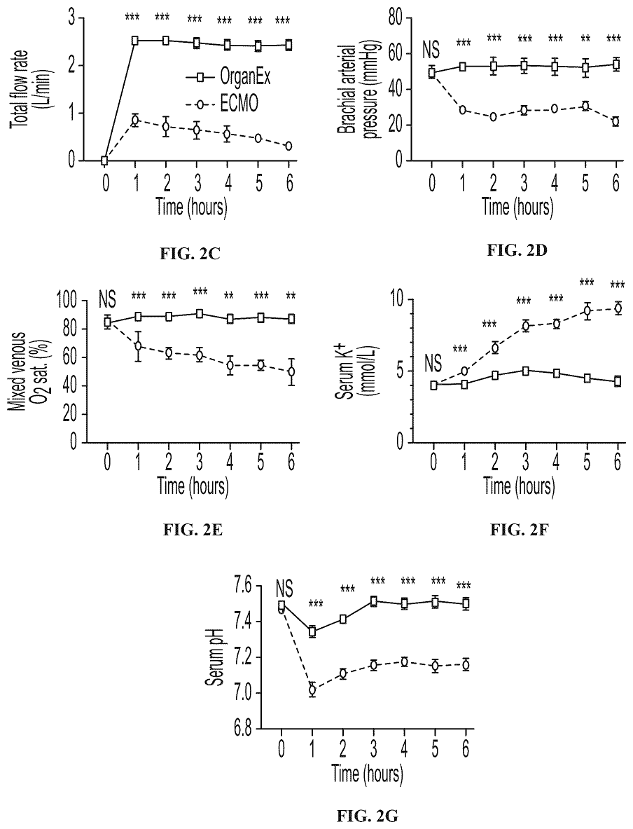

In FIGS. 2C-2E, the results are taken at the baseline (where applicable) and every hour throughout the perfusion experiment from ECMO and OrganEx perfusion groups. Each group consisted of n=6 biological replicates. In FIG. 2C, Total flow rate: P values for hours 1-6, 1h: <0.0001, 2h: <0.0001, 3h: <0.0001, 4h: <0.0001, 5h: <0.0001, 6h: <0.0001; t values for hours 1-6, 1h: 12.34, 2h: 8.120, 3h: 8.869, 4h: 9.683, 5h: 17.64, 6h: 22.96. Brachial arterial pressure: P values for hours 0-6, 0h: 0.9704, 1h: <0.0001, 2h: 0.0002, 3h: 0.0003, 4h: 0.0006, 5h: 0.0027, 6h: <0.0001; t values for hours 0-6, 0h: 0.03807, 1h: 12.24, 2h: 5.645, 3h: 5.480, 4h: 4.890, 5h: 3.953, 6h: 7.590. In FIG. 2D, Mixed venous 02 saturation: P values for hours 0-6, 0h: 0.8996, 1h: 0.0837, 2h: 0.0002, 3h: 0.0003, 4h: 0.0015, 5h: <0.0001, 6h: 0.0036; t values for hours 1-6, 0h: 0.1295, 1h: 1.920, 2h: 5.582, 3h: 5.453, 4h: 4.338, 5h: 7.691, 6h: 3.781. In FIG. 2E, Serum K⁺: P values for hours 0-6, 0h: 0.8365, 1h: 0.0001, 2h: 0.0008, 3h: <0.0001, 4h: <0.0001, 5h: <0.0001, 6h: <0.0001; t values for hours 0-6, 0h: 0.2118, 1h: 6.156, 2h: 4.707, 3h: 7.224, 4h: 12.24, 5h: 7.401, 6h: 9.358. Serum pH: P values for hours 0-6, 0h: 0.3489, 1h: <0.0001, 2h: <0.0001, 3h: <0.0001, 4h: <0.0001, 5h: <0.0001, 6h: <0.0001; t values for hours 0-6, 0h: 0.9827, 1h: 6.285, 2h: 9.256, 3h: 9.668, 4h: 8.427, 5h: 7.056, 6h: 7.034.

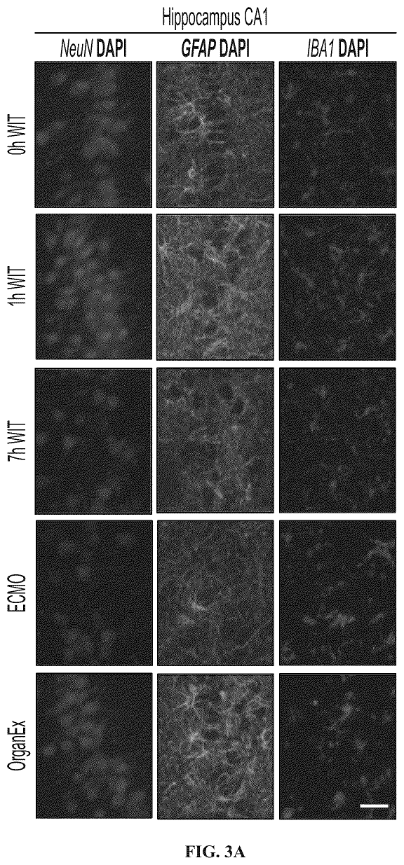

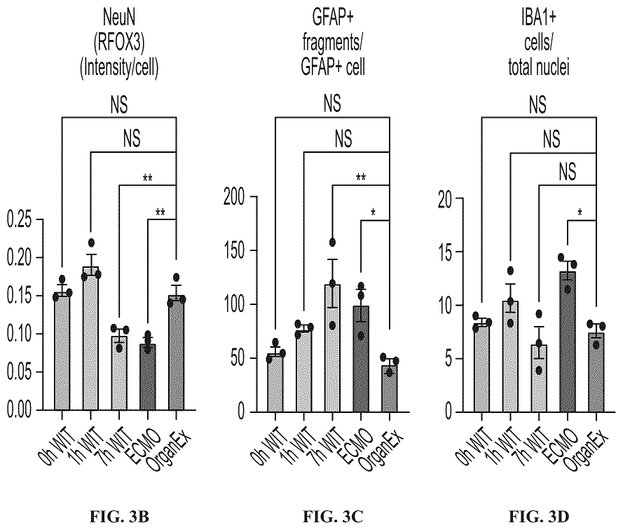

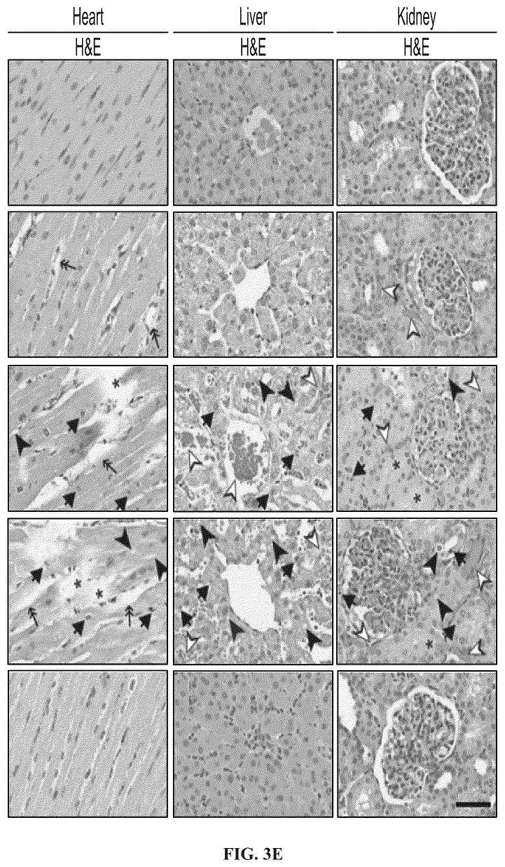

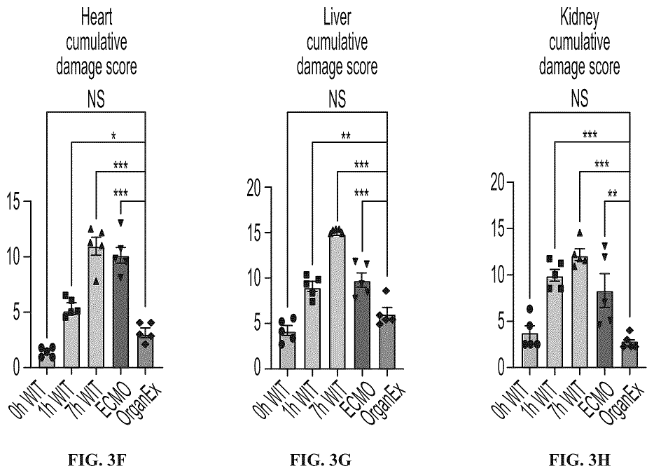

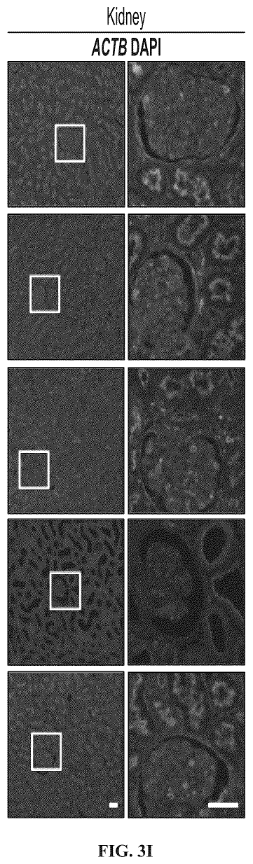

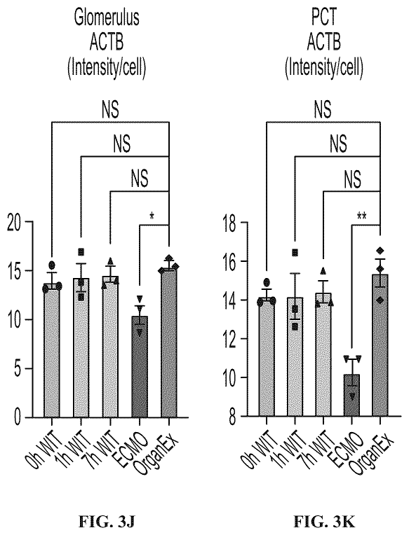

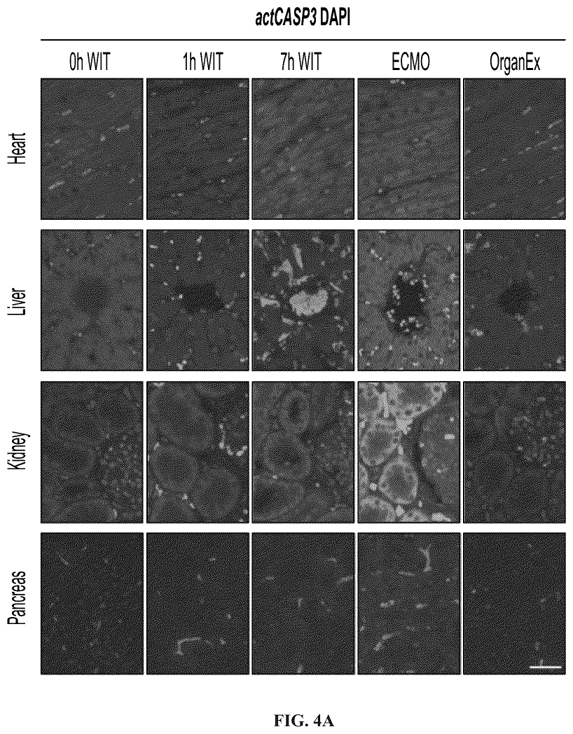

In FIGS. 3B-3D, data points are from a representative brain per condition; the experiment was repeated in n=3 independent brains per condition. In FIG. 3B, One-way ANOVA (P=0.0001, F[4, 10]=19.12) with post-hoc Dunnett's adjustment was performed; Dunnett's multiple comparisons test resulted in: OrganEx vs 7h WIT: P=0.009; OrganEx vs ECMO: P=0.003. In FIG. 3C, One-way ANOVA (P=0.0098, F[4, 10]=6.035) with post-hoc Dunnett's adjustment was performed; Dunnett's multiple comparisons test resulted in: OrganEx vs 7h WIT: P=0.0059; OrganEx vs ECMO: P=0.0356. In FIG. 3D One-way ANOVA (P=0.0076, F[4, 10]=6.495) with post-hoc Dunnett's adjustment was performed; Dunnett's multiple comparisons test resulted in: OrganEx vs ECMO: P=0.0109.

In FIGS. 3F-3H, data points are from a representative organ (heart, liver and kidney) per condition, the experiment was repeated in n=5 independent organs per condition. In FIG. 3F, One-way ANOVA (P<0.0001, F[4, 20]=52.16) with post-hoc Dunnett's adjustment was performed; Dunnett's multiple comparisons test resulted in: OrganEx vs 1h WIT: P=0.0429; OrganEx vs 7h WIT: P<0.0001; OrganEx vs ECMO: P<0.0001. In FIG. 3G, One-way ANOVA (P<0.0001, F[4,20]=50.79) with post-hoc Dunnett's adjustment was performed; Dunnett's multiple comparisons test resulted in: OrganEx vs 1h WIT: P=0.006; OrganEx vs 7h WIT: P<0.0001; OrganEx vs ECMO: P=0.0009. In FIG. 3g, One-way ANOVA (P<0.0001, F[4,20]=50.79) with post-hoc Dunnett's adjustment was performed; Dunnett's multiple comparisons test resulted in: OrganEx vs 1h WIT: P=0.006; OrganEx vs 7h WIT: P<0.0001. OrganEx vs ECMO: P=0.0009; In FIG. 3H, One-way ANOVA (P<0.0001, F[4,20]=17.79) with post-hoc Dunnett's adjustment was performed; Dunnett's multiple comparisons test resulted in: OrganEx vs 1h WIT: P=0.0001; OrganEx vs 7h WIT: P<0.0001; OrganEx vs ECMO: P=0.0022.

In FIGS. 3J-3K, data points are from a representative kidney per condition, the experiment was repeated in n=3 independent kidneys per condition In FIG. 3J, One-way ANOVA (P=0.0262, F[4,10]=4.398) with post-hoc Dunnett's adjustment was performed; Dunnett's multiple comparisons test resulted in: OrganEx vs ECMO: P=0.0103. In FIG. 3K, One-way ANOVA (P=0.0057, F[4,10]=7.082) with post-hoc Dunnett's adjustment was performed; Dunnett's multiple comparisons test resulted in: OrganEx vs ECMO: P=0.0023.

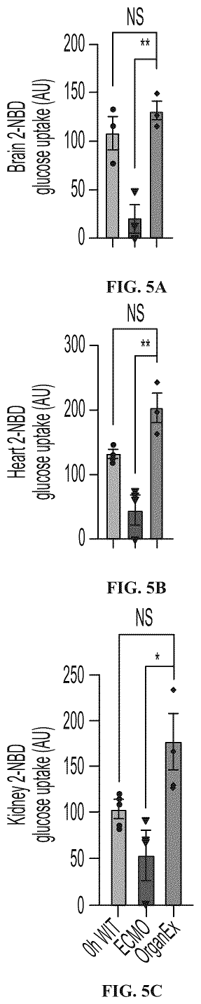

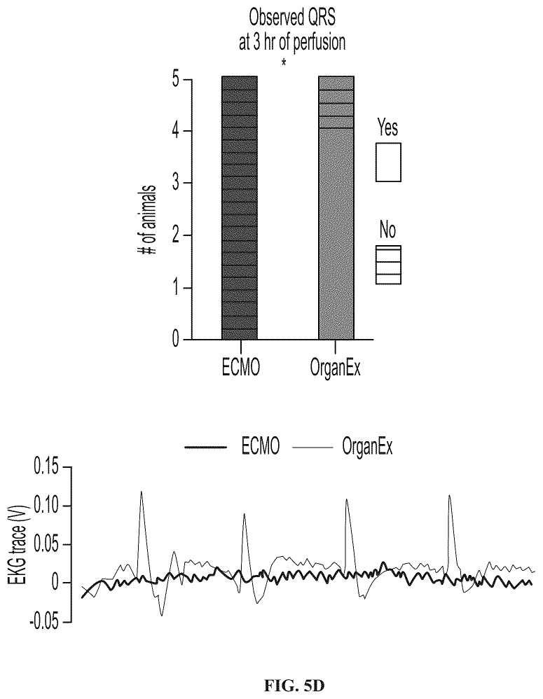

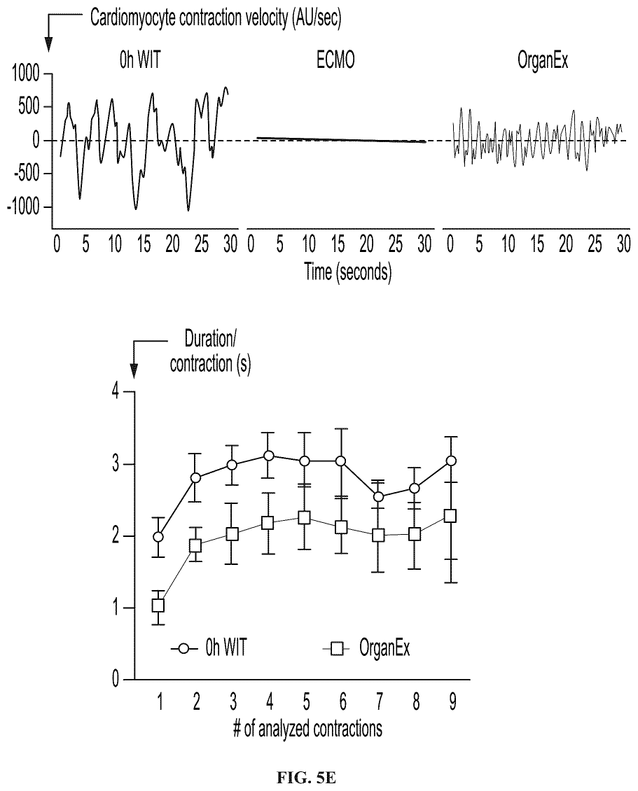

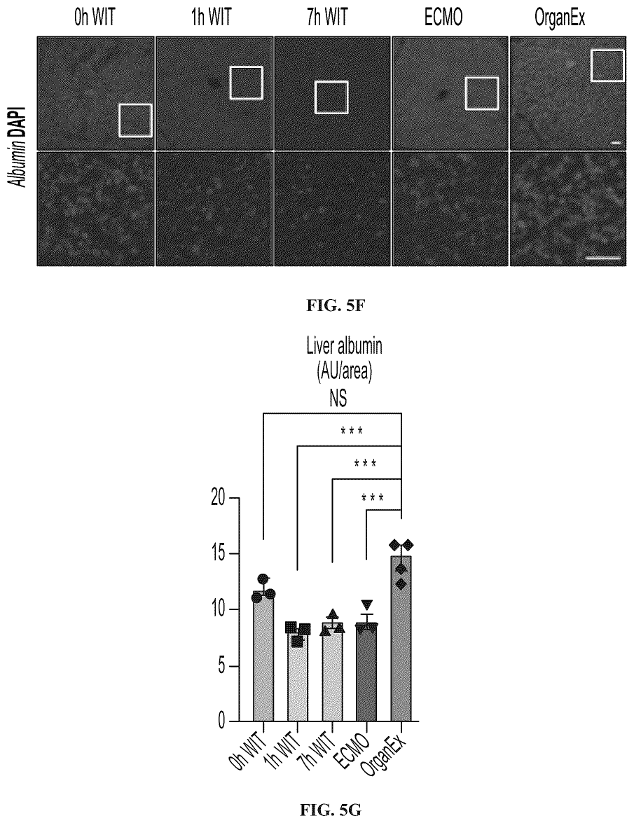

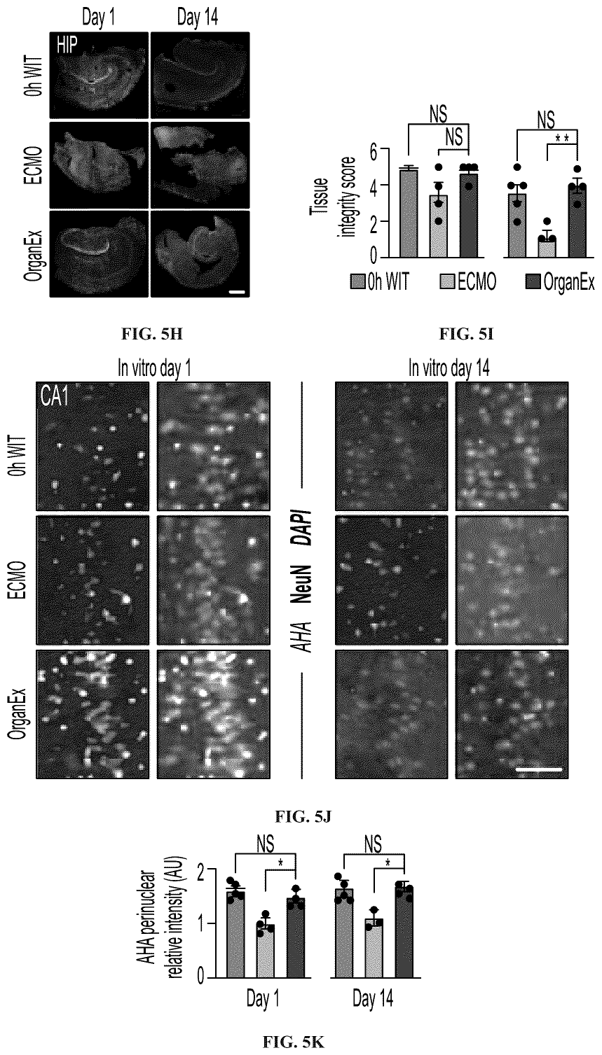

In FIGS. 5A-5C, data points are from a representative organ (brain, heart, and kidney) per condition, the experiment was repeated in n=3 independent organs per condition. In FIG. 5A, One-way ANOVA (P=0.003, F[2,6]=17,73) with post-hoc Dunnett's adjustment was performed; Dunnett's multiple comparisons test resulted in: OrganEx vs ECMO: P=0.0024. In FIG. 5B, One-way ANOVA (P=0.0033, F[2,6]=17,07) with post-hoc Dunnett's adjustment was performed; Dunnett's multiple comparisons test resulted in: OrganEx vs ECMO: P=0.0033. In FIG. 5C, One-way ANOVA (P=0.0033, F[2,6]=6.453) with post-hoc Dunnett's adjustment was performed; Dunnett's multiple comparisons test resulted in: OrganEx vs ECMO: P=0.0320.

In FIG. 5D, each data points are from representative perfusion experiment per condition, the experiment was repeated in n=5. Two-sided Fisher's exact t test was used: P=0.0476.

In FIG. 5F, each data point is from the representative liver per condition, the condition was repeated in n=3 times. One-way ANOVA (P<0.0001, F[4,10]=15.52) with post-hoc Dunnett's adjustment was performed; Dunnett's multiple comparisons test resulted in: OrganEx vs 1h WIT: P=0.0002; OrganEx vs 7h WIT: P=0.0005; OrganEx vs ECMO: P=0.0007.

In FIGS. 5H, 5J, each data point is from the representative hippocampal slice per condition, the experiment was repeated n=3-5 times per condition. In FIG. 5H, for day 1: One-way ANOVA (P=0.0352, F[2,10]=4.766) with post-hoc Dunnett's adjustment was performed; for day 14: One-way ANOVA (P=0.0027, F[2,10]=11.35) with post-hoc Dunnett's adjustment was performed, Dunnett's multiple comparisons test resulted in: OrganEx vs ECMO: P=0.0026. In FIG. 5J, for day 1: One-way ANOVA (P=0.0077, F[2,10]=8.246) with post-hoc Dunnett's adjustment was performed, Dunnett's multiple comparisons test resulted in: OrganEx vs ECMO: P=0.0216; for day 14: One-way ANOVA (P=0.0270, F[2,9]=5.544) with post-hoc Dunnett's adjustment was performed, Dunnett's multiple comparisons test resulted in: OrganEx vs ECMO: P=0.027.

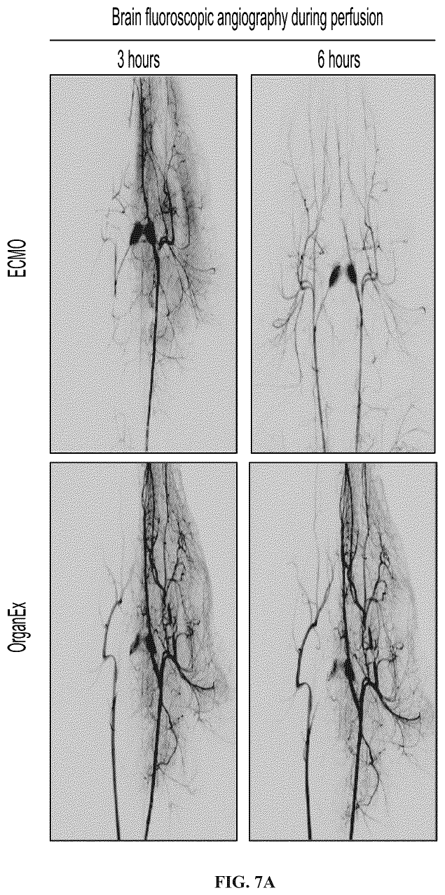

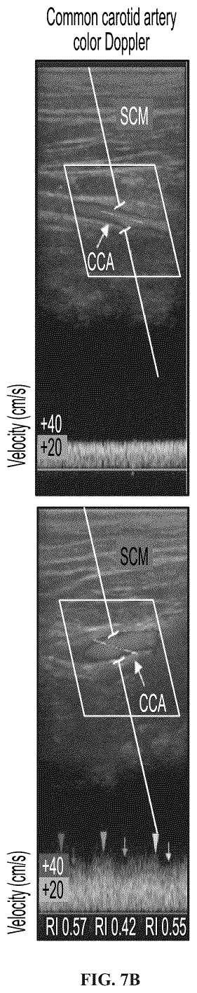

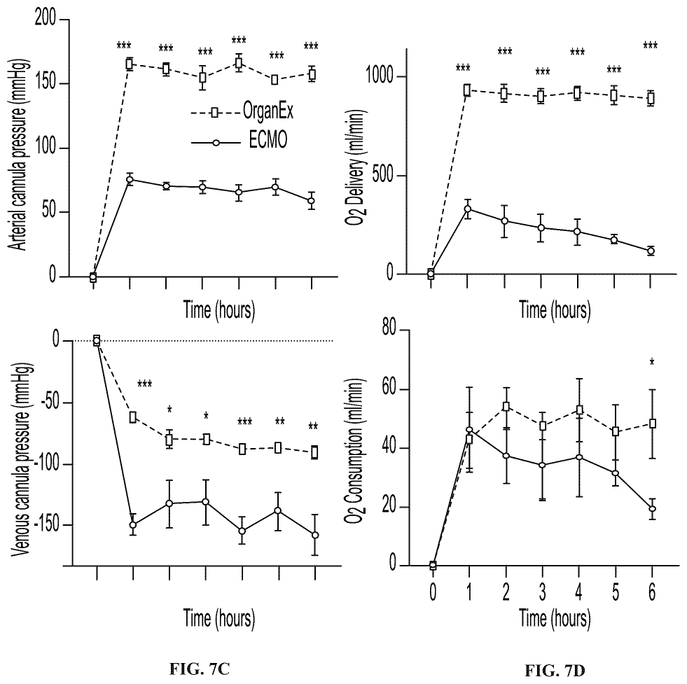

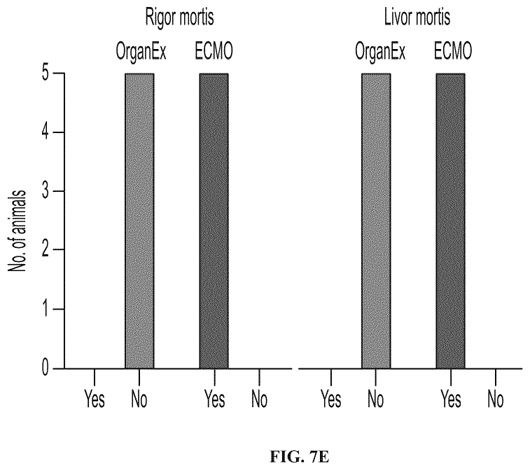

In FIG. 7C-7D, each data point is from the representative perfusion experiment per condition, the experiment was repeated in n=6. In FIG. 7C, Arterial cannula pressure: P values for hours 1-6, 1h: <0.0001, 2h: <0.0001, 3h: <0.0001, 4h: <0.0001, 5h: <0.0001, 6h: <0.0001; t values for hours 1-6, 1h: 12.34, 2h: 8.120, 3h: 8.869, 4h: 9.683, 5h: 17.64, 6h: 22.96. In FIG. 7C, Venous cannula pressure: P values for hours 1-6, 1h: <0.0001, 2h: =0.0306, 3h: =0.0237, 4h: =0.0003, 5h: =0.0069, 6h: =0.0027; t values for hours 1-6, 1h: 9.058, 2h: 2.516, 3h: 2.665, 4h: 5.516, 5h: 3.386, 6h: 3.946. In FIG. 7D, O₂ delivery: P values for hours 1-6, 1h: <0.0001, 2h: <0.0001, 3h: <0.0001, 4h: <0.0001, 5h: <0.0001, 6h: <0.0001; t values for hours 1-6, 1h: 10.44, 2h: 6.957, 3h: 8.578, 4h: 9.462, 5h: 14.32, 6h: 18.56. In FIG. 7D, O₂ consumption: P values for hours 1-6, 1h: =0.8432, 2h: =0.1815, 3h: =0.3195, 4h: =0.3667, 5h: =0.2152, 6h: =0.0394; t values for hours 1-6, 1h: 0.2030, 2h: 1.436, 3h: 1.048, 4h: 0.9455, 5h: 1.323, 6h: 2.368.

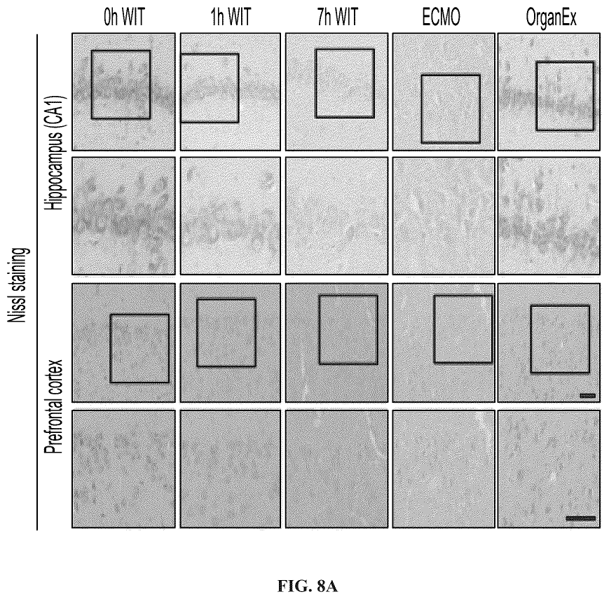

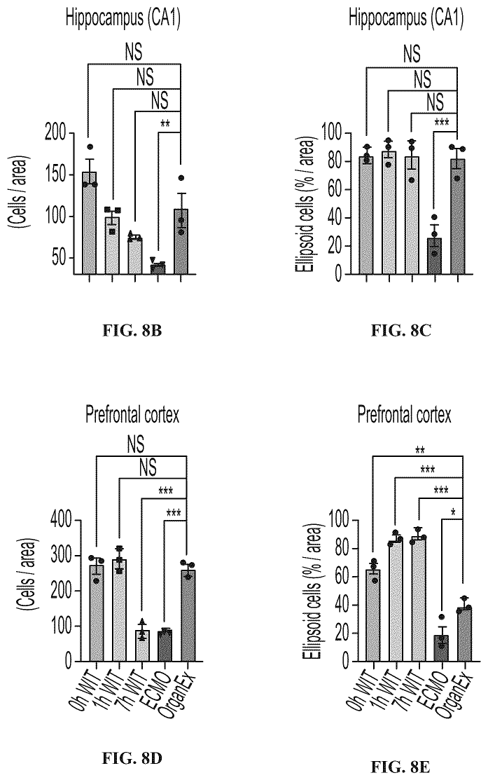

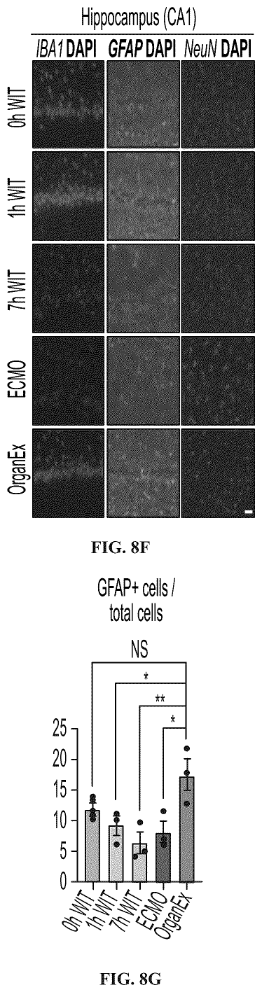

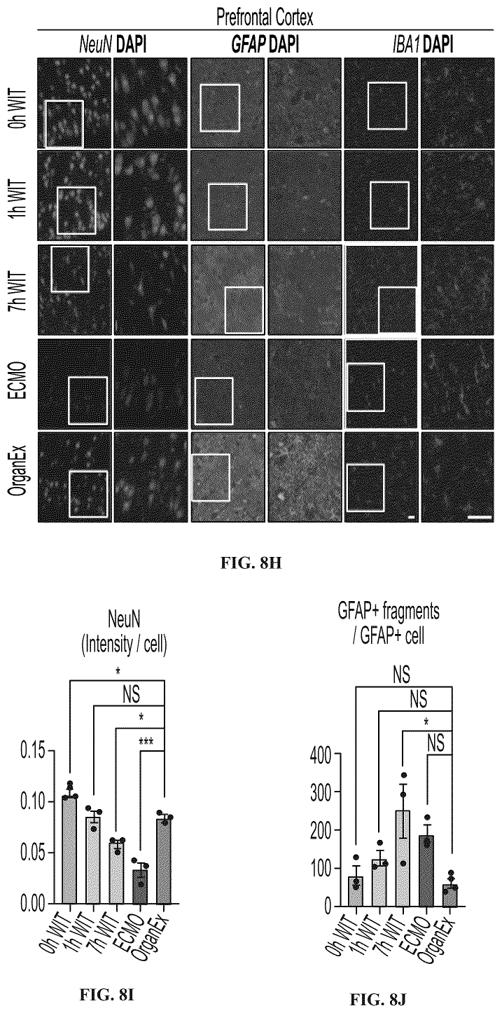

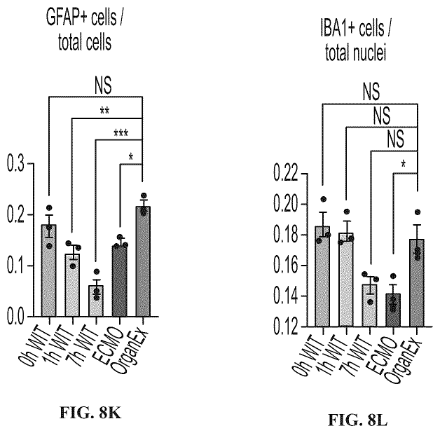

In FIGS. 8B-8E, data points are from a representative brain per condition, the experiment was repeated in n=3 independent brains per condition. In FIG. 8B, One-way ANOVA (P=0.0008, F[4,10]=11.95) with post-hoc Dunnett's adjustment was performed; Dunnett's multiple comparisons test resulted in: OrganEx vs ECMO: P=0.0097; In FIG. 8C, One-way ANOVA (P=0.0003, F[4,10]=14.64) with post-hoc Dunnett's adjustment was performed; Dunnett's multiple comparisons test resulted in: OrganEx vs ECMO: P=0.0006. In FIG. 8D, One-way ANOVA (P<0.001, F[4,10]=44.47) with post-hoc Dunnett's adjustment was performed; Dunnett's multiple comparisons test resulted in: OrganEx vs 7h WIT: P<0.001; OrganEx vs ECMO: P<0.001. In FIG. 8E, One-way ANOVA (P<0.001, F[4,10]=60.70) with post-hoc Dunnett's adjustment was performed; Dunnett's multiple comparisons test resulted in: OrganEx vs 0h WIT: P=0.0024; OrganEx vs 1h WIT: P<0.001; OrganEx vs 7h WIT: P<0.001; OrganEx vs ECMO: P=0.0159.

FIGS. 8G-8L data points are from a representative brain per condition, the experiment was repeated in n=3 independent brains per condition. In FIG. 8G, One-way ANOVA (P=0.011, F[4,10]=5.819) with post-hoc Dunnett's adjustment was performed; Dunnett's multiple comparisons test resulted in: OrganEx vs 1h WIT: P=0.0287; OrganEx vs 7h WIT: P=0.0045; OrganEx vs ECMO: P=0.0149. In FIG. 8I, One-way ANOVA (P<0.0003, F[4,10]=32.20) with post-hoc Dunnett's adjustment was performed; Dunnett's multiple comparisons test resulted in: OrganEx vs 0h WIT: P=0.0243; OrganEx vs 7h WIT: P=0.0157; OrganEx vs ECMO: P=0.0001. In FIG. 8J, One-way ANOVA (P=0.0231, F[4,10]=4.590) with post-hoc Dunnett's adjustment was performed; Dunnett's multiple comparisons test resulted in: OrganEx vs 7h WIT: P=0.0137. In FIG. 8K, One-way ANOVA (P=0.0002, F[4,10]=17.45) with post-hoc Dunnett's adjustment was performed; Dunnett's multiple comparisons test resulted in: OrganEx vs 1h WIT: P=0.09338; OrganEx vs 7h WIT: P<0.0001; OrganEx vs ECMO: P=0.0157. In FIG. 8L, One-way ANOVA (P=0.0032, F[4,10]=8.285) with post-hoc Dunnett's adjustment was performed; Dunnett's multiple comparisons test resulted in: OrganEx vs ECMO: P=0.0198.

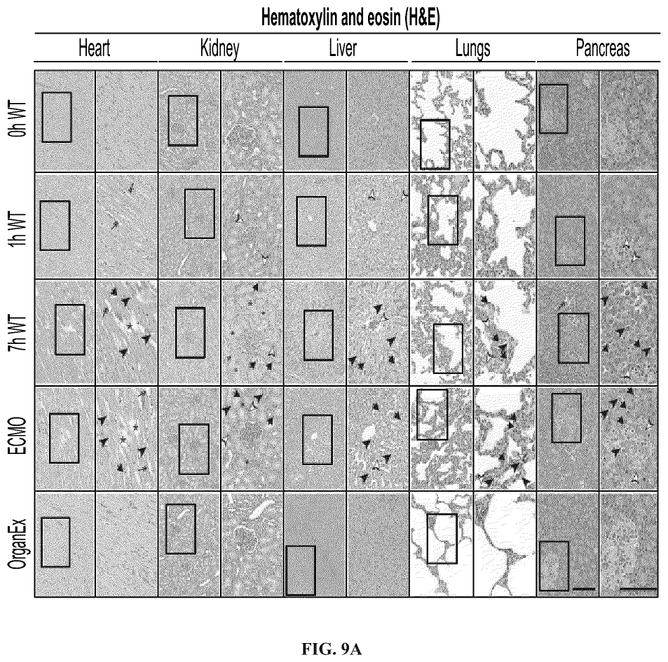

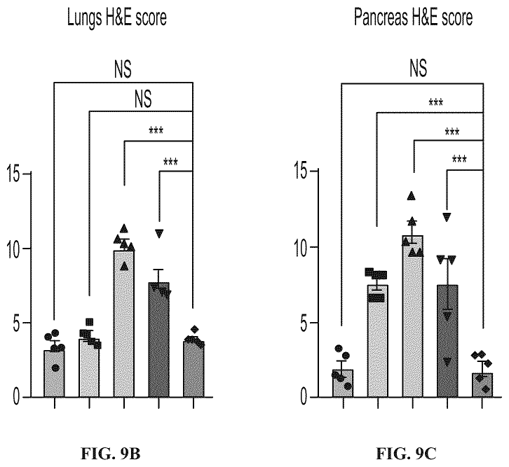

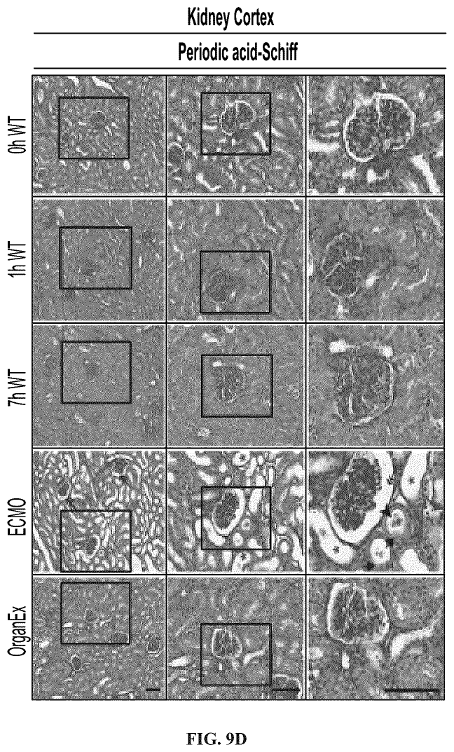

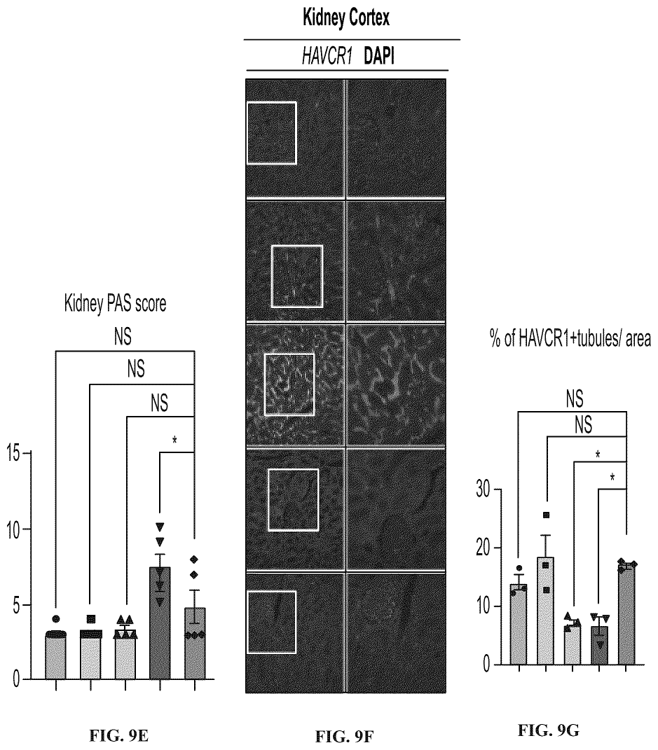

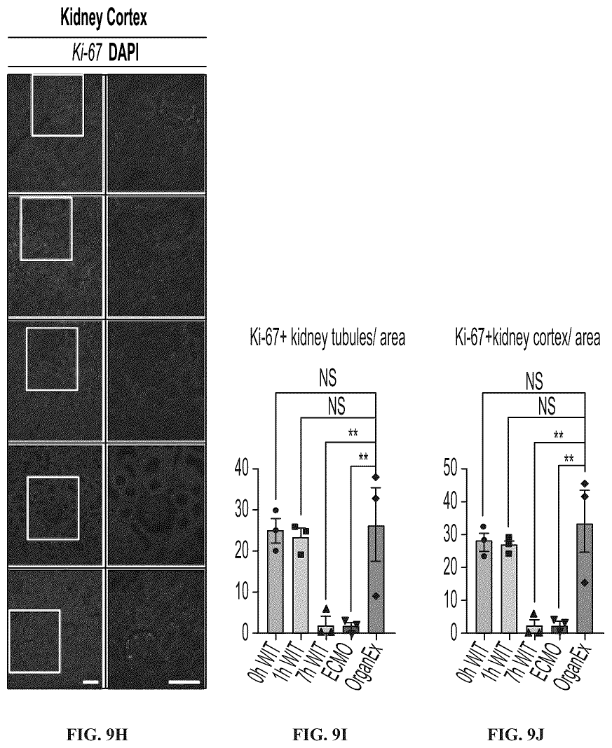

In FIGS. 9B, 9C, 9E, data points are from a representative organ (lung, pancreas, kidney) per condition, the experiment was repeated in n=5 independent organs per condition. In FIG. 9B, One-way ANOVA (P<0.0001, F[4,20]=45.78) with post-hoc Dunnett's adjustment was performed; Dunnett's multiple comparisons test resulted in: OrganEx vs 7h WIT: P<0.0001; OrganEx vs ECMO: P<0.0001. In FIG. 9C, One-way ANOVA (P<0.0001, F[4,20]=19.80) with post-hoc Dunnett's adjustment was performed; Dunnett's multiple comparisons test resulted in: OrganEx vs 1h WIT: P=0.0009; OrganEx vs 7h WIT: P<0.0001; OrganEx vs ECMO: P=0.0009. In FIG. 9E One-way ANOVA (P<0.001, F[4,20]=1.915) with post-hoc Dunnett's adjustment was performed; Dunnett's multiple comparisons test resulted in: OrganEx vs ECMO: P=0.0410.

In FIGS. 9G-9J, data points are from a representative kidney per condition, the experiment was repeated in n=5 independent kidneys per condition. In FIG. 9G, One-way ANOVA (P<0.01, F[4,10]=7.983) with post-hoc Dunnett's adjustment was performed; Dunnett's multiple comparisons test resulted in: OrganEx vs 7h WIT: P=0.0159; OrganEx vs ECMO: P=0.0118. In FIG. 9I, One-way ANOVA (P<0.01, F[4,10]=8.286) with post-hoc Dunnett's adjustment was performed; Dunnett's multiple comparisons test resulted in: OrganEx vs 7h WIT: P=0.0092; OrganEx vs ECMO: P=0.0084. In FIG. 9J, One-way ANOVA (P<0.01, F[4,10]=11.12) with post-hoc Dunnett's adjustment was performed; Dunnett's multiple comparisons test resulted in: OrganEx vs 7h WIT: P=0.002; OrganEx vs ECMO: P=0.0022.

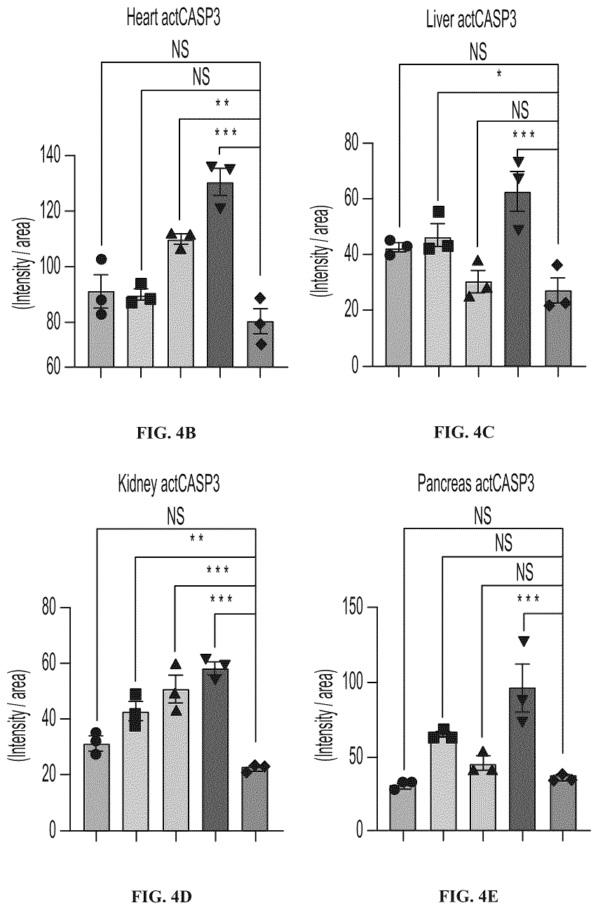

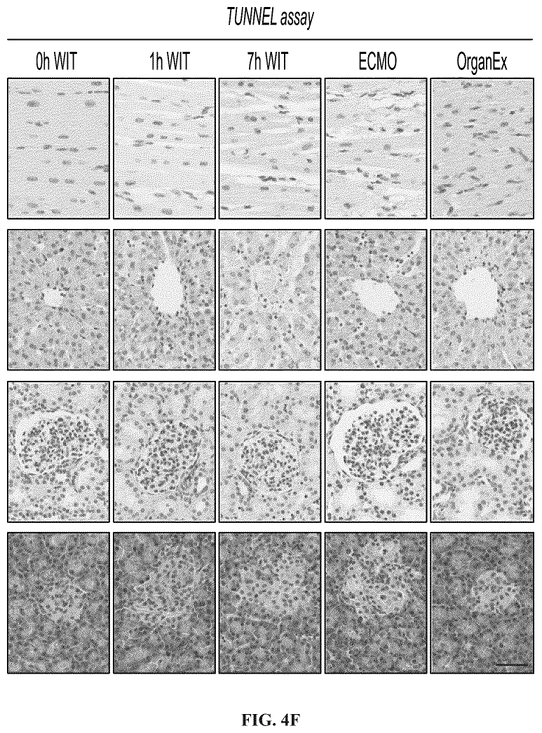

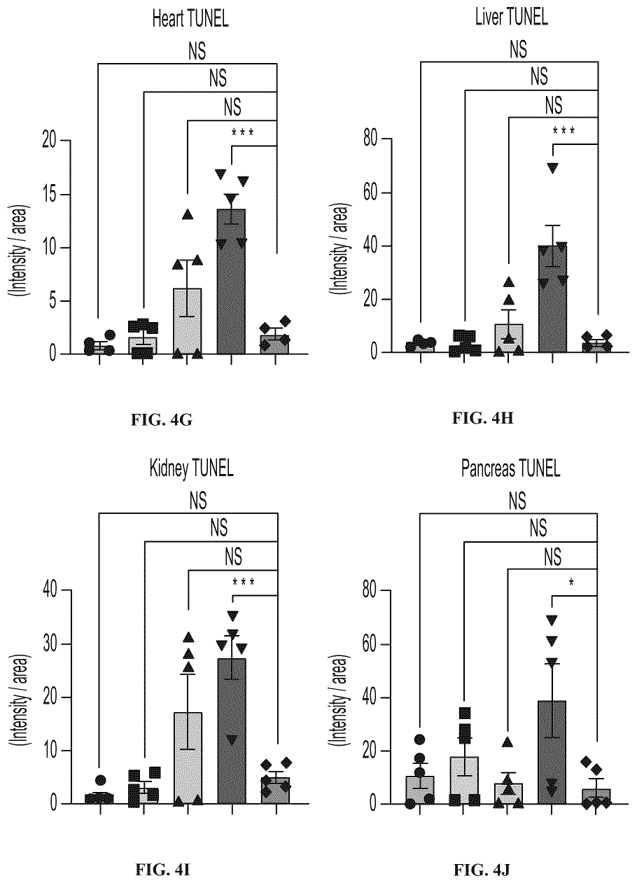

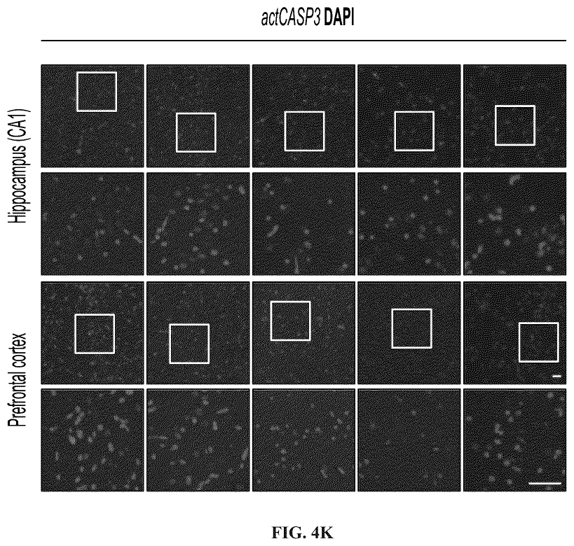

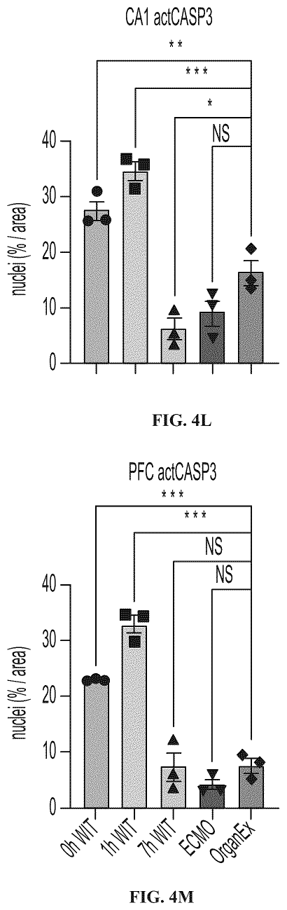

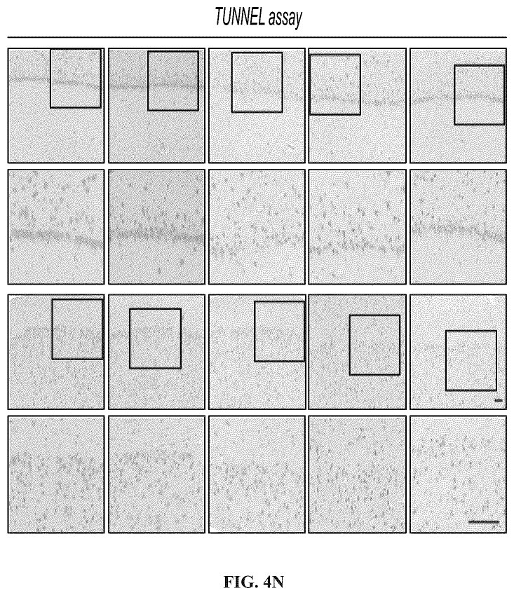

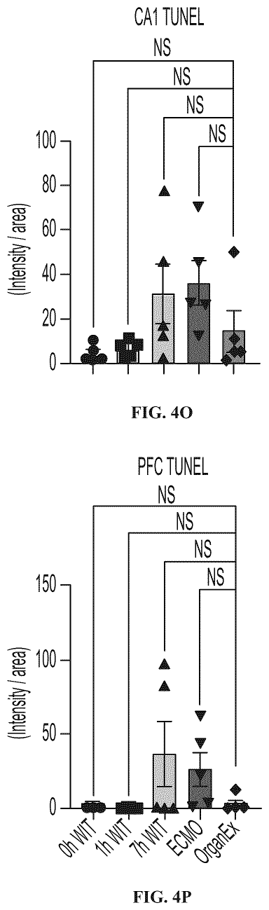

In FIGS. 4B-4E, data points are from a representative organ (heart, liver, kidney, and pancreas) per condition, the experiment was repeated in n=3 independent organs per condition. In FIG. 4B, One-way ANOVA (P<0.0001, F[4,10]=22.48) with post-hoc Dunnett's adjustment was performed; Dunnett's multiple comparisons test resulted in: OrganEx vs 7h WIT: P=0.0018; OrganEx vs ECMO: P<0.0001. In FIG. 4C, One-way ANOVA (P=0.0020, F[4,10]=9.392) with post-hoc Dunnett's adjustment was performed; Dunnett's multiple comparisons test resulted in: OrganEx vs 1h WIT: P=0.004; OrganEx vs ECMO: P=0.001. In FIG. 4D, One-way ANOVA (P<0.0001, F[4,10]=23.50) with post-hoc Dunnett's adjustment was performed; Dunnett's multiple comparisons test resulted in: OrganEx vs 1h WIT: P=0.0022; OrganEx vs 7h WIT: P=0.0002; OrganEx vs ECMO: P<0.0001. In FIG. 4E, One-way ANOVA (P=0.0008, F[4,10]=12.10) with post-hoc Dunnett's adjustment was performed; Dunnett's multiple comparisons test resulted in: OrganEx vs ECMO WIT: P=0.0008.

In FIG. 4J-4M, data points are from a representative organ (heart, liver, kidney, and pancreas) per condition, the experiment was repeated in n=5 independent organs per condition In FIG. 4J, One-way ANOVA (P<0.0001, F[4,20]=14.35) with post-hoc Dunnett's adjustment was performed; Dunnett's multiple comparisons test resulted in: OrganEx vs ECMO: P<0.0001. In FIG. 4K, One-way ANOVA (P<0.0001, F[4,20]=13.26) with post-hoc Dunnett's adjustment was performed; Dunnett's multiple comparisons test resulted in: OrganEx vs ECMO: P<0.0001. In FIG. 4L, One-way ANOVA (P=0.0002, F[4,20]=9.110) with post-hoc Dunnett's adjustment was performed; Dunnett's multiple comparisons test resulted in: OrganEx vs ECMO: P=0.0012. In FIG. 4M, One-way ANOVA (P=0.0387, F[4,20]=3.102) with post-hoc Dunnett's adjustment was performed; Dunnett's multiple comparisons test resulted in: OrganEx vs ECMO: P=0.0216.

In FIG. 4G, 4H, each data points are from a representative brain per condition, the experiment was repeated in n=3 independent brains per condition. In FIG. 4G, One-way ANOVA (P<0.0001, F[4,10]=37.55) with post-hoc Dunnett's adjustment was performed; Dunnett's multiple comparisons test resulted in: OrganEx vs 0h WIT: P=0.0083; OrganEx vs 1h WIT: P=0.0002; OrganEx vs 7h WIT: P=0.016. In FIG. 4H, One-way ANOVA (P<0.0001, F[4,10]=66.81) with post-hoc Dunnett's adjustment was performed; Dunnett's multiple comparisons test resulted in: OrganEx vs 0h WIT: P=0.0001; OrganEx vs 1h WIT: P<0.0001.

In FIGS. 4O, P, each data points are from a representative brain per condition, the experiment was repeated in n=5 independent brains per condition. In FIG. 4O, One-way ANOVA (P=0.0547, F[4,20]=2.784) with post-hoc Dunnett's adjustment was performed. In FIG. 4P, One-way ANOVA (P=0.1005, F[4,20]=2.244) with post-hoc Dunnett's adjustment was performed.

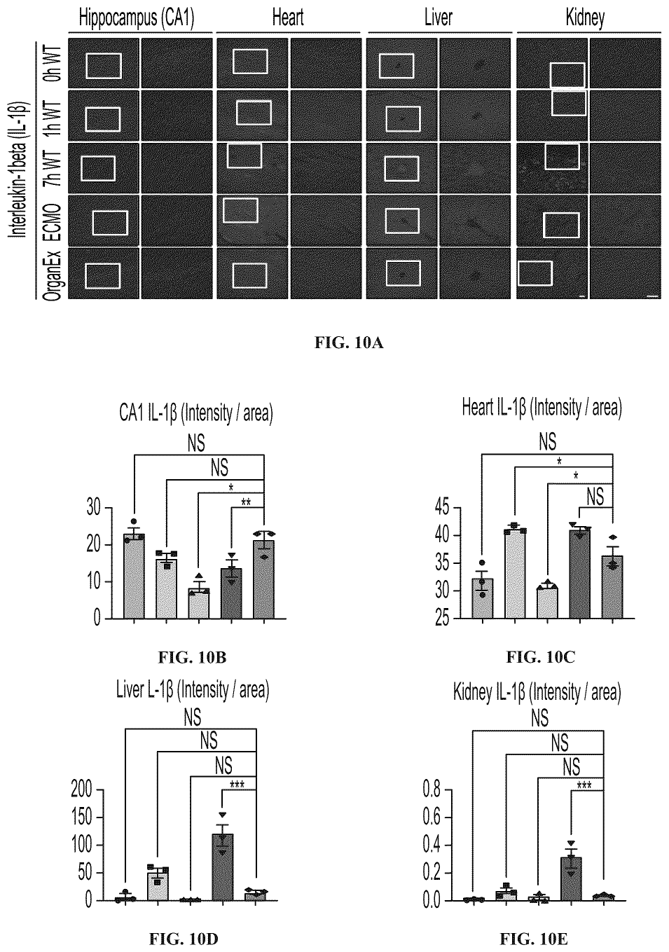

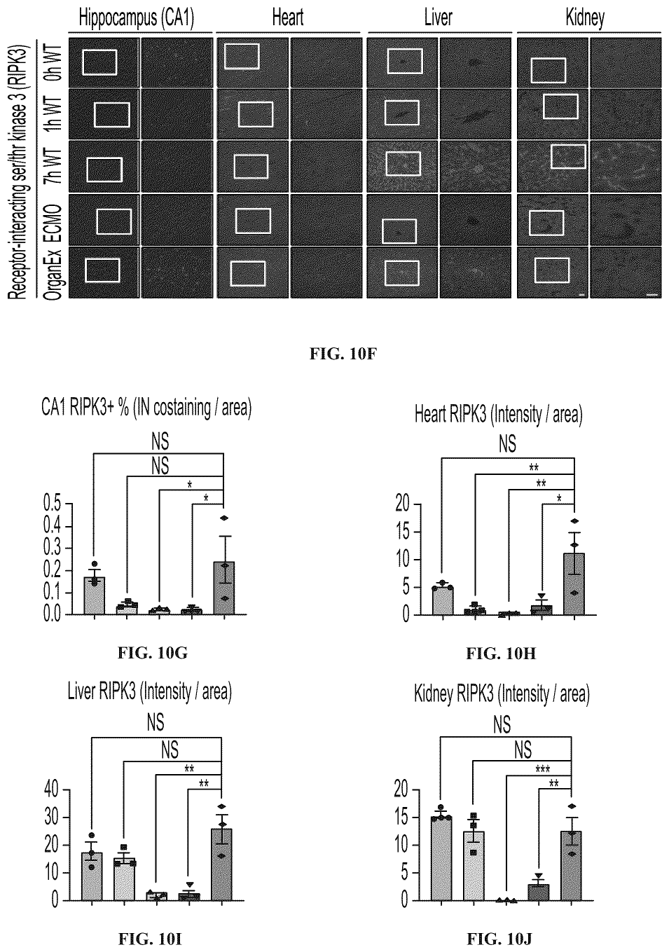

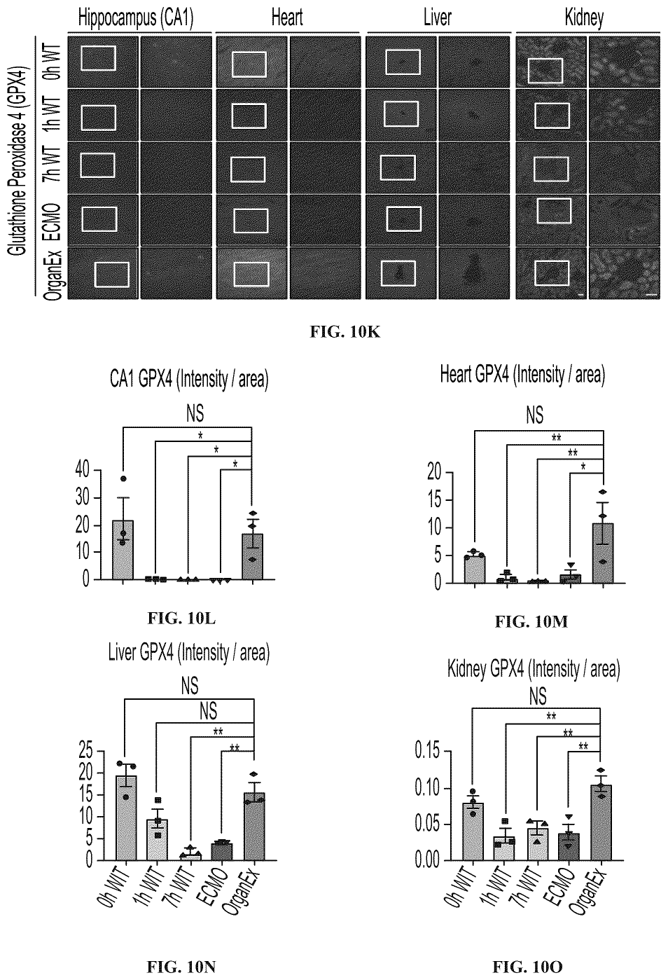

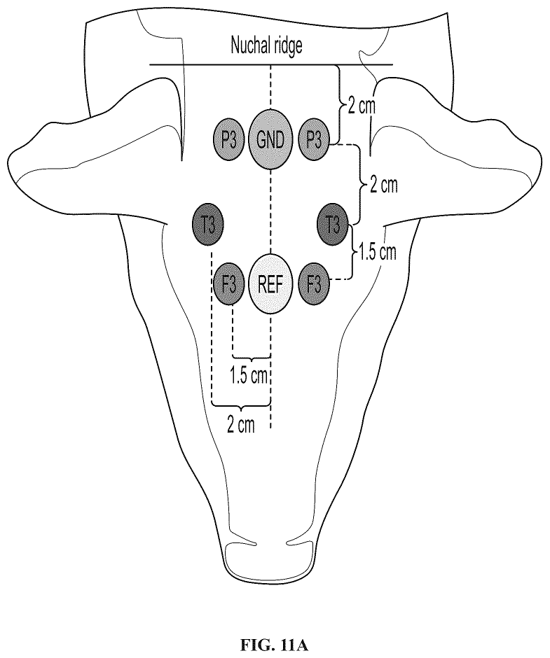

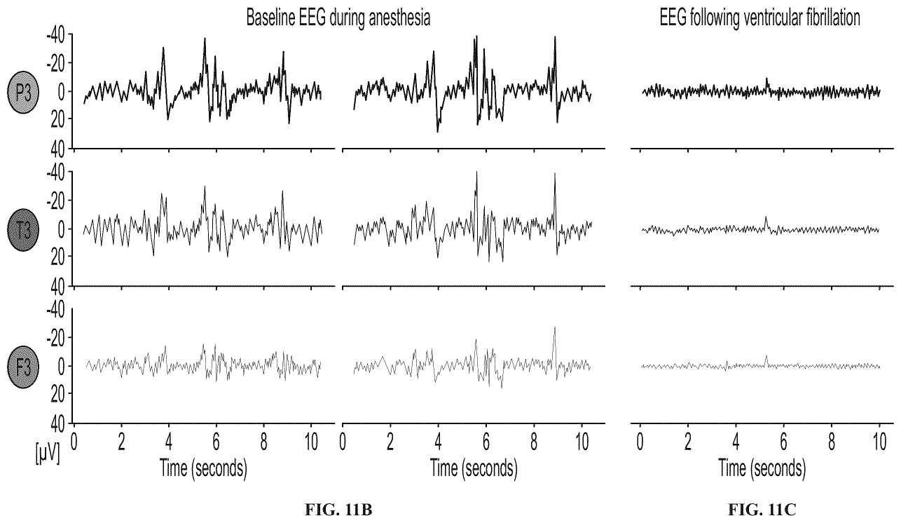

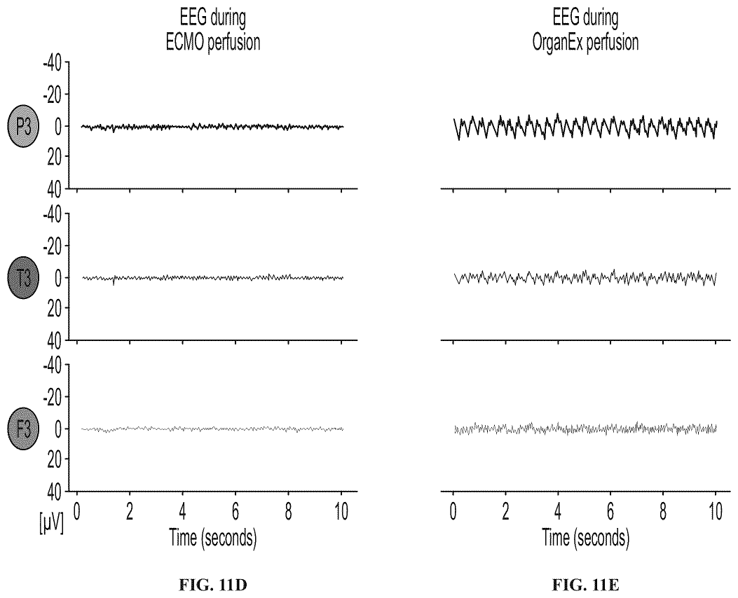

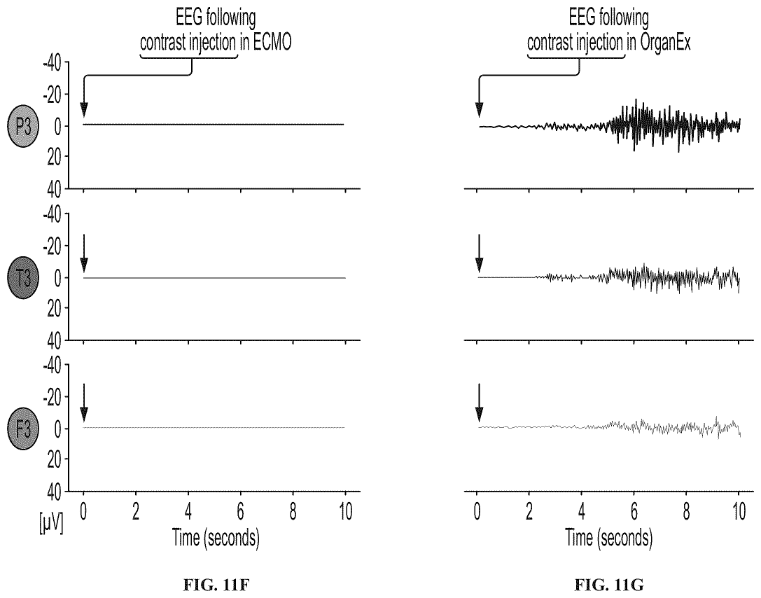

In FIGS. 10, each data point is from the representative brain specimen per condition, the experiment was repeated n=3 times per condition. In FIG. 10B, One-way ANOVA (P=0.0013, F[4,10]=10.52) with post-hoc Dunnett's adjustment was performed; Dunnett's multiple comparisons test resulted in: OrganEx vs 7h WIT: P=0.002; OrganEx vs ECMO: P=0.0418. In FIG. 10C, One-way ANOVA (P=0.0002, F[4,10]=17.06) with post-hoc Dunnett's adjustment was performed; Dunnett's multiple comparisons test resulted in: OrganEx vs 1h WIT: P=0.0386; OrganEx vs 7h WIT: P=0.0302. In FIG. 10D, One-way ANOVA (P<0.0001, F[4,10]=23.67) with post-hoc Dunnett's adjustment was performed; Dunnett's multiple comparisons test resulted in: OrganEx vs ECMO: P=0.0001. In FIG. 10E, One-way ANOVA (P=0.0003, F[4,10]=14.74) with post-hoc Dunnett's adjustment was performed; Dunnett's multiple comparisons test resulted in: OrganEx vs ECMO: P=0.0006. In FIG. 10F, One-way ANOVA (P=0.0264, F[4,10]=4.389) with post-hoc Dunnett's adjustment was performed; Dunnett's multiple comparisons test resulted in: OrganEx vs 7h WIT: P=0.0301; OrganEx vs ECMO: P=0.0270. In FIG. 10H, One-way ANOVA (P=0.0082, F[4,10]=6.365) with post-hoc Dunnett's adjustment was performed; Dunnett's multiple comparisons test resulted in: OrganEx vs 1h WIT: P=0.0082; OrganEx vs 7h WIT: P=0.005; OrganEx vs ECMO: P=0.0124. In FIG. 10I, One-way ANOVA (P=0.0012, F[4,10]=10.81) with post-hoc Dunnett's adjustment was performed; Dunnett's multiple comparisons test resulted in: OrganEx vs 7h WIT: P=0.001; OrganEx vs ECMO: P=0.0012. In FIG. 10J, One-way ANOVA (P=0.0001, F[4,10]=18.96) with post-hoc Dunnett's adjustment was performed; Dunnett's multiple comparisons test resulted in: OrganEx vs 7h WIT: P=0.0048; OrganEx vs ECMO: P=0.0049. In FIG. 10L, One-way ANOVA (P=0.0264, F[4,10]=7.430) with post-hoc Dunnett's adjustment was performed; Dunnett's multiple comparisons test resulted in: OrganEx vs 1h WIT: P=0.0437; OrganEx vs 7h WIT: P=0.0437; OrganEx vs ECMO: P=0.0437. In FIG. 10M, One-way ANOVA (P=0.0082, F[4,10]=6.365) with post-hoc Dunnett's adjustment was performed; Dunnett's multiple comparisons test resulted in: OrganEx vs 1h WIT: P=0.0082; OrganEx vs 7h WIT: P=0.005; OrganEx vs ECMO: P=0.0124. In FIG. 10N, One-way ANOVA (P=0.0002, F[4,10]=16.23) with post-hoc Dunnett's adjustment was performed; Dunnett's multiple comparisons test resulted in: OrganEx vs 7h WIT: P=0.0013; OrganEx vs ECMO: P=0.044. In FIG. 10O, One-way ANOVA (P=0.0024, F[4,10]=9.035) with post-hoc Dunnett's adjustment was performed; Dunnett's multiple comparisons test resulted in: OrganEx vs 1h WIT: P=0.0019; OrganEx vs 7h WIT: P=0.0062; OrganEx vs ECMO: P=0.0034.

In FIGS. 11J, 11K, each data point is from the representative hippocampal slice per condition, the experiment was repeated n=3-5 times per condition. In FIG. 11J, for day 1: One-way ANOVA (P=0.0078, F[2,9]=8.749) with post-hoc Dunnett's adjustment was performed, Dunnett's multiple comparisons test resulted in OrganEx vs ECMO: P=0.0245; for day 7: One-way ANOVA (P=0.0126, F[2,10]=6.995) with post-hoc Dunnett's adjustment was performed, Dunnett's multiple comparisons test resulted in: OrganEx vs ECMO: P=0.0086; for day 14: One-way ANOVA (P=0.005, F[2,9]=10.12) with post-hoc Dunnett's adjustment was performed, Dunnett's multiple comparisons test resulted in: OrganEx vs ECMO: P=0.0037. In FIG. 11K, for day 1: One-way ANOVA (P=0.0406, F[2,9]=4.670) with post-hoc Dunnett's adjustment was performed, Dunnett's multiple comparisons test resulted in OrganEx vs ECMO: P=0.0375; for day 7: One-way ANOVA (P=0.0278, F[2,9]=5.476) with post-hoc Dunnett's adjustment was performed, Dunnett's multiple comparisons test resulted in: OrganEx vs ECMO: P=0.0406; for day 14: unpaired two-tailed t-test was performed: P=0.8691, t=0.1735.

In FIG. 11M, 11O, data points are from a representative organs (heart, liver) per condition, the experiment was repeated in n=3 independent organs per condition. In FIG. 11M, One-way ANOVA (P=0.0207, F[4,10]=4.760) with post-hoc Dunnett's adjustment was performed; Dunnett's multiple comparisons test resulted in: OrganEx vs ECMO: P=0.00434. In FIG. 11O, One-way ANOVA (P=0.0063, F[4,10]=6.863) with post-hoc Dunnett's adjustment was performed; Dunnett's multiple comparisons test resulted in: OrganEx vs 7h WIT: P=0.0064; OrganEx vs ECMO: P=0.0163.

### Example 1: Overview of OrganEx Technology

The OrganEx technology consists of a perfusion system and synthetic perfusate (FIG. 1A, FIG. 1). The perfusion system consists of a computer driven custom-made pulse generator connected to a centrifugal pump, which enables reproduction of physiological pressure and flow waveforms, together with automated hemodiafiltration, gas mixer, and drug delivery systems which allow control of blood coagulation and supplementation of the perfusate. To ensure homeostasis and maintain the targeted perfusion parameters, the perfusion system is also equipped with sensors for electrolytes, blood gases, metabolic parameters, hemoglobin, vessels and cannulas pressures, and total circulatory flow rate. The perfusate is optimized for whole-body compatibility. The OrganEx perfusate is a final mixture of a custom-made priming solution (Table 1), Hemopure (HbO2 Therapeutics, Waltham, MA), custom-made dialysis exchange solution (Table 2) and the solution of pharmacological compounds (Table 3).

To evaluate OrganEx technology in large mammals, a porcine global warm ischemia model induced by cardiac arrest on anesthetized and heparinized animals (FIG. 1A) was implemented. Following cardiac arrest and cessation of the systemic circulation, animals were left dead for one hour, allowing warm ischemic damage to ensue at core temperature of 36-37° C. Subsequently, animals were connected to one of the two perfusion systems via a femoral artery/vein approach with the goal of reinstating systemic circulation and nutritive rather than functional circulation of heart and lungs (FIG. 1A). Overall, this study consisted of five groups. Three different, unperfused, control groups corresponding to important experimental timepoints and distinct warm ischemia intervals were used: (1) healthy control group with minimal (up to 10 minutes) warm ischemia time (WIT) following cardiac arrest (0h WIT), (2) an hour of warm ischemia to investigate molecular and cellular damage prior to perfusion intervention (1h WIT), and (3) seven hours of warm ischemia to investigate damage extent that happens without any intervention (7h WIT). Additionally, in two perfusion intervention groups, following one hour of warm ischemia, perfusion was performed for six hours under hypothermic conditions (28° C.) either with: (4) a clinical standard, heart-and-lung substitution perfusion device—extracorporeal membrane oxygenation system (ECMO), or (5) perfusion technology (OrganEx). Animals in ECMO group were perfused with autologous blood. In OrganEx, prior to the initiation of the perfusion protocol, autologous blood was drained into the OrganEx system and mixed with the perfusate (in for example, effective 1:1 ratio), which was then used to perfuse the animal.

### Example 2: Systemic Circulation and Metabolic Parameters

Since systemic circulation assures supply of oxygen, metabolites and potential therapeutic agents during recovery from ischemia, it was first queried whether circulation could be restored with external perfusion following one hour of warm ischemia. Perfusion of the whole-body with ECMO system invariantly resulted in low or no flow states due to circulatory collapse. In particular, utilizing fluoroscopic angiography, ECMO perfusion exhibited limited arterial filling of major conduit arteries and organs, such as kidney, liver and brain (FIG. 2A, FIG. 7A). Consistent with this finding, ECMO interventions yielded inadequate organ perfusion as indicated by Color Doppler assessment (FIG. 2B). Furthermore, systemic perfusion parameters revealed collapse of circulation as shown by extremely negative venous perfusion pressure and low arterial pressures (FIG. 2C, FIG. 7C-7D).

In contrast, robust whole-body perfusion in the OrganEx treated animals was observed, as indicated by contrast enhancement of major conduit arteries and organs, demonstrating patent circulation (FIG. 2A, FIG. 7A). In addition, Color Doppler analysis demonstrated pulsatile flow throughout the whole-body in the OrganEx group (FIG. 2B). In particular, flow in the ophthalmic artery, a proxy indicator of cerebral perfusion, was present in the OrganEx, but not in ECMO perfusion group, when analyzed at hour three of the perfusion protocols (FIG. 2B). Similar findings were observed in renal intralobular arteries (FIG. 2B), while reduced flow was detected in carotid arteries as compared to OrganEx (FIG. 7B). These data were further supported with the data from the OrganEx perfusion system sensors showing restoration of physiologic flow rates and arterial pressures (FIG. 2C).

Following successful restoration of circulation, the extent that OrganEx is capable of recovering metabolic parameters was assessed. By measuring mixed venous oxygen saturation of blood returning to the venous circulation from peripheral tissues and organs, it was confirmed that the OrganEx technology was able to deliver adequate levels of oxygen to the whole body throughout the perfusion (FIG. 2D). This was coupled by stabilization of tissue expenditure (FIG. 7E-7F) and correction of physiologic imbalances in the blood that occur during prolonged ischemia, most notably hyperkalemia and metabolic acidosis (FIG. 2F-2G). In addition, postmortem rigidity and lividity observed in ECMO animals were absent after OrganEx perfusion (FIG. 7E). Taken together, these observations indicate that following one hour of warm ischemia, OrganEx could reinstate and maintain circulation, and restore observed physiologic and metabolic parameters on a whole-body scale.

### Example 3: Histological Analysis of Tissue Integrity

Upon restoration of systemic circulation and certain key metabolic parameters following OrganEx perfusion, next investigated was its effects on organ structural integrity and cytoarchitecture. The brain's three major cell types, neurons, astrocytes, and microglia in two regions most vulnerable to ischemia, the hippocampal CA1 subregion and the prefrontal cortex (PFC) were analyzed. The intensity of immunostaining for RBFOX3 (NeuN), a pan-neuronal marker, which has previously been shown to decrease in hypoxia, was lower in the ECMO and 7h WIT groups in both regions, as compared to OrganEx (FIG. 3A, FIG. 8F, 8H). While there were no observed differences in CA1 subregion between OrganEx and 0h and 1h WIT groups, we did observe reduced immunolabeling in OrganEx and 1h WIT group compared to 0h WIT in PFC (FIGS. 8H, 8I). This finding could be a result of more pronounced protein degradation in PFC or incomplete recovery due to short OrganEx intervention time. In addition, the number of astrocytes immunolabeled for GFAP was comparable between OrganEx and 0h WIT but was decreased in other experimental groups, in both, CA1 and PFC (FIGS. 8F, 8G, 9B, 9E). The analysis of GFAP immunolabeling fragmentation was increased in the 7h WIT and ECMO groups compared to OrganEx, which was similar to the 1h, and 0h WIT groups, suggesting loss of astrocytic integrity (FIGS. 3A, 3B, FIG. 8B, 8H, 8K). A similar trend was observed in PFC, with increased fragmentation in 7h WIT and ECMO groups, but failed to demonstrate statistical significance due to increased variance in 7h WIT group (FIGS. 8H, 8J). The density of microglial populations noted by IBA1 immunolabeling was comparable among OrganEx, 0h and 1h WIT groups in both CA1 and PFC, and differed from the ECMO group (FIGS. 3A, 3D, FIGS. 8F, 8H, 8L). Collectively, these findings indicate that across different analyzed brain regions and cell types, both tissue and cellular integrities were preserved and did not sustain additional observable damage following perfusion with OrganEx, consistent with previous study in the isolated porcine brain.

After assessing highly oxygen-sensitive brain structures and cell types, next investigated were the effects of OrganEx on tissue and cellular integrity in essential peripheral organs, including heart, lungs, liver, kidney, and pancreas. Utilizing hematoxylin and eosin (H&E) staining, hemorrhage, tissue edema, nuclear pyknosis, cell vacuolization, and cellular integrity were evaluated and were combined into a cumulative damage score according to the standard pathologic criteria. Notably, the OrganEx group showed a decrease in the H&E damage score, as compared to 7h WIT and ECMO groups (FIG. 3E-3H, FIGS. 9A-9J). Furthermore, organs treated with OrganEx perfusion exhibited signs of reduced hemorrhage and tissue edema when compared to the 1h WIT group. These results are indicative of the absence of injury promotion and cytoarchitectural damage as compared to ECMO, and more importantly, a reduction of H&E damage scores towards the 0h WIT state with OrganEx perfusion.

To further assess recovery of cytoarchitecture in the OrganEx intervention group compared with possible injury promotion in ECMO group, the cytoskeletal features of the kidney that are well-studied in this regard were investigated. Expression of renal cytoskeletal 3-actin is known to be upregulated in ischemic states. However, the reintroduction of autologous blood causes risk for cell injury and cytoskeletal disruption leading to protein loss. This analysis revealed reduced 3-actin immunoreactivity in the ECMO, as compared to OrganEx and other groups, which exhibited preserved immunoreactivity (FIG. 3J-3K). These data indicate that unlike OrganEx perfusion, the ECMO perfusion with autologous blood following prolonged warm ischemia may cause risk for further tissue damage through reperfusion.

### Example 4: Analysis of Cell Death Processes

Because the OrganEx perfusate contains pharmacological suppressors of cell death and decreased cellular demise based on the histopathological analysis in the OrganEx group (FIG. 3E-3H, FIGS. 9A-9J) was observed, key proteins of key cell death pathways were next investigated by immunohistochemistry analysis. For apoptosis, immunolabeling intensity of activated caspase3 (actCASP3) and TUNEL assay was measured, and an increase was observed across peripheral organs such as heart, liver, kidney, and pancreas in the ECMO compared to the OrganEx group. Furthermore, the respective intensities of actCASP3 and TUNEL in the OrganEx were comparable to the 0h WIT group, which did not sustain ischemic injury, indicating that OrganEx perfusion diminished caspase3 activation and decreased apoptosis (FIG. 4A-4P).

The analysis of the CA1 and PFC revealed that the intensity of actCASP3 immunolabeling in the OrganEx group was lower than in the 0h and 1h WIT groups (FIGS. 4K-4M). Conversely intensity of TUNEL assay had lower trend in OrganEx group compared to the 7h WIT and ECMO groups (FIGS. 4N-4P). Thus, it is conceivable that the weak brain immunolabeling of actCASP3 in the OrganEx group can be explained by active suppression of actCAPS3 by the pharmacological compounds in the perfusate as previously reported, rather than cellular or protein destruction, as also supported by other evaluations (FIGS. 3A-3D, 6A, 8A-8L, 11H-11K, 14F,14G).

Next, to investigate pyroptosis, the cell death pathway triggered by proinflammatory signals, interleukin 1 beta (IL1B) immunohistochemistry was utilized. Across all investigated peripheral organs such as heart, liver, and kidney, IL1B immunolabeling intensity was comparable in 0h WIT and increased in ECMO when compared to OrganEx. In the brain, immunolabeling intensity was decreased in ECMO when compared to OrganEx group (FIGS. 10A-10E). Trends in both peripheral organs, and the brain are similar to the observed actCASP3 results (FIG. 4A-4E).

Finally, necroptosis and ferroptosis, two distinct cell death pathways were investigated by utilizing immunohistochemistry of their important proteins in the pathways, the receptor-interacting ser/thr kinase 3 (RIPK3) and the glutathione peroxidase 4 (GPX4), respectively. The results were consistent between the two cell death pathways and between all the organs evaluated such as brain, heart, liver, and kidney. Compared to OrganEx, immunolabeling intensity was comparable in 0h WIT and significantly decreased in 7h WIT and ECMO groups (FIGS. 10F-100).

### Example 5: Metabolic and Functional Assessment of Organs

After observing improvements in metabolic function, tissue cytoarchitecture and cell death outcomes using OrganEx, cellular energy balance was next investigated in detail. The glucose uptake was measured in highly metabolic organs (brain, heart, kidney) using the fluorescent glucose analog, 2-NBDG23. This showed comparable levels of glucose uptake in the OrganEx and 0h WIT groups in all assessed organs and reduced cellular glucose capture in the ECMO group that may indicate impaired glucose utilization of cellular leakage (FIGS. 5A-5C). Such findings imply that recovery of cellular metabolism may have a reciprocal relationship with the restoration of systemic metabolic parameters (FIGS. 2D, 2E, 7D).

Indicators of cell- and tissue-level recovery in relevant organs were next tested. Cardiac assessment with electrocardiography (ECG) demonstrated spontaneous reemergence of QRS complexes during OrganEx perfusion, indicating ventricular depolarization (FIG. 5D). However, no QRS reemergence was observed in the ECMO group. To further evaluate recovery of ventricular activity, cardiomyocyte contractility was examined using bright-field microscopy of left ventricle tissue slices acquired at the experimental endpoint. Contractions in OrganEx and 0h WIT samples were observed, but complete absence in the ECMO group (FIG. 5E). Finally, investigated were cardiomyocyte biomarkers whose immunolabeling decreases with ischemia22. Left ventricle immunohistochemistry staining for biomarker troponin I revealed decreased immunolabeling with prolonged ischemia, and significantly lower immunolabeling intensity in the ECMO vs OrganEx group (FIGS. 11L, 11M).

Liver cellular recovery was assessed using immunostaining for albumin and factor V, which are non-structural liver-synthesized proteins with great abundance and short half-life, respectively. Compared to OrganEx group, immunolabeling intensities of both proteins were comparable in 0h WIT and significantly diminished in 7h WIT and ECMO groups (FIGS. 5F, 5G, 11N, 11O).

In the kidney, although many cellular features were preserved similar to 0h WIT in OrganEx groups, including tissue integrity (FIGS. 3H, 9D,9E), cell death (FIGS. 4A-4P, 10A-100), molecular and proliferative injury responses (FIGS. 9F-9J), and cellular metabolic indicators (FIG. 5C), the primary kidney functional metric, urine output, was minimal. Yet hypothermic perfusion is known to slow kidney function in patients with healthy organs and extracorporeal perfusion circuits can perturb endocrine, humoral, and neural factors regulating glomerular filtration even when renal perfusion and cellular health is adequate. Longer recovery time also may be required since low urine output often follows shock resuscitation.

Next, continuous electroencephalography (EEG) of the brain in OrganEx and ECMO groups was conducted, and no signs of global network activity were detected (FIGS. 11A-11E). In OrganEx group, we hypothesize this could be due to either inadequate brain recovery, requirement for longer recovery duration, perfusates' neuronal activity antagonists, anesthesia, hypothermic perfusion protocol, or their combined effects. Interestingly, while receiving carotid injection of contrast for cerebrovascular fluoroscopic imaging, OrganEx-perfused animals exhibited complex, non-purposeful, non-stereotyped movements of the head, neck, and torso from coordinated agonist/antagonist actions across multiple joints and muscle units. This was not observed during imaging of sedated alive or ECMO-treated animals (FIG. 11F). EEG patterns during these movements were not interpretable due to movement-induced artifacts but were flat immediately before and after the movements (FIG. 11G). Whether these movements are initiated from preferential interruption of cerebral descending inhibition of motor patterns or from positive action at subcortical, spinal, peripheral nerve, or neuromotor unit levels is difficult to determine. However, the ability for them to be executed does indicate the preservation of efferent motor output function at least at the level of the spinal cervical cord or its roots.

Next, it was sought to investigate the longer-term actions of OrganEx perfusion on cellular viability. Yet because of regulatory constraints and inability to extend the perfusion protocol beyond 6h, organotypic hippocampal brain slice cultures (BSCs) were utilized, to monitor features of tissues previously exposed to different perfusion interventions. BSCs were prepared at the beginning of experiments (0h WIT), at the end of ECMO and OrganEx perfusions, and from time-matched 7h WIT controls, and were cultured for 14 days while assessing tissue integrity and protein synthesis using Click-iT assay screening. BSCs from the 7h WIT group failed these measures due to severe tissue degradation. Based on visual inspection and DAPI staining, BSCs from the ECMO group were more fragile than OrganEx samples, and most disintegrated by day 14 (FIGS. 5H, 5I). BSC tissue integrity was preserved through day−14 in the OrganEx group equal to 0h WIT despite having had one additional ischemic exposure (1× during initial 1h warm ischemia, 1×during brain extraction, 5-10 mins). Similarly, protein synthesis was comparable through day-14 in the OrganEx and 0h WIT groups and decreased in ECMO across different hippocampal regions (FIGS. 5J, 5K, 11H-11K).

### Example 6: Analysis of Cell Type-Specific Transcriptomic Changes

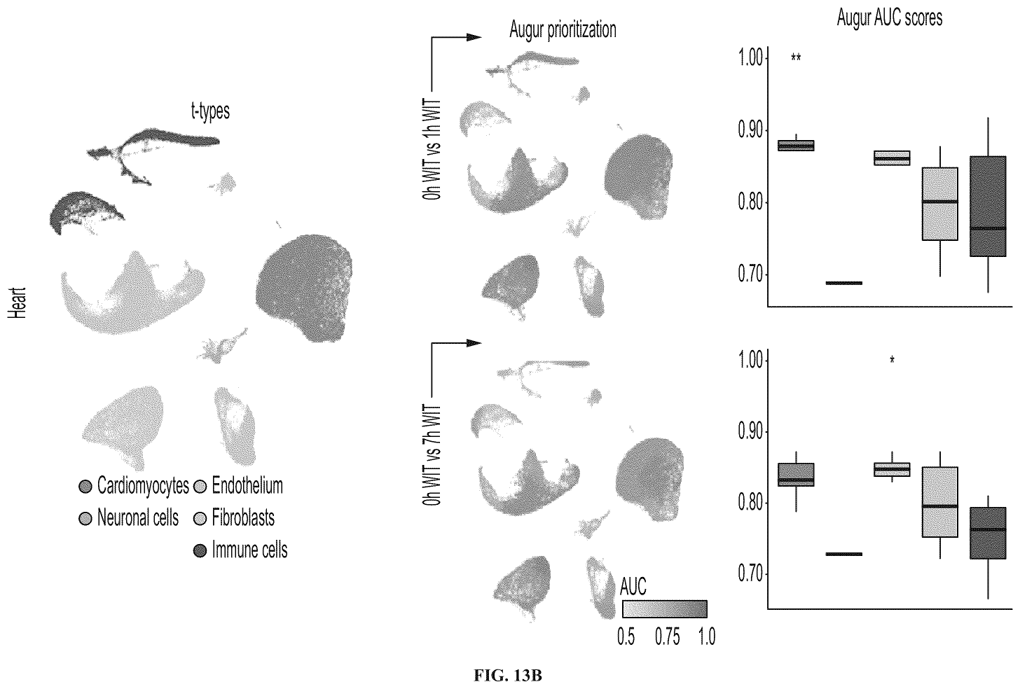

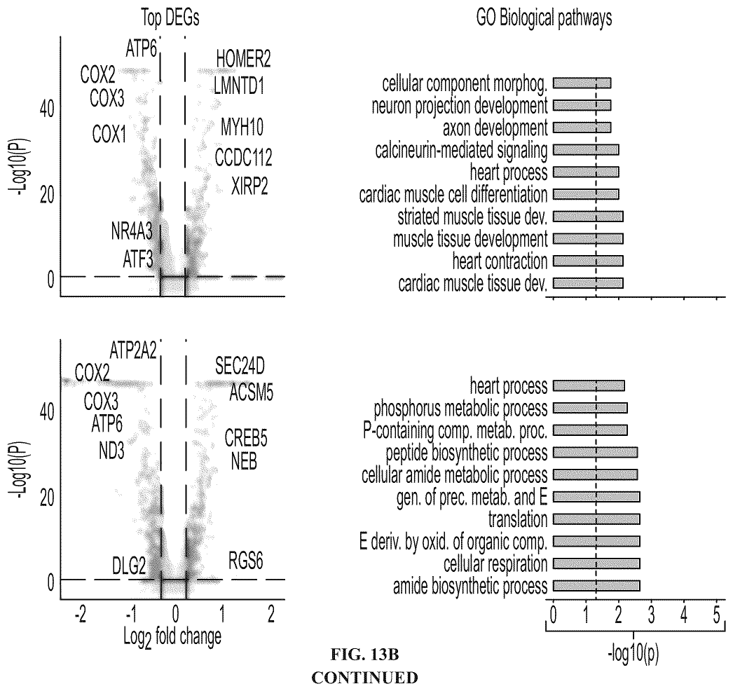

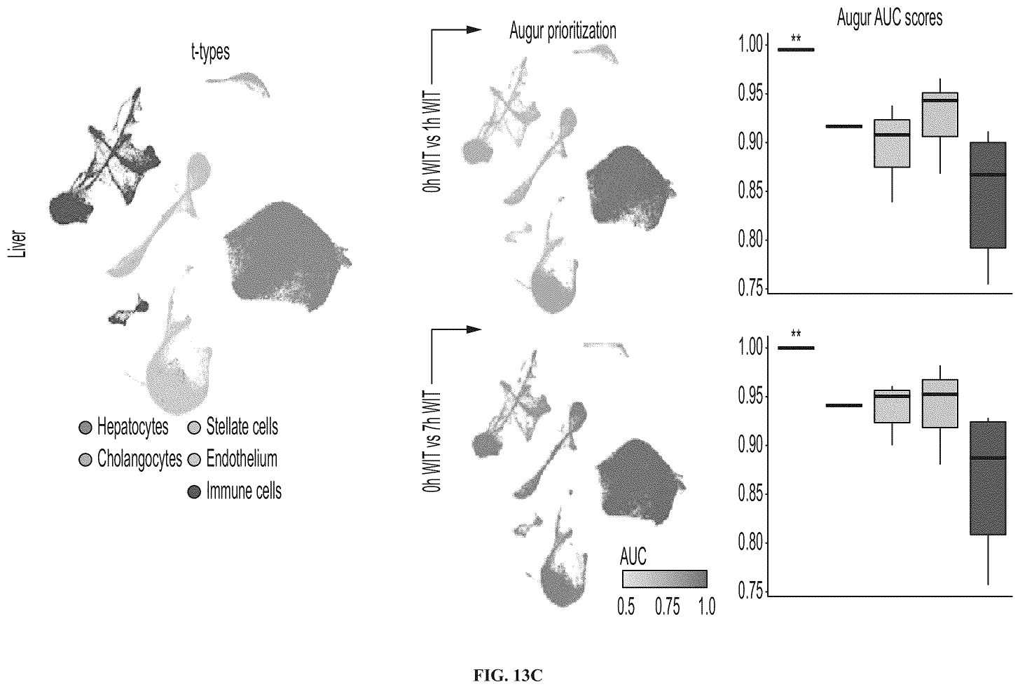

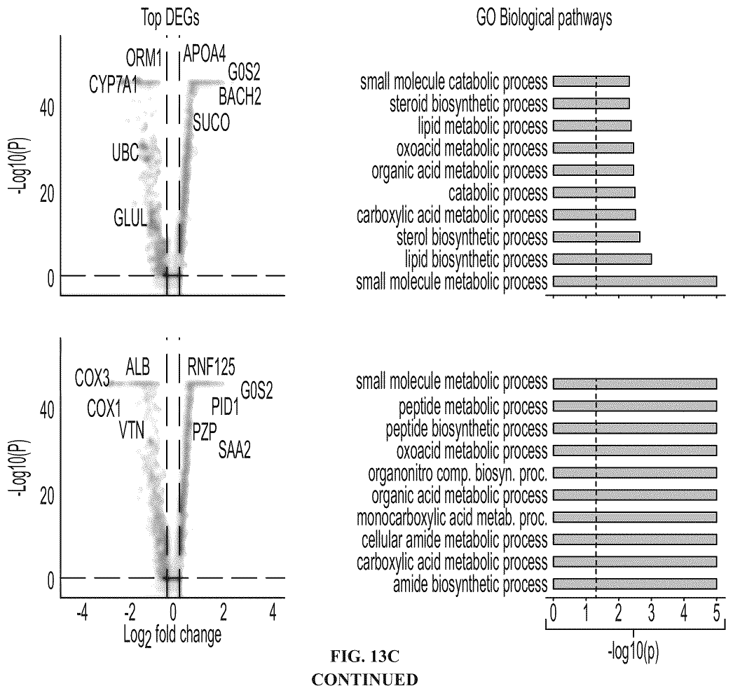

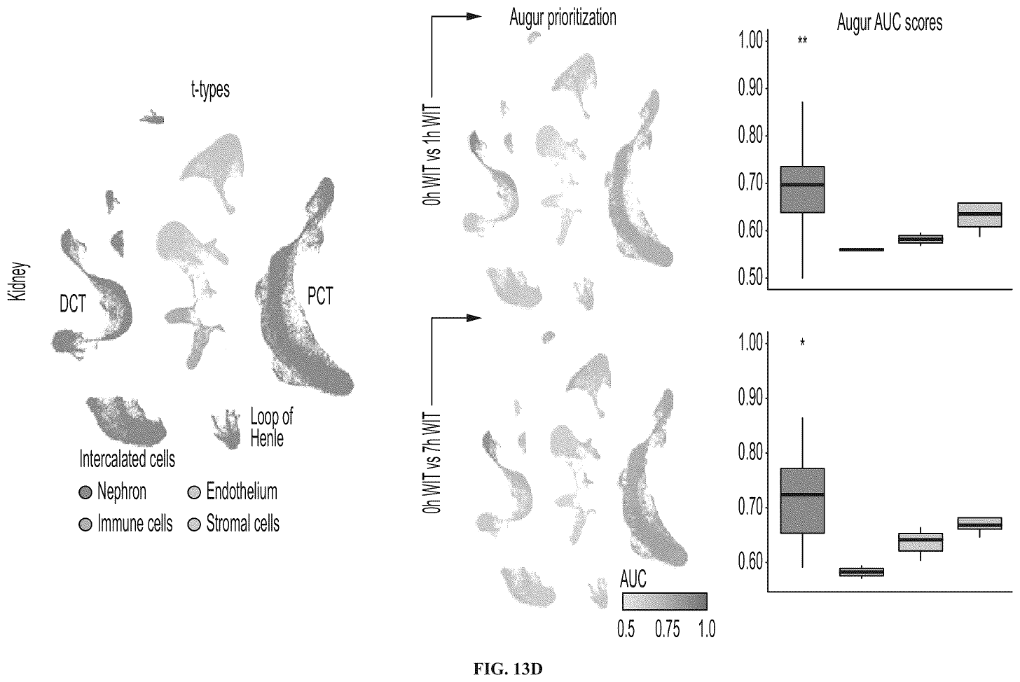

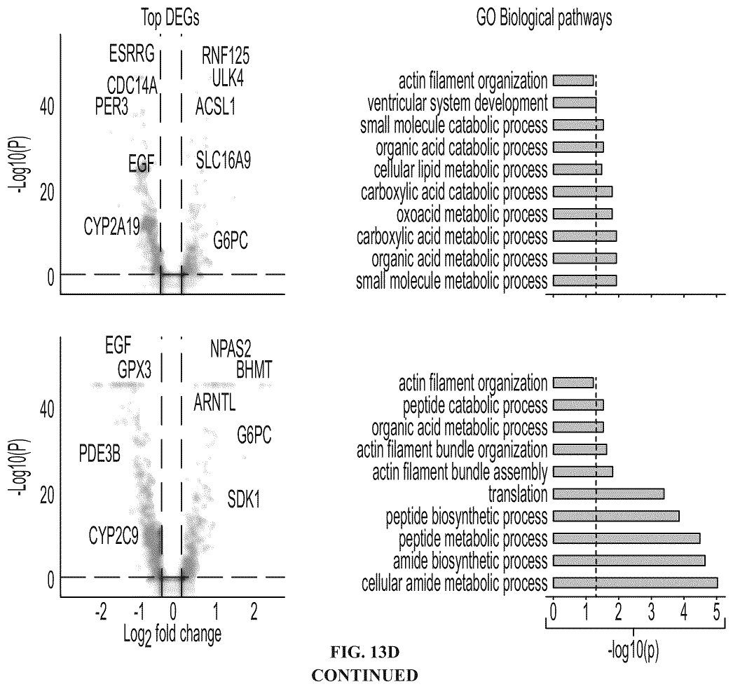

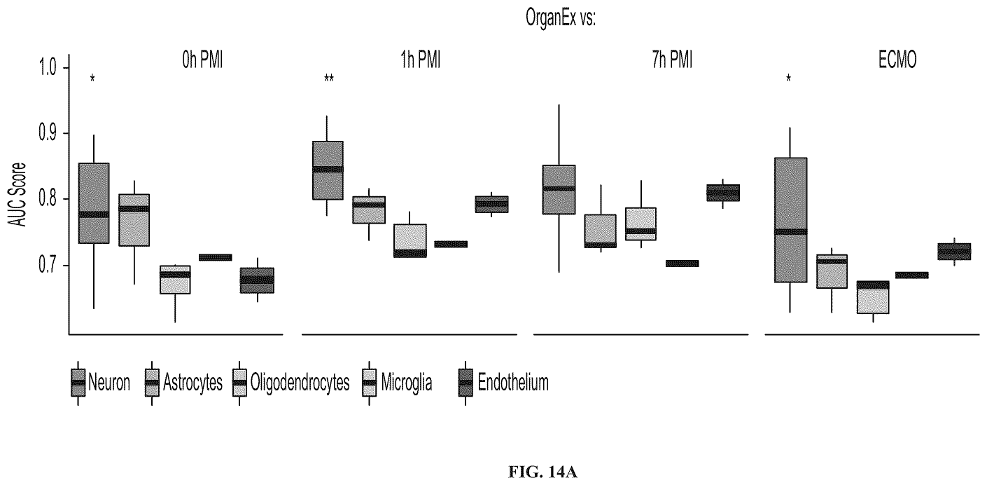

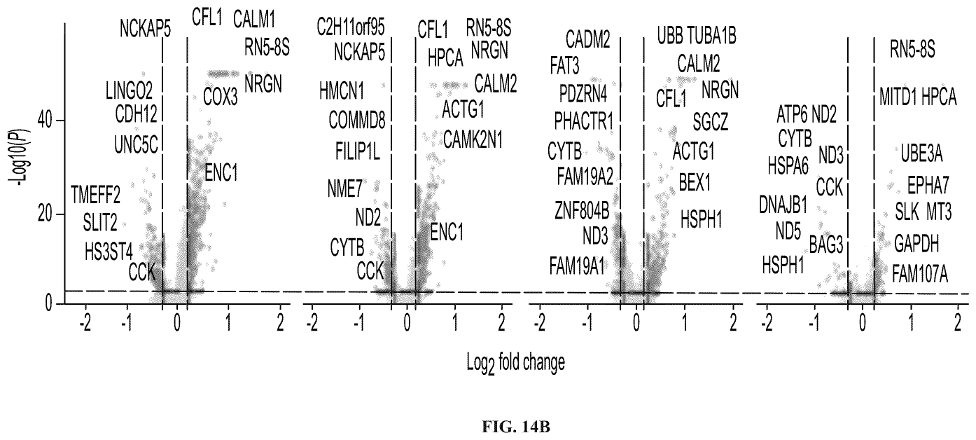

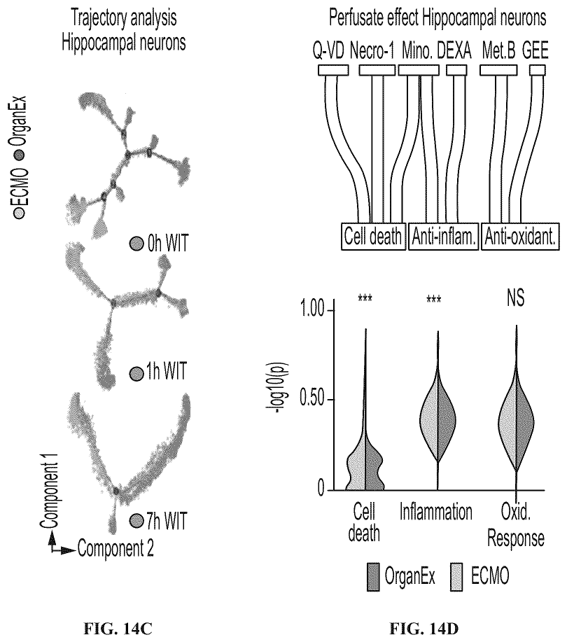

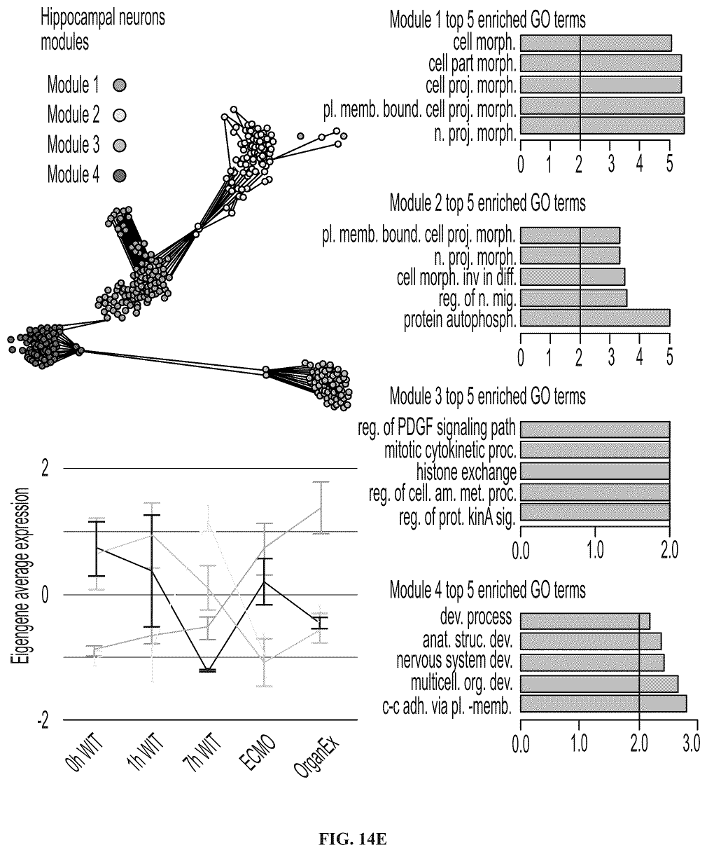

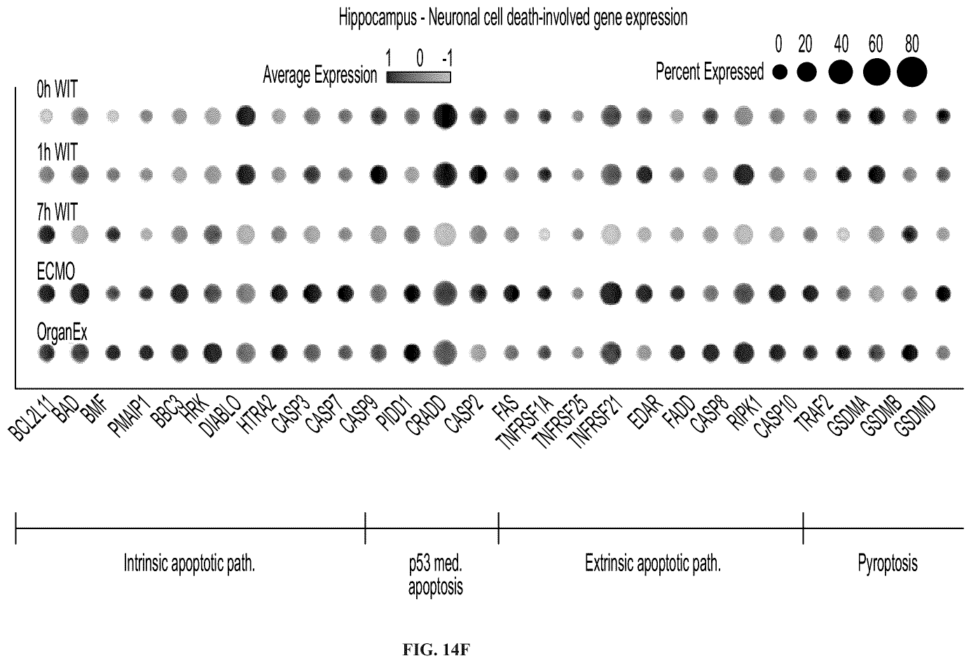

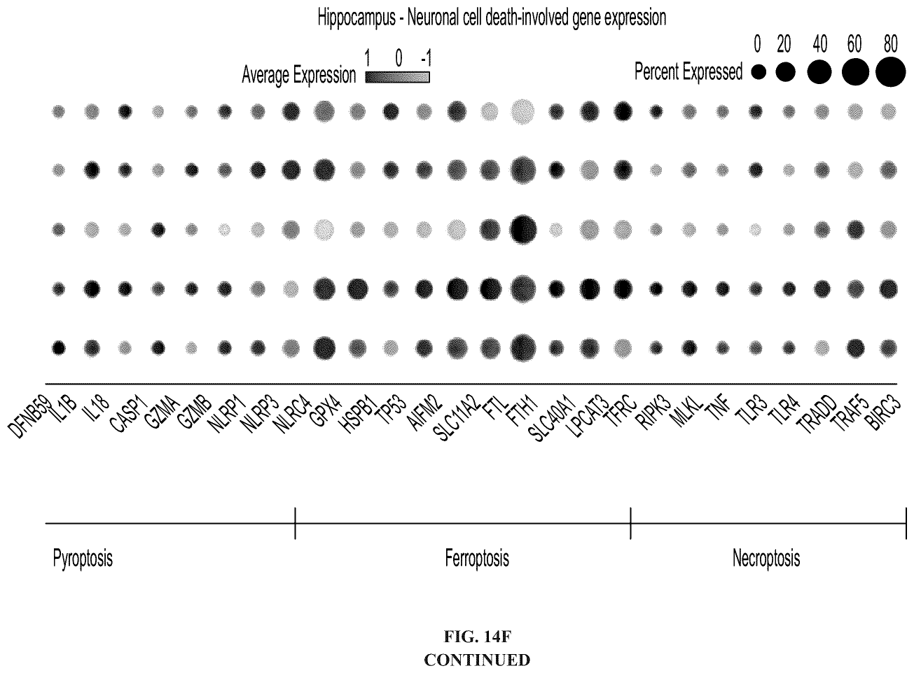

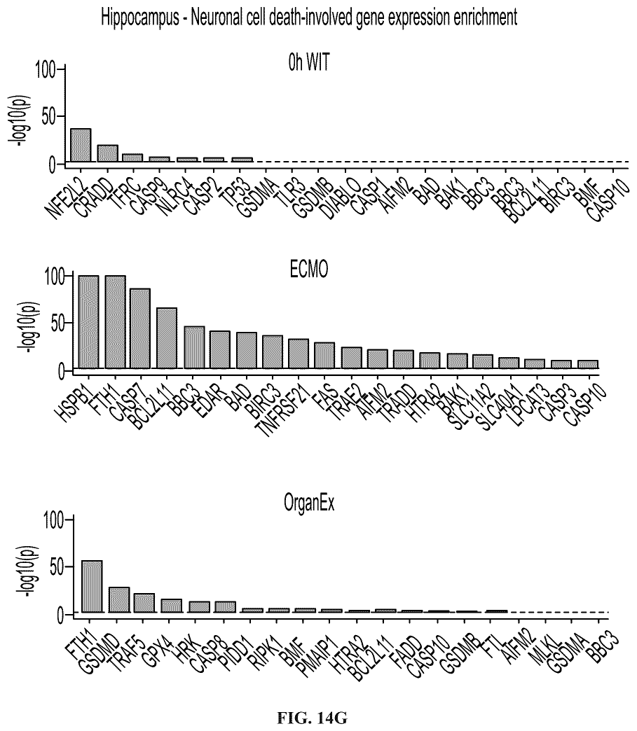

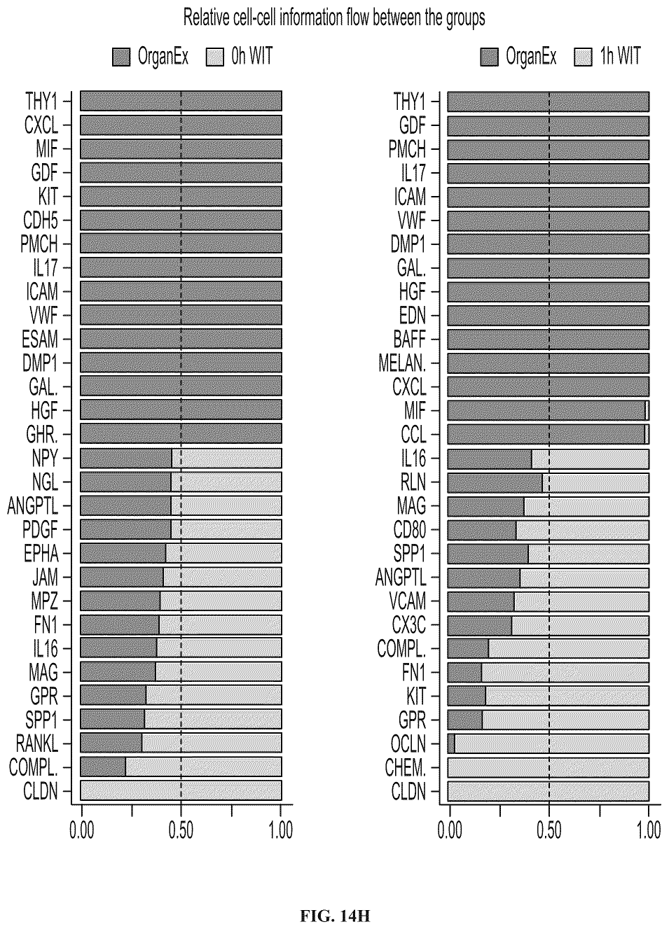

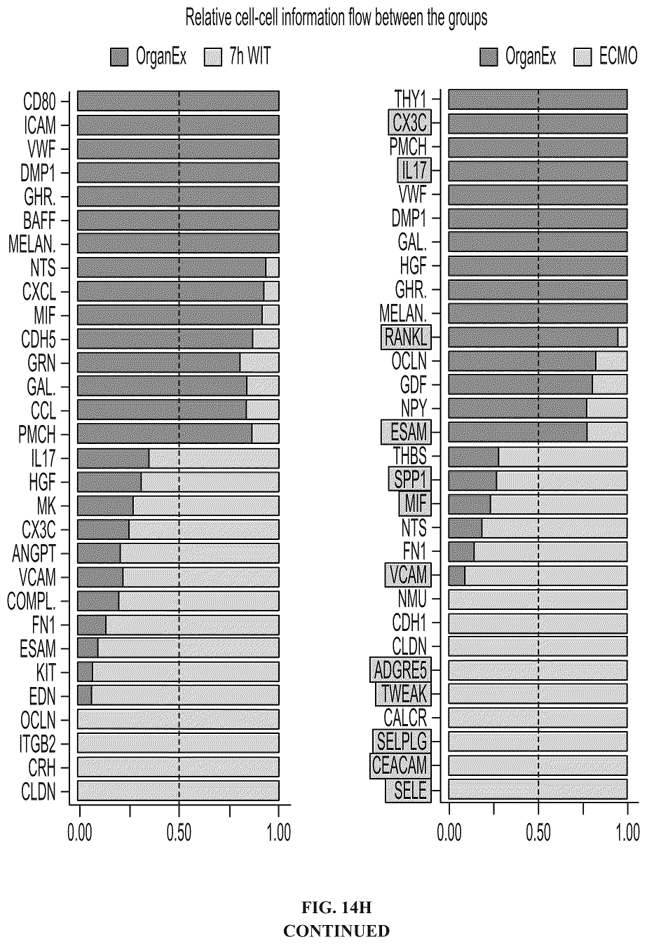

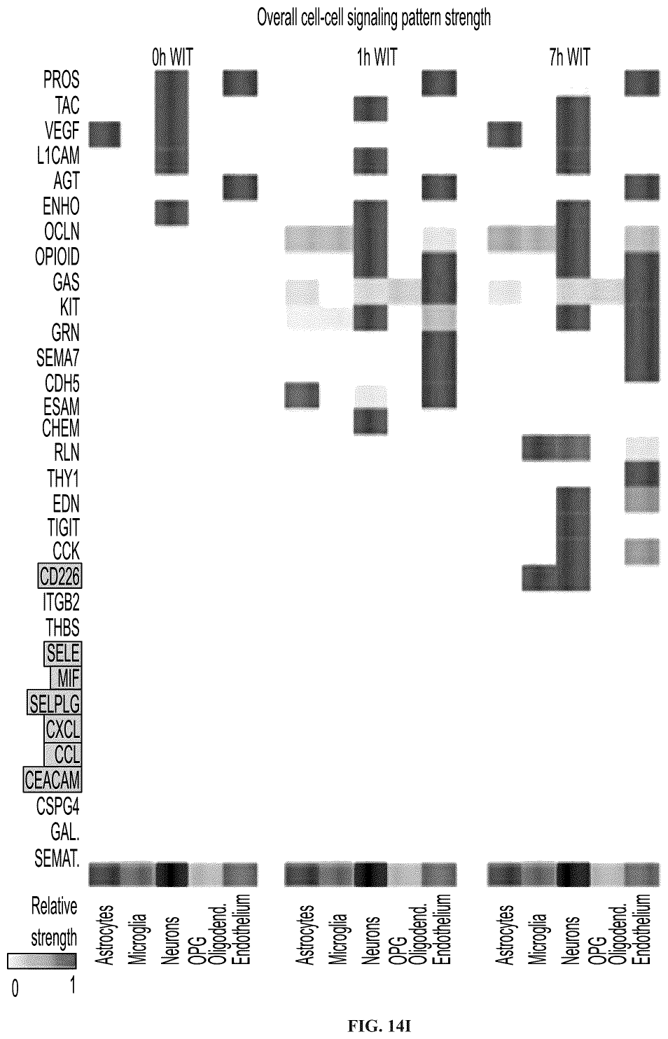

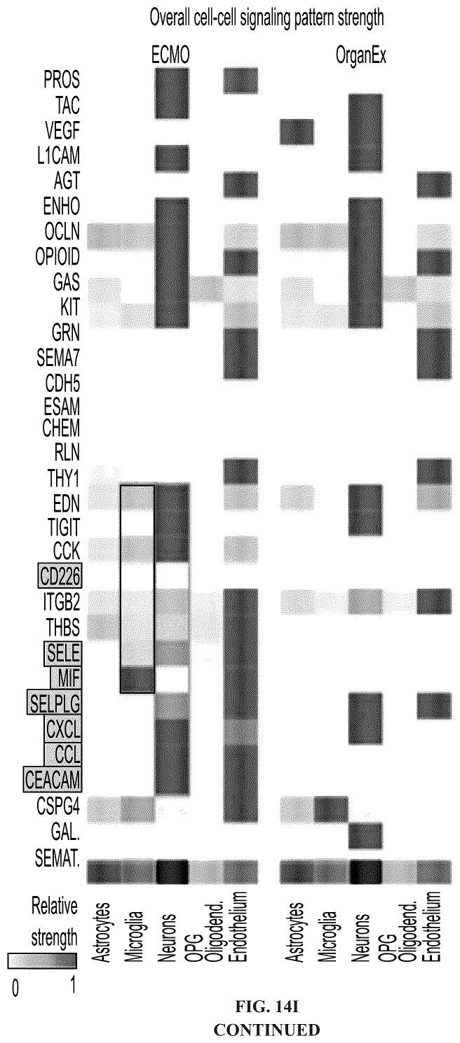

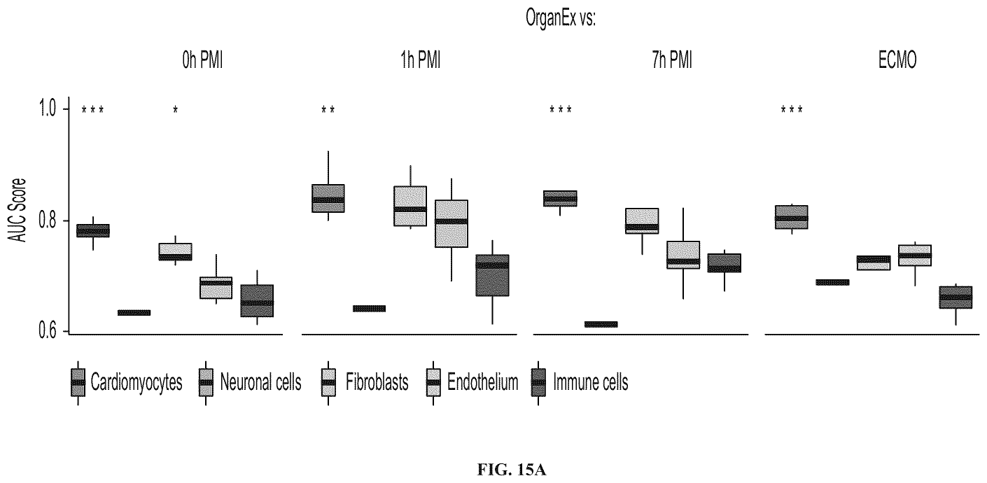

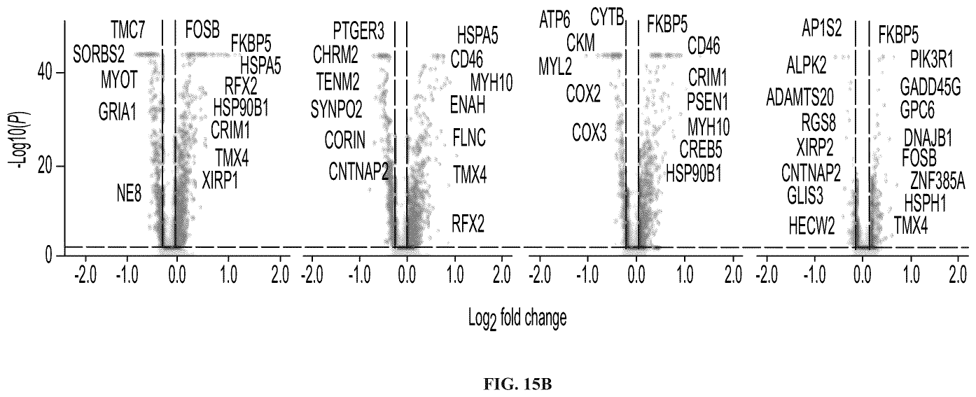

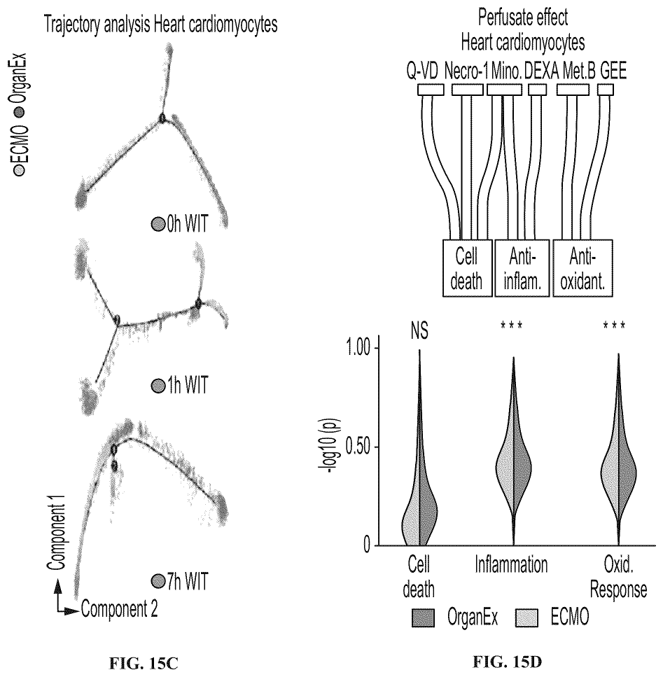

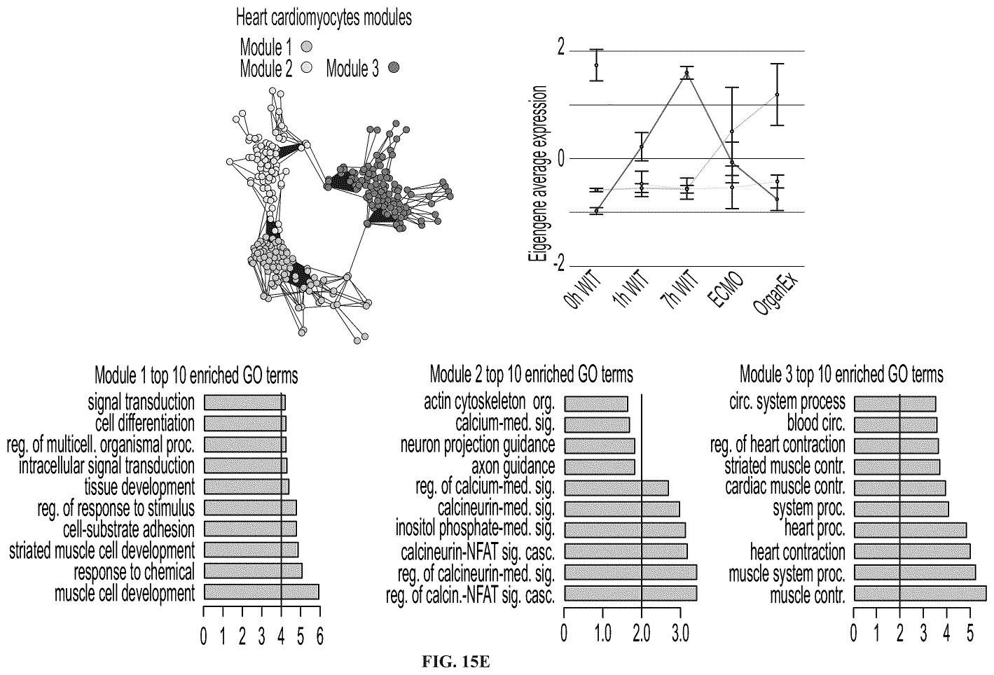

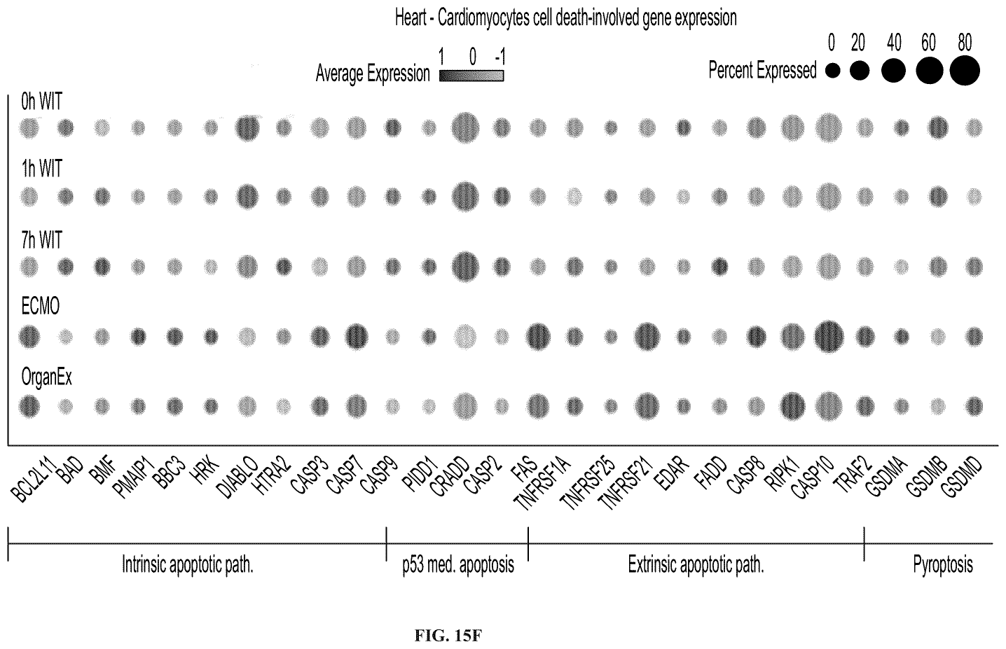

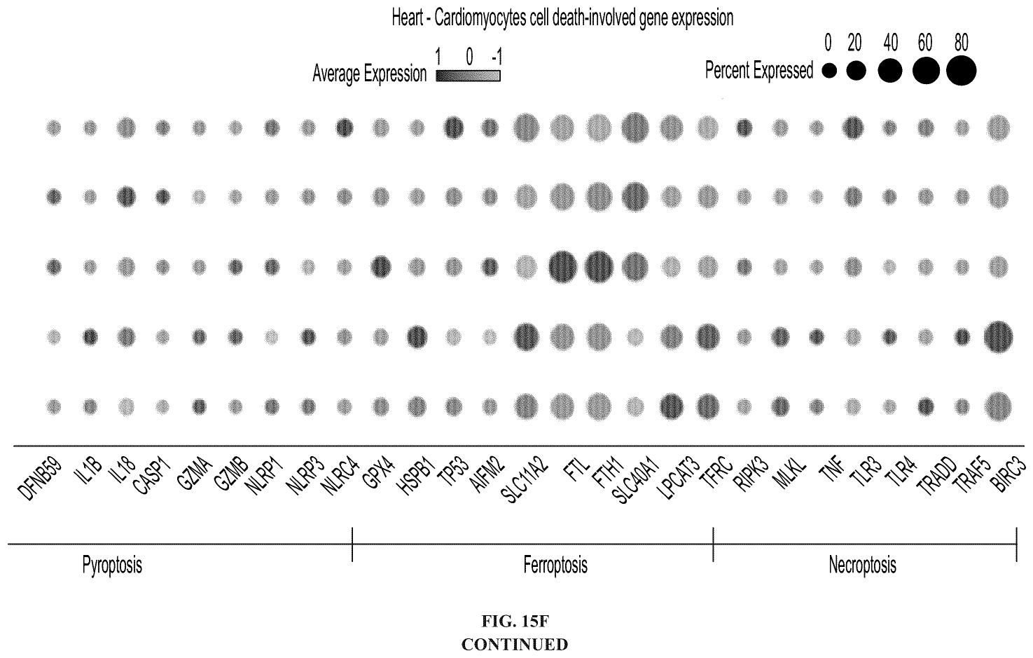

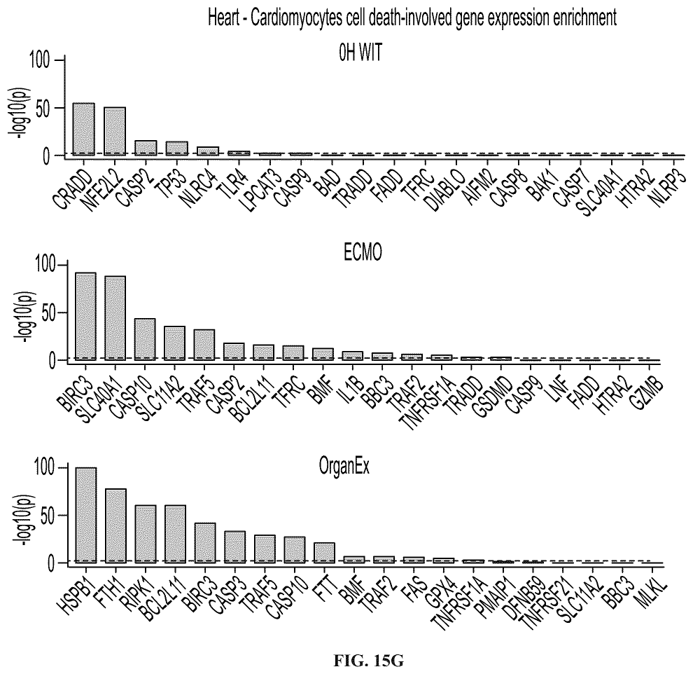

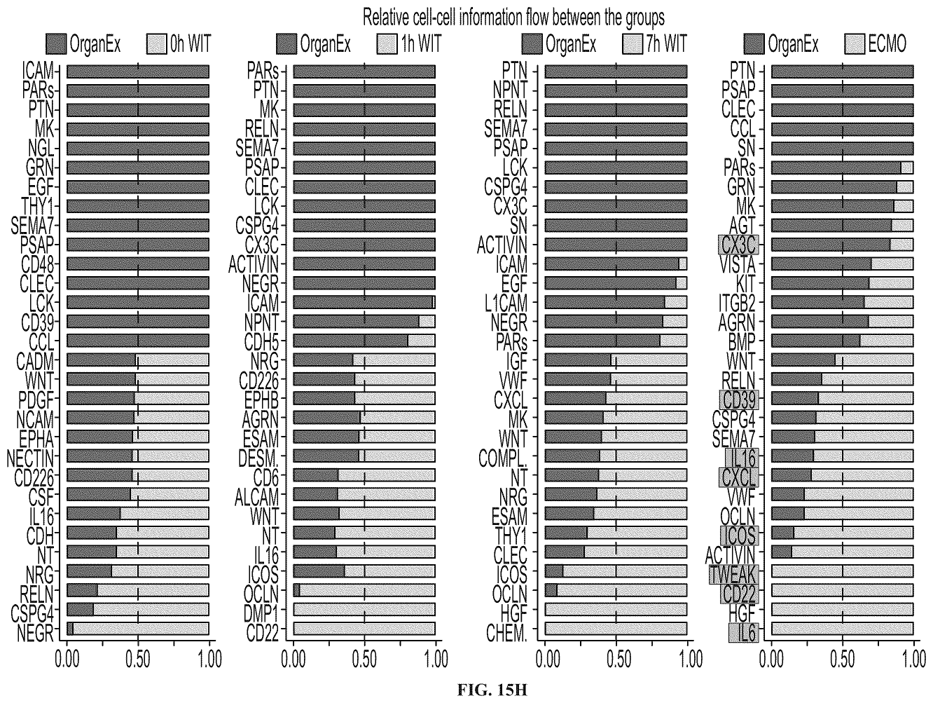

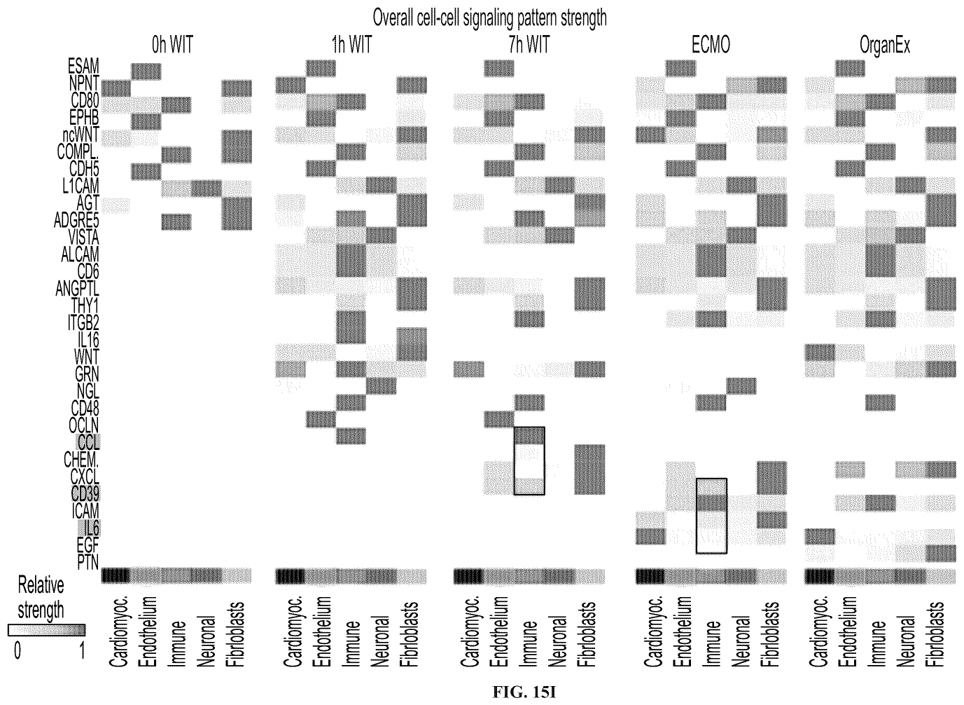

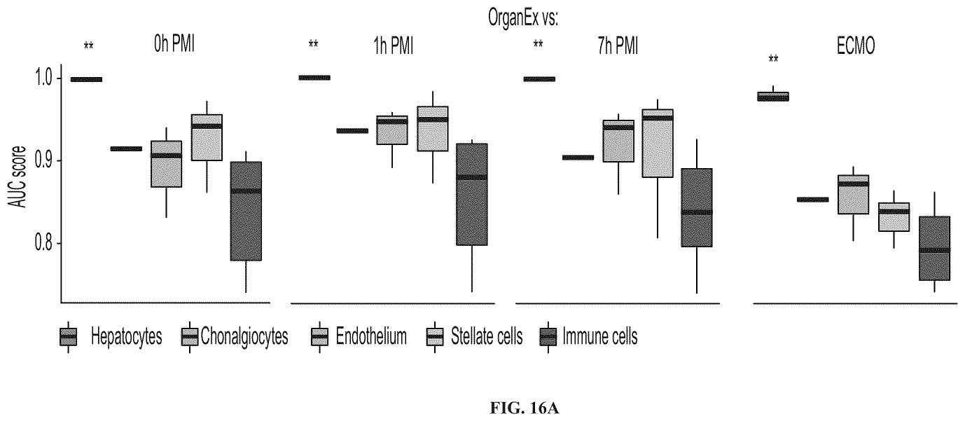

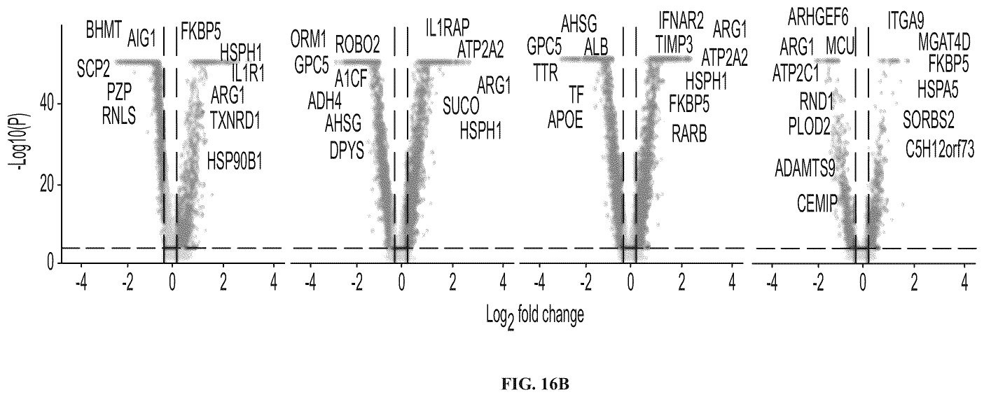

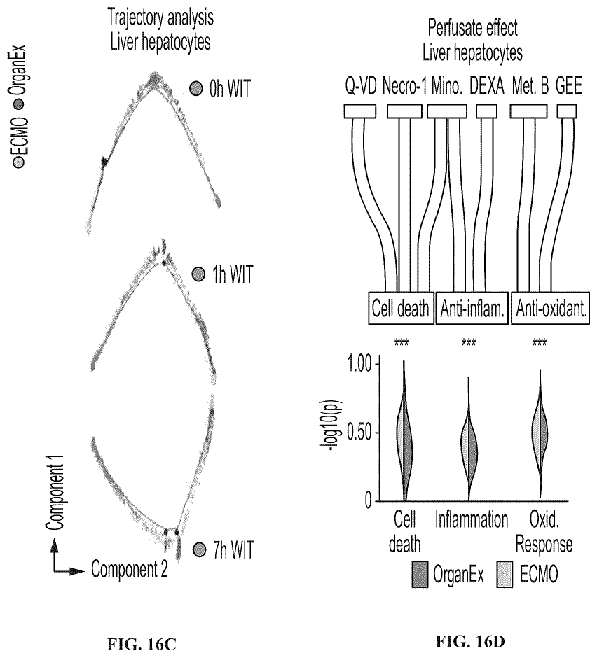

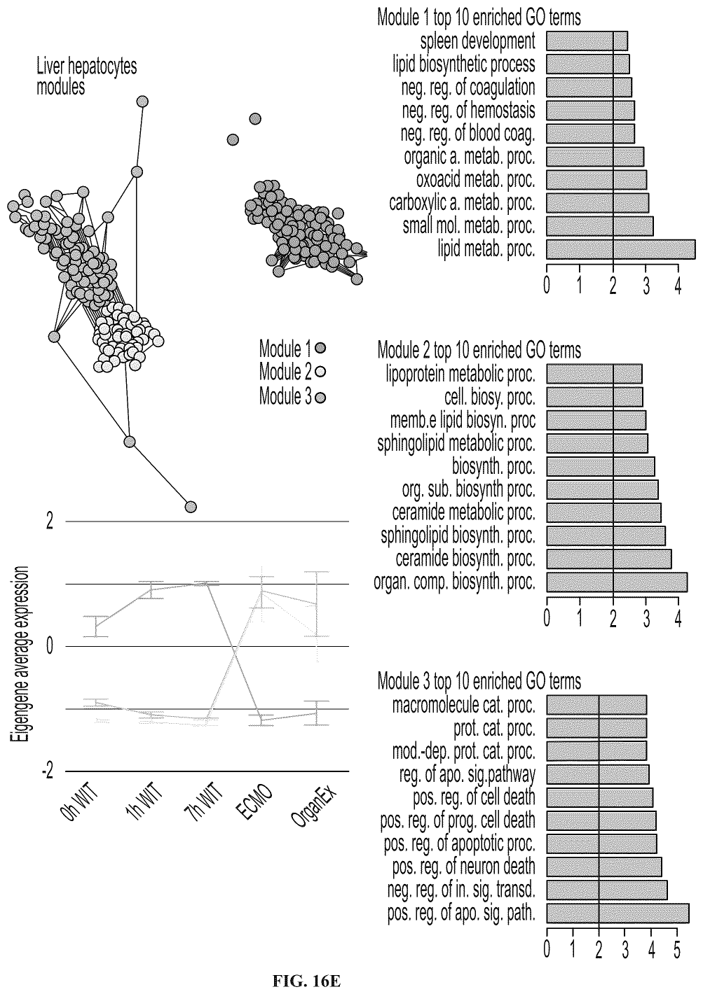

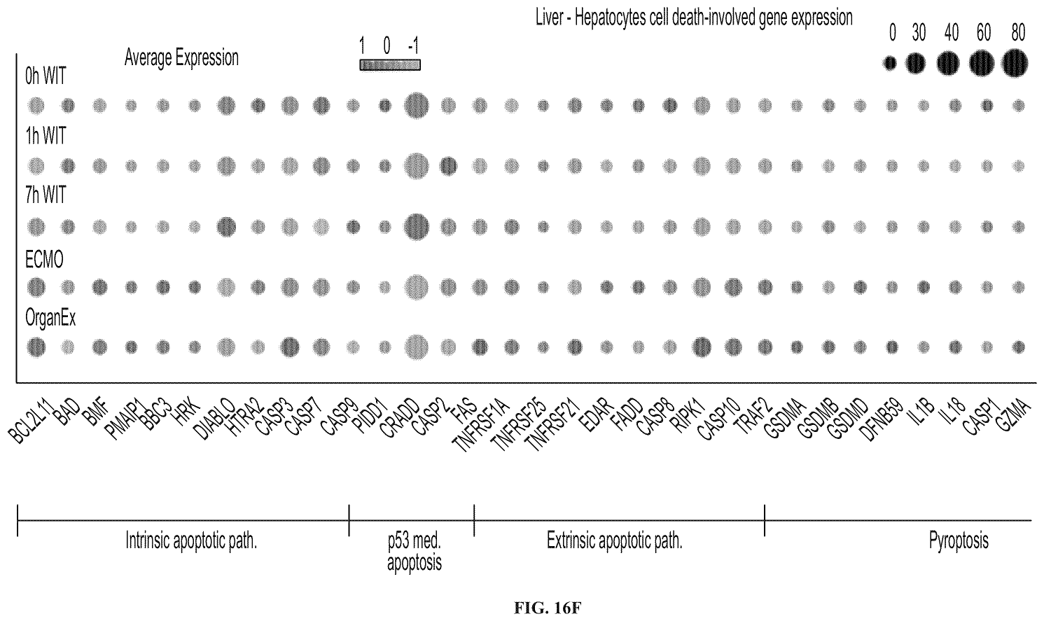

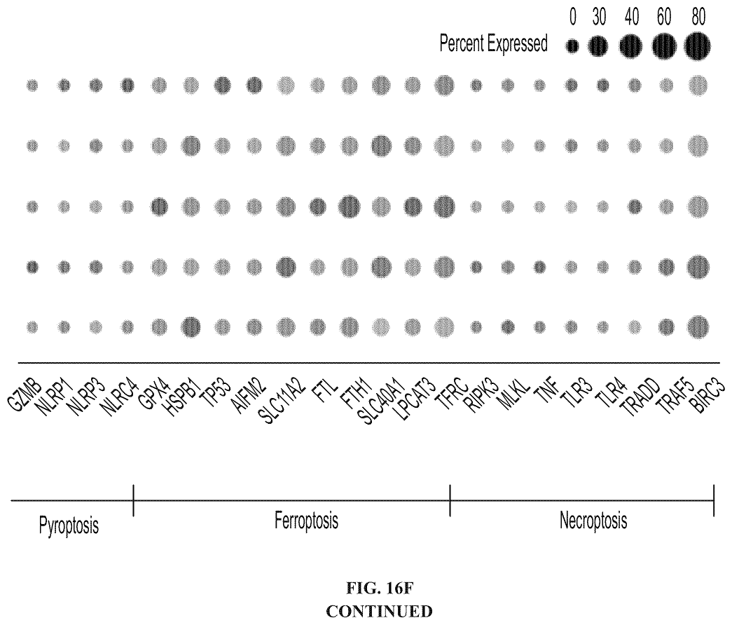

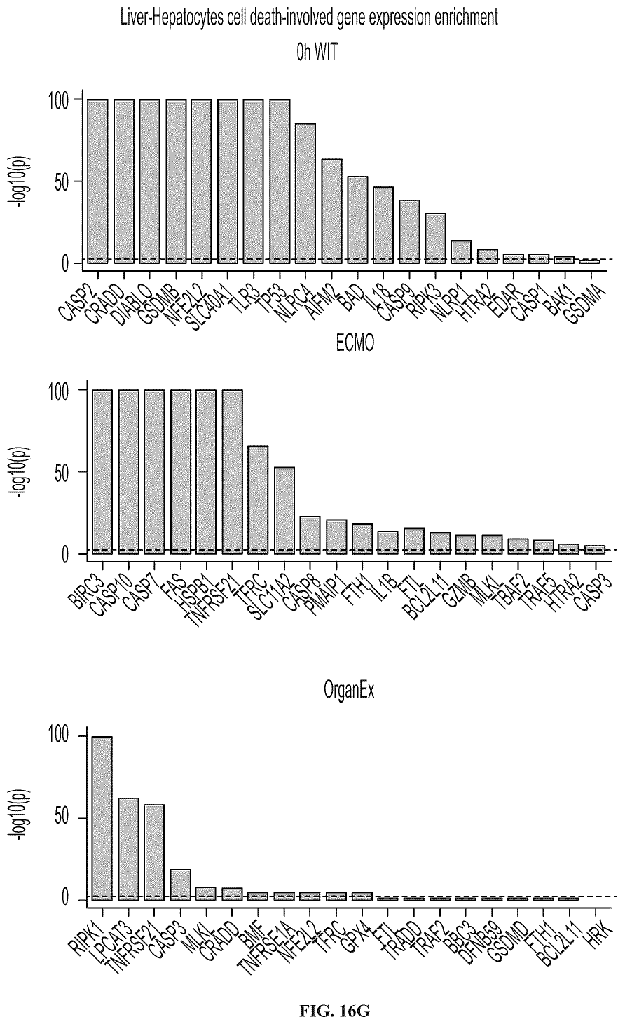

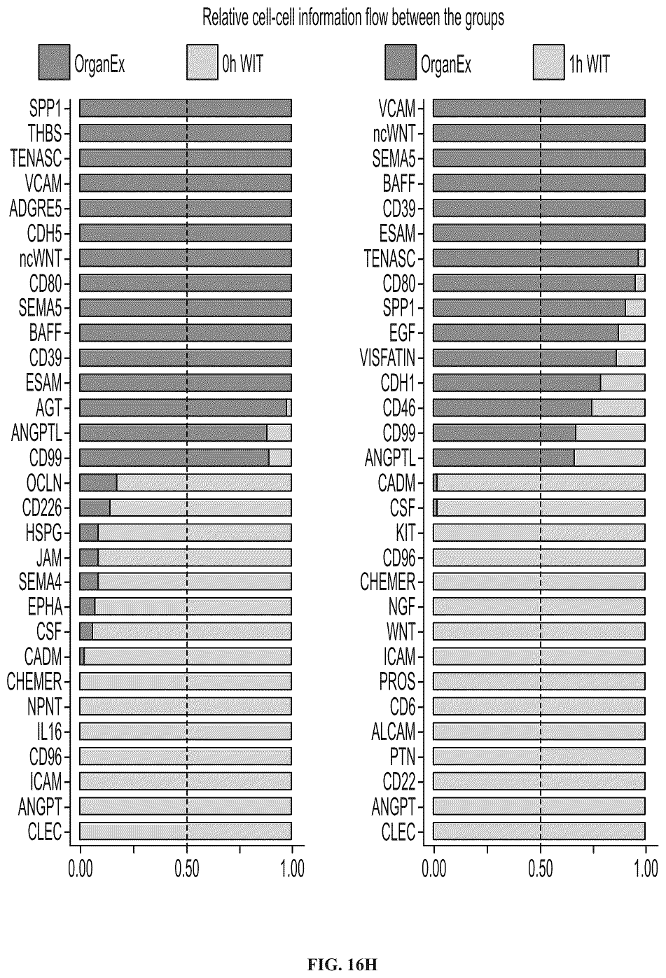

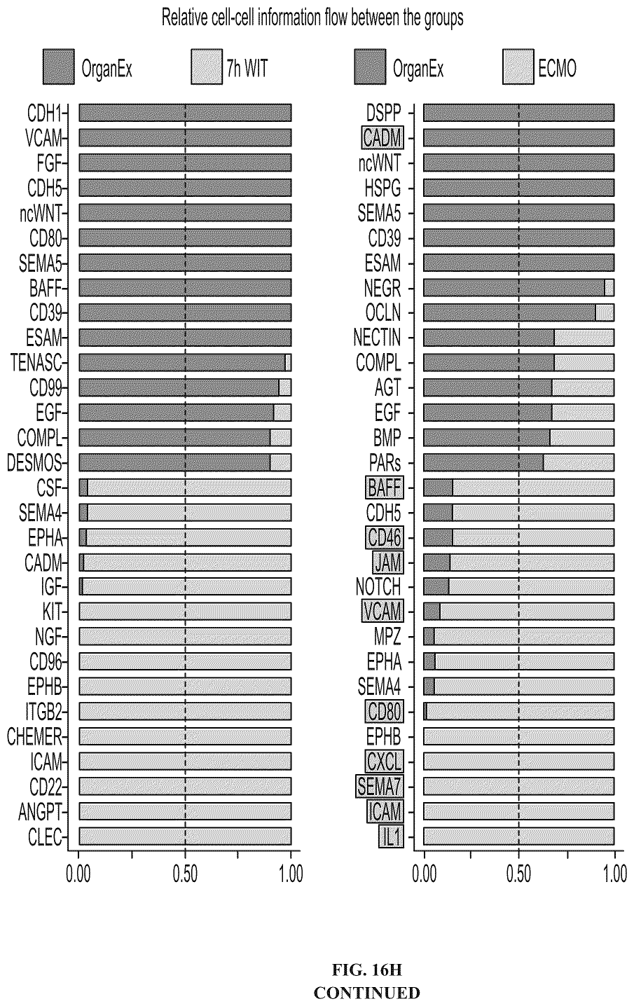

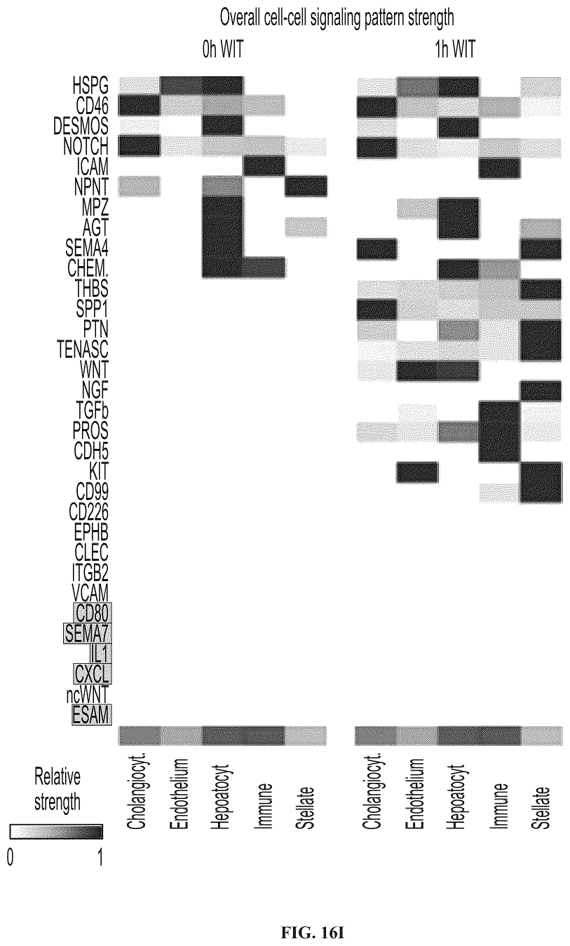

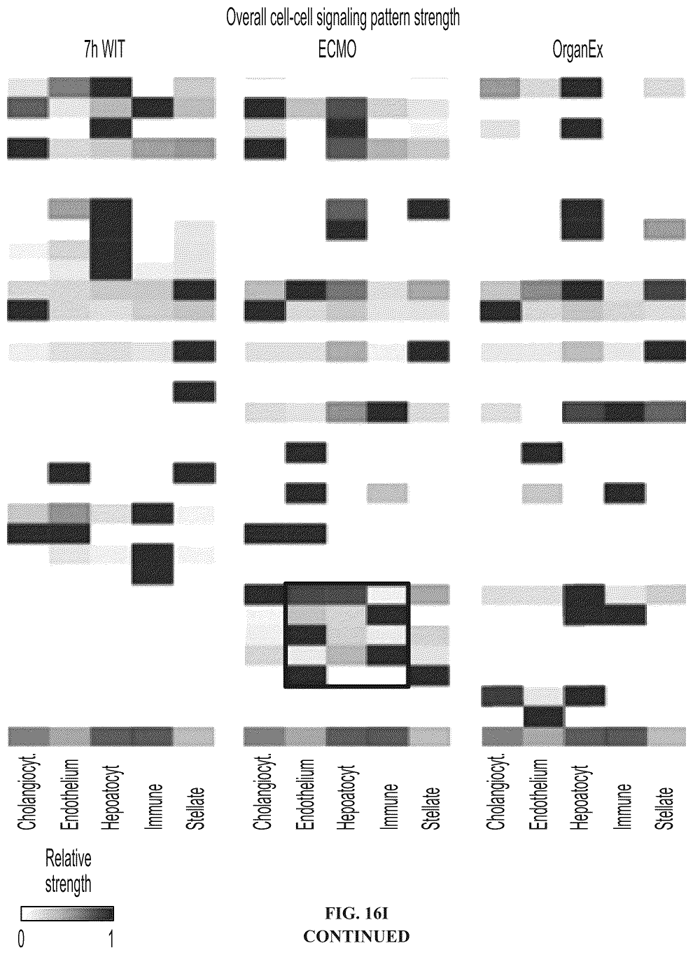

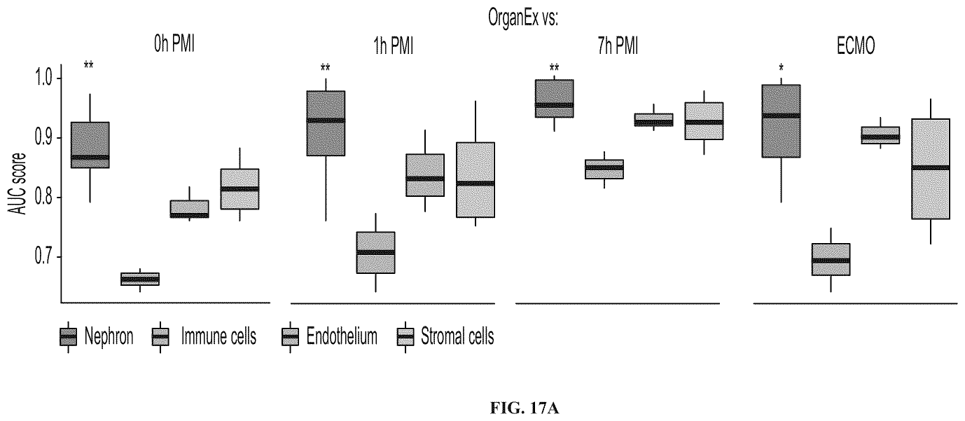

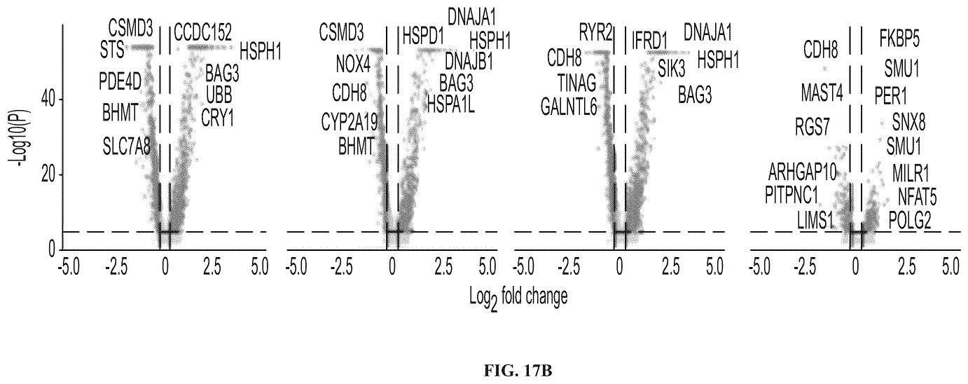

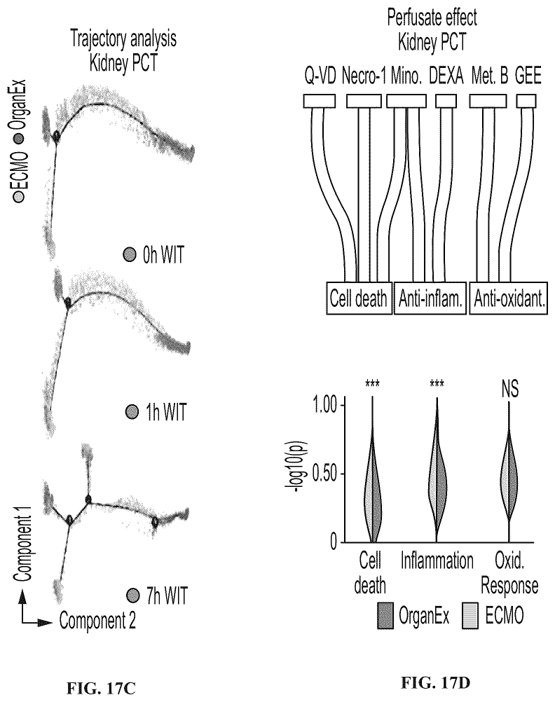

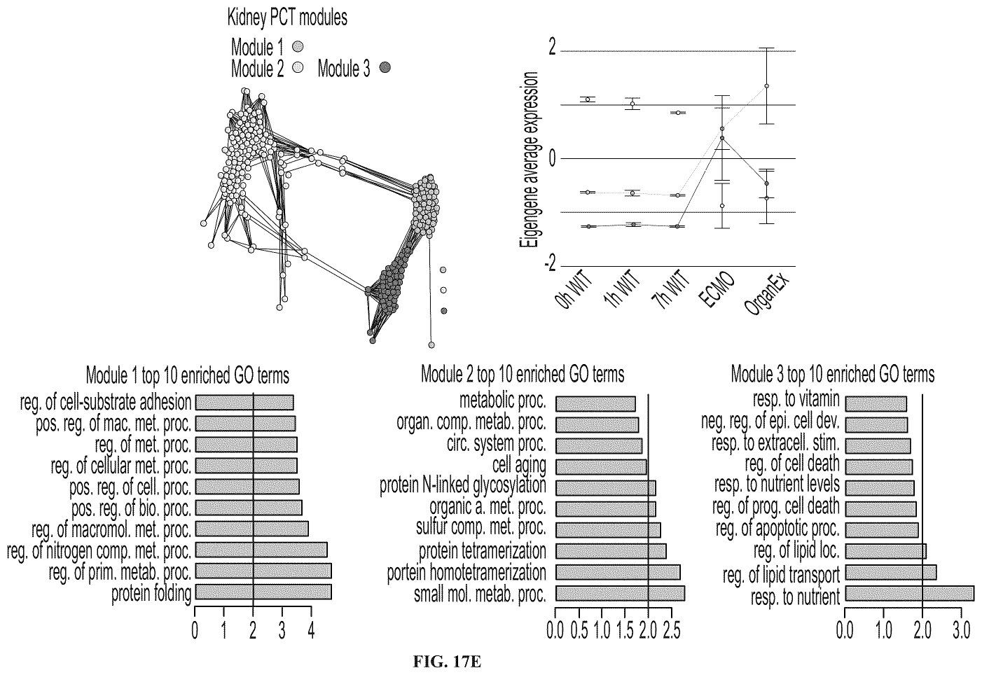

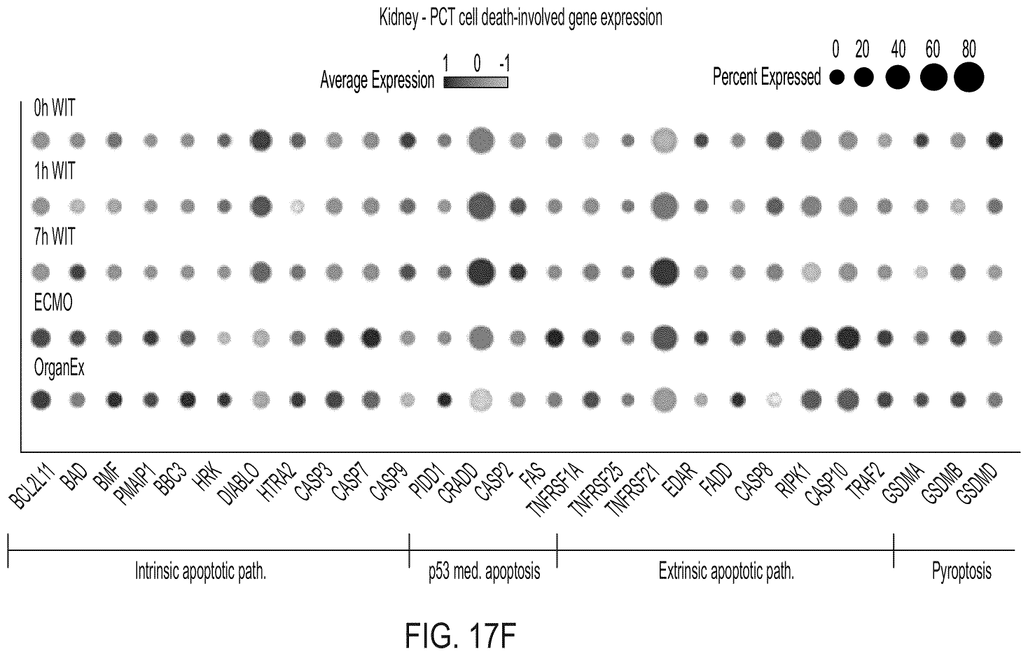

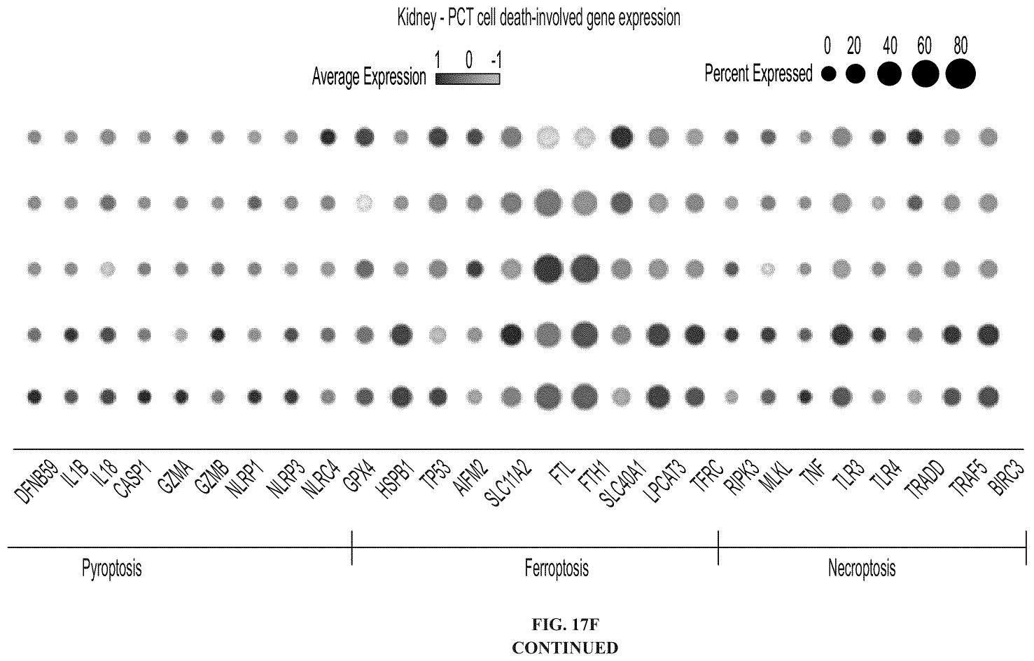

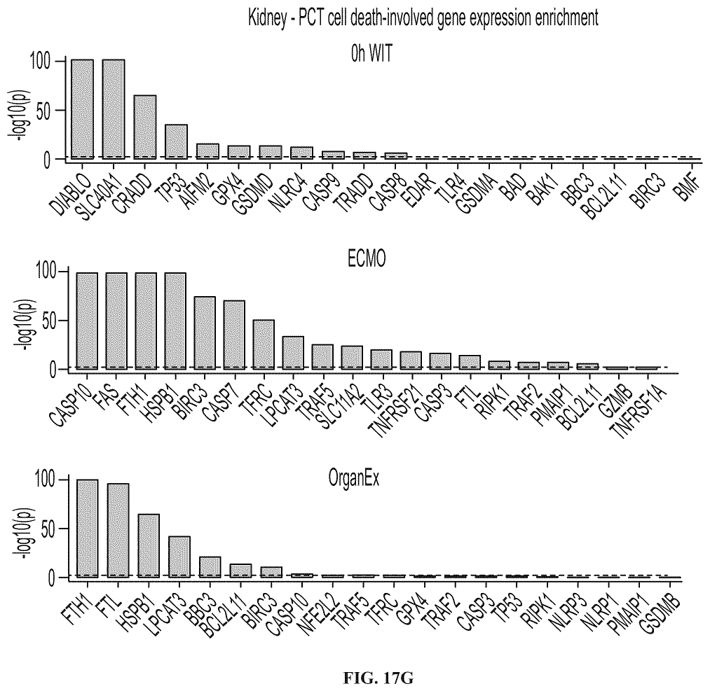

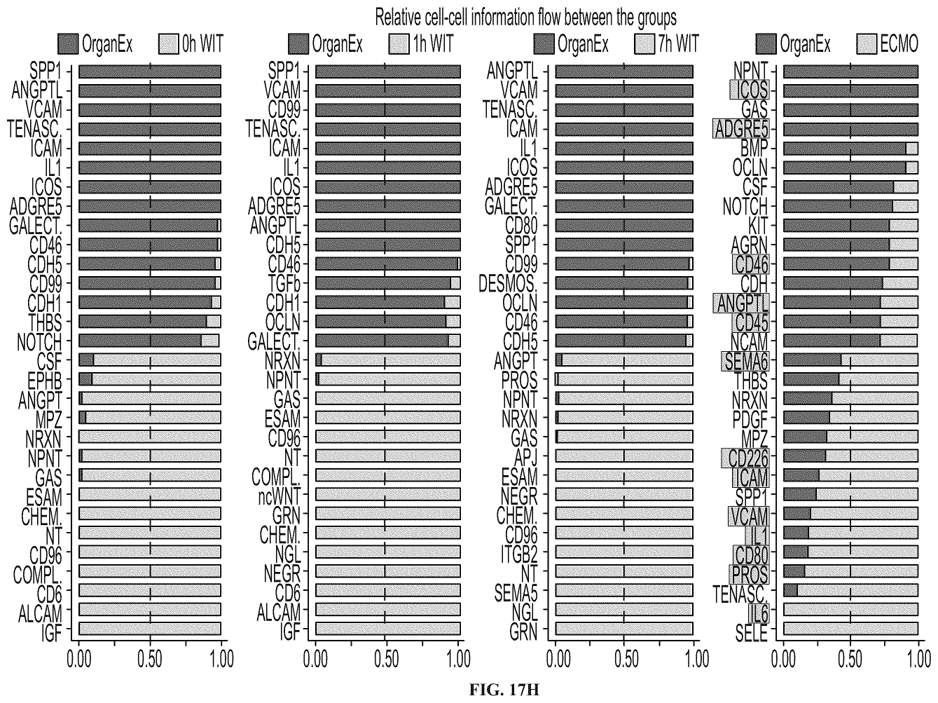

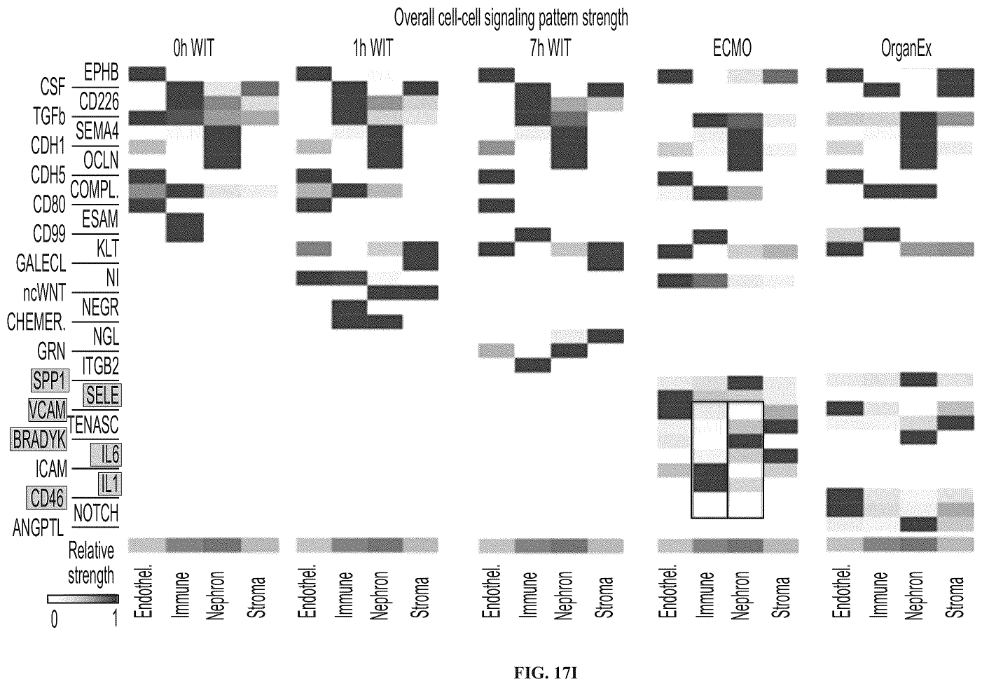

To investigate transcriptomic responses to distinct ischemic exposures and the effects of the OrganEx intervention, single-nucleus RNA-sequencing (snRNA-seq, see Methods) was performed. The computational analysis of snRNA-seq data revealed major transcriptomically-defined cell types (t-types) that were comparable to publicly available human and mouse single-cell datasets (FIGS. 12A-12F). Yet prominent transcriptomic distinctions were also identified between the same t-types across all experimental groups (FIGS. 14A-17I). This extensive cellular taxonomic resource expands upon previous studies and allows for systematic investigation of transcriptomic changes in multiple porcine organs and cell-types exposed to distinct WITs and reperfusion conditions ((FIGS. 13A-13D, 14A-17B), (Tables: 7-23) http://resources.sestanlab.org/OrganEx)).

To compare cell type responsiveness to ischemia based on transcriptomic changes across experimental groups, Augur prioritization was performed and t-types with the greatest transcriptomic divergence were highlighted. This identified prominent changes in neurons, cardiomyocytes, hepatocytes, and proximal convoluted tubule (PCT) cells, consistent with t-types validated in earlier studies and prompting detailed sub-analysis (FIG. 6A-6D). First, it was evaluated whether patterns of transcriptomic changes within OrganEx versus other groups reflect molecular and cellular changes observed in previous studied by assessing for transcriptomic enrichment of corresponding gene sets (Methods). Comparisons between OrganEx and other groups revealed significant enrichment of gene sets facilitating cytoskeletal assembly, DNA repair, ATP metabolism, and suppression of apoptosis and other major cell death pathways across all major cell types in the organs investigated (FIGS. 6A-6D, FIGS. 14A-17I). These data corroborate earlier findings by demonstrating that OrganEx both inhibited progression of cellular injury (e.g., cytoarchitecture, cell death, DNA fragmentation) and promoted repair by modulating cellular pathways on a transcriptomic level. Likewise, genes encoding proteins involved in cell-death, oxidative injury and inflammatory signaling (Tables. 4, 5, 6) are broadly regulated in favor of cell survival in the OrganEx group compared with ECMO in all organs studied. This correlates with choices of pharmacological compounds in the OrganEx perfusate, which had been included using a hypothesis-based, rational-polytherapy approach to modulate these pathways (FIGS. 14A-17D)

Further investigation of OrganEx actions upon glial inflammatory responses underlying brain injury progression after ischemia showed that hippocampal microglial pro-inflammatory transcriptional enhancement was absent in the OrganEx group. Conversely, microglial inflammatory and astrocytic pan-reactive transcriptomic signatures were upregulated in ECMO and 1h WIT groups, respectively. (FIG. 5A). Combined with immunofluorescence analyses of microglial IBA1 and astrocytic GFAP staining (FIGS. 3A-3D, FIGS. 8F-8L), these findings demonstrate that OrganEx intervention modulates the glial inflammatory response.

The transcriptomic signatures of tissue and cellular functioning in heart, liver, and kidney specimens were then evaluated. Cardiomyocytes in the OrganEx group exhibited enrichment of genes orchestrating action potential formation and the well-described shift28 toward glycolytic metabolism following ischemia signifying cardiomyocyte viability (FIG. 6B). In the liver and kidney, hepatocytes and PCT cells were enriched for cytochrome P450 and PCT transporter genes, respectively, in OrganEx versus other groups-though not quite to levels of 0h WIT controls-suggesting preservation of organ-specific functions (FIGS. 6C, 6D). Liver acute-phase reactant and kidney injury marker genes also were notably lower following OrganEx reperfusion than after ECMO or 7h WIT (FIGS. 6C,6D). These data corroborate earlier findings on tissue integrity and cellular activity (FIGS. 3E-3H, FIGS. 5B, 5G; FIGS. 9A-9J; FIGS. 11A-11O).

To identify transcriptomic patterns across experimental groups more systematically, and to determine functional gene modules, we performed co-expression analysis on differentially expressed genes across groups. Eigengenes of each module designated key gene expression trends, with subsequent gene ontology (GO) analyses highlighting relevant biological pathways (FIGS. 14A-17E, FIG. 34). Herein, OrganEx and ECMO had divergent trends across different modules. Gene modules having eigengene increases in the OrganEx group featured GO terms related to cellular upkeep and organ-specific functions. Conversely, modules having increased eigengenes in the ECMO group had GO terms related to cell death (liver and kidney). Finally, analysis of ligand/receptor pairings of t-types showed reduced interactions related to inflammatory pathways (e.g., IL1, IL6, ICAM, VCAM) in OrganEx versus ECMO groups (FIGS. 14A-17I). This mirrors earlier findings that OrganEx reperfusion following ischemia diminishes markers of inflammation, associated with overall decreased cellular injury and augmented repair/protection processes when compared to ECMO (FIGS. 10A-10E, FIGS. 14A-17D). Taken together, snRNA-seq analysis supports cellular and tissue-level findings of decreased cellular injury and the initiation of certain molecular and cellular repair processes following OrganEx intervention.

TABLE 4
Genes involved in cell death

APAF1
BCL2
BID
BIRC2
BIRC3
CASP10
CASP3
CASP6
CASP7
CASP8
CASP9
CFLAR
CHUK
DFFA
DFFB
FADD
GAS2
LMNA
MAP3K14
NFKB1
NFKBIA
RELA
RIPK1
SPTAN1
TNFRSF25
TNFSF10
TRADD
TRAF2
XIAP

TABLE 5
Genes involved in oxidative stress

ABCA1
ADAMTS13
ADIPOQ
AGER
AKR1C2
APP
BMP6
CASP3
CFTR
CLCN3
CP
CXCL8
CYBB
CYP2E1
EDN1
EGFR
ELANE
F3
FPR1
G6PD
GCLC
GPX1
GSTM1
HMOX1
HP
HSD17B10
HSPA4
HSPB1
HTATIP2
IL11
IL13
ITGA2B
ITGB3
JAK2
KPNA2
MAP2K3
MAPK1
MAPK14
MAPK3
MPO
MUC1
MUC5AC
NCF4
NFE2L2
NOS1
NOS2
NOS3
NQO1
NUP153
NUP88
OLR1
PAOX
PEPD
PLA2G7
PLD1
PON1
PRDX1
PRDX2
PRDX3
PRDX4
PRKCB
PRKCD
PRKCZ
PSEN2
PTGER4
PTK2B
RECQL4
SERPINF1
SFTPD
SMPD3
SOD1
SOD3
STAT3
STIM1
TFRC
TGFB1
THG1L
TLR4
TP53
TXN
TXN2
VCAN
VWF

TABLE 6
Genes involved in inflammation

ABCC1
ABCD2
ABHD12
ABI3BP
ABR
ACE
ACER3
ACKR1
ACKR2
ACOD1
ACP5
ADA
ADAM8
ADAM17
ADAMTS12 [symbol]
ADCY1
ADCY7
ADCY8
ADCYAP1
ADIPOQ
ADORA1
ADORA2A
ADORA2B
ADORA3
ADRA2A
ADRB2
AFAP1L2
AGER
AGT
AGTR1A
AGTR1B
AGTR2
AHCY
AHCYL
AHSG
AIM2
AIMP1
AK7
AKNA
AKT1
ALDH2
ALOX5
ALOX5AP
ALOX15
ANKRD42
ANO6
ANXA1
APOD
APP
APPL1
APPL2
AREL1
ASH1L
ATM
ATRN
AXL
B4GALT1
BAP1
BCL6
BCL6B
BCR
BDKRB1
BDKRB2
BRD4
BST1
C1QTNF3
C1QTNF12
C2CD4A
C2CD4B
C3
C5AR1
C5AR2
CALCA
CALCRL
CAMK1D
CAMK4
CAMP
CARD9
CASP1
CASP4
CASP6
CASP12
CCL1
CCL2
CCL3
CCL4
CCL5
CCL6
CCL7
CCL8
CCL9
CCL11
CCL12
CCL17
CCL19
CCL20
CCL21A
CCL21B
CCL21C
CCL22
CCL24
CCL25
CCL26
CCN3
CCN4
CCR1
CCR1L1
CCR2
CCR3
CCR4
CCR5
CCR6
CCR7
CCRL2
CD5L
CD6
CD14
CD24A
CD28
CD40
CD40LG
CD44
CD47
CD68
CD81
CD96
CD163
CD180
CD200
CD200R1
CD200R2
CD200R3
CD200R4
CD276
CD300A
CDH5
CDK19
CEBPA
CEBPB
CELA1
CELF1
CERS6
CFH
CHID1
CHIL1
CHIL3
CHIL4
CHIL5
CHIL6
CHRNA7
CHST1
CHST2
CHST4
CIITA
CLCF1
CLEC10A
CLOCK
CMA1
CMKLR1
CNR1
CNR2
CNTNAP2 [symbol]
CR2
CRH
CRHBP
CRLF2
CRP
CSF1
CSF1R
CSPG4
CSRP3
CST7
CTLA2A
CTNNBIP1 [symbol]
CTSC
CTSS
CUEDC2
CX3CL1
CX3CR1
CXCL1
CXCL2
CXCL3
CXCL5
CXCL9
CXCL10
CXCL13
CXCL15
CXCL17
CXCR2
CXCR3
CXCR6
CYBA
CYBB
CYLD
CYP19A1
CYP26B1
CYSLTR1
DAB2IP
DAGLA
DAGLB
DDT
DDX3X
DHX9
DICER1
DNASE1
DNASE1L3
DPEP1
DROSHA
DUOXA1
DUOXA2
DUSP10
ECM1
EDNRA
EDNRB
EIF2AK1
ELANE
ELF3
ENPP3
EPHA2
EPHB2
EPHB6
EPHX2
ETS1
EXT1
EZH2
F2
F2R
F2RL1
F3
F8
F12
F630003A1 [symbol]
FABP4
FANCA
FANCD2
FCER1A
FCER1G
FCGR1
FCGR2B
FCGR3
FEM1A
FEM1AL
FFAR2
FFAR3
FFAR4
FGFR1
FN1
FNDC4
FOXF1
FOXP1
FOXP3
FPR1
FPR2
FPR3
FPR-RS3
FPR-RS4
FPR-RS6
FPR-RS7
FUT7
GAL
GATA3
GBP5
GGT1
GGT5
GHRL
GHSR
GIT1
GJA1
GM5849
GPER1
GPR4
GPR17
GPR31B
GPR33
GPRC5B
GPS2
GPSM3
GPX1
GPX2
GPX4
GRN
GSDMD
GSTP1
H2BC1
HAMP
HAVCR2
HC
HCK
HDAC5
HDAC7
HDAC9
HGF
HIF1A
HK1
HMGB1
HMGB2
HMOX1
HNRNPA0
HP
HPS1
HRH4
HSPD1
HYAL1
HYAL2
HYAL3
ICAM1
IDO1
IER3
IF135
IFNG
IGF1
IGH-7
IGH-8
IGHG1
IGHG2A
IGHG2B
IL1A
IL1B
IL1F10
IL1R1
IL1R2
IL1RAP
IL1RL1
IL1RL2
IL1RN
IL2
IL2RA
IL4
IL4RA
IL5RA
IL6
IL10
IL12B
IL13
IL16
IL17A
IL17B
IL17C
IL17D
IL17F
IL17RA
IL17RB
IL17RC
IL17RE
IL18
IL18R1
IL18RAP
IL20RB
IL22
IL22RA2
IL23A
IL23R
IL25
IL27
IL31RA
IL33
IL34
IL36A
IL36B
IL36G
IL36RN
IRAK2
IRF3
IRF5
ISL1
ITGA2
ITGAM
ITGAV
ITGB1
ITGB2
ITGB2L
TGB6
ITIH4
JAK2
JAM3
KARS
KDM6B
KIT
KL
KLK1B1
KLKB1
KLRH1
KNG1
KPNA6
KRT1
KRT16
LACC1
LAT
LBP
LDLR
LEP
LGALS9
LIAS
LILRA5
LILRB4A
LILRB4B
LIPA
LOXL3
LPCAT3
LPL
LRFN5
LRRC19
LRRK2
LTA
LTB4R1
LTB4R2
LXN
LY86
LY96
LYN
MACIR
MAP2K3
MAPK8
MAPK14
MAPKAPK2
MAS1
MCPH1
MDK
MECOM
MEFV
MEP1B
METRNL
MFHAS1
MGLL
MIF
MIR21A
MIR147
MIR155
MIR301
MIR324
MIR883B
MIR7116
MIR7578
MMP8
MRGPRA3
MS4A2
MSMP
MTOR
MUC19
MVK
MYD88
MYLK3
MYO5A
NAIP1
NAIP2
NAIP5
NAIP6
NAIP7
NAPEPLD
NCF1
NDFIP1
NDST1
NDUFC2
NDUFS4
NFE2L1
NFE2L2
NFKB1
NFKBIA
NFKBIB
NFKBID
NFKBIZ
NINJ1
NKIRAS2
NLRC3
NLRC4
NLRP1A
NLRP1B
NLRP3
NLRP4A
NLRP4B
NLRP4C
NLRP4E
NLRP4F
NLRP6
NLRP9A
NLRP9B
NLRP9C
NLRP10
NLRP12
NLRX1
NMI
NOD2
NOS2
NOTCH1 [symbol]
NOTCH2 [symbol]
NPPA
NPY
NPY5R
NR1D1
NR1D2
NR1H3
NR1H4
NRROS
NT5E
NUPR1
ODAM
OLR1
ORM1
ORM2
OSM
OTULIN
P2RX1
P2RX7
PARK7
PARP4
PBK
PBXIP1
PDCD4
PDE2A
PDE5A
PER1
PF4
PGLYRP [symbol]
PGLYRP [symbol]
PIK3AP1
PIK3CD
PIK3CG
PJA2
PLA2G2 [symbol]
PLA2G2 [symbol]
PLA2G3
PLA2G4A [symbol]
PLA2G5
PLA2G7
PLA2G10 [symbol]
PLAA
PLCG2
PLD3
PLD4
PLGRKT [symbol]
PLP1
PMP22
PNMA1
POLB
PPARA
PPARD
PPARG
PPBP
PRCP
PRDX2
PRKCA
PRKCQ
PRKCZ
PRKD1
PROC
PSEN2
PSMA1
PSMB4
PSTPIP1
PTAFR
PTGDR
PTGER1
PTGER2
PTGER3
PTGER4
PTGES
PTGFR
PTGIR
PTGIS
PTGS1
PTGS2
PTN
PTPN2
PXK
PYCARD
RABGEF1
RARRES2 [symbol]
RASGRP1
RB1
RBPJ
REG3A
REG3B
REG3G
REL
RELA
RELB
RHBDD3
RICTOR
RIPK1
RORA
RPS6KA4
RPS6KA5
RPS19
RTN4
S1PR3
S100A7A
S100A8
S100A9
SAA1
SAA2
SAA3
SAA4
SBNO2
SCGB1A1
SCN9A
SCNN1B
SCYL1
SCYL3
SDC1
SEH1L
SELE
SELENOS
SELP
SEMA7A
SERPINA1B
SERPINA3N
SERPINB1A
SERPINB9
SERPINE1
SERPINF1
SERPINF2
SETD4
SGMS1
SHARPIN
SHPK
SIGIRR
SIGLECE
SIGLECG
SIRPA
SLAMF1
SLAMF8
SLC7A2
SLC11A1
SLIT2
SMAD3
SMPDL3B
SNAP23
SNCA
SNX4
SOCS3
SOCS5
SOD1
SPATA2
SPHK1
SPN
STAB1
STAP1
STARD7
STAT3
STAT5A
STAT5B
STING1
STK39
SUCNR1
SYK
SYT11
TAC1
TAC4
TAFA3
TARM1
TBC1D23
TBXA2R
TCIRG1
TFF2
TGFB1
THBS1
THEMIS2
TICAM1
TICAM2
TIMP1
TIRAP
TLR1
TLR2
TLR3
TLR4
TLR5
TLR6
TLR7
TLR8
TLR9
TLR11
TLR12
TLR13
TNF
TNFAIP3
TNFAIP6
TNFAIP8L2
TNFRSF1A
TNFRSF1B
TNFRSF4
TNFRSF11A
TNFSF4
TNFSF11
TNFSF18
TNIP1
TNIP2
TOLLIP
TPSB2
TRADD
TRAF3IP2
TREM1
TREM2
TREX1
TRIL
TRIM55
TRP73
TRPV1
TRPV4
TSLP
TSPAN2
TTBK1
TTC39AOS1
TUSC2
TYRO3
UACA
ULK4
UMOD
UNC13D
VAMP7
VAMP8
VNN1
VPS35
WDR83
WFDC1
WNT5A
XCL1
YWHAZ
ZBP1
ZC3H12A
ZFP35
ZFP36
ZFP580
ZP3 [symbol]
indicates data missing or illegible when filed

TABLE 7
Upregulated modules in the hippocampus

| 0 h PMI | UPREGULATED MODULES | | | | |
|---|---|---|---|---|---|
| vs | MODULE | TOP TERMS | Q VAL | GENES | TERMS |
| 1 h PM | M1 | protein homotetramerization | 0.00130534 | 6 | 7 |
| | | protein tetramerization | 0.00166205 | | |
| | | protein homooligomerization | 0.00399511 | | |
| | | cellular response to organonitrogen | 0.00437743 | | |
| | | compound cellular response to nitrogen | 0.00604231 | | |
| | | compound protein complex | 0.00604231 | | |
| | | oligomerization | 0.00609269 | | |
| | | response to organonitrogen compound | | | |
| | M2 | purine-containing compound metabolic | 0.00399511 | 5 | 1 |
| | | process | | | |
| 7 h PM | M1 | regulation of nervous system development | 0.01547127 | 11 | 7 |
| | | neuron development | 0.01547127 | | |
| | | central nervous system development | 0.01547127 | | |
| | | neuron differentiation | 0.01627243 | | |
| | | cellular component morphogenesis | 0.0166483 | | |
| | | generation of neurons | 0.0166483 | | |
| | | neurogenesis | 0.0166483 | | |
| | M2 | transmembrane receptor protein | 0.01547127 | 10 | 2 |
| | | tyrosine kinase signaling pathway | | | |
| | | actin cytoskeleton organization | 0.01547127 | | |

TABLE 8
Downregulated modules in the hippocampus
DOWNREGULATED MODULES

| | MODULE | TOP TERMS | Q VAL | GENES | TERMS |
|---|---|---|---|---|---|
| 1 h PM | M1 | transport along microtubule | 0.00984952 | 20 | 11 |
| | | microtubule-based transport | 0.00984952 | | |
| | | organelle transport along | 0.00984952 | | |
| | | microtubule | 0.00996313 | | |
| | | cytoskeleton-dependent | 0.01121427 | | |
| | | intracellular transport | 0.01612674 | | |
| | | microtubule-based movement | 0.01680074 | | |
| | | regulation of phosphatase | 0.01744301 | | |
| | | activity | 0.01744301 | | |
| | | regulation of dephosphorylation | 0.02451 | | |
| | | establishment of organelle | | | |
| | | localization | | | |
| | | glycoprotein metabolic process | | | |
| | | organelle localization | | | |
| | M2 | positive regulation of chemotaxis | 0.00984952 | 9 | 18 |
| | | regulation of chemotaxis | 0.01058596 | | |
| | | positive regulation of response to | 0.01058596 | | |
| | | external stimulus | | | |
| | | endothelial cell migration | 0.01121427 | | |
| | | taxis | 0.01407071 | | |
| | | chemotaxis | 0.01407071 | | |
| | | epithelial cell migration | 0.01407071 | | |
| | | tissue migration | 0.01407071 | | |
| | | epithelium migration | 0.01407071 | | |
| | | ameboidal-type cell migration | 0.01506690 | | |
| | | taxis | 0.01058596 | 7 | 4 |
| | M3 | chemotaxis | 0.01058596 | | |
| | | transmembrane receptor protein | 0.01121427 | | |
| | | tyrosine kinase signaling | 0.01407071 | | |
| | | pathway | | | |
| | | tube development | | | |
| | M4 | protein secretion | 0.01680074 | 13 | 10 |
| | | regulation of peptide secretion | 0.01680074 | | |
| | | regulation of protein secretion | 0.01680074 | | |
| | | translation | 0.01744301 | | |
| | | peptide secretion | 0.01744301 | | |
| | | peptide biosynthetic process | 0.01744301 | | |
| | | amide biosynthetic process | 0.01935849 | | |
| | | regulation of secretion by cell | 0.01935849 | | |
| | | regulation of secretion | 0.02104555 | | |
| | | peptide metabolic process | 0.02246823 | | |
| | M5 | regulation of growth | 0.01710375 | 14 | 2 |
| | | growth | 0.01744301 | | |
| | M6 | positive regulation of secretion by cell | 0.01744301 | 20 | 5 |
| | | positive regulation of secretion | 0.01744301 | | |
| | | transmembrane receptor protein | 0.02379342 | | |
| | | tyrosine kinase signaling pathway | | | |
| | | regulation of secretion by cell | 0.03027594 | | |
| | | regulation of secretion | 0.0332764 | | |
| 7 h PM | | cytoplasmic translation | 0.00025664 | | |
| | | peptide metabolic process | | | |
| | M1 | translation | 0.00307113 | 4 | 5 |
| | | amide biosynthetic process | 0.00307113 | | |
| | | peptide biosynthetic process | 0.00307113 | | |
| | | | 0.00307113 | | |
| | M2 | regulation of response to external stimulus | 0.00819095 | 8 | 1 |

TABLE 9
Upregulated modules in the heart

| | UPREGULATED MODULES | | | | |
|---|---|---|---|---|---|
| 0 h PMI vs | MODULE | TOP TERMS | Q VAL | GENES | TERMS |
| 1 h PMI | M1 | Heart process | 0.00029963 | 24 | 26 |
| | | Heart contraction | 0.00029963 | | |
| | | Muscle contraction | 0.00060022 | | |
| | | Muscle system process | 0.00068257 | | |
| | | Circulatory system process | 0.00070174 | | |
| | | Blood circulation | 0.00070174 | | |
| | | Cardiac muscle contraction | 0.00122338 | | |
| | | Striated muscle contraction | 0.00190513 | | |
| | | Regulation of system process | 0.00510668 | | |
| | | Regulation of striated muscle | 0.00533685 | | |
| | | contraction | | | |
| | M2 | viral RNA genome replication | 0.00117626 | 10 | 32 |
| | | viral genome replication | 0.00575265 | | |
| | | positive regulation of | 0.00608795 | | |
| | | endopeptidase activity | 0.00608795 | | |
| | | positive regulation of | 0.00729271 | | |
| | | peptidase activity | 0.00735398 | | |
| | | I-kappaB kinase/NF-kappaB | 0.00753531 | | |
| | | signaling | 0.00786995 | | |
| | | endothelial cell migration | 0.00786995 | | |
| | | protein localization to plasma | 0.00825285 | | |
| | | membrane | | | |
| | | peptidyl-serine | | | |
| | | phosphorylation | | | |
| | | protein localization to cell | | | |
| | | periphery | | | |
| | | peptidyl-serine modification | | | |
| | M3 | tRNA aminoacylation for | 0.00190513 | 14 | 10 |
| | | protein translation | 0.00190513 | | |
| | | tRNA aminoacylation | 0.00190513 | | |
| | | amino acid activation | 0.00608795 | | |
| | | tRNA metabolic process | 0.00789626 | | |
| | | cellular amino acid metabolic | 0.01634324 | | |
| | | process | 0.01835367 | | |
| | | ncRNA metabolic process | 0.01931046 | | |
| | | translation | 0.02409173 | | |
| | | peptide biosynthetic process | 0.02884619 | | |
| | | amide biosynthetic process | | | |
| | | peptide metabolic process | | | |
| | M4 | regulation of cell cycle G1/S | 0.00354947 | 8 | 23 |
| | | phase transition | 0.0036058 | | |
| | | regulation of heart contraction | 0.00398175 | | |
| | | regulation of blood circulation | 0.00406808 | | |
| | | heart process | 0.00406808 | | |
| | | heart contraction | 0.00406808 | | |
| | | negative regulation of cell | 0.00496187 | | |
| | | growth | 0.00496187 | | |
| | | cell cycle G1/S phase | 0.00496187 | | |
| | | transition | 0.00533685 | | |
| | | negative regulation of growth | | | |
| | | regulation of phosphatase | | | |
| | | activity regulation of | | | |
| | | dephosphorylation | | | |
| | M5 | positive regulation of protein | 0.00510668 | 6 | 15 |
| | | secretion | 0.00533685 | | |
| | | establishment of organelle | 0.00533685 | | |
| | | localization | 0.00575265 | | |
| | | positive regulation of peptide | 0.00608795 | | |
| | | secretion | 0.00608795 | | |
| | | cell division | 0.00608795 | | |
| | | organelle localization | 0.00608795 | | |
| | | positive regulation of secretion | 0.00608795 | | |
| | | by cell | 0.00616309 | | |
| | | positive regulation of secretion | | | |
| | | positive regulation of protein | | | |
| | | transport | | | |
| | | regulation of protein secretion | | | |
| | | regulation of peptide secretion | | | |
| 7 h PMI | M1 | response to muscle stretch | 0.00251036 | 18 | 6 |
| | | heart process | 0.01122943 | | |
| | | heart contraction | 0.01122943 | | |
| | | response to mechanical | 0.01122943 | | |
| | | stimulus | 0.01447972 | | |
| | | circulatory system process | 0.01447972 | | |
| | | blood circulation | | | |
| | M2 | regulation of myosin-light- | 0.00251036 | 19 | 83 |
| | | chain-phosphatase activity | 0.00891652 | | |
| | | regulation of phosphatase | 0.01122943 | | |
| | | activity | 0.01122943 | | |
| | | epithelial to mesenchymal | 0.01122943 | | |
| | | transition | 0.01122943 | | |
| | | response to hormone | 0.01122943 | | |
| | | angiogenesis | 0.01122943 | | |
| | | blood vessel morphogenesis | 0.01122943 | | |
| | | blood vessel development | 0.01122943 | | |
| | | vasculature development | | | |
| | | cardiovascular system | | | |
| | | development | | | |
| | | ameboidal-type cell migration | | | |
| | M3 | sensory perception | 0.00891652 | 15 | 47 |
| | | skeletal system development | 0.01122943 | | |
| | | cartilage development | 0.01122943 | | |
| | | connective tissue development | 0.01122943 | | |
| | | positive regulation of | 0.01122943 | | |
| | | angiogenesis | 0.01122943 | | |
| | | positive regulation of | 0.01122943 | | |
| | | vasculature development | 0.01122943 | | |
| | | nervous system process | 0.01122943 | | |
| | | sensory perception of sound | 0.01122943 | | |
| | | sensory perception of | | | |
| | | mechanical stimulus | | | |
| | | cell cycle arrest | | | |
| | M4 | response to hormone | 0.01122943 | 6 | 2 |
| | | cellular response to hormone | 0.01122943 | | |
| | | stimulus | | | |

TABLE 10
Downregulated modules in the heart
DOWNREGULATED MODULES

| | MODULE | TOP TERMS | Q VAL | GENES | TERMS |
|---|---|---|---|---|---|
| 1 h PMI | M1 | muscle cell proliferation | 0.01672329 | 21 | 11 |
| | | smooth muscle cell | 0.01672329 | | |
| | | proliferation | | | |
| | | regulation of smooth muscle | 0.01672329 | | |
| | | cell proliferation | | | |
| | | negative regulation of cell | 0.02238411 | | |
| | | adhesion | | | |
| | | second-messenger-mediated | 0.03629741 | | |
| | | signaling | | | |
| | | purine-containing compound | 0.03629741 | | |
| | | metabolic process | | | |
| | | response to organonitrogen | 0.04567474 | | |
| | | compound | | | |
| | | regulation of response to | 0.04567474 | | |
| | | external stimulus | | | |
| | | cell-cell adhesion | 0.04567474 | | |
| | | negative regulation of cell | 0.04567474 | | |
| | | proliferation | | | |
| | M2 | brain development | 0.01672329 | 18 | 9 |
| | | head development | 0.01846202 | | |
| | | central nervous system development | 0.02238411 | | |
| | | negative regulation of locomotion | 0.03629741 | | |
| | | transmembrane receptor protein tyrosine | 0.04567474 | | |
| | | kinase signaling pathway | | | |
| | | response to hormone | 0.04567474 | | |
| | | cellular response to hormone stimulus | 0.04567474 | | |
| | | amide biosynthetic process | 0.04567474 | | |
| | | tube development | 0.04821183 | | |
| | M3 | positive regulation of cytokine production | 0.01672329 | 4 | 1 |
| | M4 | cell-matrix adhesion | 0.02238411 | 20 | 3 |
| | | cell-substrate adhesion | 0.03629741 | | |
| | | membrane organization | 0.04567474 | | |
| 7 h PMI | M1 | muscle tissue morphogenesis | 0.00001171 | 15 | 28 |
| | | cardiac muscle tissue morphogenesis | 0.00001171 | | |
| | | muscle organ morphogenesis | 0.00001171 | | |
| | | circulatory system process | 0.00007087 | | |
| | | blood circulation | 0.00007087 | | |
| | | cardiac muscle tissue development | 0.00009565 | | |
| | | heart morphogenesis | 0.00010493 | | |
| | | striated muscle tissue development | 0.00014682 | | |
| | | muscle organ development | 0.00014682 | | |
| | | muscle tissue development | 0.00015933 | | |
| | M2 | carbohydrate metabolic process | 0.00147773 | 4 | 9 |
| | | purine-containing compound metabolic process | 0.0015631 | | |
| | | nucleobase-containing compound catabolic | 0.00171625 | | |
| | | process | | | |
| | | heterocycle catabolic process | 0.00185306 | | |
| | | aromatic compound catabolic process | 0.00185306 | | |
| | | cellular nitrogen compound catabolic process | 0.00185306 | | |
| | | organic cyclic compound catabolic process | 0.00198857 | | |
| | | nucleobase-containing small molecule | 0.00215788 | | |
| | | metabolic process | | | |
| | | drug metabolic process | 0.00238158 | | |

TABLE 11
Upregulated modules in the liver

| | UPREGULATED MODULES | | | | |
|---|---|---|---|---|---|
| 0 h PMI vs | MODULE | TOP TERMS | Q VAL | GENES | TERMS |
| 1 h PM | M1 | positive regulation of protein kinase B | 0.00078186 | 2 | 11 |
| | | signaling | 0.00080542 | | |
| | | protein kinase B signaling | 0.00080542 | | |
| | | regulation of protein kinase B signaling | 0.00129131 | | |
| | | regulation of Wnt signaling pathway | 0.00129131 | | |
| | | canonical Wnt signaling pathway | 0.00129131 | | |
| | | regulation of canonical Wnt signaling pathway | 0.00129131 | | |
| | | positive regulation of cellular protein | 0.00140713 | | |
| | | localization | 0.00140713 | | |
| | | Wnt signaling pathway | 0.00153183 | | |
| | | cell-cell signaling by wnt | | | |
| | | cell surface receptor signaling pathway involved | | | |
| | | in cell-cell signaling | | | |
| | M2 | alpha-amino acid metabolic process | 0.00136955 | 23 | 23 |
| | | cellular amino acid metabolic process | 0.00203971 | | |
| | | lipid localization | 0.00348064 | | |
| | | phospholipid transport | 0.004241 | | |
| | | organophosphate ester transport | 0.00645779 | | |
| | | cellular modified amino acid metabolic process | 0.0066749 | | |
| | | regulation of lipid localization | 0.0089595 | | |
| | | reactive oxygen species metabolic process | 0.01777175 | | |
| | | sulfur compound metabolic process | 0.02065402 | | |
| | | circulatory system process | 0.02065402 | | |
| | M3 | peroxisome organization | 0.00366503 | 25 | 33 |
| | | fatty acid beta-oxidation | 0.00459225 | | |
| | | fatty acid metabolic process | 0.00505183 | | |
| | | fatty acid catabolic process | 0.00645779 | | |
| | | fatty acid oxidation | 0.00645779 | | |
| | | lipid oxidation | 0.0066749 | | |
| | | monocarboxylic acid catabolic process | 0.00832782 | | |
| | | monocarboxylic acid metabolic process | 0.01343222 | | |
| | | carboxylic acid transport | 0.01718533 | | |
| | | organic acid transport | 0.01718533 | | |
| | M4 | symbiont process | 0.02325045 | 11 | 1 |
| 7 h PMI | M1 | fatty acid metabolic process | 0.0018047 | 23 | 50 |
| | | monocarboxylic acid metabolic process | 0.0053895 | | |
| | | cellular lipid catabolic process | 0.0053895 | | |
| | | lipid catabolic process | 0.00606029 | | |
| | | peroxisome organization | 0.00606029 | | |
| | | regulation of fatty acid metabolic process | 0.00632649 | | |
| | | fatty acid beta-oxidation | 0.00704627 | | |
| | | neutral lipid metabolic process | 0.00745002 | | |
| | | acylglycerol metabolic process | 0.00745002 | | |
| | | fatty acid catabolic process | 0.00808599 | | |
| | M2 | peptide hormone processing | 0.0018047 | 17 | 14 |
| | | drug transmembrane transport | 0.00606029 | | |
| | | drug transport | 0.00808599 | | |
| | | hormone metabolic process | 0.00902764 | | |
| | | protein processing | 0.01391672 | | |
| | | organic anion transport | 0.01827469 | | |
| | | protein maturation | 0.01827469 | | |
| | | regulation of hormone levels | 0.02109436 | | |
| | | anion transport | 0.02867452 | | |
| | | import into cell | 0.03054423 | | |
| | M3 | lipid localization | 0.0053895 | 17 | 17 |
| | | phospholipid transport | 0.00606029 | | |
| | | organophosphate ester transport | 0.00736024 | | |
| | | regulation of reactive oxygen species metabolic | 0.00808599 | | |
| | | process | 0.00808599 | | |
| | | regulation of lipid localization | 0.01441779 | | |
| | | reactive oxygen species metabolic process | 0.01739625 | | |
| | | response to metal ion | 0.01745934 | | |
| | | lipid transport | 0.01827469 | | |
| | | circulatory system process | 0.01827469 | | |
| | | blood circulation | | | |

TABLE 12
Downregulated modules in the liver
DOWNREGULATED MODULES

| | MODULE | TOP TERMS | Q VAL | GENES | TERMS |
|---|---|---|---|---|---|
| 1 h PMI | M1 | regulation of endocytosis | 0.00001034 | | |
| | | regulation of vesicle-mediated transport | 0.00003473 | | |
| | | regulation of receptor-mediated endocytosis | 0.00003473 | | |
| | | endocytosis | 0.00004369 | | |
| | | import into cell | 0.00004597 | 8 | 12 |
| | | positive regulation of endocytosis | 0.00004597 | | |
| | | receptor-mediated endocytosis | 0.00016189 | | |
| | | positive regulation of receptor-mediated | 0.00054826 | | |
| | | endocytosis | 0.00392522 | | |
| | | negative regulation of cellular catabolic process | 0.00453021 | | |
| | | negative regulation of catabolic process | | | |
| | M2 | bile acid biosynthetic process | 0.00013665 | 8 | 18 |
| | | bile acid metabolic process | 0.00021854 | | |
| | | small molecule biosynthetic process | 0.00080919 | | |
| | | steroid biosynthetic process | 0.00099556 | | |
| | | organic hydroxy compound biosynthetic process | 0.00154562 | | |
| | | monocarboxylic acid biosynthetic process | 0.00224344 | | |
| | | steroid metabolic process | 0.00282644 | | |
| | | organic hydroxy compound metabolic process | 0.0041208 | | |
| | | carboxylic acid biosynthetic process | 0.0041208 | | |
| | | organic acid biosynthetic process | 0.0041208 | | |
| | | negative regulation of blood coagulation | 0.00045951 | | |
| | | negative regulation of hemostasis | 0.00045951 | | 19 |
| | | negative regulation of coagulation | 0.00052135 | | |
| | | regulation of blood coagulation | 0.0005927 | | |
| | M3 | regulation of hemostasis | 0.0005927 | 9 | |
| | | negative regulation of wound healing | 0.0005927 | | |
| | | negative regulation of response to wounding | 0.0005927 | | |
| | | regulation of coagulation | 0.0006521 | | |
| | | blood coagulation | 0.00099556 | | |
| | | hemostasis | 0.00099556 | | |
| | M4 | reactive oxygen species metabolic process | 0.0021294 | 9 | 2 |
| | | transmembrane receptor protein tyrosine kinase | 0.00561605 | | |
| | | signaling pathway | 0.00000003 | | |
| | | negative regulation of very-low-density lipoprotein | | | |
| | | particle remodeling | | | |
| 7 h PM | M1 | regulation of very-low-density lipoprotein | 0.00000006 | 9 | 138 |
| | | particle remodeling | 0.00000043 | | |
| | | very-low-density lipoprotein particle | 0.00000043 | | |
| | | remodeling | 0.00000043 | | |
| | | triglyceride-rich lipoprotein particle | 0.00000043 | | |
| | | remodeling | 0.00000046 | | |
| | | sterol import | 0.00000046 | | |
| | | cholesterol import | 0.00000154 | | |
| | | phospholipid efflux | 0.00000154 | | |
| | | high-density lipoprotein particle remodeling | | | |
| | | protein-containing complex remodeling | | | |
| | | protein-lipid complex remodeling | | | |
| | M2 | cytoplasmic translation | 0.00001502 | 10 | 12 |
| | | translation | 0.00004966 | | |
| | | peptide biosynthetic process | 0.00005325 | | |
| | | amide biosynthetic process | 0.00007408 | | |
| | | peptide metabolic process | 0.00010533 | | |
| | | ribosomal small subunit biogenesis | 0.000137 | | |
| | | rRNA processing | 0.00099778 | | |
| | | rRNA metabolic process | 0.0018087 | | |
| | | ribosome biogenesis | 0.00196663 | | |
| | | ncRNA processing | 0.00482245 | | |
| | | coenzyme biosynthetic process | 0.00223213 | | |
| | | drug metabolic process | 0.00265038 | | |
| | | cofactor biosynthetic process | 0.00322454 | | |
| | | monocarboxylic acid biosynthetic process | 0.00482245 | | |
| | M3 | coenzyme metabolic process | 0.00628091 | 14 | 16 |
| | | carboxylic acid biosynthetic process | 0.010031 | | |
| | | organic acid biosynthetic process | 0.010031 | | |
| | | cofactor metabolic process | 0.01473374 | | |
| | | taxis | 0.01570123 | | |
| | | chemotaxis | 0.01570123 | | |

TABLE 13
Upregulated modules in the kidney

| | UPREGULATED MODULES | | | | |
|---|---|---|---|---|---|
| 0 h PMI | MODULE | TOP TERMS | Q VAL | GENES | TERMS |
| 1 h PM | M1 | carboxylic acid transport | 0.00045523 | 2 | 4 |
| | | organic acid transport | 0.00045523 | | |
| | | organic anion transport | 0.00047204 | | |
| | | anion transport | 0.0008361 | | |
| | M2 | transforming growth factor beta receptor | 0.00045523 | 3 | 7 |
| | | signaling pathway | | | |
| | | cellular response to transforming growth factor | 0.00046771 | | |
| | | beta stimulus | | | |
| | | forming growth factor beta transmembrane | 0.00046771 | | |
| | | receptor protein serine/threonine kinase | | | |
| | | negative regulation of cell cycle | 0.00102429 | | |
| | | cellular response to growth factor stimulus | 0.00105664 | | |
| | | response to growth factor | 0.0010605 | | |
| | | regulation of mRNA splicing, via spliceosome | 0.00047204 | | |
| | | regulation of mRNA processing | 0.0008361 | | |
| | | regulation of RNA splicing | 0.00100785 | | |
| | M3 | regulation of mRNA metabolic process | 0.00168308 | 5 | 10 |
| | | mRNA splicing, via spliceosome | 0.00176775 | | |
| | | RNA splicing, via transesterification reactions | 0.00176775 | | |
| | | with bulged adenosine as nucleophile | | | |
| | | RNA splicing, via transesterification reactions | 0.00176775 | | |
| | | RNA splicing | 0.00266268 | | |
| | | mRNA processing | 0.00266268 | | |
| | | mRNA metabolic process | 0.0056398 | | |
| | M4 | ossification | 0.00185349 | 9 | 3 |
| | | response to nutrient levels | 0.00308148 | | |
| | | response to extracellular stimulus | 0.00324251 | | |
| | M5 | monosaccharide metabolic process | 0.0056398 | 21 | 8 |
| | | negative regulation of multi-organism process | 0.01152943 | | |
| | | carbohydrate metabolic process | 0.02064605 | | |
| | | viral process | 0.03197381 | | |
| | | dephosphorylation | 0.03684634 | | |
| | | regulation of multi-organism process | 0.03769913 | | |
| | | symbiont process | 0.03877931 | | |
| | | regulation of response to external stimulus | 0.0430254 | | |
| | M6 | protein acetylation | 0.00574013 | 13 | 2 |
| | | protein acylation | 0.00818346 | | |
| | M7 | positive regulation of proteolysis | 0.00818346 | 9 | 1 |
| 7 H PMI | M1 | autophagy | 0.00179789 | 3 | 4 |
| | | positive regulation of cellular catabolic process | 0.00179789 | | |
| | | process utilizing autophagic mechanism | 0.00179789 | | |
| | | positive regulation of catabolic process | 0.00179789 | | |
| | M2 | muscle system process | 0.00437296 | 9 | 2 |
| | | nervous system process | 0.00738351 | | |
| | M3 | drug metabolic process | 0.0056181 | 6 | 3 |
| | | cell morphogenesis | 0.0056181 | | |
| | | cellular component morphogenesis | 0.0056181 | | |

TABLE 14
Downregulated modules in the kidney
DOWNREGULATED MODULES

| | MODULE | TOP TERMS | Q VAL | GENES | TERMS |
|---|---|---|---|---|---|
| 1 h PMI | M1 | sulfur compound metabolic process | 0.00574843 | 3 | 1 |
| | M2 | reactive oxygen species metabolic | 0.00826615 | 10 | 3 |
| | | process | | | |
| | | drug metabolic process | 0.02068022 | | |
| | | protein complex oligomerization | 0.02083823 | | |
| | | reactive oxygen species metabolic | 0.00826615 | 13 | 3 |
| | M3 | process | 0.00903419 | | |
| | | sulfur compound metabolic process | 0.01045815 | | |
| | | regulation of anatomical structure size | | | |
| | M4 | import into cell | 0.00826615 | 17 | 9 |
| | | carbohydrate metabolic process | 0.00826615 | | |
| | | regulation of carbohydrate metabolic | 0.00826615 | | |
| | | process | | | |
| | | regulation of vesicle-mediated transport | 0.00826615 | | |
| | | positive regulation of endocytosis | 0.00826615 | | |
| | | monosaccharide metabolic process | 0.00965095 | | |
| | | negative regulation of secretion | 0.01045815 | | |
| | | regulation of endocytosis | 0.01250846 | | |
| | | regulation of canonical Wnt signaling | 0.01664705 | | |
| | | pathway | | | |
| | | regulation of small molecule metabolic | 0.01790918 | | |
| | | process | | | |
| | M5 | response to lipid | 0.02155897 | 10 | 1 |
| 7 h PMI | M1 | cytoplasmic translation | 0.00085548 | 5 | 5 |
| | | translation | 0.00358328 | | |
| | | peptide biosynthetic process | 0.00358328 | | |
| | | amide biosynthetic process | 0.00377282 | | |
| | | peptide metabolic process | 0.00426545 | | |
| | M2 | response to peptide | 0.00125726 | 3 | 6 |
| | | cellular response to peptide | 0.00125726 | | |
| | | cellular response to organonitrogen | 0.0028522 | | |
| | | compound | | | |
| | | membrane receptor protein tyrosine kinase | 0.00303333 | | |
| | | signaling p | | | |
| | | cellular response to nitrogen | 0.00324142 | | |
| | | compound | | | |
| | | response to organonitrogen | 0.00358328 | | |
| | | compound | | | |
| | M3 | cell part morphogenesis | 0.00359538 | 9 | 6 |
| | | cellular response to hormone stimulus | 0.00972276 | | |
| | | negative regulation of protein | 0.00972276 | | |
| | | phosphorylation | | | |
| | | negative regulation of phosphorylation | 0.00972276 | | |
| | | response to hormone | 0.01170763 | | |
| | | cellular component morphogenesis | 0.0119158 | | |
| | M4 | positive regulation of secretion | 0.00813972 | | |
| | | carbohydrate derivative biosynthetic | 0.00972276 | 10 | 3 |
| | | process | | | |
| | | regulation of secretion | 0.01262213 | | |

TABLE 15
Upregulated modules in the hippocampus for OrganEx vs other experimental conditions

| OrganEx | UPREGULATED MODULES | | | | |
|---|---|---|---|---|---|
| vs | MODULE | TOP TERMS | Q VAL | GENES | TERMS |
| 0 h PMI | M1 | regulation of calcium ion transmembrane | 0.00000148 | 3 | 62 |
| | | transporter | | | |
| | | regulation of protein dephosphorylation | 0.00000264 | | |
| | | regulation of calcium ion transmembrane | 0.00000264 | | |
| | | transport | | | |
| | | regulation of ion transmembrane transporter | 0.00000836 | | |
| | | activity | | | |
| | | regulation of calcium ion transport | 0.00000836 | | |
| | | regulation of dephosphorylation | 0.00000836 | | |
| | | regulation of transmembrane transporter | 0.00000836 | | |
| | | activity | | | |
| | | regulation of transporter activity | 0.00000836 | | |
| | | regulation of cytosolic calcium ion | 0.00000933 | | |
| | | concentration | | | |
| | | protein dephosphorylation | 0.00000946 | | |
| | M2 | postsynapse organization | 0.00002732 | 5 | 11 |
| | | synapse organization | 0.00013829 | | |
| | | modification by host of symbiont | 0.00034457 | | |
| | | morphology or physiology | | | |
| | | interaction with symbiont | 0.00036868 | | |
| | | modification of morphology or physiology | 0.00051716 | | |
| | | of other organism involved in symbiotic | | | |
| | | interaction | | | |
| | | modification of morphology or physiology | 0.00105763 | | |
| | | of other organism | | | |
| | | positive regulation of multi-organism | 0.00113891 | | |
| | | process | | | |
| | | regulation of symbiosis, encompassing | 0.00225101 | | |
| | | mutualism through parasitism | | | |
| | | actin cytoskeleton organization regulation of | 0.00477062 | | |
| | | multi-organism process | | | |
| | | regulation of multi-organism process | 0.00477062 | | |
| | M3 | positive regulation of cell-matrix adhesion | 0.00034457 | 11 | 14 |
| | | integrin-mediated signaling pathway | 0.00061588 | | |
| | | positive regulation of cell-substrate adhesion | 0.00094367 | | |
| | | regulation of cell-matrix adhesion | 0.00133405 | | |
| | | regulation of cell-substrate adhesion | 0.00321521 | | |
| | | cell-matrix adhesion | 0.00370559 | | |
| | | cell-substrate adhesion | 0.00705772 | | |
| | | positive regulation of cell adhesion | 0.00740948 | | |
| | | regulation of ion transport | 0.01072926 | | |
| | | positive regulation of cellular component | 0.01473763 | | |
| | | biogenesis | | | |
| | M4 | establishment of organelle localization | 0.0015234 | 5 | 2 |
| | | organelle localization | 0.00260079 | | |
| | M5 | negative regulation of protein | 0.00197 | 11 | 11 |
| | | serine/threonine kinase activity | | | |
| | | negative regulation of protein kinase activity | 0.00521825 | | |
| | | negative regulation of kinase activity | 0.00598533 | | |
| | | negative regulation of transferase activity | 0.00750629 | | |
| | | negative regulation of protein | 0.01223217 | | |
| | | phosphorylation | | | |
| | | regulation of protein serine/threonine kinase | 0.01355639 | | |
| | | activity | | | |
| | | negative regulation of phosphorylation | 0.01365901 | | |
| | | peptidyl-tyrosine phosphorylation | 0.01407734 | | |
| | | peptidyl-tyrosine modification | 0.01429091 | | |
| | | positive regulation of apoptotic process | 0.01976654 | | |
| | M6 | cellular response to metal ion | 0.00394424 | 15 | 15 |
| | | sodium ion transmembrane transport | 0.00440242 | | |
| | | cellular response to inorganic substance | 0.00445226 | | |
| | | sodium ion transport | 0.00471215 | | |
| | | regulation of ion transmembrane transporter | 0.00611441 | | |
| | | activity | | | |
| | | regulation of transmembrane transporter | 0.006273 | | |
| | | activity | | | |
| | | regulation of transporter activity | 0.00705772 | | |
| | | response to metal ion | 0.00750629 | | |
| | | regulation of cation transmembrane | 0.00830569 | | |
| | | transport | | | |
| | | regulation of ion transmembrane transport | 0.0108403 | | |
| | M7 | purine ribonucleotide metabolic process | 0.00483097 | 8 | 9 |
| | | ribonucleotide metabolic process | 0.00492221 | | |
| | | purine nucleotide metabolic process | 0.00501519 | | |
| | | ribose phosphate metabolic process | 0.00507639 | | |
| | | purine-containing compound metabolic | 0.00598533 | | |
| | | process | | | |
| | | nucleotide metabolic process | 0.00737697 | | |
| | | nucleoside phosphate metabolic process | 0.00742628 | | |
| | | organic cyclic compound catabolic process | 0.00819991 | | |
| | | nucleobase-containing small molecule | 0.00928187 | | |
| | | metabolic process | | | |
| 1 h PMI | M1 | negative regulation of protein | 0.00632916 | 8 | 11 |
| | | serine/threonine kinase activity | | | |
| | | negative regulation of protein kinase activity | 0.0068929 | | |
| | | negative regulation of kinase activity | 0.0068929 | | |
| | | negative regulation of transferase activity | 0.00705237 | | |
| | | negative regulation of protein | 0.00848203 | | |
| | | phosphorylation | | | |
| | | regulation of protein serine/threonine kinase | 0.00859112 | | |
| | | activity | | | |
| | | negative regulation of phosphorylation | 0.00859112 | | |
| | | peptidyl-tyrosine phosphorylation | 0.00859112 | | |
| | | peptidyl-tyrosine modification | 0.00859112 | | |
| | | positive regulation of apoptotic process | 0.01171437 | | |
| | M2 | postsynapse organization | 0.00632916 | 17 | 28 |
| | | synapse organization | 0.00632916 | | |
| | | positive regulation of viral process | 0.00776015 | | |
| | | neuron projection morphogenesis | 0.00848203 | | |
| | | plasma membrane bounded cell projection | 0.00859112 | | |
| | | morphogenesis | | | |
| | | cell projection morphogenesis | 0.00859112 | | |
| | | cell part morphogenesis | 0.00982363 | | |
| | | positive regulation of multi-organism | 0.01171437 | | |
| | | process | | | |
| | | establishment of organelle localization | 0.01496545 | | |
| | | regulation of membrane potential | 0.01570713 | | |
| | M3 | regulated exocytosis | 0.00632916 | 12 | 2 |
| | | exocytosis | 0.00705237 | | |
| | M4 | heart process | 0.00632916 | 9 | 28 |
| | | regulation of heart contraction | 0.00632916 | | |
| | | regulation of heart rate | 0.00632916 | | |
| | | striated muscle contraction | 0.00632916 | | |
| | | response to calcium ion | 0.00632916 | | |
| | | regulation of blood circulation | 0.00632916 | | |
| | | heart contraction | 0.00632916 | | |
| | | cardiac muscle contraction | 0.00632916 | | |
| | | muscle contraction | 0.0068929 | | |
| | | calcium-mediated signaling | 0.0068929 | | |
| | M5 | calcium-mediated signaling | 0.00632916 | 5 | 3 |
| | | second-messenger-mediated signaling | 0.0068929 | | |
| | | supramolecular fiber organization | 0.00763474 | | |
| 7 h PMI | M1 | central nervous system myelination | 0.00010421 | 12 | 21 |
| | | axon ensheathment in central nervous | 0.00010421 | | |
| | | system | | | |
| | | oligodendrocyte development | 0.00062285 | | |
| | | oligodendrocyte differentiation | 0.00062285 | | |
| | | ensheathment of neurons | 0.00065125 | | |
| | | axon ensheathment | 0.00065125 | | |
| | | myelination | 0.00065125 | | |
| | | glial cell development | 0.00067302 | | |
| | | glial cell differentiation | 0.0011697 | | |
| | | gliogenesis | 0.00260164 | | |
| | M2 | postsynapse organization | 0.00132508 | 17 | 91 |
| | | supramolecular fiber organization | 0.00258787 | | |
| | | symbiont process | 0.00258787 | | |
| | | positive regulation of multi-organism | 0.00290139 | | |
| | | process | | | |
| | | synapse organization | 0.00614214 | | |
| | | cellular response to interferon-gamma | 0.00614214 | | |
| | | regulation of symbiosis, encompassing | 0.00669687 | | |
| | | mutualism through parasitism | | | |
| | | regulation of protein secretion | 0.00820244 | | |
| | | regulation of peptide secretion | 0.0090486 | | |
| | | negative regulation of supramolecular fiber | 0.00947399 | | |
| | | organization | | | |
| | M3 | establishment of protein localization to | 0.00338045 | 4 | 2 |
| | | organelle | | | |
| | | regulation of cellular protein localization | 0.00669687 | | |
| | M4 | negative regulation of MAPK cascade | 0.01245222 | 17 | 10 |
| | | negative regulation of protein kinase activity | 0.0163574 | | |
| | | negative regulation of kinase activity | 0.01741828 | | |
| | | regulation of ERKI and ERK2 cascade | 0.01784267 | | |
| | | ERK1 and ERK2 cascade | 0.01998293 | | |
| | | negative regulation of transferase activity | 0.02039825 | | |
| | | negative regulation of proteolysis | 0.02214126 | | |
| | | negative regulation of protein | 0.0262057 | | |
| | | phosphorylation | | | |
| | | negative regulation of phosphorylation | 0.02835256 | | |
| | | negative regulation of intracellular signal | 0.03926507 | | |
| | | transduction | | | |
| ECMO | M1 | membrane depolarization | 0.00022659 | 13 | 40 |
| | | regulation of membrane potential | 0.00140469 | | |
| | | regulation of membrane depolarization | 0.00140469 | | |
| | | regulation of metal ion transport | 0.00140469 | | |
| | | regulation of cation transmembrane | 0.00140469 | | |
| | | transport | | | |
| | | regulation of ion transmembrane transporter | 0.00140469 | | |
| | | activity | | | |
| | | regulation of transmembrane transporter | 0.00140469 | | |
| | | activity | | | |
| | | regulation of transporter activity | 0.00140469 | | |
| | | regulation of ion transmembrane transport | 0.00140469 | | |
| | | amyloid precursor protein metabolic process | 0.00140469 | | |
| | M2 | cellular metal ion homeostasis | 0.00140469 | 11 | 15 |
| | | glutamate receptor signaling pathway | 0.00140469 | | |
| | | cellular ion homeostasis | 0.00141584 | | |
| | | cellular cation homeostasis | 0.00141584 | | |
| | | metal ion homeostasis | 0.00141584 | | |
| | | cellular chemical homeostasis | 0.00162159 | | |
| | | cation homeostasis | 0.00162159 | | |
| | | inorganic ion homeostasis | 0.00162159 | | |
| | | cellular homeostasis | 0.00183596 | | |
| | | positive regulation of cytosolic calcium ion | 0.00468417 | | |
| | | concentration | | | |
| | M3 | vesicle organization | 0.00140469 | 4 | 3 |
| | | endocytosis | 0.00162159 | | |
| | | import into cell | 0.00183596 | | |
| | M4 | regulation of cytoskeleton organization | 0.00210499 | 3 | 1 |
| | M5 | supramolecular fiber organization | 0.01236373 | 8 | 1 |
| | M6 | glycerolipid metabolic process | 0.01362313 | 16 | 1 |

TABLE 16
Downregulated modules in the hippocampus for OrganEx vs other experimental conditions

| | MODULE | TOP TERMS | Q VAL | GENES | TERMS |
|---|---|---|---|---|---|
| 0 h PMI | M1 | cell-cell adhesion via plasma-membrane adhesion | 0.0004168 | 6 | 2 |
| | | molecules | | | |
| | | cell-cell adhesion | 0.00377508 | | |
| | M2 | action potential | 0.0004168 | 7 | 3 |
| | | multicellular organismal signaling | 0.0004168 | | |
| | | regulation of membrane potential | 0.00146461 | | |
| | M3 | cellular response to growth factor stimulus | 0.00261105 | 5 | 2 |
| | | response to growth factor | 0.00261637 | | |
| 1 h PMI | M1 | cell-matrix adhesion | 0.0023427 | 9 | 3 |
| | | cell-substrate adhesion | 0.00483403 | | |
| | | regulation of cytoskeleton organization | 0.00966187 | | |
| | M2 | cellular component morphogenesis | 0.029452 | 18 | 1 |
| 7 h PMI | M1 | substrate-dependent cell migration | 0.00021131 | 11 | 4 |
| | | integrin-mediated signaling pathway | 0.00086613 | | |
| | | cell-matrix adhesion | 0.00370986 | | |
| | | cell-substrate adhesion | 0.00607305 | | |
| | M2 | monovalent inorganic cation transport | 0.00090489 | 13 | 6 |
| | | potassium ion transport | 0.00441522 | | |
| | | potassium ion transmembrane transport | 0.00441522 | | |
| | | cellular potassium ion transport | 0.00441522 | | |
| | | regulation of ERKI and ERK2 cascade | 0.00621087 | | |
| | | ERK1 and ERK2 cascade | 0.00684584 | | |
| ECMO | M1 | negative regulation of transcription from RNA | 0.00050986 | 10 | 28 |
| | | polymerase II promoter in response to stress | | | |
| | | negative regulation of inclusion body assembly | 0.00050986 | | |
| | | regulation of inclusion body assembly | 0.00058354 | | |
| | | inclusion body assembly | 0.00088919 | | |
| | | regulation of protein ubiquitination | 0.00112921 | | |
| | | regulation of protein modification by small protein | 0.00122915 | | |
| | | conjugation or removal | | | |
| | | signal transduction involved in cell cycle checkpoint | 0.00123005 | | |
| | | signal transduction involved in DNA integrity | 0.00123005 | | |
| | | checkpoint | | | |
| | | signal transduction involved in DNA damage | 0.00123005 | | |
| | | checkpoint | | | |
| | | regulation of transcription from RNA polymerase II | 0.00180748 | | |
| | | promoter in response to stress | | | |
| | M2 | negative regulation of transcription from RNA | 0.00050986 | 6 | 23 |
| | | polymerase II promoter in response to stress | | | |
| | | regulation of transcription from RNA polymerase II | 0.00112921 | | |
| | | promoter in response to stress | | | |
| | | regulation of DNA-templated transcription in response | 0.00117198 | | |
| | | to stress | | | |
| | | cellular response to reactive oxygen species | 0.00199647 | | |
| | | response to reactive oxygen species | 0.00248837 | | |
| | | negative regulation of viral process | 0.00248837 | | |
| | | transforming growth factor beta receptor signaling | 0.00261758 | | |
| | | pathway | | | |
| | | cellular response to transforming growth factor beta | 0.00375985 | | |
| | | stimulus | | | |
| | | response to transforming growth factor beta | 0.00375985 | | |
| | | cellular response to oxidative stress | 0.00375985 | | |
| | M3 | regulation of substrate adhesion-dependent cell | 0.003814 | 23 | 39 |
| | | spreading | | | |
| | | regulation of cell development | 0.00762995 | | |
| | | substrate adhesion-dependent cell spreading | 0.00762995 | | |
| | | nervous system process | 0.00836972 | | |
| | | cognition | 0.00836972 | | |
| | | regulation of cell morphogenesis involved in | 0.00870874 | | |
| | | differentiation | | | |
| | | regulation of cell-substrate adhesion | 0.01664242 | | |
| | | positive regulation of supramolecular fiber organization | 0.01744524 | | |
| | | cellular response to peptide | 0.01745126 | | |
| | | response to peptide | 0.02004258 | | |
| | M4 | positive regulation of cell migration | 0.0197859 | 13 | 3 |
| | | positive regulation of cellular component movement | 0.02026998 | | |
| | | positive regulation of cell motility | 0.02026998 | | |

TABLE 17
Upregulated modules in the heart for OrganEx vs other experimental conditions

| | UPREGULATED MODULES | | | | |
|---|---|---|---|---|---|
| OrganEx vs | MODULE | TOP TERMS | Q VAL | GENES | TERMS |
| 0 h PMI | M1 | regulation of extrinsic apoptotic signaling pathway | 0.00002361 | 24 | 179 |
| | | via death domain receptors | | | |
| | | positive regulation of extrinsic apoptotic signaling | 0.00002361 | | |
| | | pathway via death domain receptors | | | |
| | | extrinsic apoptotic signaling pathway via death | 0.00011521 | | |
| | | domain receptors | | | |
| | | positive regulation of TRAIL-activated apoptotic | 0.00029119 | | |
| | | signaling pathway | | | |
| | | regulation of extrinsic apoptotic signaling pathway | 0.00032231 | | |
| | | positive regulation of extrinsic apoptotic signaling | 0.00032313 | | |
| | | pathway | | | |
| | | regulation of apoptotic signaling pathway | 0.00034095 | | |
| | | regulation of TRAIL-activated apoptotic signaling | 0.00034095 | | |
| | | pathway | | | |
| | | positive regulation of smooth muscle cell | 0.00034095 | | |
| | | proliferation | | | |
| | | oncostatin-M-mediated signaling pathway | 0.00034095 | | |
| | M2 | establishment of protein localization to organelle | 0.00029119 | 24 | 122 |
| | | regulation of cellular protein localization | 0.00029119 | | |
| | | positive regulation of cellular protein localization | 0.00029119 | | |
| | | positive regulation of protein import into nucleus | 0.00029119 | | |
| | | positive regulation of protein import | 0.00029119 | | |
| | | protein import | 0.00032231 | | |
| | | regulation of protein import into nucleus | 0.00034095 | | |
| | | regulation of protein import | 0.00034095 | | |
| | | positive regulation of nucleocytoplasmic transport | 0.00039583 | | |
| | | positive regulation of protein localization to | 0.00051733 | | |
| | | nucleus | | | |
| | M3 | positive regulation of MAP kinase activity | 0.01239568 | 19 | 10 |
| | | regulation of MAP kinase activity | 0.01929366 | | |
| | | activation of protein kinase activity | 0.02032994 | | |
| | | positive regulation of protein serine/threonine | 0.02046562 | | |
| | | kinase activity | | | |
| | | regulation of protein serine/threonine kinase | 0.0339451 | | |
| | | activity | | | |
| | | positive regulation of MAPK cascade | 0.03835472 | | |
| | | positive regulation of protein kinase activity | 0.0393374 | | |
| | | positive regulation of kinase activity | 0.04337171 | | |
| | | cellular response to growth factor stimulus | 0.04459056 | | |
| | | response to growth factor | 0.04609302 | | |
| | | positive regulation of smooth muscle cell | 0.00016128 | | |
| | | proliferation | | | |
| 1 h PMI | M1 | smooth muscle cell proliferation | 0.00017323 | 9 | 109 |
| | | regulation of smooth muscle cell proliferation | 0.00017323 | | |
| | | negative regulation of plasminogen activation | 0.00017323 | | |
| | | negative regulation of fibrinolysis | 0.00017323 | | |
| | | muscle cell proliferation | 0.00019664 | | |
| | | regulation of fibrinolysis | 0.00020283 | | |
| | | regulation of plasminogen activation | 0.00023696 | | |
| | | positive regulation of blood coagulation | 0.00029821 | | |
| | | positive regulation of coagulation | 0.00029821 | | |
| | | chaperone-mediated protein folding | 0.00116853 | | |
| | M2 | positive regulation of ATPase activity | 0.00156601 | 17 | 52 |
| | | positive regulation of cell cycle process | 0.00176265 | | |
| | | positive regulation of cytokinesis | 0.00196763 | | |
| | | negative regulation of MAP kinase activity | 0.00203168 | | |
| | | positive regulation of cell division | 0.00222306 | | |
| | | regulation of translational initiation | 0.00235603 | | |
| | | establishment of protein localization to organelle | 0.00235603 | | |
| | | regulation of ATPase activity | 0.00235603 | | |
| | | establishment of protein localization to | 0.0027263 | | |
| | | mitochondrion | | | |
| | | cell morphogenesis involved in differentiation | 0.00446098 | | |
| | M3 | calcium ion transport | 0.00759034 | 10 | 8 |
| | | negative regulation of cell differentiation | 0.00835761 | | |
| | | divalent metal ion transport | 0.00879372 | | |
| | | divalent inorganic cation transport | 0.00879372 | | |
| | | cell morphogenesis | 0.0116237 | | |
| | | membrane organization | 0.01258141 | | |
| | | cellular component morphogenesis | 0.01358402 | | |
| | | extrinsic apoptotic signaling pathway via death | 0.0001177 | | |
| | | domain receptors | | | |
| 7 h PMI | M1 | regulation of extrinsic apoptotic signaling pathway | 0.00031453 | 21 | 179 |
| | | via death domain receptors | | | |
| | | positive regulation of angiogenesis | 0.00031453 | | |
| | | positive regulation of vasculature development | 0.00031453 | | |
| | | regulation of transcription from RNA polymerase | 0.00033563 | | |
| | | II promoter in response to stress | | | |
| | | oncostatin-M-mediated signaling pathway | 0.00033563 | | |
| | | extrinsic apoptotic signaling pathway | 0.00033998 | | |
| | | regulation of DNA-templated transcription in | 0.00033998 | | |
| | | response to stress | | | |
| | | negative regulation of plasminogen activation | 0.00033998 | | |
| | | negative regulation of fibrinolysis | 0.00033998 | | |
| | | calcium ion transport | 0.0001177 | | |
| | M2 | divalent metal ion transport | 0.0001177 | 16 | 49 |
| | | divalent inorganic cation transport | 0.0001177 | | |
| | | calcium ion transmembrane transport | 0.00033563 | | |
| | | cell activation involved in immune response | 0.00693738 | | |
| | | intracellular protein transport | 0.00854985 | | |
| | | cell-cell adhesion | 0.00854985 | | |
| | | cellular homeostasis | 0.00854985 | | |
| | | regulation of phosphatase activity | 0.00991974 | | |
| | | central nervous system development | 0.01151349 | | |
| | | positive regulation of ATPase activity | 0.00174275 | | |
| | M3 | regulation of ATPase activity | 0.0026367 | 12 | 5 |
| | | establishment of protein localization to | 0.00293313 | | |
| | | mitochondrion | | | |
| | | protein localization to mitochondrion | 0.00293313 | | |
| | | establishment of protein localization to organelle | 0.01267787 | | |
| | | cellular component assembly involved in | 0.00109672 | | |
| | | morphogenesis 0.00109672 myofibril assembly | | | |
| ECMO | M1 | striated muscle cell development | 0.00109672 | 11 | 16 |
| | | muscle cell development | 0.00116538 | | |
| | | striated muscle cell differentiation | 0.00215042 | | |
| | | actomyosin structure organization | 0.00378743 | | |
| | | muscle cell differentiation | 0.00378743 | | |
| | | muscle structure development | 0.00695858 | | |
| | | actin filament organization | 0.00909936 | | |
| | | regulation of ion transport | 0.01399645 | | |
| | | positive regulation of ATPase activity | 0.00109672 | | |
| | M2 | regulation of ATPase activity | 0.00109672 | 5 | 3 |
| | | negative regulation of intracellular signal | 0.00755618 | | |
| | | transduction | | | |
| | | positive regulation of vascular smooth muscle cell | 0.00109672 | | |
| | | proliferation | | | |
| | M3 | regulation of vascular smooth muscle cell | 0.00132483 | 11 | 24 |
| | | proliferation | | | |
| | | vascular smooth muscle cell proliferation | 0.00132483 | | |
| | | positive regulation of smooth muscle cell | 0.00146751 | | |
| | | proliferation | | | |
| | | regulation of transcription from RNA polymerase | 0.00161299 | | |
| | | II promoter in response to stress | | | |
| | | regulation of DNA-templated transcription in | 0.00180501 | | |
| | | response to stress | | | |
| | | smooth muscle cell proliferation | 0.00369898 | | |
| | | regulation of smooth muscle cell proliferation | 0.00369898 | | |
| | | positive regulation of apoptotic process | 0.00378743 | | |
| | | positive regulation of programmed cell death | 0.00378743 | | |
| | | negative regulation of intracellular signal | 0.00109672 | | |
| | | transduction | | | |
| | M4 | negative regulation of intrinsic apoptotic signaling | 0.00132483 | 5 | 14 |
| | | pathway | | | |
| | | negative regulation of cellular amide metabolic | 0.00146751 | | |
| | | process | | | |
| | | regulation of intrinsic apoptotic signaling pathway | 0.00200305 | | |
| | | negative regulation of apoptotic signaling pathway | 0.00281162 | | |
| | | intrinsic apoptotic signaling pathway | 0.00378743 | | |
| | | regulation of cellular amide metabolic process | 0.00419639 | | |
| | | positive regulation of cellular catabolic process | 0.00484584 | | |
| | | regulation of apoptotic signaling pathway | 0.00494742 | | |
| | | positive regulation of cytokine production | 0.00573414 | | |
| | | ATP hydrolysis coupled ion transmembrane | 0.00132483 | | |
| | | transport | | | |
| | M5 | ATP hydrolysis coupled cation transmembrane | 0.00132483 | 15 | 25 |
| | | transport | | | |
| | | ATP hydrolysis coupled transmembrane transport | 0.00132483 | | |
| | | mitochondrial membrane organization | 0.00420776 | | |
| | | mitochondrial transport | 0.00747788 | | |
| | | cellular response to nutrient levels | 0.00795956 | | |
| | | cellular response to extracellular stimulus | 0.00843396 | | |
| | | response to nutrient levels | 0.01107734 | | |
| | | response to extracellular stimulus | 0.01193224 | | |
| | | regulation of membrane potential | 0.01399645 | | |
| | | angiogenesis | 0.01930628 | | |
| | M6 | blood vessel morphogenesis | 0.02092554 | 12 | 7 |
| | | blood vessel development | 0.0214603 | | |
| | | vasculature development | 0.02245115 | | |
| | | cardiovascular system development | 0.02245115 | | |
| | | tube morphogenesis | 0.02341853 | | |
| | | tube development | 0.02626913 | | |

TABLE 18
Downregulated modules in the heart for OrganEx vs other experimental conditions

| | MODULE | TOP TERMS | Q VAL | GENES | TERMS |
|---|---|---|---|---|---|
| 0 h PMI | M1 | muscle tissue morphogenesis | 0.00266479 | 23 | 25 |
| | | ventricular cardiac muscle tissue development | 0.00266479 | | |
| | | cardiac ventricle morphogenesis | 0.00266479 | | |
| | | ventricular cardiac muscle tissue morphogenesis | 0.00266479 | | |
| | | cardiac muscle tissue morphogenesis | 0.00266479 | | |
| | | muscle organ morphogenesis | 0.00266479 | | |
| | | cardiac ventricle development | 0.00329834 | | |
| | | tissue morphogenesis | 0.00337476 | | |
| | | cardiac chamber morphogenesis | 0.00337476 | | |
| | | cardiac chamber development | 0.00369421 | | |
| | M2 | ncRNA metabolic process | 0.03335282 | 23 | 6 |
| | | cellular response to hormone stimulus | 0.03860622 | | |
| | | transmembrane receptor protein tyrosine kinase | 0.03860622 | | |
| | | signaling pathway | | | |
| | | response to hormone | 0.04748336 | | |
| | | positive regulation of cellular component biogenesis | 0.0495055 | | |
| | | supramolecular fiber organization | 0.0495055 | | |
| 1 h PMI | M1 | muscle tissue morphogenesis | 0.00004401 | 22 | 51 |
| | | ventricular cardiac muscle tissue development | 0.00004401 | | |
| | | cardiac ventricle morphogenesis | 0.00004401 | | |
| | | ventricular cardiac muscle tissue morphogenesis | 0.00004401 | | |
| | | cardiac muscle tissue morphogenesis | 0.00004401 | | |
| | | muscle organ morphogenesis | 0.00004401 | | |
| | | cardiac ventricle development | 0.00005839 | | |
| | | cardiac chamber morphogenesis | 0.00007473 | | |
| | | cardiac chamber development | 0.00010322 | | |
| | | tissue morphogenesis | 0.00021319 | | |
| | M2 | regulation of embryonic development | 0.00236854 | 18 | 30 |
| | | cell-substrate adherens junction assembly | 0.00482378 | | |
| | | focal adhesion assembly | 0.00482378 | | |
| | | cell-substrate junction assembly | 0.0050007 | | |
| | | adherens junction assembly | 0.0050007 | | |
| | | adherens junction organization | 0.00613601 | | |
| | | positive regulation of cell migration | 0.0109135 | | |
| | | positive regulation of cell motility | 0.0110496 | | |
| | | positive regulation of cellular component movement | 0.0111725 | | |
| | | cell junction assembly | 0.01299785 | | |
| | M3 | regulation of cell-matrix adhesion | 0.00669186 | 17 | 13 |
| | | actomyosin structure organization | 0.00944387 | | |
| | | regulation of cell-substrate adhesion | 0.01221792 | | |
| | | cell-matrix adhesion | 0.01271436 | | |
| | | negative regulation of cell migration | 0.02123794 | | |
| | | cell-substrate adhesion | 0.02143607 | | |
| | | negative regulation of cell motility | 0.02143607 | | |
| | | actin filament organization | 0.02143607 | | |
| | | negative regulation of cellular component movement | 0.02176389 | | |
| | | negative regulation of locomotion | 0.02255749 | | |
| 7 h PMI | M1 | muscle tissue morphogenesis | 0.00000104 | 10 | 29 |
| | | ventricular cardiac muscle tissue development | 0.00000104 | | |
| | | cardiac ventricle morphogenesis | 0.00000104 | | |
| | | ventricular cardiac muscle tissue morphogenesis | 0.00000104 | | |
| | | cardiac muscle tissue morphogenesis | 0.00000104 | | |
| | | muscle organ morphogenesis | 0.00000104 | | |
| | | cardiac ventricle development | 0.00000138 | | |
| | | cardiac chamber morphogenesis | 0.00000177 | | |
| | | muscle structure development | 0.00000226 | | |
| | | cardiac chamber development | 0.00000226 | | |
| | M2 | regulation of transmembrane transport | 0.00425822 | 9 | 1 |
| ECMO | M1 | negative regulation of chondrocyte differentiation | 0.00185788 | 24 | 23 |
| | | negative regulation of cartilage development | 0.00185788 | | |
| | | regulation of chondrocyte differentiation | 0.00196174 | | |
| | | regulation of cartilage development | 0.00277101 | | |
| | | chondrocyte differentiation | 0.00385886 | | |
| | | cartilage development | 0.0057775 | | |
| | | connective tissue development | 0.00633818 | | |
| | | cellular response to interleukin-1 | 0.00633818 | | |
| | | cellular response to BMP stimulus | 0.00803146 | | |
| | | response to BMP | 0.00803146 | | |
| | M2 | response to peptide | 0.00185788 | 14 | 15 |
| | | cellular response to peptide | 0.00185788 | | |
| | | cellular response to organonitrogen compound | 0.00385886 | | |
| | | cellular response to nitrogen compound | 0.00554635 | | |
| | | response to organonitrogen compound | 0.00626552 | | |
| | | cellular response to peptide hormone stimulus | 0.00626552 | | |
| | | response to peptide hormone | 0.00633818 | | |
| | | regulation of supramolecular fiber organization | 0.01274967 | | |
| | | actin filament organization | 0.0131885 | | |
| | | cellular response to hormone stimulus | 0.02135503 | | |
| | M3 | regulation of cysteine-type endopeptidase activity | 0.00336961 | 17 | 32 |
| | | involved in apoptotic process | | | |
| | | regulation of cysteine-type endopeptidase activity | 0.00385886 | | |
| | | glycosaminoglycan metabolic process | 0.00469825 | | |
| | | aminoglycan metabolic process | 0.0050056 | | |
| | | negative regulation of response to DNA damage | 0.0057775 | | |
| | | stimulus | | | |
| | | regulation of endopeptidase activity | 0.00619305 | | |
| | | regulation of peptidase activity | 0.00633818 | | |
| | | positive regulation of cysteine-type endopeptidase | 0.00790601 | | |
| | | activity involved in apoptotic process | | | |
| | | ossification | 0.00816077 | | |
| | | morphogenesis of an epithelium | 0.00844225 | | |

TABLE 19
Upregulated modules in the liver for OrganEx vs other experimental condition

| | UPREGULATED MODULES | | | | |
|---|---|---|---|---|---|
| OrganEx vs | MODULE | TOP TERMS | Q VAL | GENES | TERMS |
| 0 h PMI | M1 | endoplasmic reticulum calcium ion homeostasis | 0.00398603 | 22 | 70 |
| | | positive regulation of ATPase activity | 0.00546596 | | |
| | | cellular response to extracellular stimulus | 0.00546596 | | |
| | | response to nutrient levels | 0.00546596 | | |
| | | cellular response to nutrient levels | 0.00546596 | | |
| | | intracellular protein transport | 0.00546596 | | |
| | | retrograde protein transport, ER to cytosol | 0.00546596 | | |
| | | endoplasmic reticulum to cytosol transport | 0.00546596 | | |
| | | response to extracellular stimulus | 0.00549078 | | |
| | | cellular response to external stimulus | 0.00664246 | | |
| | M2 | positive regulation of cell-matrix adhesion | 0.0050993 | 12 | 31 |
| | | regulation of apoptotic signaling pathway | 0.00546596 | | |
| | | positive regulation of cell-substrate adhesion | 0.0054697 | | |
| | | negative regulation of intrinsic apoptotic signaling | 0.00664246 | | |
| | | pathway | | | |
| | | positive regulation of cellular component biogenesis | 0.00669441 | | |
| | | regulation of cell-matrix adhesion | 0.00669441 | | |
| | | negative regulation of intracellular signal transduction | 0.00764427 | | |
| | | positive regulation of apoptotic signaling pathway | 0.00798568 | | |
| | | regulation of intrinsic apoptotic signaling pathway | 0.00896691 | | |
| | | regulation of cell-substrate adhesion | 0.00951003 | | |
| | M3 | positive regulation of protein tyrosine kinase activity | 0.00546596 | 18 | 25 |
| | | regulation of protein tyrosine kinase activity | 0.00669441 | | |
| | | positive regulation of protein kinase activity | 0.00859284 | | |
| | | positive regulation of kinase activity | 0.00959975 | | |
| | | positive regulation of apoptotic process | 0.01084788 | | |
| | | positive regulation of programmed cell death | 0.01090735 | | |
| | | positive regulation of peptidyl-tyrosine | 0.0138127 | | |
| | | phosphorylation | | | |
| | | negative regulation of cell adhesion | 0.01681081 | | |
| | | regulation of peptidyl-tyrosine phosphorylation | 0.01949561 | | |
| | | protein dephosphorylation | 0.01949561 | | |
| 1 h PMI | M1 | sphingomyelin biosynthetic process | 0.0000256 | 12 | 14 |
| | | sphingomyelin metabolic process | 0.00028511 | | |
| | | sphingolipid biosynthetic process | 0.00189297 | | |
| | | membrane lipid biosynthetic process | 0.00286198 | | |
| | | phospholipid biosynthetic process | 0.00314563 | | |
| | | sphingolipid metabolic process | 0.00354297 | | |
| | | ammonium ion metabolic process | 0.0045515 | | |
| | | membrane lipid metabolic process | 0.0045515 | | |
| | | phospholipid metabolic process | 0.00838847 | | |
| | | positive regulation of GTPase activity | 0.0088777 | | |
| | M2 | protein refolding | 0.00041268 | 12 | 73 |
| | | positive regulation of interleukin-10 production | 0.00114827 | | |
| | | regulation of interleukin-10 production | 0.00169446 | | |
| | | interleukin-10 production | 0.0017855 | | |
| | | T cell mediated immunity | 0.00190809 | | |
| | | lymphocyte activation involved in immune response | 0.00223168 | | |
| | | regulation of protein stability | 0.00237253 | | |
| | | protein folding | 0.00314563 | | |
| | | adaptive immune response based on somatic | 0.00368494 | | |
| | | recombination of immune receptors built from | | | |
| | | immunoglobulin superfamily domains | | | |
| | | supramolecular fiber organization | 0.0045515 | | |
| | M3 | response to interferon-beta | 0.00169446 | 17 | 29 |
| | | cellular response to cytokine stimulus | 0.00186289 | | |
| | | type I interferon signaling pathway | 0.00186289 | | |
| | | cellular response to type I interferon | 0.00189297 | | |
| | | negative regulation of intracellular signal transduction | 0.00195108 | | |
| | | response to type I interferon | 0.00237253 | | |
| | | cellular response to interferon-gamma | 0.00368494 | | |
| | | cytokine-mediated signaling pathway | 0.00398415 | | |
| | | cellular response to interleukin-1 | 0.0045515 | | |
| | | response to interferon-gamma | 0.00485815 | | |
| | M4 | negative regulation of cell adhesion | 0.0045515 | 7 | 4 |
| | | defense response to other organism | 0.00838847 | | |
| | | positive regulation of apoptotic process | 0.01173558 | | |
| | | positive regulation of programmed cell death | 0.01184138 | | |
| 7 h PMI | M1 | negative regulation of intracellular signal transduction | 0.00098861 | 24 | 103 |
| | | cellular response to cytokine stimulus | 0.00156467 | | |
| | | response to interferon-beta | 0.00589599 | | |
| | | type I interferon signaling pathway | 0.00737842 | | |
| | | cellular response to type I interferon | 0.00740207 | | |
| | | retrograde protein transport, ER to cytosol | 0.00889139 | | |
| | | endoplasmic reticulum to cytosol transport | 0.00889139 | | |
| | | response to type I interferon | 0.00893024 | | |
| | | negative regulation of ERK1 and ERK2 cascade | 0.00898752 | | |
| | | cellular response to interferon-gamma | 0.01325363 | | |
| | M2 | peptide hormone processing | 0.00105037 | 13 | 12 |
| | | hormone metabolic process | 0.01325363 | | |
| | | cell killing | 0.01445314 | | |
| | | protein processing | 0.01445314 | | |
| | | negative regulation of cell adhesion | 0.01560983 | | |
| | | protein maturation | 0.0162748 | | |
| | | regulation of hormone levels | 0.01877447 | | |
| | | defense response to other organism | 0.02386996 | | |
| | | symbiont process | 0.02927052 | | |
| | | peptide metabolic process | 0.03190489 | | |
| | M3 | phosphatidylethanolamine biosynthetic process | 0.00105037 | 27 | 27 |
| | | regulation of transcription from RNA polymerase II | 0.00105037 | | |
| | | promoter in response to stress | | | |
| | | regulation of DNA-templated transcription in response | 0.00105037 | | |
| | | to stress | | | |
| | | phosphatidylethanolamine metabolic process | 0.00150736 | | |
| | | negative regulation of transcription from RNA | 0.00193167 | | |
| | | polymerase II promoter in response to stress | | | |
| | | glycerophospholipid biosynthetic process | 0.01445314 | | |
| | | protein folding | 0.01445314 | | |
| | | calcium ion transport | 0.01560983 | | |
| | | phospholipid biosynthetic process | 0.01560983 | | |
| | | divalent metal ion transport | 0.0162748 | | |
| ECMO | M1 | positive regulation of fat cell differentiation | 0.00276459 | 21 | 12 |
| | | regulation of fat cell differentiation | 0.00425328 | | |
| | | fat cell differentiation | 0.00436295 | | |
| | | connective tissue development | 0.00521535 | | |
| | | regulation of ossification | 0.00707335 | | |
| | | ossification | 0.01007525 | | |
| | | neuron death | 0.01007525 | | |
| | | skeletal system development | 0.01199471 | | |
| | | hematopoietic or lymphoid organ development | 0.03558779 | | |
| | | negative regulation of hydrolase activity | 0.03792403 | | |
| | M2 | drug metabolic process | 0.00276459 | 16 | 32 |
| | | reactive oxygen species metabolic process | 0.00276459 | | |
| | | regulation of reactive oxygen species biosynthetic | 0.00361438 | | |
| | | process | | | |
| | | neurotransmitter metabolic process | 0.00425328 | | |
| | | reactive oxygen species biosynthetic process | 0.00425328 | | |
| | | dicarboxylic acid metabolic process | 0.00425328 | | |
| | | cofactor metabolic process | 0.00425328 | | |
| | | protein homotetramerization | 0.00425328 | | |
| | | cellular response to organonitrogen compound | 0.00425328 | | |
| | | cellular modified amino acid metabolic process | 0.00515027 | | |
| | M3 | response to inorganic substance | 0.00425328 | 4 | 2 |
| | | response to drug | 0.00575263 | | |

TABLE 20
Downregulated modules in the liver for OrganEx vs other experimental conditions

| | MODULE | TOP TERMS | Q VAL | GENES | TERMS |
|---|---|---|---|---|---|
| 0 h PMI | M1 | bile acid biosynthetic process | 0.00031886 | 9 | 13 |
| | | bile acid metabolic process | 0.00032782 | | |
| | | small molecule biosynthetic process | 0.00146015 | | |
| | | steroid biosynthetic process | 0.00146015 | | |
| | | organic hydroxy compound biosynthetic | 0.00191362 | | |
| | | process | | | |
| | | monocarboxylic acid biosynthetic process | 0.00271082 | | |
| | | steroid metabolic process | 0.00314049 | | |
| | | organic hydroxy compound metabolic process | 0.00434973 | | |
| | | carboxylic acid biosynthetic process | 0.00434973 | | |
| | | organic acid biosynthetic process | 0.00434973 | | |
| | M2 | tube morphogenesis | 0.00783003 | 7 | 2 |
| | | tube development | 0.00835127 | | |
| 1 h PMI | M1 | organic hydroxy compound metabolic process | 0.00041564 | 10 | 19 |
| | | small molecule catabolic process | 0.00041564 | | |
| | | lipid catabolic process | 0.00041564 | | |
| | | alcohol catabolic process | 0.00041564 | | |
| | | organic hydroxy compound catabolic process | 0.00042391 | | |
| | | monocarboxylic acid metabolic process | 0.00082197 | | |
| | | fatty acid catabolic process | 0.00082197 | | |
| | | monocarboxylic acid catabolic process | 0.0010703 | | |
| | | cellular lipid catabolic process | 0.00184709 | | |
| | | viral genome replication | 0.00184709 | | |
| | M2 | small molecule biosynthetic process | 0.00155758 | 11 | 4 |
| | | carboxylic acid biosynthetic process | 0.00560495 | | |
| | | organic acid biosynthetic process | 0.00560495 | | |
| | | regulation of response to external stimulus | 0.01648373 | | |
| 7 h PMI | M1 | regulation of endocytosis | 0.0000003 | 9 | 75 |
| | | regulation of receptor-mediated endocytosis | 0.00000079 | | |
| | | regulation of vesicle-mediated transport | 0.00000184 | | |
| | | endocytosis | 0.00000265 | | |
| | | import into cell | 0.00000376 | | |
| | | receptor-mediated endocytosis | 0.00000837 | | |
| | | regulation of very-low-density lipoprotein | 0.00002529 | | |
| | | particle clearance | | | |
| | | negative regulation of very-low-density | 0.00002529 | | |
| | | lipoprotein particle clearance | | | |
| | | chylomicron remnant clearance | 0.00002529 | | |
| | | triglyceride-rich lipoprotein particle clearance | 0.00002529 | | |
| | M2 | small molecule catabolic process | 0.00045254 | 11 | 20 |
| | | carboxylic acid biosynthetic process | 0.00048814 | | |
| | | organic acid biosynthetic process | 0.00048814 | | |
| | | alpha-amino acid catabolic process | 0.00071999 | | |
| | | cellular amino acid catabolic process | 0.00079081 | | |
| | | small molecule biosynthetic process | 0.00144404 | | |
| | | coenzyme biosynthetic process | 0.00167035 | | |
| | | cofactor biosynthetic process | 0.00246223 | | |
| | | alpha-amino acid metabolic process | 0.00284418 | | |
| | | monocarboxylic acid biosynthetic process | 0.00357393 | | |
| | M3 | primary alcohol metabolic process | 0.00062878 | 12 | 4 |
| | | alcohol metabolic process | 0.0043331 | | |
| | | organic hydroxy compound metabolic process | 0.00773974 | | |
| | | drug metabolic process | 0.01946794 | | |
| | M4 | negative regulation of angiogenesis | 0.00071999 | 6 | 13 |
| | | negative regulation of blood vessel | 0.00073575 | | |
| | | morphogenesis | | | |
| | | negative regulation of vasculature development | 0.00079436 | | |
| | | regulation of angiogenesis | 0.00284418 | | |
| | | regulation of vasculature development | 0.00312124 | | |
| | | angiogenesis | 0.0043331 | | |
| | | blood vessel morphogenesis | 0.00467035 | | |
| | | blood vessel development | 0.00505178 | | |
| | | small molecule biosynthetic process | 0.00536108 | | |
| | | vasculature development | 0.00537425 | | |
| ECMO | M1 | negative regulation of innate immune response | 0.02465491 | 19 | 40 |
| | | negative regulation of immune response | 0.02898008 | | |
| | | regulation of calcium ion transmembrane | 0.02898008 | | |
| | | transport | | | |
| | | protein processing | 0.03198994 | | |
| | | regulation of response to external stimulus | 0.03198994 | | |
| | | negative regulation of defense response | 0.03198994 | | |
| | | protein maturation | 0.03198994 | | |
| | | negative regulation of cell adhesion | 0.03198994 | | |
| | | circulatory system process | 0.03198994 | | |
| | | calcium ion transmembrane transport | 0.03198994 | | |
| | M2 | cellular component morphogenesis | 0.03198994 | 21 | 14 |
| | | positive regulation of cell development | 0.03198994 | | |
| | | response to endoplasmic reticulum stress | 0.03308581 | | |
| | | regulation of cell morphogenesis | 0.03500906 | | |
| | | positive regulation of cell adhesion | 0.03583142 | | |
| | | actin filament organization | 0.03583142 | | |
| | | regulation of protein stability | 0.040358 | | |
| | | regulation of cell development | 0.04147795 | | |
| | | cell morphogenesis | 0.04906785 | | |
| | | dephosphorylation | 0.05084461 | | |
| | M3 | transmembrane receptor protein tyrosine kinase | 0.03198994 | 9 | 5 |
| | | signaling pathway | | | |
| | | metal ion homeostasis | 0.03198994 | | |
| | | cation homeostasis | 0.03198994 | | |
| | | inorganic ion homeostasis | 0.03198994 | | |
| | | endomembrane system organization | 0.03198994 | | |

TABLE 21
Upregulated modules in the kidney for OrganEx vs other experimental conditions

| | UPREGULATED MODULES | | | | |
|---|---|---|---|---|---|
| OrganEx vs | MODULE | TOP TERMS | Q VAL | GENES | TERMS |
| 0 h PMI | M1 | protein folding | 0.00000036 | 21 | 205 |
| | | chaperone-mediated protein complex assembly | 0.00000036 | | |
| | | telomerase holoenzyme complex assembly | 0.00000332 | | |
| | | protein refolding | 0.00001041 | | |
| | | telomere maintenance via telomerase | 0.00002933 | | |
| | | RNA-dependent DNA biosynthetic process | 0.00004271 | | |
| | | telomere maintenance via telomere | 0.00004271 | | |
| | | lengthening | | | |
| | | regulation of DNA biosynthetic process | 0.00008467 | | |
| | | regulation of protein stability | 0.00009942 | | |
| | | telomere maintenance | 0.00013759 | | |
| | M2 | actin cytoskeleton reorganization | 0.00065265 | 5 | 11 |
| | | negative regulation of MAPK cascade | 0.00128835 | | |
| | | regulation of intracellular transport | 0.00387651 | | |
| | | regulation of vesicle-mediated transport | 0.00421455 | | |
| | | positive regulation of cellular catabolic process | 0.00441408 | | |
| | | negative regulation of protein phosphorylation | 0.00465765 | | |
| | | negative regulation of phosphorylation | 0.00512152 | | |
| | | positive regulation of catabolic process | 0.0051579 | | |
| | | actin cytoskeleton organization | 0.00616101 | | |
| | | regulation of cellular protein localization | 0.00617329 | | |
| | M3 | protein tetramerization | 0.00677288 | 16 | 17 |
| | | innate immune response-activating signal | 0.00723792 | | |
| | | transduction | | | |
| | | pattern recognition receptor signaling pathway | 0.00723792 | | |
| | | activation of innate immune response | 0.00985675 | | |
| | | positive regulation of innate immune response | 0.01317658 | | |
| | | immune response-activating signal | 0.01453597 | | |
| | | transduction | | | |
| | | immune response-regulating signaling | 0.01572326 | | |
| | | pathway | | | |
| | | regulation of innate immune response | 0.0158122 | | |
| | | positive regulation of defense response | 0.0158122 | | |
| | | activation of immune response | 0.01950167 | | |
| | M4 | dephosphorylation | 0.01918327 | 11 | 1 |
| 1 h PMI | M1 | telomerase holoenzyme complex assembly | 0.0000033 | 20 | 76 |
| | | chaperone-mediated protein complex assembly | 0.00002344 | | |
| | | positive regulation of telomerase activity | 0.00021783 | | |
| | | regulation of telomerase activity | 0.00033656 | | |
| | | protein folding | 0.0004495 | | |
| | | positive regulation of DNA biosynthetic | 0.0004551 | | |
| | | process | | | |
| | | telomere maintenance via telomerase | 0.00049629 | | |
| | | protein refolding | 0.00058816 | | |
| | | RNA-dependent DNA biosynthetic process | 0.00058816 | | |
| | | telomere maintenance via telomere | 0.00058816 | | |
| | | lengthening | | | |
| | M2 | vitamin transmembrane transport | 0.00089093 | 20 | 17 |
| | | negative regulation of anoikis | 0.00095354 | | |
| | | regulation of anoikis | 0.001341 | | |
| | | vitamin transport | 0.00159456 | | |
| | | anoikis | 0.00160714 | | |
| | | positive regulation of apoptotic signaling | 0.00960626 | | |
| | | pathway | | | |
| | | regulation of extrinsic apoptotic signaling | 0.01003699 | | |
| | | pathway | | | |
| | | extrinsic apoptotic signaling pathway | 0.01707675 | | |
| | | negative regulation of cellular catabolic | 0.02206534 | | |
| | | process | | | |
| | | negative regulation of catabolic process | 0.02460865 | | |
| 7 h PMI | M1 | protein folding | 0.00007133 | 22 | 164 |
| | | chaperone-mediated protein complex assembly | 0.00007133 | | |
| | | mitotic spindle assembly | 0.00120742 | | |
| | | telomere maintenance via telomerase | 0.00120742 | | |
| | | protein refolding | 0.00120742 | | |
| | | negative regulation of transcription from RNA | 0.00120742 | | |
| | | polymerase II promoter in response to stress | | | |
| | | negative regulation of inclusion body assembly | 0.00120742 | | |
| | | telomerase holoenzyme complex assembly | 0.00120742 | | |
| | | ‘de novo’ protein folding | 0.00120742 | | |
| | | ‘de novo’ posttranslational protein folding | 0.00120742 | | |
| | M2 | positive regulation of cellular protein | 0.0043662 | 5 | 4 |
| | | localization | | | |
| | | positive regulation of protein transport | 0.00532702 | | |
| | | positive regulation of establishment of protein | 0.00552056 | | |
| | | localization | | | |
| | | regulation of cellular protein localization | 0.00744499 | | |
| | M3 | regulation of intrinsic apoptotic signaling | 0.00476218 | 11 | 5 |
| | | pathway | | | |
| | | negative regulation of apoptotic signaling | 0.00591462 | | |
| | | pathway | | | |
| | | regulation of response to DNA damage | 0.00744499 | | |
| | | stimulus | | | |
| | | intrinsic apoptotic signaling pathway | 0.00828698 | | |
| | | regulation of apoptotic signaling pathway | 0.01219699 | | |
| | M4 | actin cytoskeleton organization | 0.00744499 | 5 | 1 |
| ECMO | M1 | response to thyroid hormone | 0.00289388 | 23 | 49 |
| | | cellular response to thyroid hormone stimulus | 0.00289388 | | |
| | | regulation of transcription from RNA | 0.00774834 | | |
| | | polymerase II promoter in response to stress | | | |
| | | cellular response to glucose starvation | 0.00774834 | | |
| | | regulation of DNA-templated transcription in | 0.00774834 | | |
| | | response to stress | | | |
| | | circadian rhythm | 0.00774834 | | |
| | | rhythmic process | 0.00774834 | | |
| | | maintenance of location | 0.00774834 | | |
| | | positive regulation of smooth muscle cell | 0.00774834 | | |
| | | proliferation | | | |
| | | smooth muscle cell proliferation | 0.01219399 | | |
| | M2 | protein polyubiquitination | 0.00774834 | 6 | 1 |
| | M3 | protein stabilization | 0.00774834 | 6 | 3 |
| | | regulation of protein stability | 0.00924636 | | |
| | | regulation of chromosome organization | 0.01025647 | | |
| | M4 | positive regulation of intracellular transport | 0.00774834 | 7 | 3 |
| | | regulation of intracellular transport | 0.00858876 | | |
| | | microtubule cytoskeleton organization | 0.01406365 | | |
| | M5 | cellular response to oxidative stress | 0.00774834 | 9 | 5 |
| | | response to oxidative stress | 0.01179599 | | |
| | | regulation of transmembrane transport | 0.01219399 | | |
| | | regulation of protein catabolic process | 0.01406365 | | |
| | | response to organonitrogen compound | 0.02146332 | | |
| | M6 | mRNA processing | 0.00774834 | 5 | 2 |
| | | mRNA metabolic process | 0.00924636 | | |

TABLE 22
Downregulated modules in the kidney for OrganEx vs other experimental conditions

| MODULE | TOP TERMS | Q VAL | GENES | TERMS |
|---|---|---|---|---|
| M1 | gluconeogenesis | 0.00022628 | 9 | 9 |
| | hexose biosynthetic process | 0.00022628 | | |
| | monosaccharide biosynthetic process | 0.00022628 | | |
| | glucose metabolic process | 0.00127219 | | |
| | carbohydrate biosynthetic process | 0.00146553 | | |
| | hexose metabolic process | 0.00146553 | | |
| | monosaccharide metabolic process | 0.00223435 | | |
| | carbohydrate metabolic process | 0.00637556 | | |
| | small molecule biosynthetic process | 0.01099155 | | |
| M2 | carboxylic acid transport | 0.00090887 | 19 | 13 |
| | organic acid transport | 0.00090887 | | |
| | organic anion transport | 0.00223435 | | |
| | fatty acid transport | 0.00223435 | | |
| | monocarboxylic acid transport | 0.00293503 | | |
| | anion transport | 0.0051951 | | |
| | regulation of stress-activated MAPK cascade | 0.0138882 | | |
| | regulation of stress-activated protein kinase | 0.0138882 | | |
| | signaling | | | |
| | cascade | | | |
| | stress-activated MAPK cascade | 0.01395919 | | |
| | stress-activated protein kinase signaling cascade | 0.01395919 | | |
| M3 | fatty acid oxidation | 0.00293503 | 18 | 12 |
| | lipid oxidation | 0.0029948 | | |
| | monosaccharide metabolic process | 0.00783306 | | |
| | alpha-amino acid metabolic process | 0.00833857 | | |
| | cellular amino acid metabolic process | 0.01341131 | | |
| | fatty acid metabolic process | 0.01395919 | | |
| | lipid modification | 0.01395919 | | |
| | carbohydrate metabolic process | 0.02064605 | | |
| | anion transport | 0.02908113 | | |
| | monocarboxylic acid metabolic process | 0.02956181 | | |
| M1 | sialylation | 0.00023984 | 14 | 7 |
| | response to insulin | 0.00117307 | | |
| | response to peptide hormone | 0.00227106 | | |
| | response to peptide | 0.00415449 | | |
| | carbohydrate metabolic process | 0.00958846 | | |
| | response to hormone | 0.01872034 | | |
| | response to organonitrogen compound | 0.0236127 | | |
| M1 | positive regulation of endocytosis | 0.00068494 | 12 | 24 |
| | positive regulation of receptor internalization | 0.00098167 | | |
| | regulation of endocytosis | 0.00098167 | | |
| | regulation of receptor internalization | 0.00168554 | | |
| | positive regulation of receptor-mediated | 0.00168554 | | |
| | endocytosis | | | |
| | regulation of vesicle-mediated transport | 0.00168554 | | |
| | endocytosis | 0.00219667 | | |
| | import into cell | 0.00228672 | | |
| | receptor internalization | 0.00228672 | | |
| | regulation of receptor-mediated endocytosis | 0.00228672 | | |
| M2 | sulfur amino acid metabolic process | 0.00084666 | 7 | 4 |
| | alpha-amino acid metabolic process | 0.00228672 | | |
| | cellular amino acid metabolic process | 0.00389259 | | |
| | sulfur compound metabolic process | 0.0039442 | | |
| M3 | carbohydrate phosphorylation | 0.00168554 | 16 | 21 |
| | intrinsic apoptotic signaling pathway by p53 class | 0.00228672 | | |
| | mediator | | | |
| | ammonium ion metabolic process | 0.00549372 | | |
| | monosaccharide metabolic process | 0.00664531 | | |
| | signal transduction by p53 class mediator | 0.00679184 | | |
| | cellular carbohydrate metabolic process | 0.00731867 | | |
| | regulation of proteasomal protein catabolic process | 0.00796902 | | |
| | positive regulation of binding | 0.00924769 | | |
| | regulation of proteolysis involved in cellular protein | 0.01072861 | | |
| | catabolic process | | | |
| | regulation of cellular protein catabolic process | 0.0133162 | | |
| M1 | gliogenesis | 0.00241591 | 7 | 2 |
| | neurogenesis | 0.01454255 | | |
| M2 | cellular response to transforming growth factor beta | 0.01057722 | 11 | 17 |
| | stimulus | | | |
| | response to transforming growth factor beta | 0.01057722 | | |
| | cell-cell junction organization | 0.01057722 | | |
| | cell junction organization | 0.01057722 | | |
| | cellular divalent inorganic cation homeostasis | 0.01454255 | | |
| | divalent inorganic cation homeostasis | 0.01454255 | | |
| | cellular metal ion homeostasis | 0.01650704 | | |
| | cellular ion homeostasis | 0.01650766 | | |
| | cellular cation homeostasis | 0.01650766 | | |
| | metal ion homeostasis | 0.01650766 | | |
| M3 | nervous system process | 0.01966787 | 6 | 1 |

TABLE 23
List of Enriched GO terms.

| | GO. ID | Term | Annotated | Significant | Expected | classicFisher | module |
|---|---|---|---|---|---|---|---|
| 1 | GO:0048812 | neuron projection morphogenesis | 157 | 7 | 0.67 | 2.80E−06 | ME_1 |
| 2 | GO:0120039 | plasma membrane bounded cell projection . . . | 161 | 7 | 0.68 | 3.30E−06 | ME_1 |
| 3 | GO:0048858 | cell projection morphogenesis | 162 | 7 | 0.69 | 3.40E−06 | ME_1 |
| 4 | GO:0032990 | cell part morphogenesis | 166 | 7 | 0.7 | 4.00E−06 | ME_1 |
| 5 | GO:0000902 | cell morphogenesis | 264 | 8 | 1.12 | 8.50E−06 | ME_1 |
| 6 | GO:0032989 | cellular component morphogenesis | 203 | 7 | 0.86 | 1.50E−05 | ME_1 |
| 7 | GO:0048667 | cell morphogenesis involved in neuron di . . . | 145 | 6 | 0.62 | 2.50E−05 | ME_1 |
| 8 | GO:0031175 | neuron projection development | 231 | 7 | 0.98 | 3.50E−05 | ME_1 |
| 9 | GO:0022008 | neurogenesis | 429 | 9 | 1.82 | 4.00E−05 | ME_1 |
| 10 | GO:0051649 | establishment of localization in cell | 553 | 10 | 2.35 | 4.60E−05 | ME_1 |
| 11 | GO:0007399 | nervous system development | 571 | 10 | 2.42 | 6.10E−05 | ME_1 |
| 12 | GO:0051049 | regulation of transport | 453 | 9 | 1.92 | 6.10E−05 | ME_1 |
| 13 | GO:1902903 | regulation of supramolecular fiber organ . . . | 105 | 5 | 0.45 | 6.60E−05 | ME_1 |
| 14 | GO:0120036 | plasma membrane bounded cell projection . . . | 351 | 8 | 1.49 | 6.70E−05 | ME_1 |
| 15 | GO:0030182 | neuron differentiation | 353 | 8 | 1.5 | 7.00E−05 | ME_1 |
| 16 | GO:0030030 | cell projection organization | 357 | 8 | 1.51 | 7.60E−05 | ME_1 |
| 17 | GO:0051179 | localization | 1714 | 17 | 7.27 | 7.60E−05 | ME_1 |
| 18 | GO:0048666 | neuron development | 266 | 7 | 1.13 | 8.70E−05 | ME_1 |
| 19 | GO:0007409 | axonogenesis | 115 | 5 | 0.49 | 0.0001 | ME_1 |
| 20 | GO:0000904 | cell morphogenesis involved in different . . . | 187 | 6 | 0.79 | 0.0001 | ME_1 |
| 21 | GO:0048699 | generation of neurons | 399 | 8 | 1.69 | 0.00017 | ME_1 |
| 22 | GO:1904062 | regulation of cation transmembrane trans . . . | 68 | 4 | 0.29 | 0.00017 | ME_1 |
| 23 | GO:0061564 | axon development | 129 | 5 | 0.55 | 0.00018 | ME_1 |
| 24 | GO:0051128 | regulation of cellular component organiz . . . | 647 | 10 | 2.74 | 0.00018 | ME_1 |
| 25 | GO:0032879 | regulation of localization | 704 | 10 | 2.99 | 0.00036 | ME_1 |
| 26 | GO:0046777 | protein autophosphorylation | 56 | 4 | 0.14 | 7.90E−06 | ME_2 |
| 27 | GO:2001222 | regulation of neuron migration | 11 | 2 | 0.03 | 0.0003 | ME_2 |
| 28 | GO:0048667 | cell morphogenesis involved in neuron di . . . | 145 | 4 | 0.35 | 0.00033 | ME_2 |
| 29 | GO:0048812 | neuron projection morphogenesis | 157 | 4 | 0.38 | 0.00045 | ME_2 |
| 30 | GO:0120039 | plasma membrane bounded cell projection . . . | 161 | 4 | 0.39 | 0.0005 | ME_2 |
| 31 | GO:0048858 | cell projection morphogenesis | 162 | 4 | 0.4 | 0.00051 | ME_2 |
| 32 | GO:0032990 | cell part morphogenesis | 166 | 4 | 0.41 | 0.00056 | ME_2 |
| 33 | GO:0000904 | cell morphogenesis involved in different . . . | 187 | 4 | 0.46 | 0.00088 | ME_2 |
| 34 | GO:0032989 | cellular component morphogenesis | 203 | 4 | 0.5 | 0.0012 | ME_2 |
| 35 | GO:0030001 | metal ion transport | 213 | 4 | 0.52 | 0.00143 | ME_2 |
| 36 | GO:0010959 | regulation of metal ion transport | 95 | 3 | 0.23 | 0.00143 | ME_2 |
| 37 | GO:0031175 | neuron projection development | 231 | 4 | 0.57 | 0.00193 | ME_2 |
| 38 | GO:0051928 | positive regulation of calcium ion trans . . . | 28 | 2 | 0.07 | 0.00204 | ME_2 |
| 39 | GO:0048813 | dendrite morphogenesis | 29 | 2 | 0.07 | 0.00218 | ME_2 |
| 40 | GO:0006811 | ion transport | 426 | 5 | 1.04 | 0.00265 | ME_2 |
| 41 | GO:0006796 | phosphate-containing compound metabolic . . . | 884 | 7 | 2.16 | 0.00283 | ME_2 |
| 42 | GO:0006793 | phosphorus metabolic process | 898 | 7 | 2.2 | 0.0031 | ME_2 |
| 43 | GO:0000902 | cell morphogenesis | 264 | 4 | 0.65 | 0.00315 | ME_2 |
| 44 | GO:0048666 | neuron development | 266 | 4 | 0.65 | 0.00324 | ME_2 |
| 45 | GO:0006812 | cation transport | 288 | 4 | 0.7 | 0.00432 | ME_2 |
| 46 | GO:0032879 | regulation of localization | 704 | 6 | 1.72 | 0.00451 | ME_2 |
| 47 | GO:0001764 | neuron migration | 43 | 2 | 0.11 | 0.00476 | ME_2 |
| 48 | GO:0016358 | dendrite development | 49 | 2 | 0.12 | 0.00615 | ME_2 |
| 49 | GO:0043269 | regulation of ion transport | 164 | 3 | 0.4 | 0.00675 | ME_2 |
| 50 | GO:0043270 | positive regulation of ion transport | 53 | 2 | 0.13 | 0.00717 | ME_2 |
| 51 | GO:0010738 | regulation of protein kinase A signaling | 10 | 1 | 0.01 | 0.0098 | ME_3 |
| 52 | GO:0033238 | regulation of cellular amine metabolic p . . . | 10 | 1 | 0.01 | 0.0098 | ME_3 |
| 53 | GO:0043486 | histone exchange | 10 | 1 | 0.01 | 0.0098 | ME_3 |
| 54 | GO:1902410 | mitotic cytokinetic process | 10 | 1 | 0.01 | 0.0098 | ME_3 |
| 55 | GO:0010640 | regulation of platelet-derived growth fa . . . | 11 | 1 | 0.01 | 0.0107 | ME_3 |
| 56 | GO:0030520 | intracellular estrogen receptor signalin . . . | 11 | 1 | 0.01 | 0.0107 | ME_3 |
| 57 | GO:0042133 | neurotransmitter metabolic process | 11 | 1 | 0.01 | 0.0107 | ME_3 |
| 58 | GO:0060065 | uterus development | 12 | 1 | 0.01 | 0.0117 | ME_3 |
| 59 | GO:0006884 | cell volume homeostasis | 13 | 1 | 0.01 | 0.0127 | ME_3 |
| 60 | GO:0032506 | cytokinetic process | 13 | 1 | 0.01 | 0.0127 | ME_3 |
| 61 | GO:0046850 | regulation of bone remodeling | 13 | 1 | 0.01 | 0.0127 | ME_3 |
| 62 | GO:0061756 | leukocyte adhesion to vascular endotheli . . . | 13 | 1 | 0.01 | 0.0127 | ME_3 |
| 63 | GO:0010737 | protein kinase A signaling | 14 | 1 | 0.01 | 0.0136 | ME_3 |
| 64 | GO:0070936 | protein K48-linked ubiquitination | 15 | 1 | 0.01 | 0.0146 | ME_3 |
| 65 | GO:0006635 | fatty acid beta-oxidation | 16 | 1 | 0.02 | 0.0156 | ME_3 |
| 66 | GO:0022602 | ovulation cycle process | 16 | 1 | 0.02 | 0.0156 | ME_3 |
| 67 | GO:0034103 | regulation of tissue remodeling | 16 | 1 | 0.02 | 0.0156 | ME_3 |
| 68 | GO:0045123 | cellular extravasation | 16 | 1 | 0.02 | 0.0156 | ME_3 |
| 69 | GO:0033143 | regulation of intracellular steroid horm . . . | 17 | 1 | 0.02 | 0.0165 | ME_3 |
| 70 | GO:0043044 | ATP-dependent chromatin remodeling | 17 | 1 | 0.02 | 0.0165 | ME_3 |
| 71 | GO:0045453 | bone resorption | 17 | 1 | 0.02 | 0.0165 | ME_3 |
| 72 | GO:0030104 | water homeostasis | 19 | 1 | 0.02 | 0.0185 | ME_3 |
| 73 | GO:0001541 | ovarian follicle development | 21 | 1 | 0.02 | 0.0204 | ME_3 |
| 74 | GO:0048008 | platelet-derived growth factor receptor . . . | 21 | 1 | 0.02 | 0.0204 | ME_3 |
| 75 | GO:0007193 | adenylate cyclase-inhibiting G protein-c . . . | 22 | 1 | 0.02 | 0.0214 | ME_3 |
| 76 | GO:0098742 | cell-cell adhesion via plasma-membrane a . . . | 48 | 2 | 0.06 | 0.0016 | ME_4 |
| 77 | GO:0007275 | multicellular organism development | 1360 | 6 | 1.78 | 0.0022 | ME_4 |
| 78 | GO:0007399 | nervous system development | 571 | 4 | 0.75 | 0.0038 | ME_4 |
| 79 | GO:0048856 | anatomical structure development | 1548 | 6 | 2.02 | 0.0044 | ME_4 |
| 80 | GO:0032502 | developmental process | 1677 | 6 | 2.19 | 0.0069 | ME_4 |
| 81 | GO:0030182 | neuron differentiation | 353 | 3 | 0.46 | 0.0085 | ME_4 |
| 82 | GO:0034329 | cell junction assembly | 113 | 2 | 0.15 | 0.0088 | ME_4 |
| 83 | GO:0048731 | system development | 1217 | 5 | 1.59 | 0.01 | ME_4 |
| 84 | GO:0048699 | generation of neurons | 399 | 3 | 0.52 | 0.012 | ME_4 |
| 85 | GO:0007157 | heterophilic cell-cell adhesion via plas . . . | 10 | 1 | 0.01 | 0.013 | ME_4 |
| 86 | GO:0032501 | multicellular organismal process | 1926 | 6 | 2.51 | 0.0144 | ME_4 |
| 87 | GO:0022008 | neurogenesis | 429 | 3 | 0.56 | 0.0146 | ME_4 |
| 88 | GO:0044331 | cell-cell adhesion mediated by cadherin | 12 | 1 | 0.02 | 0.0156 | ME_4 |
| 89 | GO:0071300 | cellular response to retinoic acid | 12 | 1 | 0.02 | 0.0156 | ME_4 |
| 90 | GO:0060612 | adipose tissue development | 13 | 1 | 0.02 | 0.0169 | ME_4 |
| 91 | GO:1903580 | positive regulation of ATP metabolic pro . . . | 13 | 1 | 0.02 | 0.0169 | ME_4 |
| 92 | GO:0034330 | cell junction organization | 163 | 2 | 0.21 | 0.0177 | ME_4 |
| 93 | GO:0034332 | adherens junction organization | 14 | 1 | 0.02 | 0.0181 | ME_4 |
| 94 | GO:0097009 | energy homeostasis | 14 | 1 | 0.02 | 0.0181 | ME_4 |
| 95 | GO:0099054 | presynapse assembly | 16 | 1 | 0.02 | 0.0207 | ME_4 |
| 96 | GO:0099172 | presynapse organization | 18 | 1 | 0.02 | 0.0233 | ME_4 |
| 97 | GO:0050873 | brown fat cell differentiation | 19 | 1 | 0.02 | 0.0245 | ME_4 |
| 98 | GO:0006140 | regulation of nucleotide metabolic proce . . . | 20 | 1 | 0.03 | 0.0258 | ME_4 |
| 99 | GO:1900542 | regulation of purine nucleotide metaboli . . . | 20 | 1 | 0.03 | 0.0258 | ME_4 |
| 100 | GO:0098609 | cell-cell adhesion | 203 | 2 | 0.26 | 0.0268 | ME_4 |

### Example 7

Here, the OrganEx technology and its potential to support recovery of key molecular and cellular processes in multiple porcine organs after prolonged global warm ischemia are described. This also demonstrates the underappreciated capacity of the large mammalian body for restoration of hemodynamic and metabolic parameters following circulatory arrest or other severe ischemic stress. This application of the OrganEx technology demonstrates that cellular demise can be halted, and their state be shifted towards recovery at molecular and cellular levels, even following prolonged warm ischemia. Additionally, a comprehensive single-cell transcriptomic analysis of multiple porcine organs was generated to provide a unique resource for future studies on cell-types, ischemia, and reperfusion.

Although some cellular viability can be restored following prolonged ischemia in tissue cultures or isolated organs, clinical scenarios typically involve shorter-duration ischemia in the setting of cardiac arrest or regional perfusions in the setting of organ transplant. By employing a rational polytherapy approach built upon optimized perfusion dynamics and augmentations to an acellular synthetic perfusate, the OrganEx technology was able to bridge prior clinical-translational gaps by restoring circulation and metabolic homeostasis across the whole body. This quells deleterious processes caused by disturbed cellular environments and lack of oxygen, representing a distinct feature of this technology and an essential control for multiple nonspecific injury mechanisms affecting end-organ recovery and overall prognosis after global ischemia. Potential applications of this technology are manifold and could provide novel pathways in ischemia research and advance related clinical disciplines. OrganEx has the potential to extend limits of allowable warm ischemia times through regional abdominal/thoracic reperfusion, thereby increasing organ availability for transplantation. This approach would require obligatory antecedent clamping of the aorta/carotid arteries to prevent brain recirculation in the organ donor. Conversely, if any future refinements of the technology could be aimed at the recovery of the brain function after injury, then the brain circulation would remain patent. In this regard, OrganEx technology may improve outcomes in extracorporeal cardiopulmonary resuscitation with the needed circulatory support, or the OrganEx perfusate could aid in recovery wherein cardiac function is preserved but the brain is damaged, as seen in stroke. Thus, a clear distinction should be made before possible utilization of OrganEx technology, relative to inclusion of the brain circulation.

While these studies demonstrate important cellular protection and repair processes in vital organs across meaningful timepoints, questions remain concerning organ recovery over an extended timeframe. Since repeating all experiments over additional, extended durations to comprise a full, longitudinal study were not feasible under current regulatory constraints, long-term organotypic slice culture preparations of hippocampus were employed to study post-perfusion cell survivability in the most ischemia-sensitive tissue possible. This demonstrated that OrganEx intervention provides enduring effects on cellular recovery following transfer to the extended survival conditions.

### Enumerated Embodiments

The following enumerated embodiments are provided, the numbering of which is not to be construed as designating levels of importance.

Embodiment 1 provides an isolated perfusate mixture comprising:

an inorganic salt solution;
an artificial oxygen carrier; and
autologous blood.

Embodiment 2 provides the isolated perfusate mixture of embodiment 1, wherein the one or more artificial oxygen carriers is selected from the group consisting of hemoglobin glutamer-250, isolated cell-free hemoglobin, cross-linked hemoglobin, polymerized hemoglobin, encapsulated hemoglobin, and perfluorocarbon oxygen carriers.

Embodiment 3 provides the isolated perfusate mixture of embodiments 1-2, wherein the artificial oxygen carrier is hemoglobin glutamer-250.

Embodiment 4 provides the isolated perfusate mixture of embodiments 1-3, wherein the one or more inorganic salts are selected from the group consisting of sodium chloride, sodium bicarbonate, magnesium chloride, and calcium chloride.

Embodiment 5 provides the isolated perfusate mixture of embodiments 1-4, wherein the perfusate comprises a priming solution containing one or more sugars.

Embodiment 6 provides the isolated perfusate mixture of embodiments 1-5 wherein the one or more sugars are glucose or dextrane.

Embodiment 7 provides the isolated perfusate mixture of embodiments 1-6, further comprising one or more amino acids.

Embodiment 8 provides the isolated perfusate mixture of embodiments 1-7, wherein the one or more amino acids are selected from the group consisting of glycine, L-alanyl-glutamine, L-arginine, L-cysteine, L-histidine, L-isoleucine, L-leucine, L-lysine, L-methionine, L-phenylalanine, L-serine, L-threonine, L-tryptophan, L-tyrosine, L-valine and salts and solvates thereof.

Embodiment 9 provides the isolated perfusate mixture of embodiments 1-8, wherein the mixture further comprises one or more vitamins.

Embodiment 10 provides the isolated perfusate mixture of embodiments 1-9, wherein the one or more vitamins are selected from the group consisting of choline, D-calcium pantothenate, folic acid, niacinamide, pyridoxine, riboflavin, thiamine, i-inositol and salts and solvates thereof.

Embodiment 11 provides the isolated perfusate mixture of embodiments 1-10, wherein the mixture further comprises, ferric nitrate, magnesium sulfate, potassium chloride, sodium phosphate, and derivatives thereof.

Embodiment 12 provides the isolated perfusate mixture of embodiments 1-11, wherein the mixture further comprises an anti-clotting agent.

Embodiment 13 provides the isolated perfusate mixture of embodiments 1-12, wherein the anti-clotting agent is heparin.

Embodiment 14 provides the isolated perfusate mixture of embodiments 1-13, wherein the percentage of autologous blood in the mixture is between 10% and 50%.

Embodiment 15 provides the isolated perfusate mixture of embodiments 1-14, wherein the percentage of autologous blood in the mixture is approximately 28%.

Embodiment 16 provides the isolated perfusate mixture of embodiments 1-15, wherein the mixture is dialyzed against a solution comprising inorganic salts.

Embodiment 17 provides the isolated perfusate mixture of embodiments 1-16, wherein the mixture is dialyzed against plasma.

Embodiment 18 provides the isolated perfusate mixture of embodiments 1-17, wherein the mixture comprises electrolytes and oncotic agents at levels comparable to those in autologous blood.

Embodiment 19 provides the isolated perfusate mixture of embodiments 1-18, wherein the perfusate further comprises cytoprotective agents.

Embodiment 20 provides the isolated perfusate mixture of embodiments 1-19, wherein the cytoprotective agents are selected from the group consisting of 2-Iminobiotin, Necrostatin-1, sodium 3-hydroxybutryate, glutathione, minocycline, lamotrigine, QVE-Oph, methylene blue, and or any salts, solvates, tautomers, and prodrugs thereof.

Embodiment 21 provides the isolated perfusate mixture of embodiments 1-20, wherein the mixture further comprises antibiotics.

Embodiment 22 provides the isolated perfusate mixture of embodiments 1-21, wherein the antibiotic is ceftriazone.

Embodiment 23 provides the isolated perfusate mixture of embodiments 1-22, wherein the mixture comprises one or more anti-inflammatory agents.

Embodiment 24 provides the isolated perfusate mixture of embodiments 1-23, wherein the one or more the anti-inflammatory agents is dexamathazone or cetirizine.

Embodiment 25 provides the isolated perfusate mixture of embodiments 1-24, wherein the temperature of the mixture is approximately 28° C.

Embodiment 26 provides a system for the hypothermic preservation of organs in a mammal, the system comprising:

a perfusion device for the perfusion of an isolated perfusate mixture into the mammal, the perfusion device comprising:

a perfusion loop; and

a controller programmed to regulate at least a perfusate temperature within the perfusion loop to maintain hypothermic conditions; and the isolated perfusate mixture of any of embodiments 1-25.

Embodiment 27 provides the system of embodiment 26, wherein the perfusion loop further comprises at least one pulse generator programmed to generate a pressure pulse within the perfusate within the perfusion loop.

Embodiment 28 provides the system of embodiments 26-27, wherein the perfusion loop comprises a venous loop, a filtration loop and an arterial loop, wherein:

the venous loop comprises at least one perfusion pump;

the filtration loop comprises at least one perfusion pump, and at least one hemodiafiltration unit adapted and configured to equilibrate the perfusate;

the arterial loop comprises at least one gas exchange source and at least one gas mixer adapted and configured to supply oxygen and carbon dioxide to the perfusate;

wherein the mammal, the venous loop, the filtration loop and the arterial loop are in fluidic communication such that the perfusate can be carried from the mammal, through the venous loop, through the filtration loop, through the arterial loop and back to the mammal.

Embodiment 29 provides the system of embodiments 26-28, wherein one or more components selected from the group consisting of the venous loop, the filtration loop and the arterial loop further comprise a reservoir containing excess perfusate.

Embodiment 30 provides the system of embodiments 26-29, wherein one or more components selected from the group consisting of the mammal, the venous loop, the filtration loop and the arterial loop further comprise one or more elements selected from the group consisting of:
one or more valves adapted and configured to regulate the flow of the perfusate;
one or more filters adapted and configured to filter the perfusate; and
one or more sensors for measuring one or more properties of the perfusate selected from the group consisting of pH, dissolved oxygen concentration, dissolved carbon dioxide concentration, dissolved metabolite concentration, temperature, pressure, and flow rate.

Embodiment 31 provides the system of embodiments 26-30, wherein the one or more sensors measure the concentration of at least one dissolved metabolite selected from the group consisting of nitric oxide, lactate, bicarbonate, oxygen, carbon dioxide, total hemoglobin, methemoglobin, oxyhemoglobin, carboxyhemoglobin, sodium, potassium, chloride, calcium, glucose, urea, ammonia, and creatinine.

Embodiment 32 provides the system of embodiments 26-31, wherein the mammal perfusion apparatus comprises one or more sensors for measuring one or more properties of the perfusate selected from the group consisting of pressure and flow rate.

Embodiment 33 provides the system of embodiments 26-32, wherein one or more components selected from the group consisting of the mammal, the venous loop, the filtration loop and the arterial loop comprise one or more heat exchange units comprising:

one or more heat exchangers;

one or more temperature regulation units;

one or more temperature regulating pumps;

a thermoregulation fluid; and

one or more pipes configured and adapted to transport the thermoregulation fluid, wherein the one or more pipes are in fluidic communication with the one or more heat exchangers, the one or more temperature regulation units and the one or more temperature regulating pumps.

Embodiment 34 provides the system of embodiments 26-33, wherein the one or more components selected from the group consisting of the brain enclosure unit, the venous loop, the filtration loop and the arterial loop comprise one or more sensors adapted and configured to measure the temperature within the perfusion device.

Embodiment 35 provides the system of embodiments 26-34, wherein the one or more sensor(s) is/are adapted and configured to measure the temperature within the perfusion device, the one or more temperature regulation units and the one or more temperature regulating pumps are in electronic communication with a computer programmed to regulate the temperature of the thermoregulation fluid and the specified flow rate of the one or more temperature regulating pumps to maintain a specified temperature within the perfusion device.

Embodiment 36 provides the system of embodiments 26-35, wherein the hemodiafiltration unit is adapted and configured to supply one or more nutrients to the perfusate, selected from the group consisting of Glycine, L-Alanyl-Glutamine, L-Arginine hydrochloride, L-Cystine, L-Histidine hydrochloride-H2O, L-Isoleucine, L-Leucine, L-Lysine hydrochloride, L-Methionine, L-Phenylalanine, L-Serine, L-Threonine, L-Tryptophan, L-Tyrosine, L-Valine, Choline chloride, D-Calcium pantothenate, Folic Acid, Niacinamide, Pyridoxine hydrochloride, Riboflavin, Thiamine hydrochloride, i-Inositol, Calcium Chloride (CaCl₂)-2H2O), Ferric Nitrate (Fe(N03)3 9H2O), Magnesium Sulfate (MgSO4-7H2O), Potassium Chloride (KCl), Sodium Bicarbonate (NaHCO3), Sodium Chloride (NaCl), Sodium Phosphate monobasic (NaH2PO4-2H2O), D-Glucose (Dextrose), Phenol Red, Sodium Pyruvate, free fatty acids, cholesterol and nucleic acid constitutes.

Embodiment 37 provides the system of embodiments 26-36, wherein the system is configured to perfuse the mammal with the perfusate at a cardiac pulsatile pressure of about 20 mmHg to about 140 mmHg.

Embodiment 38 provides the system of embodiments 26-37, wherein the system is configured to perfuse the organs in the mammal with the perfusate through the pulse generator at a rate of about 40 to about 180 beats per minute.

Embodiment 39 provides the system of embodiments 26-38, further comprising a controller in electronic communication with one or more elements of the system.

Embodiment 40 provides a mammal perfused with the isolated perfusate composition of any of embodiments 1-25, wherein mammalian organs are perfused under hypothermic conditions.

Embodiment 41 provides the mammal of embodiment 40, wherein the mammal is a deceased mammal.

Embodiment 42 provides the mammal of embodiments 40-41, wherein the mammal is a human.

Embodiment 43 provides the mammal of embodiments 40-42, wherein the deceased mammal is deceased for longer than 1 hour.

Embodiment 44 provides the mammal of embodiments 40-45, wherein the deceased mammal has been deceased for longer than 4 hours.

Embodiment 45 provides the mammal of embodiments 40-45, wherein the mammal died of cardiac arrest.

Embodiment 46 provides the mammal of embodiments 40-45, wherein the organs in the deceased mammal are ischemic prior to perfusion with the isolated perfusate mixture of any of claims 1-25.

Embodiment 47 provides the mammal of embodiments 40-46, wherein rigor mortis is prevented.

Embodiment 48 provides the mammal of embodiments 40-47, wherein rigor mortis is reversed.

Embodiment 49 provides the mammal of embodiments 40-48, wherein the perfusate mixture flows into the ophthalmic artery.

Embodiment 50 provides the mammal of embodiments 40-49, wherein the perfusate mixture flows into the renal intralobular arteries.

Embodiment 51 provides perfused organs in a diseased mammal, wherein the perfused organs maintain one or more properties selected from the group consisting of an in vivo level of cell function and viability, and an in vivo level of morphology.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An isolated perfusate mixture comprising:
an inorganic salt solution;
an artificial oxygen carrier; and
autologous blood.

2. The isolated perfusate mixture of claim 1, wherein the one or more artificial oxygen carrier(s) is/are selected from the group consisting of hemoglobin glutamer-250, isolated cell-free hemoglobin, cross-linked hemoglobin, polymerized hemoglobin, encapsulated hemoglobin, and perfluorocarbon oxygen carriers.

3. The isolated perfusate mixture of claim 2, wherein the artificial oxygen carrier is hemoglobin glutamer-250.

4. The isolated perfusate mixture of claim 1, wherein the one or more inorganic salts are selected from the group consisting of sodium chloride, sodium bicarbonate, magnesium chloride, and calcium chloride.

5. The isolated perfusate mixture of claim 1, wherein the perfusate comprises a priming solution containing one or more sugars.

6. The isolated perfusate mixture of claim 5, wherein the one or more sugars are glucose or dextrane.

7. The isolated perfusate mixture of claim 1, further comprising one or more amino acids.

8. The isolated perfusate mixture of claim 7, wherein the one or more amino acids are selected from the group consisting of glycine, L-alanyl-glutamine, L-arginine, L-cysteine, L-histidine, L-isoleucine, L-leucine, L-lysine, L-methionine, L-phenylalanine, L-serine, L-threonine, L-tryptophan, L-tyrosine, L-valine and salts and solvates thereof.

9. The isolated perfusate mixture of claim 1, further comprising one or more vitamins.

10. The isolated perfusate mixture of claim 9, wherein the one or more vitamin(s) is/are selected from the group consisting of choline, D-calcium pantothenate, folic acid, niacinamide, pyridoxine, riboflavin, thiamine, i-inositol and salts and solvates thereof.

11. The isolated perfusate mixture of claim 1, further comprising, ferric nitrate, magnesium sulfate, potassium chloride, sodium phosphate, and derivatives thereof.

12. The isolated perfusate mixture of claim 1, further comprising an anti-clotting agent.

13. The isolated perfusate mixture of claim 12, wherein the anti-clotting agent is heparin.

14. The isolated perfusate mixture of claim 1 wherein the percentage of autologous blood in the mixture is between about 10% and about 50%.

15. The isolated perfusate mixture of claim 14, wherein the percentage of autologous blood in the mixture is approximately 28%.

16. The isolated perfusate mixture of any of claims 1-15, wherein the mixture is dialyzed against a solution comprising inorganic salts.

17. The isolated perfusate mixture of any of claims 1-15, wherein the mixture is dialyzed against plasma.

18. The isolated perfusate mixture of any of claims 1-15, wherein the mixture comprises electrolytes and oncotic agents at levels comparable to those in autologous blood.

19. The isolated perfusate mixture of claim 1, wherein the perfusate further comprises cytoprotective agents.

20. The isolated perfusate mixture of claim 19, wherein the cytoprotective agents are selected from the group consisting of 2-Iminobiotin, Necrostatin-1, sodium 3-hydroxybutryate, glutathione, minocycline, lamotrigine, QVE-Oph, methylene blue, and or any salts, solvates, tautomers, and prodrugs thereof.

21. The isolated perfusate mixture of claim 1, wherein the mixture further comprises antibiotics.

22. The isolated perfusate mixture of claim 21, wherein the antibiotic is ceftriazone.

23. The isolated perfusate mixture of claim 1, wherein the mixture comprises one or more anti-inflammatory agent(s).

24. The isolated perfusate mixture of claim 1, wherein the one or more the anti-inflammatory agent(s) is dexamathazone or cetirizine.

25. The perfusate mixture of any of claims 1-24, wherein the temperature of the mixture is approximately 28° C.

26. A system for the hypothermic preservation of organs in a mammal, the system comprising:
a perfusion device for the perfusion of an isolated perfusate mixture into the mammal, the perfusion device comprising:
a perfusion loop; and
a controller programmed to regulate at least a perfusate temperature within the perfusion loop to maintain hypothermic conditions; and the isolated perfusate mixture of any of claims 1-25.

27. The system of claim 26, wherein the perfusion loop further comprises at least one pulse generator programmed to generate a pressure pulse within the perfusate within the perfusion loop.

28. The system of claim 26, wherein the perfusion loop comprises a venous loop, a filtration loop and an arterial loop, wherein:
the venous loop comprises at least one perfusion pump;
the filtration loop comprises at least one perfusion pump, and at least one hemodiafiltration unit adapted and configured to equilibrate the perfusate;
the arterial loop comprises at least one gas exchange source and at least one gas mixer adapted and configured to supply oxygen and carbon dioxide to the perfusate;
wherein the mammal, the venous loop, the filtration loop and the arterial loop are in fluidic communication such that the perfusate can be carried from the mammal, through the venous loop, through the filtration loop, through the arterial loop and back to the mammal.

29. The system of claim 28, wherein one or more components selected from the group consisting of the venous loop, the filtration loop and the arterial loop further comprise a reservoir containing excess perfusate.

30. The system of claim 28, wherein one or more components selected from the group consisting of the mammal, the venous loop, the filtration loop and the arterial loop further comprise one or more elements selected from the group consisting of:
one or more valves adapted and configured to regulate the flow of the perfusate;
one or more filters adapted and configured to filter the perfusate; and
one or more sensors for measuring one or more properties of the perfusate selected from the group consisting of pH, dissolved oxygen concentration, dissolved carbon dioxide concentration, dissolved metabolite concentration, temperature, pressure, and flow rate.

31. The system of claim 28, wherein the one or more sensors measure the concentration of at least one dissolved metabolite selected from the group consisting of nitric oxide, lactate, bicarbonate, oxygen, carbon dioxide, total hemoglobin, methemoglobin, oxyhemoglobin, carboxyhemoglobin, sodium, potassium, chloride, calcium, glucose, urea, ammonia, and creatinine.

32. The system of claim 28, wherein the mammal perfusion apparatus comprises one or more sensors for measuring one or more properties of the perfusate selected from the group consisting of pressure and flow rate.

33. The system of claim 28, wherein one or more components selected from the group consisting of the mammal, the venous loop, the filtration loop and the arterial loop comprise one or more heat exchange units comprising:
one or more heat exchangers;
one or more temperature regulation units;
one or more temperature regulating pumps;
a thermoregulation fluid; and
one or more pipes configured and adapted to transport the thermoregulation fluid, wherein the one or more pipes are in fluidic communication with the one or more heat exchangers, the one or more temperature regulation units and the one or more temperature regulating pumps.

34. The system of claim 28, wherein the one or more components selected from the group consisting of the brain enclosure unit, the venous loop, the filtration loop and the arterial loop comprise one or more sensors adapted and configured to measure the temperature within the perfusion device.

35. The system of claim 28, wherein the one or more sensor(s) is/are adapted and configured to measure the temperature within the perfusion device, the one or more temperature regulation units and the one or more temperature regulating pumps are in electronic communication with a computer programmed to regulate the temperature of the thermoregulation fluid and the specified flow rate of the one or more temperature regulating pumps to maintain a specified temperature within the perfusion device.

36. The system of claim 28, wherein the hemodiafiltration unit is adapted and configured to supply one or more nutrients to the perfusate, selected from the group consisting of Glycine, L-Alanyl-Glutamine, L-Arginine hydrochloride, L-Cystine, L-Histidine hydrochloride-H2O, L-Isoleucine, L-Leucine, L-Lysine hydrochloride, L-Methionine, L-Phenylalanine, L-Serine, L-Threonine, L-Tryptophan, L-Tyrosine, L-Valine, Choline chloride, D-Calcium pantothenate, Folic Acid, Niacinamide, Pyridoxine hydrochloride, Riboflavin, Thiamine hydrochloride, i-Inositol, Calcium Chloride (CaCl₂)-2H2O), Ferric Nitrate (Fe(NO3)3 9H2O), Magnesium Sulfate (MgSO4-7H2O), Potassium Chloride (KCl), Sodium Bicarbonate (NaHCO₃), Sodium Chloride (NaCl), Sodium Phosphate monobasic (NaH2PO4-2H2O), D-Glucose (Dextrose), Phenol Red, Sodium Pyruvate, free fatty acids, cholesterol and nucleic acid constitutes.

37. The system of any of claims 26-36, wherein the system is configured to perfuse the mammal with the perfusate at a cardiac pulsatile pressure of about 20 mmHg to about 140 mmHg.

38. The system of any of claims 26-36, wherein the system is configured to perfuse the organs in the mammal with the perfusate through the pulse generator at a rate of about 40 to about 180 beats per minute.

39. The system of any of claim 26-36, further comprising a controller in electronic communication with one or more elements of the system.

40. A mammal perfused with the isolated perfusate mixture of any of claims 1-25, wherein mammalian organs are perfused under hypothermic conditions.

41. The mammal of claim 40, wherein the mammal is a deceased mammal.

42. The mammal of claim 40, wherein the mammal is a human.

43. The mammal of claim 41 wherein the deceased mammal is deceased for longer than 1 hour.

44. The deceased mammal of claim 43, wherein the deceased mammal has been deceased for longer than 4 hours.

45. The deceased animal of claim 41, wherein the mammal died of cardiac arrest.

46. The deceased mammal of claim 41, wherein the organs in the deceased mammal are ischemic prior to perfusion with the isolated perfusate mixture of any of claims 1-25.

47. The deceased mammal of claim 41, wherein rigor mortis is prevented.

48. The deceased mammal of claim 41, wherein rigor mortis is reversed.

49. The diseased mammal of claim 41, wherein the perfusate mixture flows into the ophthalmic artery.

50. The diseased mammal of claim 41, wherein the perfusate mixture flows into the renal intralobular arteries.

51. The perfused organs in a diseased mammal, wherein the perfused organs maintain one or more properties selected from the group consisting of an in vivo level of cell function and viability, and an in vivo level of morphology.
